(19) United States
(12) Patent Application Publication
(10) Pub. No.: US 2025/0179690 A1
(43) Pub. Date: Jun. 5, 2025

(54) Split interleukin mimetics and their use

(71) Applicant: University of Washington, Seattle, WA (US)

(72) Inventors: Alfredo QUIJANO RUBIO, Seattle, WA (US); Daniel Adriano SILVA MANZANO, Seattle, WA (US); David BAKER, Seattle, WA (US); Umut ULGE, Seattle, WA (US); Marc Joseph LAJOIE, Seattle, WA (US)

(21) Appl. No.: 19/016,343

(22) Filed: Jan. 10, 2025

Related U.S. Application Data
(63) Continuation of application No. 17/294,807, filed on May 18, 2021, now Pat. No. 12,234,572.
(60) Provisional application No. 62/770,152, filed on Nov. 20, 2018.

(51) Int. Cl. C40B 40/10; A61K 38/20; A61K 48/00; A61P 35/00; A61P 37/04; C07K 14/54; C07K 14/55

(52) U.S. Cl. CPC C40B 40/10; A61K 38/2013; A61K 38/2026; A61K 38/2086; A61P 35/00; A61P 37/04; C07K 14/5406; C07K 14/5443; C07K 14/55; A61K 48/00; C07K 2319/30

(57) ABSTRACT

Conditionally active receptor agonists that, when activated, bind to IL-2 receptor β [symbol] _{c} heterodimer (IL-2Rβ [symbol] _{c}), IL-4 receptor α [symbol] _{c}heterodimer (IL-4Rα [symbol] _{c}), or IL-13 receptor α subunit (IL-13Rα) are disclosed, as are components of the conditionally active receptor agonists and methods for using the conditionally active receptor agonists.

### SEQUENCE LISTING STATEMENT

A computer readable form of the Sequence Listing is filed with this application by electronic submission and is incorporated into this application by reference in its entirety. The Sequence Listing is contained in the file created on Nov. 4, 2024 having the file name “18-1641-WO-US-CON.xml” and is 637,842 bytes in size.

### BACKGROUND

The considerable potential of central immune cytokine interleukins such as IL-2 and IL-4 for cancer treatment has sparked numerous efforts to improve their therapeutic properties by mutation and/or chemical modification. However, because these approaches are closely tied to native IL-2 or IL-4, they cannot eliminate undesirable properties such as low stability and binding to the IL-2 receptor α subunit (IL-2Rα), to IL-4 receptor α [symbol] _{c}heterodimer (IL-4Rα [symbol] _{c}), or to IL-13 receptor α subunit (IL-13Rα).

### SUMMARY

In one aspect the disclosure provides non-naturally occurring conditionally active receptor agonists, comprising a first polypeptide component and a second polypeptide component, wherein the first polypeptide component and the second polypeptide component are not present in a fusion protein, wherein in total the first polypeptide component and the second polypeptide component comprise domains X1, X2, X3, and X4, wherein:
(a) X1 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (PKKKIQ)LHAEHALYDA(MILNI) (SEQ ID NO: 4);
(b) X2 is any helical peptide domain at least 8 amino acids in length;
(c) X3 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (LE)DYAFNFELI(LEEIARLFESG) (SEQ ID NO:5); and
(d) X4 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (EDEQEEMANAI)ITILQSWIF(S) (SEQ ID NO:6).
wherein:
   (i) amino acid residues in parentheses may be present or absent;
   (ii) the first polypeptide component comprises at least one of X1, X2, X3, and X4 but does not comprise each of X1, X2, X3, and X4; and
   (iii) the second polypeptide component comprises each of X1, X2, X3, and X4 that is not present in the first polypeptide component;
wherein the first polypeptide component and the second polypeptide component are not active receptor agonists individually, and wherein the first polypeptide component and the second polypeptide interact to form an active agonist of IL-2 receptor β [symbol] _{c} heterodimer (IL-2Rβ [symbol] _{c}), IL-4 receptor α [symbol] _{c}heterodimer (IL-4Rα [symbol] _{c}), IL-13 alpha, or IL-4Ralpha/IL13Ralpha heterodimer. Numerous embodiments of the first and second polypeptides are provided herein. In exemplary embodiments,
(i) the first polypeptide component includes one of X1, X2, X3, and X4, and the second polypeptide component includes the three of X1, X2, X3, and X4 that are not present in the first polypeptide component; or
(ii) the first polypeptide component includes two of X1, X2, X3, and X4, and the second polypeptide component includes the two of X1, X2, X3, and X4 that are not present in the first polypeptide component. In other exemplary embodiments,
(i) the first polypeptide comprises X1 and the second polypeptide comprises X2, X3, and X4;
(ii) the first polypeptide comprises X2 and the second polypeptide comprises X1, X3, and X4;
(iii) the first polypeptide comprises X3 and the second polypeptide comprises X1, X2, and X4;
(iv) the first polypeptide comprises X4 and the second polypeptide comprises X1, X2, and X3;
(v) the first polypeptide comprises X1 and X2, and the second polypeptide comprises X3 and X4;
(vi) the first polypeptide comprises X1 and X3, and the second polypeptide comprises X2 and X4;
(vii) the first polypeptide comprises X1 and X4, and the second polypeptide comprises X2 and X3;
(viii) the first polypeptide comprises X2 and X3, and the second polypeptide comprises X1 and X4;
(ix) the first polypeptide comprises X2 and X4, and the second polypeptide comprises X1 and X3;
(x) the first polypeptide comprises X3 and X4, and the second polypeptide comprises X1 and X2;
(xi) the first polypeptide comprises X1, X2, and X3 and the second polypeptide comprises X4;
(xii) the first polypeptide comprises X1, X2, and X4 and the second polypeptide comprises X3;
(xiii) the first polypeptide comprises X1, X3, and X4 and the second polypeptide comprises X2; or
(xiv) the first polypeptide comprises X2, X3, and X4 and the second polypeptide comprises X1.

In other exemplary embodiments, the first polypeptide component and the second polypeptide component may be non-covalently associated, and/or the first polypeptide component and the second polypeptide component may be indirectly bound to each other through a receptor. In a further exemplary embodiment, the first polypeptide component further comprises a first targeting domain and/or the second polypeptide component further comprises a second targeting domain; in some embodiments, the first targeting domain, when present, is a translational fusion with the first polypeptide, and wherein the second targeting domain, when present, is a translational fusion with the second polypeptide. In some embodiments, the targeting domains may bind to a cell surface protein.

In another aspect, the disclosure provides polypeptides comprising 1, 2, or 3, but not all 4 domains X1, X2, X3, and X4, wherein:
(a) X1, when present, is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (PKKKIQ)LHAEHALYDAL(MILNI); (SEQ ID NO: 4);
(b) X2, when present, is any helical peptide domain;
(c) X3, when present, is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (LE)DYAFNFELILEE(IARLFESG) (SEQ ID NO:5); and
(d) X4, when present, is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (EDEQEEMANAI)ITILQSWIF(S) (SEQ ID NO:6);
amino acid residues in parentheses may be present or absent. Numerous embodiments of the polypeptides are provided. In some exemplary embodiments, the polypeptide comprises:
(i) a polypeptide comprising X1 and excluding X2, X3, and X4;
(ii) a polypeptide comprising X2 and excluding X1, X3, and X4;
(iii) a polypeptide comprising X3 and excluding X1, X2, and X4;
(iv) a polypeptide comprising X4 and excluding X1, X2, and X3;
(v) a polypeptide comprising X1 and X2, and excluding X3 and X4;
(vi) a polypeptide comprising X1 and X3, and excluding X2 and X4;
(vii) a polypeptide comprising X1 and X4, and excluding X2 and X3;
(viii) a polypeptide comprising X2 and X3, and excluding X1 and X4;
(ix) a polypeptide comprising X2 and X4, and excluding X1 and X3;
(x) a polypeptide comprising X3 and X4, and excluding X1 and X2;
(xi) a polypeptide comprising X1, X2, and X3 and excluding X4;
(xii) a polypeptide comprising X1, X2, and X4 and excluding X3;
(xiii) a polypeptide comprising X1, X3, and X4 and excluding X2; and
(xiv) a polypeptide comprising X2, X3, and X4 and excluding XL.

In another exemplary embodiment, the polypeptide further comprises a targeting domain, including but not limited to the targeting domain being a translational fusion with the polypeptide. In some embodiments, the targeting domains may bind to a cell surface protein.

In other aspects, the disclosure provides nucleic acids encoding the polypeptide, first polypeptide, or second polypeptide of any embodiment disclosed; expression vectors comprising the nucleic acids operatively linked to a promoter; host cells comprising the nucleic acids and/or expression vectors disclosed herein, and pharmaceutical composition, comprising the conditionally active receptor agonist, polypeptide, nucleic acid, expression vector, or host cell of any embodiment disclosed, and a pharmaceutically acceptable carrier.

The disclosure also provides methods for treating cancer, comprising administering to a subject in need thereof the conditionally active receptor agonist of any embodiment disclosed herein, under conditions wherein the first polypeptide component and the second polypeptide component interact at cells of the tumor to treat the cancer.

In another aspect, the disclosure provides the conditionally active receptor agonist, polypeptide, nucleic acid, expression vector, host cell, or pharmaceutical composition of any embodiment disclosed for use as a medicament for treating cancer and/or for modulating an immune response in a subject.

In a further aspect, the disclosure provides methods for agonizing the IL-2 receptor or the IL-4 receptor, comprising administering to a subject the conditionally active receptor agonist of any embodiment disclosed herein, under conditions wherein the first polypeptide component and the second polypeptide component interact at the receptor.

### DESCRIPTION OF THE DRAWINGS

The following figures are in accordance with example embodiments:

FIG. 1A-1C. Computational design of de novo cytokine mimetics. FIG. 1A) The designed non-split mimetics have four helices; three mimetic IL-2 interactions with hIL-2Rβ [symbol] _{c}, while the fourth holds the first three in place. Top: in the first generation of designs, each of the core elements of IL-2 (helices H1-H4) were independently idealized using fragment-assembly from a clustered ideal fragment database (size: 4 a.a.); bottom: in the second generation of designs the core elements were instead built using parametric equations that recapitulate the shape of each disembodied helix, allowing changes in the length of each helix by +/−8 a.a.; FIG. 1B) Pairs of helices were reconnected using ideal loop fragments (size: 4 a.a. or 7 a.a., for gen-1 and gen-2 respectively, see Methods), representative examples are shown with newly built elements connecting each pair of helices; FIG. 1C) The helix hairpins generated in FIG. 1B were assembled in all possible combinations to generate fully connected protein backbones.

FIG. 2C. Characterization of neoleukin-2/15. Binding experiments (Biolayer Interferometry) show that neoleukin-2/15 can be incubated for 2 hours at 80° C. without any noticeable loss of binding, whereas human and murine IL-2 quickly lose activity.

FIG. 3A-3C. Reengineering of neoleukin-2/15 into a human interleukin-4 (hIL-4) mimetic (neoleukin-4). FIG. 3A) Neo-2/15 structurally aligned into the structure of IL-4 in complex with IL-4Rα and [symbol] _{c} (from PDB code 3BPL). Fourteen IL-4 residues that contact IL-4Ra and that were grafted into Neo-2/15 are labeled. FIG. 3B) Neoleukin-4 (Neo-4), a new protein with sixteen amino acid mutations compared to Neo-2/15. These mutations are labeled; thirteen of these were derived from the IL-4 residues depicted in panel “a)” that mediate contact with IL-4Rα, and three of them (H8M, K68I and I98F, underlined in the figure) were introduced by directed evolution using random mutagenesis and screening for high binding affinity variants. FIG. 3C) Biolayer interferometry data showing that Neo-4, like IL-4, binds to IL-4Rα alone, has no affinity for [symbol] _{c} alone, but binds to [symbol] _{c} when IL-4Rα is present in solution.

FIG. 4A-4D. Overall sequence conservation in binding residues for each of the four common helices, combining information from three different de novo-designed IL-2 mimics. Sequence logos were generated using combined data from binding experiments (using the heterodimeric mouse IL-2Rβ [symbol] c) from three independent SSM mutagenesis libraries for G2_neo2_40_1F_seq27, G2_neo2_40_1F_seq29 and G2_neo2_40_1F_seq36 (FIGS. 11-13). All of these proteins are functional high-affinity de novo mimetics of mouse and human IL-2, some having topologies that differ from that of Neo-2/15, but all containing the four Helices H1 (FIG. 4A; Neo-2/15 1-22 is SEQ ID NO:04, IL-2 6-27 is SEQ ID NO:248, IL-15 1-15 is SEQ ID NO:249), H3 (FIG. 4B; Neo-2/15 34-55 is SEQ ID NO:05, IL-2 82-103 is SEQ ID NO:250, IL-15 59-80 is SEQ ID NO:251), H2′ (FIG. 4C; Neo-2/15 58-76 is SEQ ID NO:07, IL-2 50-68 is SEQ ID NO:252, IL-15 34-52 is SEQ ID NO:253) and H4 (FIG. 4D; Neo-2/15 80-100 is SEQ ID NO:06, IL-2 111-131 is SEQ ID NO:254, IL-15 93-113 is SEQ ID NO:255). The logos show the combined information for each helix independently. Below each logo, a line graph shows the probability score (higher means more conserved) for each amino acid in the Neo-2/15 sequence. The solid horizontal line highlights positions where the Neo-2/15 amino acid has a probability score ≥30% (that is, these amino acids contribute more generally to receptor binding as they are globally enriched in the binding populations across all of the de novo IL-2 mimics tested). The topology of each helix in Neo-2/15 is shown left of each logo. The sequences of the Neo-2/15 helices and those of the corresponding helices (structurally aligned) in human IL-2 and IL-15 are shown below the graphs, highlighting the distinctiveness of the Neo-2/15 helices and binding interfaces.

FIG. 5A-5D. Experimental optimization of G1_neo2_40. FIG. 5A-5C) Heatmaps for G1_neo2_40 single-site mutagenesis library showing enrichment at specific positions after consecutive rounds of increasing selection with FIG. 5A) 50 nM, FIG. 5B) 2 nM, and FIG. 5C) 0.1 nM IL-2Rβ [symbol] _{c} heterodimer. Based on these enrichment data, a combinatorial library was designed with nucleotide diversity 1.5×10⁶. FIG. 5D) Amino acid residues available in the initial combinatorial library are depicted indicating residues predicted to be advantageous (shown above the original sequence) and deleterious (shown below the original sequence; in the depiction of the original sequence, black indicates residues that are represented in the combinatorial library and gray, residues not represented in the combinatorial library.

FIG. 6A-6E. Experimental optimization of G2_neo2_40_1F_seq27. Heatmaps for G2_neo2_40_1F_seq27 single-site mutagenesis library showing enrichment at specific positions after consecutive rounds of increasing selection with FIG. 6A) 10 nM, FIG. 6B) 1 nM, FIG. 6C) 0.1 nM, and FIG. 6D) 0.1 nM IL-2Rβ [symbol] _{c} heterodimer. Based on these enrichment data, a combinatorial library was designed with nucleotide diversity 5.3×10⁶. FIG. 6E) Amino acid residues available in the initial combinatorial library are depicted indicating residues predicted to be advantageous; black indicates residues in the starting sequence represented in the combinatorial library.

FIG. 7A-7E. Experimental optimization of G2_neo2_40_1F_seq29. Heatmaps for G2_neo2_40_1F_seq29 single-site mutagenesis library showing enrichment at specific positions after consecutive rounds of increasing selection with FIG. 7A) 10 nM, FIG. 7B) 1 nM, FIG. 7C) 0.1 nM, and FIG. 7D) 0.1 nM IL-2Rβ [symbol] _{c} heterodimer. Based on these enrichment data, a combinatorial library was designed with nucleotide diversity 2.9×10⁶. FIG. 7E) Amino acid residues available in the initial combinatorial library are depicted indicating residues predicted to be advantageous; black indicates residues in the starting sequence represented in the combinatorial library.

FIG. 8A-8E. Experimental optimization of G2_neo2_40_1F_seq36. Heatmaps for G2_neo2_40_1F_seq36 single-site mutagenesis library showing enrichment at specific positions after consecutive rounds of increasing selection with FIG. 8A) 10 nM, FIG. 8B) 1 nM, FIG. 8C) 0.1 nM, and FIG. 8D) 0.1 nM IL-2Rβ [symbol] _{c} heterodimer. Based on these enrichment data, a combinatorial library was designed with nucleotide diversity 2.7×10⁶. FIG. 8E) Amino acid residues available in the initial combinatorial library are depicted indicating residues predicted to be advantageous; black indicates residues in the starting sequence represented in the combinatorial library.

FIG. 9A-9B. Circular Dichroism (CD) thermal denaturation experiments for multiple IL-2/IL-15 de novo designed mimetics, generation-1. FIG. 9A) Thermal denaturation curves and FIG. 9B) wavelength scans.

FIG. 10A-10B. Circular Dichroism (CD) thermal denaturation experiments for multiple IL-2/IL-15 de novo designed mimetics, generation-1 experimentally optimized. FIG. 10A) Thermal denaturation curves and FIG. 10B) wavelength scans.

FIG. 11A-11D. Circular dichroism thermal melts for IL-2/IL-15 mimetic designs generation-2. FIG. 11A and FIG. 11C) Thermal denaturation curves and FIG. 11B and FIG. 11D) wavelength scans.

FIG. 12A-12C. Expression, purification, and thermal denaturation characterization of neoleukin-2/15. FIG. 12A) SDS Tris-Tricine gel electrophoresis showing expression and purification over affinity column. FIG. 12B) Circular dichroism at 222 nm during thermal melting from 25° C. to 95° C., showing robust temperature stability. FIG. 12C) Circular dichroism wavelength scans at 25° C., 95° C. and then again 25° C., showing that neoleukin-2/15 does not fully melt at 95° C. and refolds fully after cooling back to 25° C.

FIG. 13A-13B. Robustness of neoleukin-2/15 to single-point cysteine mutants on non-binding interface positions. FIG. 13A) Schematic showing point mutant positions in neolukin-2/15 that can individually be mutated to cysteine without interfering with expression of the protein or binding to IL-2Rβγc. Positions were chosen to avoid interference with receptor binding. FIG. 13B) Association kinetics of Neolukin-2/15 cysteine mutants with IL-2Rβγ_{c} measured using biolayer interferometry. All of the variants associate with receptor approximately similarly to Neo-2/15.

FIG. 14A-14C. Expression, purification, and thermal denaturation characterization of neoleukin-4. FIG. 14A) SDS Tris-Tricine gel electrophoresis showing expression and purification over affinity column. FIG. 14B) Circular dichroism at 222 nm during thermal melting from 25° C. to 95° C., showing robust temperature stability. FIG. 14C) Circular dichroism wavelength scans at 25° C., 95° C. and then again 25° C., showing that neoleukin-4 does not fully melt at 95° C. and refolds fully after cooling back to 25° C.

FIG. 15A-C. Split-Neo-2/15 variants can reconstitute Neoleukin-2/15 activity by binding to the human IL-2 receptor. a. Bio-Layer Interferometry binding assay of Neo-2/15 split between helices H1+H3-H2′-H4 (Neo2A1 and Neo2B1 respectively) to human IL-2 Receptor. Binding data were collected in an Octet RED96 (ForteBio) and processed using ForteBio™ Data Analysis Software version 9.0.0.10. Biotinylated target receptor human γc was immobilized on streptavidin-coated biosensors (SA ForteBio) at 1 μg/ml in binding buffer (10 mM HEPES (pH 7.4), 150 mM NaCl, 3 mM EDTA, 0.05% surfactant P20, 0.5% non-fat dry milk) for 300 seconds. After loading the γc target receptor onto the biosensor, baseline measurement was performed dipping the biosensors in binding buffer alone, then, the binding kinetics were monitored by dipping the biosensors in wells containing the target analyte protein (association step) and then dipping the sensors back into baseline/buffer (dissociation). For the association step, analyte proteins (i.e. Neoleukin-2/15, Neo2A1, Neo2B1, and an equimolar ratio of Neo2A1+Neo2B1) were diluted from concentrated stocks into binding buffer to a final concentration of 100 nM. Human IL-2Rβ was also added in solution at saturating concentration (250 nM). b. Bio-Layer Interferometry binding assay of Neo-2/15 split into helixes H1-H3+H2′-H4 (Neo2A2 and Neo2B2 respectively), following the aforementioned experimental protocol. c. Bio-Layer Interferometry binding assay of Neo-2/15 split into helixes H1-H3-H2′+H4 (Neo2A3 and Neo2B3 respectively), following the aforementioned experimental protocol.

FIG. 16A-C. Split-Neo-2/15 variants stimulate cell signaling by STAT5 phosphorylation in murine CTLL-2 cell lines. a. CTLL-2 cells were starved in cytokine-free media (RPMI, 10% FBS, +1 mM sodium pyruvate, +2 mM L-glutamine, 1% P/S) for 2 hours before the assay. Cells were plated in a 96-well plate and re-suspended in RPMI medium containing serial dilutions of protein samples: Neoleukin-2/15, Neo2A1, Neo2B1, and Neo2A1+Neo2B1 at equimolar ratios. Cells were stimulated for 15 min at 37° C. and immediately fixed by addition of formaldehyde to 1.5% and 10 min incubation at room temperature. Cells were permeabilized by resuspension in cold 100% methanol for 30 min at 4° C. Fixed and permeabilized cells were washed twice with FACS buffer (PBS pH 7.2 containing 0.1% bovine serum albumin) and incubated with Alexa™ Fluor 647-conjugated anti-STAT5 pY694 (BD Biosciences) diluted 1:50 in FACS buffer for 2 h at room temperature. Cells were then washed twice in FACS buffer and mean fluorescence intensity (MFI) was determined on a Guava easyCyte™ flow cytometer (Millipore). Dose-response curves were fitted to a logistic model and half-maximal effective concentration (EC50 values) were calculated using GraphPad Prism data analysis software after subtraction of the MFI of unstimulated cells and normalization to the maximum signal intensity. b. Bio-Layer Interferometry binding assay of Neo-2/15 split into helixes H1-H3+H2′-H4 (Neo2A3 and Neo2B3 respectively), following the aforementioned experimental protocol. c. Bio-Layer Interferometry binding assay of Neo-2/15 split into helixes H1-H3-H2′+H4 (Neo2A2 and Neo2B2 respectively), following the aforementioned experimental protocol.

FIG. 17A-B. Fusions of split-Neo2/15 variants to targeting domains bind to the IL-2Receptor. a. Neo2A and Neo2B split protein variants fused to anti-EGFR and anti-Her2 DARPin targeting domains. b. Bio-Layer Interferometry binding assay of split-Neo-2/15 protein fusions to mouse IL-2 Receptor. Binding data were collected in an Octet RED96 (ForteBio) and processed using ForteBio™ Data Analysis Software version 9.0.0.10. Biotinylated target receptor human γc was immobilized on streptavidin-coated biosensors (SA ForteBio) at 1 μg/ml in binding buffer (10 mM HEPES (pH 7.4), 150 mM NaCl, 3 mM EDTA, 0.05% surfactant P20, 0.5% non-fat dry milk) for 300 seconds. After loading the target receptor onto the biosensor, baseline measurement was performed dipping the biosensors in binding buffer alone, then, the binding kinetics were monitored by dipping the biosensors in wells containing the target protein (association step) and then dipping the sensors back into baseline/buffer (dissociation). For the association step, analyte proteins (i.e. aHer2Neo2A1+Neo2B1-aEGFR, aHer2Neo2A2+Neo2B2-aEGFR, aHer2Neo2A3+Neo2B3-aEGFR) were diluted in equimolar amounts from concentrated stocks into binding buffer to a final concentration of 100 nM. Mouse IL-2Rβ was also added in solution at saturating concentration (250 nM).

FIG. 18A-C. Targeted reconstitution of split-Neoleukin-2/15 on the surface of K562 cells expressing Her2 and EGFR. a. Neo2A and Neo2B split protein variants fused to anti-EGFR and anti-Her2 DARPin targeting domains. b. Engineered K562 tumor cell lines transduced for expression of Her2+/eGFP+, EGFR+/iRFP+ or Her2+/eGFP+ and EGFR+/iRFP+surface markers. c. Functional reconstitution assay of Her2-targeted Neo2A variants (aHer2Neo2A1, aHer2Neo2A2 and aHer2Neo2A3) and EGFR-targeted Neo2B variants (Neo2B1-aEGFR, Neo2B2-aEGFR and Neo2B3-aEGFR) to the surface of the four aforementioned K562 cell lines. All cell lines were mixed in equivalent ratio (50,000 of each cell type per well) and transferred to a V-bottom plate at 200.000 cells/well. The cells were incubated for 30 minutes at room temperature with an equimolar ratio of anti-Her2-Neo2, anti-Her2-Neo2A+anti-EGFR-Neo2B variants, to a final concentration of 10 nM in FACS buffer (PBS (Gibco), 0.5% BSA). The cells were then washed twice (PBS (Gibco), 0.5% BSA) and subsequently incubated for 5 minutes with the following components: 50 nM of biotinylated soluble human common gamma receptor, 50 nM of soluble human IL-2Rβ and 15 nM of streptavidin-phycoerythrin conjugate (SAPE). Cells were washed again and analyzed by flow cytometry in a LSRII instrument. Her2+ cells were sorted for eGFP fluorescence (Ex/Em=488/508), EGFR+ cells were sorted for iRFP fluorescence (Ex/Em=637/670), Her2+/EGFR+ cells were identified by simultaneous expression of eGFP and iRFP. Reconstitution of Neoleukin-2/15 activity and binding to the IL-2 receptor was identified by analyzing PE (Ex/Em=561/582) fluorescence on the surface of each subset of cells.

FIG. 19A-C. Both fragments of the split-Neo-2/15 variants are necessary to enable functional reconstitution on the surface of K562 cells expressing Her2 and EGFR. a. Functional reconstitution assay of Her2-targeted Neo2A variants (aHer2-Neo2A1) and EGFR-targeted Neo2B variants (Neo2B1-aEGFR) to the surface of four K562 cell lines expressing the following markers: None, Her2+/eGFP+, EGFR+/iRFP+, Her2+/eGFP+/EGFR+/iRFP+. All cell lines were mixed in equivalent ratio (50,000 of each cell type per well) and transferred to a V-bottom plate at 200.000 cells/well. The cells were incubated for 30 minutes at room temperature with anti-Her2-Neo2A1, Neo2B1-antiEGFR or anti-Her2-Neo2A1+Neo2B1-antiEGFR to a final concentration of 810 nM in FACS buffer (PBS (Gibco), 0.5% BSA). The cells were then washed twice (PBS (Gibco), 0.5% BSA) and subsequently incubated for 5 minutes with the following components: 50 nM of biotinylated soluble human common gamma receptor, 50 nM of soluble human IL-2Rβ and 15 nM of streptavidin-phycoerythrin conjugate (SAPE). Cells were washed again and analyzed by flow cytometry in a LSRII instrument. Her2+ cells were sorted for eGFP fluorescence (Ex/Em=488/508), EGFR+ cells were sorted for iRFP fluorescence (Ex/Em=637/670), Her2+/EGFR+ cells were identified by simultaneous expression of eGFP and iRFP. Reconstitution of Neoleukin-2/15 activity and binding to the IL-2 receptor was identified by analyzing PE (Ex/Em=561/582) fluorescence on the surface of each subset of cells. As observed, both fragments are necessary to reconstitute functional Neoleukin-2/15 activity on the surface of double positive cells. b. Same assay described in (a) using aHer2-Neo2A2 and Neo2B2-aEGFR. c. Same assay described in (a) using aHer2-Neo2A3 and Neo2B3-aEGFR.

FIG. 20A-B. Illustration of alternative modes of action of the splitNeo-2 platform for highly-specific activation of immune cell subtypes. a. Selective targeting of splitNeo-2 to two surface markers to stimulate the expansion of a specific immune cell subtype (e.g. CD8+, CAR T cells or Regulatory T cells) b. Simultaneous tumor and T cell targeting by the split Neoleukin-2/15 system to induce immune cell subset specific proliferation in the environment of target tumor cells.

FIG. 21. Neoleukin-4 split fragments can reconstitute full activity when combined. Bio-Layer Interferometry binding assay of Neo-4 split into helices H1 (Neo4A1)+H3-H2′-H4 (Neo4B1) to human IL-4 Receptor. Binding data were collected in an Octet RED96 (ForteBio) and processed using ForteBio™ Data Analysis Software version 9.0.0.10. Biotinylated target receptor human γc was immobilized on streptavidin-coated biosensors (SA ForteBio) at 1 μg/ml in binding buffer (10 mM HEPES (pH 7.4), 150 mM NaCl, 3 mM EDTA, 0.05% surfactant P20, 0.5% non-fat dry milk) for 300 seconds. After loading the target receptor onto the biosensor, baseline measurement was performed in binding buffer alone, then, the binding kinetics were monitored by dipping the biosensors in wells containing the target protein at the indicated concentration (association step) and then dipping the sensors back into baseline/buffer (dissociation). For the association step, analyte proteins (i.e. Neoleukin-4, Neo4A1, Neo4B1 and an equimolar ratio of Neo4A1+Neo4B1) were diluted from concentrated stocks into binding buffer to a final concentration of 100 nM, human IL-4Rα was also added in solution at saturating concentration (250 nM).

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In a first aspect, the disclosure provides non-naturally occurring conditionally active receptor agonists, comprising a first polypeptide component and a second polypeptide component, wherein the first polypeptide component and the second polypeptide component are not present in a fusion protein, wherein in total the first polypeptide component and the second polypeptide component comprise domains X1, X2, X3, and X4, wherein:
(a) X1 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (PKKKIQ)LHAEHALYDAL(MILNI) (SEQ ID NO: 4);
(b) X2 is any helical peptide domain;
(c) X3 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (LE)DYAFNFELILEE(IARLFESG) (SEQ ID NO:5); and
(d) X4 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (EDEQEEMANAI)ITILQSWIF(S) (SEQ ID NO:6).
wherein:
   (i) amino acid residues in parentheses may be present or absent;
   (ii) the first polypeptide component comprises at least one of X1, X2, X3, and X4 but does not comprise each of X1, X2, X3, and X4; and
   (iii) the second polypeptide component comprises each of X1, X2, X3, and X4 that is not present in the first polypeptide component;
wherein the first polypeptide component and the second polypeptide component are not active receptor agonists individually, and wherein the first polypeptide component and the second polypeptide interact to form an active agonist of IL-2 receptor β [symbol] _{c} heterodimer (IL-2Rβ [symbol] _{c}), IL-4 receptor α [symbol] _{c}heterodimer (IL-4Rα [symbol] _{c}), IL-13 alpha, or IL-4Ralpha/IL13Ralpha heterodimer.

As shown in the examples that follow, as described in detail in PCT application serial no. PCT/US2019/038703 filed Jun. 29, 2019, and as described in Silva et al., Nature 565, pg. 186, Jan. 10, 2019, polypeptides that include all of X1-X4 were previously shown to be (a) mimetics of IL-2 and interleukin-15 (IL-15) that bind to the IL-2 receptor [symbol] _{c}V, heterodimer (IL-2Rβ [symbol] _{c}), but have no binding site for IL-2Rα or IL-15Rα, or (b) mimetics of IL-4 that bind to the IL-4 receptor α [symbol] _{c} heterodimer (IL-4Rα [symbol] _{c}) or IL-13 receptor α subunit (IL-13Rα) (natural IL-4 and the IL-4 mimetics described herein cross-react with IL-13 receptor, forming an IL-4Rα/IL13Rα heterodimer). The full length polypeptides were shown to be hyper-stable, bind to human and mouse IL-2Rβ [symbol] _{c} or IL-4Rα [symbol] _{c} with higher affinity than the natural cytokines, and elicit downstream cell signaling independent of IL-2Rα and IL-15Rα, or independent of IL-13Rα. The full length polypeptides can be used, for example, to treat cancer.

In contrast, the present disclosure surprisingly demonstrates conditionally active receptor agonists comprising the recited separate first and second polypeptides that individually are not receptor agonists, but which can interact non-covalently to form an active agonist of IL-2 receptor β [symbol] _{c} heterodimer (IL-2Rβ [symbol] _{c}), IL-4 receptor α [symbol] _{c}heterodimer (IL-4Rα [symbol] _{c}), IL-13 alpha, or IL-4Ralpha/IL13Ralpha heterodimer. The affinity of this non-covalent interaction between the “split components” (i.e.: the first polypeptide and the second polypeptide) is such that the interaction only occurs in the presence of the appropriate receptor, and also only when both split components are co-localized. Thus, the conditionally active receptor agonists of the current disclosure can be used for any uses that the polypeptides that include all of X1-X4 can be used for. Furthermore, the conditionally active receptor agonists enable co-localization-dependent reconstitution of the agonist, and thus, conditional-activation of the receptor.

The term protein mimetic as used herein refers to a protein that imitates certain aspects of the function of another protein. The two proteins typically have different amino acid sequence and/or different structures. Provided herein, among other things, conditionally active mimetics of IL-2 and IL-15. The aspects of the function of IL-2 and IL-15 that these conditionally active mimetics imitate is the induction of heterodimerization of IL-2Rβ [symbol] _{c}, leading to phosphorylation of STAT5. Because IL-2 and IL-15 both signal through heterodimerization of IL-2Rβ [symbol] _{c}, these conditionally active mimetics imitate this biological function of both IL-2 and IL-15. These conditionally active mimetics may be referred to herein as mimetics of IL-2, of IL-15, or of both IL-2 and IL-15.

Also provided are conditionally active mimetics of IL-4. These conditionally active mimetics are capable of imitating certain functions of IL-4. The function of IL-4 that these mimetics imitate is the induction of heterodimerization of IL-4Rα [symbol] _{c} (and/or heterodimerization of IL-4Rα/IL-13Rα).

In one embodiment, the first polypeptide component and the second polypeptide interact to form an agonist of the IL-2 receptor β [symbol] _{c} heterodimer (IL-2Rβ [symbol] _{c}). In another embodiment, the first polypeptide component and the second polypeptide interact to form an agonist of the IL-4 receptor α [symbol] _{c}heterodimer (IL-4Rα [symbol] _{c}), IL-13 alpha, or IL-4Ralpha/IL13Ralpha heterodimer.

Native hIL-2 comprises four helices connected by long irregular loops. The N-terminal helix (H1) interacts with both the beta and gamma subunits, the third helix (H3) interacts with the beta subunit, and the C-terminal helix (H4) with the gamma subunit; the alpha subunit interacting surface is formed by the irregular second helix (H2) and two long loops, one connecting H1 to H2 and the other connecting H3 and H4. Idealized proteins were designed and produced in which H1, H3 and H4 are replaced by idealized structural domains, including but not limited to helices and beta strands (referred to as domains X1, X3 and X4, respectively) displaying an IL-2Rβ [symbol] _{c} or IL-4Rα [symbol] _{c} interface inspired by H1, H3 and H4, and in which H2 is replaced with an idealized helix (referred to as domain X2) that offers better packing. As shown in the examples, extensive mutational studies have been carried out, demonstrating that the amino acid sequence of each peptide domain each can be extensively modified without loss of binding to the IL-2 or IL-4 receptor, and that the domains can be placed in any order while retaining conditional binding to the IL-2 or IL-4 receptor. The polypeptides may comprise L amino acids and glycine, D-amino acids and glycine, or combinations thereof. As described herein, the idealized proteins can be split into two polypeptides that separately have negligible binding to the relevant receptor but when mixed together can reconstitute receptor activity. The proteins are typically split at sites that won't interfere with the function of the protein (e.g., linker sections in embodiments with linkers). In addition, just as the X1, X2, X3, and X4 domains in the non-split proteins can be looped together in any order, the split proteins can comprise any combination of the domains.

Thus, X1, X2, X3, and X4 may be in any order in the first and second polypeptide; in non-limiting embodiments:
(i) the first polypeptide comprises X1 and the second polypeptide comprises X2, X3, and X4;
(ii) the first polypeptide comprises X2 and the second polypeptide comprises X1, X3, and X4;
(iii) the first polypeptide comprises X3 and the second polypeptide comprises X1, X2, and X4;
(iv) the first polypeptide comprises X4 and the second polypeptide comprises X1, X2, and X3;
(v) the first polypeptide comprises X1 and X2, and the second polypeptide comprises X3 and X4;
(vi) the first polypeptide comprises X1 and X3, and the second polypeptide comprises X2 and X4;
(vii) the first polypeptide comprises X1 and X4, and the second polypeptide comprises X2 and X3;
(viii) the first polypeptide comprises X2 and X3, and the second polypeptide comprises X1 and X4;
(ix) the first polypeptide comprises X2 and X4, and the second polypeptide comprises X1 and X3;
(x) the first polypeptide comprises X3 and X4, and the second polypeptide comprises X1 and X2;
(xi) the first polypeptide comprises X1, X2, and X3 and the second polypeptide comprises X4;
(xii) the first polypeptide comprises X1, X2, and X4 and the second polypeptide comprises X3;
(xiii) the first polypeptide comprises X1, X3, and X4 and the second polypeptide comprises X2; or
(xiv) the first polypeptide comprises X2, X3, and X4 and the second polypeptide comprises X1.

When the first polypeptide and/or the second polypeptide include more than one domain of X1, X2, X3, and X4, the domains may in some embodiments be separated by amino acid linkers of any suitable length or amino acid composition. There is no requirement for linkers; in one embodiment there are no linkers present between any of the domains. In other embodiments, an amino acid linker may be present between 0, 1, or 2 junctions between domains X1, X2, X3, and X4 in the first polypeptide and/or the second polypeptide. The amino acid linkers may be of any length as deemed appropriate for an intended use In some aspects, a linker is at the N terminus or C terminus and is referred to as a linker despite not linking two domains together. In some aspects, the linker is referred to as a linker as it was present in a non-split protein linking two domains together.

In all of these embodiments, X1, X3, and X4 may be any suitable length, meaning each domain may contain any suitable number of additional amino acids other than the peptides of SEQ ID NOS:4, 5, and 6, respectively. The residues in parentheses are optional, and thus may be present or absent. As will be understood by those of skill in the art, this means, for example, that the N-terminal 6 amino acids and the C-terminal 5 amino acid residues of (PKKKIQ)LHAEHALYDAL(MILNI) (SEQ ID NO: 4) are optional. As will be further understood by those of skill in the art: (i) if one N-terminal amino acid is absent, it will be the N-terminal most amino acid (i.e.: the N-terminal P residue in SEQ ID NO:4); (ii) if two N-terminal amino acids are absent, it will be the two N-terminal most amino acids (i.e.: the N-terminal PK dipeptide in SEQ ID NO:4); (iii) if one C-terminal amino acid residue is absent, it will be the C-terminal most amino acid (i.e.: the C-terminal I residue in SEQ ID NO:4); (iv) if two C-terminal amino acid residues are absent, it will be the two C-terminal most amino acids (i.e.: the C-terminal NI dipeptide in SEQ ID NO:4), etc. Thus, it will be clear to those of skill in the art that one or more optional amino acid residues may be absent, and that absent amino acids from the optional residues are contiguous from the relevant peptide terminus, as exemplified above.

In one embodiment, X1 comprises a peptide with identity to the full length of peptide ((PKKKIQ)LHAEHALYDAL(MILNI) (SEQ ID NO: 4); X3 comprises a peptide with identity to the full length of peptide (LE)DYAFNFELILEE(IARLFESG) (SEQ ID NO:5); and X4 comprises a peptide with identity to the full length of peptide to the full length of peptide (EDEQEEMANAI)ITILQSWIF(S) (SEQ ID NO:6), respectively, of at least 25%, 27%, 30%, 35%, 40%, 45%, 50%, 55%, 60%, 65%, 70%, 75%, 80%, 85%, 90%, 95%, 98%, or 100%.

In various embodiments, X1 is a peptide comprising an amino acid sequence with identity to the full length of peptide ((PKKKIQ)LHAEHALYDAL(MILNI) (SEQ ID NO: 4) or (PKKKI)QLHAEHALYDAL(MILNI (SEQ ID NO:4), X3 is a peptide comprising an amino acid sequence with identity to the full length of peptide (LE)DYAFNFELILEE(IARLFESG) (SEQ ID NO:5) or LEDYAFNFELILEEIARLFES(G) (SEQ ID NO:5); and X4 is a peptide comprising an amino acid sequence with identity to the full length of peptide to the full length of peptide (EDEQEEMANAI)ITILQSWIF(S) (SEQ ID NO:6) or (E)DEQEEMANAIITILQSWIFS (SEQ ID NO:6), respectively, of at least 25%, 27%, 30%, 35%, 40%, 45%, 50%, 55%, 60%, 65%, 70%, 75%, 80%, 85%, 90%, 95%, 98%, or 100%.

In specific embodiments;
(i) X1 is a peptide comprising an amino acid sequence with identity to the full length of peptide (PKKKIQ)LHAEHALYDAL(MILNI) (SEQ ID NO: 4) or (PKKKI)QLHAEHALYDAL(MILNI (SEQ ID NO:4) of at least 70%, X3 is a peptide comprising an amino acid sequence with identity to the full length of peptide (LE)DYAFNFELILEE(IARLFESG) (SEQ ID NO:5) or LEDYAFNFELILEEIARLFES(G) (SEQ ID NO:5) of at least 70%; and X4 is a peptide comprising an amino acid sequence with identity to the full length of peptide to the full length of peptide (EDEQEEMANAI)ITILQSWIF(S) (SEQ ID NO:6) or (E)DEQEEMANAIITILQSWIFS (SEQ ID NO:6) of at least 70%;
(ii) X1 is a peptide comprising an amino acid sequence with identity to the full length of peptide (PKKKIQ)LHAEHALYDAL(MILNI) (SEQ ID NO: 4) or (PKKKI)QLHAEHALYDAL(MILNI (SEQ ID NO:4) of at least 70%, X3 is a peptide comprising an amino acid sequence with identity to the full length of peptide (LE)DYAFNFELILEE(IARLFESG) (SEQ ID NO:5) or LEDYAFNFELILEEIARLFES(G) (SEQ ID NO:5) of at least 70%; and X4 is a peptide comprising an amino acid sequence with identity to the full length of peptide to the full length of peptide (EDEQEEMANAI)ITILQSWIF(S) (SEQ ID NO:6) or (E)DEQEEMANAIITILQSWIFS (SEQ ID NO:6) of at least 70%;
(iii) X1 is a peptide comprising an amino acid sequence with identity to the full length of peptide (PKKKIQ)LHAEHALYDAL(MILNI) (SEQ ID NO: 4) or (PKKKI)QLHAEHALYDAL(MILNI (SEQ ID NO:4) of at least 85%, X3 is a peptide comprising an amino acid sequence with identity to the full length of peptide (LE)DYAFNFELILEE(IARLFESG) (SEQ ID NO:5) or LEDYAFNFELILEEIARLFES(G) (SEQ ID NO:5) of at least 85%; and X4 is a peptide comprising an amino acid sequence with identity to the full length of peptide to the full length of peptide (EDEQEEMANAI)ITILQSWIF(S) (SEQ ID NO:6) or (E)DEQEEMANAIITILQSWIFS (SEQ ID NO:6) of at least 85%;
(iv) X1 is a peptide comprising an amino acid sequence with 100% identity to the full length of peptide (PKKKIQ)LHAEHALYDAL(MILNI) (SEQ ID NO: 4) or (PKKKI)QLHAEHALYDAL(MILNI (SEQ ID NO:4), X3 is a peptide comprising an amino acid sequence with 100% identity to the full length of peptide (LE)DYAFNFELILEE(IARLFESG) (SEQ ID NO:5) or LEDYAFNFELILEEIARLFES(G) (SEQ ID NO:5); and X4 is a peptide comprising an amino acid sequence with 100% identity to the full length of peptide to the full length of peptide (EDEQEEMANAI)ITILQSWIF(S) (SEQ ID NO:6) or (E)DEQEEMANAIITILQSWIFS (SEQ ID NO:6);
(v) X1 is a peptide comprising an amino acid sequence with identity to the full length of peptide (PKKKIQ)LHAEHALYDAL(MILNI) (SEQ ID NO: 4) of at least 85%, X3 is a peptide comprising an amino acid sequence with identity to the full length of peptide (LE)DYAFNFELILEE(IARLFESG) (SEQ ID NO:5) of at least 85%; and X4 is a peptide comprising an amino acid sequence with identity to the full length of peptide to the full length of peptide (EDEQEEMANAI)ITILQSWIF(S) (SEQ ID NO:6) of at least 85%; or
(vi) X1 is a peptide comprising an amino acid sequence with 100% identity to the full length of peptide (PKKKIQ)LHAEHALYDAL(MILNI) (SEQ ID NO: 4), X3 is a peptide comprising an amino acid sequence with 100% identity to the full length of peptide (LE)DYAFNFELILEE(IARLFESG) (SEQ ID NO:5); and X4 is a peptide comprising an amino acid sequence with 100% identity to the full length of peptide to the full length of peptide (EDEQEEMANAI)ITILQSWIF(S) (SEQ ID NO:6).

In these embodiments, different versions of SEQ ID NO: 4, 5, and 6 are shown that have the same primary amino acid sequence but differ in the position of optional residues as noted by the parentheses.

In further embodiments:
X1 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide QLHAEHALYDAL(MILNI (SEQ ID NO:320);
X3 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide LEDYAFNFELILEEIARLFES (SEQ ID NO:321); and/or X4 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide DEQEEMANAIITILQSWIF(S) (SEQ ID NO:322).

In another embodiment where the optional residues of SEQ ID NO:4, SEQ ID NO:5, and SEQ ID NO:6 are present,
(a) X1 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide PKKKIQLHAEHALYDAL(MILNI (SEQ ID NO: 4);
(c) X3 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide LEDYAFNFELILEEIARLFESG (SEQ ID NO:5); and
(d) X4 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide EDEQEEMANAIITILQSWIFS (SEQ ID NO:6).

In another embodiment where select optional residues of SEQ ID NO:4, SEQ ID NO:5, and SEQ ID NO:6 are present,
(a) X1 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide QLHAEHALYDAL(MILNI (SEQ ID NO:320);
(c) X3 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide LEDYAFNFELILEEIARLFES (SEQ ID NO:321); and
X4 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide or DEQEEMANAIITILQSWIF(S) (SEQ ID NO:322).

In various embodiments where the optional residues of SEQ ID NO:4, SEQ ID NO:5, and SEQ ID NO:6 are present, X1 is a peptide comprising an amino acid sequence with identity to the full length of peptide PKKKIQLHAEHALYDAL(MILNI (SEQ ID NO: 4), X3 is a peptide comprising an amino acid sequence with identity to the full length of peptide LEDYAFNFELILEEIARLFESG (SEQ ID NO:5) or LEDYAFNFELILEEIARLFES (SEQ ID NO:321); and X4 is a peptide comprising an amino acid sequence with identity to the full length of peptide to the full length of peptide EDEQEEMANAIITILQSWIF(S) (SEQ ID NO:6) or DEQEEMANAIITILQSWIF(S) (SEQ ID NO:322), respectively, of at least 25%, 27%, 30%, 35%, 40%, 45%, 50%, 55%, 60%, 65%, 70%, 75%, 80%, 85%, 90%, 95%, 98%, or 100%.

In specific embodiments;
(i) X1 is a peptide comprising an amino acid sequence with identity to the full length of peptide PKKKIQLHAEHALYDAL(MILNI (SEQ ID NO: 4) or QLHAEHALYDAL(MILNI (SEQ ID NO:320) of at least 65%; X3 is a peptide comprising an amino acid sequence with identity to the full length of peptide LEDYAFNFELILEEIARLFESG (SEQ ID NO:5) or LEDYAFNFELILEEIARLFES (SEQ ID NO:321) of at least 65%; and X4 is a peptide comprising an amino acid sequence with identity to the full length of peptide EDEQEEMANAIITILQSWIF(S) (SEQ ID NO:6) or DEQEEMANAIITILQSWIF(S) (SEQ ID NO:322), of at least 65%;
(ii) X1 is a peptide comprising an amino acid sequence with identity to the full length of peptide PKKKIQLHAEHALYDAL(MILNI (SEQ ID NO: 4) or QLHAEHALYDAL(MILNI (SEQ ID NO:320) of at least 75%; X3 is a peptide comprising an amino acid sequence with identity to the full length of peptide LEDYAFNFELILEEIARLFESG (SEQ ID NO:5) or LEDYAFNFELILEEIARLFES (SEQ ID NO:321) of at least 75%; and X4 is a peptide comprising an amino acid sequence with identity to the full length of peptide EDEQEEMANAIITILQSWIF(S) (SEQ ID NO:6) or DEQEEMANAIITILQSWIF(S) (SEQ ID NO:322), of at least 75%;
(iii) X1 is a peptide comprising an amino acid sequence with identity to the full length of peptide PKKKIQLHAEHALYDAL(MILNI (SEQ ID NO: 4) or QLHAEHALYDAL(MILNI (SEQ ID NO:320) of at least 80%; X3 is a peptide comprising an amino acid sequence with identity to the full length of peptide LEDYAFNFELILEEIARLFESG (SEQ ID NO:5) or LEDYAFNFELILEEIARLFES (SEQ ID NO:321) of at least 80%; and X4 is a peptide comprising an amino acid sequence with identity to the full length of peptide EDEQEEMANAIITILQSWIF(S) (SEQ ID NO:6) or DEQEEMANAIITILQSWIF(S) (SEQ ID NO:322), of at least 80%;
(iv) X1 is a peptide comprising an amino acid sequence with identity to the full length of peptide PKKKIQLHAEHALYDAL(MILNI (SEQ ID NO: 4) or QLHAEHALYDAL(MILNI (SEQ ID NO:320) of at least 90%; X3 is a peptide comprising an amino acid sequence with identity to the full length of peptide LEDYAFNFELILEEIARLFESG (SEQ ID NO:5) or LEDYAFNFELILEEIARLFES (SEQ ID NO:321) of at least 90%; and X4 is a peptide comprising an amino acid sequence with identity to the full length of peptide EDEQEEMANAIITILQSWIF(S) (SEQ ID NO:6) or DEQEEMANAIITILQSWIF(S) (SEQ ID NO:322), of at least 90%;
(v) X1 is a peptide comprising an amino acid sequence with identity to the full length of peptide PKKKIQLHAEHALYDAL(MILNI (SEQ ID NO: 4) or QLHAEHALYDAL(MILNI (SEQ ID NO:320) of 100%; X3 is a peptide comprising an amino acid sequence with identity to the full length of peptide LEDYAFNFELILEEIARLFESG (SEQ ID NO:5) or LEDYAFNFELILEEIARLFES (SEQ ID NO:321) of 100%; and X4 is a peptide comprising an amino acid sequence with identity to the full length of peptide EDEQEEMANAIITILQSWIF(S) (SEQ ID NO:6) or DEQEEMANAIITILQSWIFS (SEQ ID NO:322), of 100%.
(vi) X1 is a peptide comprising an amino acid sequence with identity to the full length of peptide PKKKIQLHAEHALYDAL(MILNI (SEQ ID NO: 4) of at least 80%; X3 is a peptide comprising an amino acid sequence with identity to the full length of peptide LEDYAFNFELILEEIARLFESG (SEQ ID NO:5) of at least 80%; and X4 is a peptide comprising an amino acid sequence with identity to the full length of peptide EDEQEEMANAIITILQSWIF(S) (SEQ ID NO:6) of at least 80%;
(vii) X1 is a peptide comprising an amino acid sequence with identity to the full length of peptide PKKKIQLHAEHALYDAL(MILNI (SEQ ID NO: 4) of at least 90%; X3 is a peptide comprising an amino acid sequence with identity to the full length of peptide LEDYAFNFELILEEIARLFESG (SEQ ID NO:5) of at least 90%; and X4 is a peptide comprising an amino acid sequence with identity to the full length of peptide EDEQEEMANAIITILQSWIF(S) (SEQ ID NO:6), of at least 90%;
(viii) X1 is a peptide comprising an amino acid sequence with identity to the full length of peptide PKKKIQLHAEHALYDAL(MILNI (SEQ ID NO: 4) of 100%; X3 is a peptide comprising an amino acid sequence with identity to the full length of peptide LEDYAFNFELILEEIARLFESG (SEQ ID NO:5) of 100%; and X4 is a peptide comprising an amino acid sequence with identity to the full length of peptide EDEQEEMANAIITILQSWIF(S) (SEQ ID NO:6) of 100%;
(ix) X1 is a peptide comprising an amino acid sequence with identity to the full length of peptide QLHAEHALYDAL(MILNI (SEQ ID NO:320) of at least 80%; X3 is a peptide comprising an amino acid sequence with identity to the full length of peptide LEDYAFNFELILEEIARLFES (SEQ ID NO:321) of at least 80%; and X4 is a peptide comprising an amino acid sequence with identity to the full length of peptide DEQEEMANAIITILQSWIF(S) (SEQ ID NO:322), of at least 80%;
(x) X1 is a peptide comprising an amino acid sequence with identity to the full length of peptide QLHAEHALYDAL(MILNI (SEQ ID NO:320) of at least 90%; X3 is a peptide comprising an amino acid sequence with identity to the full length of peptide LEDYAFNFELILEEIARLFES (SEQ ID NO:321) of at least 90%; and X4 is a peptide comprising an amino acid sequence with identity to the full length of peptide DEQEEMANAIITILQSWIF(S) (SEQ ID NO:322), of at least 90%; or
(xi) X1 is a peptide comprising an amino acid sequence with identity to the full length of peptide QLHAEHALYDAL(MILNI (SEQ ID NO:320) of 100%; X3 is a peptide comprising an amino acid sequence with identity to the full length of peptide LEDYAFNFELILEEIARLFES (SEQ ID NO:321) of 100%; and X4 is a peptide comprising an amino acid sequence with identity to the full length of peptide DEQEEMANAIITILQSWIFS (SEQ ID NO:322), of 100%.

In one embodiment, the conditionally active receptor agonists are conditionally active receptor agonists IL-2/15 mimetics and
(i) X1 includes 1, 2, 3, 4, or all 5 of the following: L at residue 7, H at residue 8, H at residue 11, Y at residue 14; M at residue 18 wherein the numbering is relative to SEQ ID NO:4 based on the optional residues being present; and/or
(ii) X3 includes 1, 2, 3, 4, 5, 6, 7, or all 8 of the following: D at residue 3, Y at residue 4, F at residue 6, N at residue 7, L at residue 10, I at residue 11, E at residue 13, or E at residue 14 wherein the numbering is relative to SEQ ID NO:5 based on the optional residues being present. In a further embodiment, (iii) X4 includes I at residue 19 wherein the numbering is relative to SEQ ID NO:6 based on the optional residues being present.

In SEQ ID NO:4, 5, and 6, a number of amino acid residues are marked in bold font. In (PKKKIQ)LHAEHALYDAL(MILNI) (SEQ ID NO: 4): amino acid residues E10, L13, Y14, D15, and L17 (numbered based on optional residues being present) are marked in bold font; In (LE)DYAFNFELILEE(IARLFESG) (SEQ ID NO:5): amino acid residues L1, Y4, N7, L10, I11, and I15 (numbered based on optional residues being present) are marked in bold font; and in (EDEQEEMANAI)ITILQSWIF(S) (SEQ ID NO:6) amino acid residues I12, Q16, and W18 (numbered based on optional residues being present) are marked in bold font.

In one embodiment:
(a) amino acid (AA) substitutions in X1 relative to the AA sequence of SEQ ID NO:4 occur at no more than 3 AA residues marked in bold font, or occur at no more than 2 AA residues marked in bold font, or occur at no more than 1 AA residue marked in bold font, or do not occur at AA residues marked in bold font;
(b) AA substitutions in X3 relative to the AA sequence of SEQ ID NO:5 occur at no more than 3 AA residues marked in bold font, or occur at no more than 2 AA residues marked in bold font, or occur at no more than 1 AA residue marked in bold font, or do not occur at AA residues marked in bold font; and/or
(c) AA substitutions in X4 relative to the AA sequence of SEQ ID NO:6 occur at no more than 2 AA residues marked in bold font, or occur at no more than 1 AA residue marked in bold font, or do not occur at AA residues marked in bold font.

In another embodiment, AA substitutions in X2 relative to the AA sequence of SEQ ID NO:7 do not occur at AA residues marked in bold font.

In another embodiment of conditionally active receptor agonists IL-2 mimetics, amino acid substitutions relative to the reference peptide domains (i.e.: SEQ ID NOS: 4, 5, or 6) do not occur at AA residues marked in bold font. As shown below, SEQ ID NOS:4, 5, and 6 each include residues in bold font that are involved in binding to the receptor:
(PKKKIQ)LHAEHALYDAL(MILNI) (SEQ ID NO: 4): amino acid residues E10, L13, Y14, D15, and L17 (numbered based on optional residues being present) are invariant in this embodiment;
(LE)DYAFNFELILEE(IARLFESG) (SEQ ID NO:5) amino acid residues L1, Y4, N7, L10, I11, and I15 (numbered based on optional residues being present) are invariant in this embodiment; and
(EDEQEEMANAI)ITILQSWIF(S) (SEQ ID NO:6) amino acid residues I12, Q16, and W18 (numbered based on optional residues being present) are invariant in this embodiment.

In a further embodiment, amino acid residue W13 is invariant when X2 comprises is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide KDEAEKAKRMKEWMKRIK(T) (SEQ ID NO:7), wherein residues in parentheses are optional. In one embodiment, the optional residue is present; in another embodiment the optional residue is absent.

In another embodiment of conditionally active receptor agonists IL-2 mimetics, amino acid substitutions relative to the reference peptide domains (i.e.: SEQ ID NOS: 4, 5, or 6) do not occur at more than 3, 2, or 1 AA residues marked in bold font.

In another embodiment, the conditionally active receptor agonists are conditionally active receptor agonists IL-4/IL-13 mimetics, and
X1 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQIMAEEALKDALSILNI (SEQ ID NO: 8);
X3 is a peptide comprising the amino acid sequence at least 37% 40%, 45%, 50%, 55%, 60%, 65%, 70%, 75%, 80%, 85%, 90%, 95%, 98%, or 100% identical along its length the peptide LERFAKRFERNLWGIARLFESG (SEQ ID NO: 9); and
X4 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide EDEQEEMANAIITILQSWFFS (SEQ ID NO: 10).
wherein
(i) X1 includes I at residue 7, T or M at residue 8, E at residue 11, K at residue 14 and S at residue 18; and
(ii) X3 includes R at residue 3, F at residue 4, K at residue 6, R at residue 7, R at residue 10, N at residue 11, W at residue 13, and G at residue 14.

In a further embodiment, (iii) X4 includes F at residue 19.

In various embodiments, X1 is a peptide comprising the amino acid sequence having identity to the full length of PKKKIQIMAEEALKDALSILNI (SEQ ID NO:8), X3 is a peptide comprising the amino acid sequence having identity to the full length of LERFAKRFERNLWGIARLFESG (SEQ ID NO: 9), and X4 is a peptide comprising the amino acid sequence having identity to the full length of EDEQEEMANAIITILQSWFFS (SEQ ID NO: 10) that are each at least 25%, 27%, 30%, 35%, 40%, 45%, 50%, 55%, 60%, 65%, 70%, 75%, 80%, 85%, 90%, 95%, 98%, or 100%. In specific embodiments,
(i) X1 is a peptide comprising the amino acid sequence at least 65% identical along its length to the peptide PKKKIQIMAEEALKDALSILNI (SEQ ID NO:8); X3 is a peptide comprising the amino acid sequence at least 65% identical along its length the peptide LERFAKRFERNLWGIARLFESG (SEQ ID NO:9); and X4 is a peptide comprising the amino acid sequence at least 65% identical along its length to the peptide EDEQEEMANAIITILQSWFFS (SEQ ID NO:10);
(ii) X1 is a peptide comprising the amino acid sequence at least 75% identical along its length to the peptide PKKKIQIMAEEALKDALSILNI (SEQ ID NO:8); X3 is a peptide comprising the amino acid sequence at least 75% identical along its length the peptide LERFAKRFERNLWGIARLFESG (SEQ ID NO:9); and X4 is a peptide comprising the amino acid sequence at least 75% identical along its length to the peptide EDEQEEMANAIITILQSWFFS (SEQ ID NO:10);
(iii) X1 is a peptide comprising the amino acid sequence at least 80% identical along its length to the peptide PKKKIQIMAEEALKDALSILNI (SEQ ID NO:8); X3 is a peptide comprising the amino acid sequence at least 80% identical along its length the peptide LERFAKRFERNLWGIARLFESG (SEQ ID NO:9); and X4 is a peptide comprising the amino acid sequence at least 80% identical along its length to the peptide EDEQEEMANAIITILQSWFFS (SEQ ID NO:10); or
(iv) X1 is a peptide comprising the amino acid sequence at least 90% identical along its length to the peptide PKKKIQIMAEEALKDALSILNI (SEQ ID NO:8); X3 is a peptide comprising the amino acid sequence at least 90% identical along its length the peptide LERFAKRFERNLWGIARLFESG (SEQ ID NO:9); and X4 is a peptide comprising the amino acid sequence at least 90% identical along its length to the peptide EDEQEEMANAIITILQSWFFS (SEQ ID NO:10);
in each case, wherein
(A) X1 includes I at residue 7, T or M at residue 8, E at residue 11, K at residue 14 and S at residue 18; and (B) X3 includes R at residue 3, F at residue 4, K at residue 6, R at residue 7, R at residue 10, N at residue 11, W at residue 13, and G at residue 14.

In another embodiment of conditionally active receptor agonists IL-4/IL-13 mimetics, amino acid substitutions relative to the reference peptide domains (i.e.: SEQ ID NOS: 8, 9, or 10) do not occur at AA residues marked in bold font. As shown below, SEQ ID NOS:8, 9, and 10 each include residues in bold font:
PKKKIQIMAEEALKDALSILNI (SEQ ID NO:8): amino acid residues E10, E11, A12, L13, K14, D15, A16, and L17 are invariant in this embodiment
LERFAKRFERNLWGIARLFESG (SEQ ID NO: 9): amino acid residues F4, A5, K6, R7, F8, E9, R10, and N11 are invariant in this embodiment
EDEQEEMANAIITILQSWFFS (SEQ ID NO: 10): amino acid residues I11, I12, T13, I14, L15, Q16, S17, W18, F19, and F20 are invariant in this embodiment

In a further embodiment, amino acid residue W13 is invariant when X2 is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide KDEAEKAKRMKEWMKRIK(T) (SEQ ID NO:7).

In another embodiment, amino acid substitutions relative to the reference peptide domains are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In one embodiment, amino acid residues in X1 relative to SEQ ID NO:4 are selected from the group consisting of:

| | | | | | | | | | | |
|---|---|---|---|---|---|---|---|---|---|---|
| Position 01: | A | F | I | L | M | P | Q | R | S | W |
| Position 02: | A | D | E | G | V | K | | | | |
| Position 03: | D | E | F | W | K | | | | | |
| Position 04: | D | E | K | N | P | R | W | | | |
| Position 05: | D | E | H | I | K | L | M | S | | |
| Position 06: | A | D | E | G | L | P | S | W | Q | |
| Position 07: | D | E | L | Q | Y | I | | | | |
| Position 08: | A | F | H | W | Y | M | T | | | |
| Position 09: | C | F | P | A | | | | | | |
| Position 10: | C | D | E | F | K | P | | | | |
| Position 11: | D | F | H | E | | | | | | |
| Position 12: | A | D | E | P | S | T | V | | | |
| Position 13: | H | I | L | M | P | R | V | W | | |
| Position 14: | F | R | W | Y | K | | | | | |
| Position 15: | D | E | N | Y | | | | | | |
| Position 16: | A | C | L | M | S | | | | | |
| Position 17: | F | I | L | M | P | R | | | | |
| Position 18: | G | M | Q | Y | S | | | | | |
| Position 19: | I | L | M | P | Q | V | | | | |
| Position 20: | A | K | L | M | Q | R | S | | | |
| Position 21: | G | K | N | P | R | S | W | | | |
| Position 22: | D | E | I | K | M | N | W | Y | | |

In one embodiment the conditionally active receptor agonists are conditionally active IL-4 mimetics, and position 7 is I, position 8 is M or T, position 11 is E, position 14 is K, and position 18 is S.

In another embodiment the conditionally active receptor agonists are conditionally active IL-2 mimetics, and 1, 2, 3, 4, or 5 of the following are not true: position 7 is I, position 8 is M or T, position 11 is E, position 14 is K, and position 18 is S.

In another embodiment, amino acid residues in X3 relative to SEQ ID NO:5 are selected from the group consisting of:

| | | | | | | | | | | | | | |
|---|---|---|---|---|---|---|---|---|---|---|---|---|---|
| Position 01: | A | L | | | | | | | | | | | |
| Position 02: | D | E | G | K | M | T | | | | | | | |
| Position 03: | D | E | N | Y | R | | | | | | | | |
| Position 04: | C | D | G | T | Y | F | | | | | | | |
| Position 05: | A | F | H | S | V | W | Y | | | | | | |
| Position 06: | A | F | I | M | T | V | Y | K | | | | | |
| Position 07: | D | K | N | S | T | R | | | | | | | |
| Position 08: | A | C | G | L | M | S | V | F | | | | | |
| Position 09: | C | H | K | L | R | S | T | V | E | | | | |
| Position 10: | F | I | L | M | Y | R | | | | | | | |
| Position 11: | I | L | N | T | Y | | | | | | | | |
| Position 12: | F | K | L | M | S | V | | | | | | | |
| Position 13: | A | D | F | G | I | N | P | Q | S | T | E | W | |
| Position 14: | A | E | F | G | H | S | V | | | | | | |
| Position 15: | C | I | L | M | V | W | | | | | | | |
| Position 16: | A | D | G | S | T | V | | | | | | | |
| Position 17: | H | K | L | N | R | | | | | | | | |
| Position 18: | C | D | G | I | L | Q | R | T | W | | | | |
| Position 19: | D | F | M | N | W | | | | | | | | |
| Position 20: | A | C | E | F | G | M | S | Y | | | | | |
| Position 21: | D | E | G | H | L | M | R | S | T | | | V | W |
| Position 22: | A | D | G | K | N | S | Y | | | | | | |

In another embodiment, the conditionally active receptor agonists are conditionally active IL-4/IL-13 mimetics and position 3 is R, position 4 is F, position 6 is K, position 7 is R, position 10 is R, position 11 is N, position 13 is W, and position 14 is G.

In another embodiment, the conditionally active receptor agonists are conditionally active IL-2 mimetics and 1, 2, 3, 4, 5, 6, 7, or all 8 of the following are not true: position 3 is R, position 4 is F, position 6 is K, position 7 is R, position 10 is R, position 11 is N, position 13 is W, and position 14 is G.

In any of such embodiments, the conditionally active receptor agonists further allows for a cysteine at position 17 relative to SEQ ID NO:5 in addition to the amino acid residues of H, K, L, N and R, or at position 20 relative to SEQ ID NO:5 in addition to the amino acid residues of A, C, E, F, G, M, S, and Y. Accordingly, in this embodiment amino acid residues in X3 relative to SEQ ID NO:5 can be selected from the group consisting of:

| | | | | | | | | | | | | | | |
|---|---|---|---|---|---|---|---|---|---|---|---|---|---|---|
| Position 01: | A | L | | | | | | | | | | | | |
| Position 02: | D | E | G | K | M | T | | | | | | | | |
| Position 03: | D | E | N | Y | R | | | | | | | | | |
| Position 04: | C | D | G | T | Y | F | | | | | | | | |
| Position 05: | A | F | H | S | V | W | Y | | | | | | | |
| Position 06: | A | F | I | M | T | V | Y | | K | | | | | |
| Position 07: | D | K | N | S | T | R | | | | | | | | |
| Position 08: | A | C | G | L | M | S | V | | F | | | | | |
| Position 09: | C | H | K | L | R | S | T | | V | | | E | | |
| Position 10: | F | I | L | M | Y | R | | | | | | | | |
| Position 11: | I | L | N | T | Y | | | | | | | | | |
| Position 12: | F | K | L | M | S | V | | | | | | | | |
| Position 13: | A | D | F | G | I | N | P | Q | S | T | E | W | | |
| Position 14: | A | E | F | G | H | S | V | | | | | | | |
| Position 15: | C | I | L | M | V | W | | | | | | | | |
| Position 16: | A | D | G | S | T | V | | | | | | | | |
| Position 17: | H | K | L | N | R | C | | | | | | | | |
| Position 18: | C | D | G | I | L | Q | R | | T | | | W | | |
| Position 19: | D | F | M | N | W | | | | | | | | | |
| Position 20: | A | C | E | F | G | M | S | | Y | | | C | | |
| Position 21: | D | E | G | H | L | M | R | | S | | | T | V | W |
| Position 22: | A | D | G | K | N | S | Y | | | | | | | |

In another embodiment, amino acid residues in X4 relative to SEQ ID NO:6 are selected from the group consisting of:

| | | | | | | | | | | |
|---|---|---|---|---|---|---|---|---|---|---|
| Position 01: | D | E | G | K | V | | | | | |
| Position 02: | D | I | M | S | | | | | | |
| Position 03: | E | G | H | K | | | | | | |
| Position 04: | E | G | I | K | Q | R | S | | | |
| Position 05: | A | D | E | G | H | S | V | | | |
| Position 06: | C | D | E | G | I | M | Q | R | T | V |
| Position 07: | C | E | L | M | P | R | T | | | |
| Position 08: | A | F | L | M | W | | | | | |
| Position 09: | A | G | L | N | Q | R | T | | | |
| Position 10: | A | C | D | E | F | H | I | W | | |
| Position 11: | I | M | N | S | V | W | | | | |
| Position 12: | I | K | L | S | V | | | | | |
| Position 13: | C | L | M | R | S | T | | | | |
| Position 14: | I | L | P | T | Y | | | | | |
| Position 15: | F | G | I | L | M | N | V | | | |
| Position 16: | H | K | Q | R | | | | | | |
| Position 17: | C | F | K | S | W | Y | | | | |
| Position 18: | K | Q | T | W | | | | | | |
| Position 19: | C | G | N | I | | | | | | |
| Position 20: | C | F | G | L | Y | | | | | |
| Position 21: | A | F | G | H | S | Y | | | | |

In another embodiment, the conditionally active receptor agonists are conditionally active IL-4/IL-13 mimetics and position 19 is I. In another embodiment, the conditionally active receptor agonists are conditionally active IL-2 mimetics and position 19 is not I.

In any of such embodiments, the conditionally active receptor agonists further allows for a cysteine at position 3 relative to SEQ ID NO:6 in addition to the amino acid residues of E, G, H and K. Accordingly, in this embodiment, amino acid residues in X4 relative to SEQ ID NO:6 can be selected from the group consisting of:

| | | | | | | | | | | |
|---|---|---|---|---|---|---|---|---|---|---|
| Position 01: | D | E | G | K | V | | | | | |
| Position 02: | D | I | M | S | | | | | | |
| Position 03: | E | G | H | K | C | | | | | |
| Position 04: | E | G | I | K | Q | R | S | | | |
| Position 05: | A | D | E | G | H | S | V | | | |
| Position 06: | C | D | E | G | I | M | Q | R | T | V |
| Position 07: | C | E | L | M | P | R | T | | | |
| Position 08: | A | F | L | M | W | | | | | |
| Position 09: | A | G | L | N | Q | R | T | | | |
| Position 10: | A | C | D | E | F | H | I | W | | |
| Position 11: | I | M | N | S | V | W | | | | |
| Position 12: | I | K | L | S | V | | | | | |
| Position 13: | C | L | M | R | S | T | | | | |
| Position 14: | I | L | P | T | Y | | | | | |
| Position 15: | F | G | I | L | M | N | V | | | |
| Position 16: | H | K | Q | R | | | | | | |
| Position 17: | C | F | K | S | W | Y | | | | |
| Position 18: | K | Q | T | W | | | | | | |
| Position 19: | C | G | N | I | | | | | | |
| Position 20: | C | F | G | L | Y | | | | | |
| Position 21: | A | F | G | H | S | Y | | | | |

As noted herein, domain X2 is a structural domain, and thus any amino acid sequence that connects (i.e.: in the same polypeptide or upon non-covalent interaction of the first and second polypeptide) the relevant other domains and allows them to fold can be used. The length required will depend on the specifics of the first polypeptide and the second polypeptide being used and can be 8 amino acids or longer. In one exemplary and non-limiting embodiment, X2 is a peptide comprising the amino acid sequence at least 20%, 27%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to KDEAEKAKRMKEWMKRIK(T) (SEQ ID NO:7) or KDEAEKAKRMKEWMKRIKT (SEQ ID NO:7). In one embodiment, amino acid changes relative to the amino acid sequence of SEQ ID NO:7 are conservative amino acid substitutions. In another embodiment, the W13 amino acid residue is invariant. In a further embodiment, amino acid residues in X2 relative to SEQ ID NO:7 are selected from the group consisting of:

| | | | | | | | | | | |
|---|---|---|---|---|---|---|---|---|---|---|
| Position 01: | A | H | L | M | R | S | V | K | | |
| Position 02: | A | D | E | Q | R | S | T | V | W | Y |
| Position 03: | C | E | G | K | L | N | Q | R | W | |
| Position 04: | A | F | G | N | S | T | V | Y | | |
| Position 05: | A | E | G | I | M | R | V | | | |
| Position 06: | C | E | K | L | N | R | V | | | |
| Position 07: | A | C | E | I | L | S | T | V | W | |
| Position 08: | H | K | L | M | S | T | W | Y | | |
| Position 09: | A | I | L | M | Q | S | R | | | |
| Position 10: | A | I | M | S | W | Y | | | | |
| Position 11: | C | I | K | L | S | V | | | | |
| Position 12: | C | E | K | L | P | Q | R | T | | |
| Position 13: | A | D | H | N | W | | | | | |
| Position 14: | A | C | G | I | L | S | T | V | M | |
| Position 15: | A | E | G | I | K | L | M | R | V | |
| Position 16: | G | H | L | R | S | T | V | | | |
| Position 17: | A | I | L | V | | | | | | |
| Position 18: | A | C | D | E | G | H | I | K | M | S |
| Position 19: | D | E | G | L | N | V | T | | | |

In another embodiment, the polypeptides are IL-4/IL-13 mimetics and position 11 is I. In another embodiment, the polypeptides are IL-2 mimetics and position 11 is not I.

In any of such embodiments, the polypeptide further allows for a cysteine at positions 5 or 16 relative to SEQ ID NO:7.

Alternatively, in any of such embodiments, the polypeptide further allows for a cysteine at positions 1, 2, 5, 9 or 16 relative to SEQ ID NO:7

Accordingly, amino acid residues in X2 relative to SEQ ID NO:7 can be selected from the group consisting of:

| | | | | | | | | | | | |
|---|---|---|---|---|---|---|---|---|---|---|---|
| Position 01: | A | H | L | M | R | S | V | K | C | | |
| Position 02: | A | D | E | Q | R | S | T | V | W | Y | C |
| Position 03: | C | E | G | K | L | N | Q | R | W | | |
| Position 04: | A | F | G | N | S | T | V | Y | | | |
| Position 05: | A | E | G | I | M | R | V | C | | | |
| Position 06: | C | E | K | L | N | R | V | | | | |
| Position 07: | A | C | E | I | L | S | T | V | W | | |
| Position 08: | H | K | L | M | S | T | W | Y | | | |
| Position 09: | A | I | L | M | Q | S | R | C | | | |
| Position 10: | A | I | M | S | W | Y | | | | | |
| Position 11: | C | I | K | L | S | V | | | | | |
| Position 12: | C | E | K | L | P | Q | R | T | | | |
| Position 13: | A | D | H | N | W | | | | | | |
| Position 14: | A | C | G | I | L | S | T | V | M | | |
| Position 15: | A | E | G | I | K | L | M | R | V | | |
| Position 16: | G | H | L | R | S | T | V | C | | | |
| Position 17: | A | I | L | V | | | | | | | |
| Position 18: | A | C | D | E | G | H | I | K | M | S | |
| Position 19: | D | E | G | L | N | V | T | | | | |

In various specific embodiments:
(i) X2 is a peptide comprising the amino acid sequence at least 70% identical along its length to KDEAEKAKRMKEWMKRIK(T) (SEQ ID NO:7);
(ii) X2 is a peptide comprising the amino acid sequence at least 80% identical along its length to KDEAEKAKRMKEWMKRIK(T) (SEQ ID NO:7);
(iii) X2 is a peptide comprising the amino acid sequence at least 90% identical along its length to KDEAEKAKRMKEWMKRIK(T) (SEQ ID NO:7);
(iv) X2 is a peptide comprising the amino acid sequence having identity to the full length of peptide KDEAEKAKRMKEWMKRIK(T) (SEQ ID NO:7) of at least 65%; X1 is a peptide comprising the amino acid sequence having identity to the full length of peptide PKKKIQLHAEHALYDAL(MILNI (SEQ ID NO: 4) or QLHAEHALYDAL(MILNI (SEQ ID NO:320) of at least 65%; X3 is a peptide comprising the amino acid sequence having identity to the full length of peptide LEDYAFNFELILEEIARLFESG (SEQ ID NO:5) or LEDYAFNFELILEEIARLFES (SEQ ID NO:321) of at least 65%; and X4 is a peptide comprising the amino acid sequence having identity to the full length of peptide EDEQEEMANAIITILQSWIF(S) (SEQ ID NO:6) or DEQEEMANAIITILQSWIFS (SEQ ID NO:322) of at least 65%;
(v) X2 is a peptide comprising the amino acid sequence having identity to the full length of peptide KDEAEKAKRMKEWMKRIK(T) (SEQ ID NO:7) of at least 75%; X1 is a peptide comprising the amino acid sequence having identity to the full length of peptide PKKKIQLHAEHALYDAL(MILNI (SEQ ID NO: 4) or QLHAEHALYDAL(MILNI (SEQ ID NO:320) of at least 75%; X3 is a peptide comprising the amino acid sequence having identity to the full length of peptide LEDYAFNFELILEEIARLFESG (SEQ ID NO:5) or LEDYAFNFELILEEIARLFES (SEQ ID NO:321) of at least 75%; and X4 is a peptide comprising the amino acid sequence having identity to the full length of peptide EDEQEEMANAIITILQSWIF(S) (SEQ ID NO:6) or DEQEEMANAIITILQSWIFS (SEQ ID NO:322) of at least 75%;
(vi) X2 is a peptide comprising the amino acid sequence having identity to the full length of peptide KDEAEKAKRMKEWMKRIK(T) (SEQ ID NO:7) of at least 80%; X1 is a peptide comprising the amino acid sequence having identity to the full length of peptide PKKKIQLHAEHALYDAL(MILNI (SEQ ID NO: 4) or QLHAEHALYDAL(MILNI (SEQ ID NO:320) of at least 80%; X3 is a peptide comprising the amino acid sequence having identity to the full length of peptide LEDYAFNFELILEEIARLFESG (SEQ ID NO:5) or LEDYAFNFELILEEIARLFES (SEQ ID NO:321) of at least 80%; and X4 is a peptide comprising the amino acid sequence having identity to the full length of peptide EDEQEEMANAIITILQSWIF(S) (SEQ ID NO:6) or DEQEEMANAIITILQSWIFS (SEQ ID NO:322) of at least 80%;
(vii) X2 is a peptide comprising the amino acid sequence having identity to the full length of peptide KDEAEKAKRMKEWMKRIK(T) (SEQ ID NO:7) of at least 90%; X1 is a peptide comprising the amino acid sequence having identity to the full length of peptide PKKKIQLHAEHALYDAL(MILNI (SEQ ID NO: 4) or QLHAEHALYDAL(MILNI (SEQ ID NO:320) of at least 90%; X3 is a peptide comprising the amino acid sequence having identity to the full length of peptide LEDYAFNFELILEEIARLFESG (SEQ ID NO:5) or LEDYAFNFELILEEIARLFES (SEQ ID NO:321) of at least 90%; and X4 is a peptide comprising the amino acid sequence having identity to the full length of peptide EDEQEEMANAIITILQSWIF(S) (SEQ ID NO:6) or DEQEEMANAIITILQSWIFS (SEQ ID NO:322) of at least 90%; or
(viii) X2 is a peptide comprising the amino acid sequence having 100% identity to the full length of peptide KDEAEKAKRMKEWMKRIK(T) (SEQ ID NO:7); X1 is a peptide comprising the amino acid sequence having 100% identity to the full length of peptide PKKKIQLHAEHALYDAL(MILNI (SEQ ID NO: 4) or QLHAEHALYDAL(MILNI (SEQ ID NO:320); X3 is a peptide comprising the amino acid sequence having 100% identity to the full length of peptide LEDYAFNFELILEEIARLFESG (SEQ ID NO:5) or LEDYAFNFELILEEIARLFES (SEQ ID NO:321); and X4 is a peptide comprising the amino acid sequence having 100% identity to the full length of peptide EDEQEEMANAIITILQSWIF(S) (SEQ ID NO:6) or DEQEEMANAIITILQSWIFS (SEQ ID NO:322).

In exemplary embodiments of (i) through (viii) above, the listed optional amino acid residues in SEQ ID NO:7 are present. In exemplary embodiments of (i) through (viii) above, the peptides for X1, X3, and X4 are shown in SEQ ID Nos. 4, 5, and 6. In exemplary embodiments of (i) through (viii) above, the peptides for X1, X3, and X4 are shown in SEQ ID Nos. 320, 321, and 322.

In various embodiments:
(i) the first polypeptide component includes one of X1, X2, X3, and X4, and the second polypeptide component includes the three of X1, X2, X3, and X4 that are not present in the first polypeptide component; or
(ii) the first polypeptide component includes two of X1, X2, X3, and X4, and the second polypeptide component includes the two of X1, X2, X3, and X4 that are not present in the first polypeptide component.

In further embodiments;
(a) the first polypeptide comprises X1 and excludes X2, X3, and X4; and the second polypeptide is a fusion protein comprising X3-Z1-X2-Z2-X4 and excluding X1;
(b) the first polypeptide comprises X4 and excludes X1, X2, and X3; and the second polypeptide is a fusion protein comprising X1-Z1-X3-Z2-X2 and excluding X4; or
(c) the first polypeptide is a fusion protein comprising X1-Z1-X3 and excluding X2 and X4; and the second polypeptide is a fusion protein comprising X2-Z1-X4 and excluding X1 and X3;
wherein each of Z1 and Z2 independently are an optional amino acid linker. Z1 and/or Z2 may comprise any number of amino acid residues to separate domains within the first and/or second polypeptide as deemed appropriate for an intended use. The 1 and/or Z2 linker may be of any suitable length and amino acid composition. In one embodiment, Z1 and Z2 are both absent; in another embodiment, 1 and 2 are both present; in a further embodiment, one of Z1 sand Z2 is present and the other is absent.

In other embodiments, X1, X2, X3, and X4, respectively comprise a peptide at least 25%, 27%, 30%, 35%, 40%, 45%, 50%, 55%, 60%, 65%, 70%, 75%, 80%, 85%, 90%, 95%, 98%, or 100% identical, respectively, to the full length of X1, X2, X3, and X4 domains shown below (SEQ ID NOS: 4-7), where residues in parentheses may be present or absent:

X1:
(SEQ ID NO: 4)
PKKKIQLHAEHALYDALMILNI
X2:
(SEQ ID NO: 7)
(K)DEAEKAKRMKEWMKRIKT
X3:
(SEQ ID NO: 5)
(LE)DYAFNFELILEEIARLF(ESG)
X4:
(SEQ ID NO: 6)
(E)DFQEEMANAIITILQSWIFS

In other embodiments, X1, X2, X3, and X4 are peptides comprising amino acid sequences at least 80% identical, respectively, to the full length of X1, X2, X3, and X4 domains shown below (SEQ ID NOS: 4-7), where residues in parentheses may be present or absent:

X1:
(SEQ ID NO: 4)
PEKKIQLHAEHALYDALMILNI
X2:
(SEQ ID NO: 7)
(K)DEAEKAKRMKEWMKRIKT
X3:
(SEQ ID NO: 5)
(LE)DYAFNFELILEEIARLF(ESG)
X4:
(SEQ ID NO: 6)
(E)DEQEEMANAIITILQSWIFS

In other embodiments, X1, X2, X3, and X4 are peptides comprising amino acid sequences at least 90% identical, respectively, to the full length of X1, X2, X3, and X4 domains shown below (SEQ ID NOS: 4-7), where residues in parentheses may be present or absent:

X1:
(SEQ ID NO: 4)
PKKYIQLHAEHALYDALMILNI
X2:
(SEQ ID NO: 7)
(K)DEAEKAKRMKEWMKRIKT
X3:
(SEQ ID NO: 5)
(LE)DYAFNFELILEEIARLF(ESG)
X4:
(SEQ ID NO: 6)
(E)DEQEEMANAIITILQSWIFS.

In other embodiments, X1, X2, X3, and X4, respectively are 25%, 27%, 30%, 35%, 40%, 45%, 50%, 55%, 60%, 65%, 70%, 75%, 80%, 85%, 90%, 95%, 98%, or 100% identical, respectively, to the full length of X1, X2, X3, and X4 domains shown below, where residues in parentheses may be present or absent:

X1:
(SEQ ID NO: 320)
QLHAEHALYDALMILNI
X2:
(SEQ ID NO: 7)
(K)DEAEKAKRMKEWMKRIKT
X3:
(SEQ ID NO: 321)
LEDYAFNFELILEEIARLFES
X4:
(SEQ ID NO: 322)
DEQEEMANAIITILQSWIFS.

In other embodiments, X1, X2, X3, and X4, respectively are peptides comprising amino acid sequences at least 80% identical, respectively, to the full length of X1, X2, X3, and X4 domains shown below, where residues in parentheses may be present or absent:

X1:
(SEQ ID NO: 320)
QLHAEHALYDALMILNI
X2:
(SEQ ID NO: 7)
(K)DEAEKAKRMKEWMKRIKT
X3:
(SEQ ID NO: 321)
LEDYAFNFELILEEIARLFES
X4:
(SEQ ID NO: 322)
DEQEEMANAIITILQSWIFS.

In other embodiments, X1, X2, X3, and X4, respectively are peptides comprising amino acid sequences at least 90% identical, respectively, to the full length of X1, X2, X3, and X4 domains shown below, where residues in parentheses may be present or absent:

X1:
(SEQ ID NO: 320)
QLHAEHALYDALMILNI
X2:
(SEQ ID NO: 7)
(K)DEAEKAKRMKEWMKRIKT
X3:
(SEQ ID NO: 321)
LEDYAFNFELILEEIARLFES
X4:
(SEQ ID NO: 322)
DEQEEMANAIITILQSWIFS.

In one embodiment, one or more or all of the optional amino acids are present; in another embodiment, one or more or all of the optional amino acids are absent. In other embodiments:
(i) X1 is a peptide comprising the amino acid sequence having identity to the full length of the amino acid sequence of SEQ ID NO:4 or SEQ ID NO:320 of at least 55%; X2 is a peptide comprising the amino acid sequence having identity to the full length of the amino acid sequence of SEQ ID NO:7 of at least 55%, X3 is a peptide comprising the amino acid sequence having identity to the full length of the amino acid sequence of SEQ ID NO:5 or SEQ ID NO:321 of at least 55%; and X4 is a peptide comprising the amino acid sequence having identity to the full length of the amino acid sequence of SEQ ID NO:6 or SEQ ID NO:322 of at least 55%;
(ii) X1 is a peptide comprising the amino acid sequence having identity to the full length of the amino acid sequence of SEQ ID NO:4 or SEQ ID NO:320 of at least 75%; X2 is a peptide comprising the amino acid sequence having identity to the full length of the amino acid sequence of SEQ ID NO:7 of at least 75%, X3 is a peptide comprising the amino acid sequence having identity to the full length of the amino acid sequence of SEQ ID NO:5 or SEQ ID NO:321 of at least 75%; and X4 is a peptide comprising the amino acid sequence having identity to the full length of the amino acid sequence of SEQ ID NO:6 or SEQ ID NO:322 of at least 75%;
(iii) X1 is a peptide comprising the amino acid sequence having identity to the full length of the amino acid sequence of SEQ ID NO:4 or SEQ ID NO:320 of at least 80%; X2 is a peptide comprising the amino acid sequence having identity to the full length of the amino acid sequence of SEQ ID NO:7 of at least 80%, X3 is a peptide comprising the amino acid sequence having identity to the full length of the amino acid sequence of SEQ ID NO:5 or SEQ ID NO:321 of at least 80%; and X4 is a peptide comprising the amino acid sequence having identity to the full length of the amino acid sequence of SEQ ID NO:6 or SEQ ID NO:322 of at least 80%;
(iv) X1 is a peptide comprising the amino acid sequence having identity to the full length of the amino acid sequence of SEQ ID NO:4 or SEQ ID NO:320 of at least 90%; X2 is a peptide comprising the amino acid sequence having identity to the full length of the amino acid sequence of SEQ ID NO:7 of at least 90%, X3 is a peptide comprising the amino acid sequence having identity to the full length of the amino acid sequence of SEQ ID NO:5 or SEQ ID NO:321 of at least 90%; and X4 is a peptide comprising the amino acid sequence having identity to the full length of the amino acid sequence of SEQ ID NO:6 or SEQ ID NO:322 of at least 90%; or
(vi) X1 is a peptide comprising the amino acid sequence having 100% identity to the full length of the amino acid sequence of SEQ ID NO:4 or SEQ ID NO:320; X2 is a peptide comprising the amino acid sequence having 100% identity to the full length of the amino acid sequence of SEQ ID NO:7; X3 is a peptide comprising the amino acid sequence having 100% identity to the full length of the amino acid sequence of SEQ ID NO:5 or SEQ ID NO:321; and X4 is a peptide comprising the amino acid sequence having 100% identity to the full length of the amino acid sequence of SEQ ID NO:6 or SEQ ID NO:322.

In exemplary embodiments of (i) through (vi) above, the peptides for X1, X3, and X4 are shown in SEQ ID NOs. 4, 5, and 6. In exemplary embodiments of (i) through (vi) above, the peptides for X1, X3, and X4 are shown in SEQ ID NOs. 320, 321, and 322.

In another embodiment, the first polypeptide and the second polypeptide, are at least 25%, 27%, 30%, 35%, 40%, 45%, 50%, 55%, 60%, 65%, 70%, 75%, 80%, 85%, 90%, 95%, 98%, or 100% identical to a pair of first and second polypeptides shown below (underlined residues or “X” residues” are optional and each residue of the optional domain, when present, may comprise any amino acid):

(i)
First polypeptide X1 (Neo2A)
(SEQ ID NO: 256)
PKKKIQLHAEHALYDALMILNIVKTNS
and
Second polypeptide: X3-X2′-X4 (Neo2B)
(SEQ ID NO: 257)
TNSPPAEEKLEDYAFNFELILEEIARLFESGDQKDEAEKAKRMKEWMKRIKTTASEDEQEEMANAIIT
ILQWIFS
(ii)
First polypeptide X1-X3-X2′
(SEQ ID NO: 258)
PKKKIQLHAEHALYDALMILNIVKTNSPPAEEKLEDYAFNFELILEEIARLFESGDQKDEAEKAKRMK
EWMKRIKTTAS
and
Second polypeptide X4
(SEQ ID NO: 259)
TTASEDEQEEMANAIITILQSWIFS;
(iii)
First polypeptide X1-X3
(SEQ ID NO: 260)
PKKKIQLHAEHALYDALMILNIVKTNSPPAEEKLEDYAFNFELILEEIARLFESGD
and
Second polypeptide X2-X4
(SEQ ID NO: 261)
DQKDEAEKAKRMKEWMKRIKTTASEDEQEEMANAIITILQSWIFS
(iv)
First polypeptide X1 (Neo4A)
(SEQ ID NO: 262)
PKKKIQIMAEEALKDALSILNIVKTNS
Second polypeptide X3-X2′-X4 (Neo4B)
(SEQ ID NO: 263)
TNSPPAEEQLERFAKRFERNLWGIARLFESGDQKDEAEKAKRMIEWMKRIKTTASEDEQEEMANAIIT
ILQSWFFS
(v)
First polypeptide X1 (Neo2A)
(SEQ ID NO: 311)
PKKKIQLHAEHALYDALMILNIXXXXX
and
Second polypeptide: X3-X2′-X4 (Neo2B)
(SEQ ID NO: 264)
XXXXXXXXXLEDYAFNFELILEEIARLFESGXXKDEAEKAKRMKEWMKRIKTXXXEDEQEEMANAIIT
ILQSWIFS
(vi)
First polypeptide X1-X3-X2′
(SEQ ID NO: 265)
PKKKIQLHAEHALYDALMILNIXXXXXXXXXXXLEDYAFNFELILEEIARLFESGXXKDEAEKAKRMK
EWMKRIKTTAS
and
Second polypeptide X4
(SEQ ID NO: 266)
XXXXXDEQEEMANAIITILQSWIFS;
(vii)
First polypeptide X1-X3
(SEQ ID NO: 267)
PKKKIQLHAEHALYDALMILNIXXXXXXXXXXXLEDYAFNFELILEEIARLFESXXGD
and
Second polypeptlde X2-X4
(SEQ ID NO: 268)
DQKDEAFKAKRMKEWMKRIKTXXXEDEQEEMANAIITILQSWIFS
(viii)
First polypeptide X1 (Neo4A)
(SEQ ID NO: 269)
PKKKIQIMAEEALKDALSILNIXXXXX
Second polypeptide X3-X2′-X4 (Neo4B)
(SEQ ID NO: 270)
XXXXXXXXQLERFAKRFERNLWGIARLFESGXXKDEAEKAKRMIEWMKRIKTXXXEDEQEEMANAIIT
ILQSWFFS
(ix)
First polypeptide >Neo4_H1-H3′
(SEQ ID NO: 312)
PKKKIQIMAEEALKDALSILNIVKTNSPPAEEQLERFAKRFERNLWGIARLFESGD
Second polypeptide >Neo4_H2-H4
(SEQ ID NO: 313)
DQKDEAEKAKRMIEWMKRIKTTASEDEQEEMANAIITILQSWFFS
(x)
First polypeptide >Neo4_H1-H3′
(SEQ ID NO: 314)
PKKKIQIMAEEALKDALSILNIXXXXXXXXXXQLERFAKRFERNLWGIARLFESXX
Second polypeptide >Neo4_H2-H4
(SEQ ID NO: 315)
XXKDEAEKAKRMIEWMKRIKXXXXEDEQEEMANAIITILQSWFFS
(xi)
First polypeptide Neo4_H1-H3′-H2
(SEQ ID NO: 316)
PKKKIQIMAEEALKDALSILNIVKTNSPPAEEQLERFAKRFERNLWGIARLFESGDQKDEAEKAKRMI
EWMKRIKTTA
Second polypeptide Neo4_H4
(SEQ ID NO: 317)
TTASEDEQEEMANAIITILQSWFFS
(xii)
First polypeptide Neo4_H1-H3′-H2
(SEQ ID NO: 318)
PKKKIQIMAEEALKDALSILNIXXXXXXXXXXXLERFAKRFERNLWGIARLFESXXXKDEAEKAKRMI
EWMKRIKXXX
Second polypeptide Neo4_H4
(SEQ ID NO: 319)
XXXXXDEQEEMANAIITILQSWFFS
(xiii)
First polypeptide (X1)
(SEQ ID NO: 323)
PKKKIQLHAEHALYDALMILNIVGGSS,
or
(SEQ ID NO: 324)
SKEAIQLHAEHALYDALMILNIVKTNS,
or
(SEQ ID NO: 325)
PIQLHAEHALYDALMILNIV
Second polypeptide (X3-X2′-X4)
(SEQ ID NO: 326)
PKLEDYAFNFELILEEIARLFESGDQKDEAEKAKRMKEWMKRIKTTASEDEQEEMANAIITILQSWI
FS;
or
(SEQ ID NO: 327)
GGSSGGLEDYAFNFELILEEIARLFESGGSSGGKDEAEKAKRMKEWMKRITGGSSGGDEQEEMANAI
ITILQSWIFS;
or
(SEQ ID NO: 328)
GGSSGGLEDYAFNFELILEEIARLFESGGSSGGGGEAEKAKRMKEWMKRIGGSSGGDEQEEMANAIIT
ILQSWIFS.

In exemplary embodiments, the first polypeptide and the second polypeptide are peptides comprising an amino acid sequence at least 80% identical to a pair of first and second polypeptides shown in embodiments (i)-(viii) above (underlined residues or “X” residues” are optional and each residue of the optional domain, when present, may comprise any amino acid).

In exemplary embodiments, the first polypeptide and the second polypeptide are peptides comprising an amino acid sequence at least 90% identical to a pair of first and second polypeptides shown in embodiments (i)-(viii) above (underlined residues or “X” residues” are optional and each residue of the optional domain, when present, may comprise any amino acid).

In exemplary embodiments, the first polypeptide and the second polypeptide are peptides comprising an amino acid sequence 100% identical to a pair of first and second polypeptides shown in embodiments (i)-(viii) above (underlined residues or “X” residues” are optional and each residue of the optional domain, when present, may comprise any amino acid).

In various further embodiments, X1, X2, X3, and X4, respectively, comprise a peptide at least 25%, 27%, 30%, 35%, 40%, 45%, 50%, 55%, 60%, 65%, 70%, 75%, 80%, 85%, 90%, 95%, 98%, or 100% identical to, respectively, X1, X2, X3, and X4 domains (as defined in Table 1 (though listed as H1, H2, H3, and H4 domains) present within the amino acid sequence of SEQ ID NO:11-94, 190-216, 247, and SEQ ID NOS:275-300.

Table 1 provides two SEQ ID NOs for many of the variants: a first SEQ ID NO: that lists the linker positions as optional and variable (shown by underlining in the table), and a second SEQ ID NO: that includes the linker positions as required. Table 1 shows the domain arrangement for the polypeptide of SEQ ID NOS:11-94, 190-216, 247, and SEQ ID NOS: 275-300 (see the second column), while the sequence shows underlined amino acid linkers separating domains. See, for example, SEQ ID NO:11, having the domain arrangement H1->H4->H2′->H3 (corresponding to an X1-X4-X2-X3 arrangement):

(SEQ ID NO: 11)
STKKWQLQAEHALLDWQMALNKSPEPNENLNRAITAAQSWISTGKIDLDK
AEDIRRNSDQARREAEKRGIDVRDLISNAQVILLEAR

As will be apparent to those of skill in the art based on this arrangement, in SEQ ID NO:11 the X1 domain is STKKWQLQAEHALLDWQMALNK (SEQ ID NO:271), the X4 domain is ENLNRAITAAQSWIS (SEQ ID NO:272), the X2 domain is LDKAEDIRRNSDQARREAEK (SEQ ID NO:273), and the X3 domain is RDLISNAQVILLEAR (SEQ ID NO:274). Similarly, the amino acid sequence of each X1, X2, X3, and X4 domains SEQ ID NOS:11-94, 190-216, 247, and SEQ ID NOS: 275-300 will be clear to those of skill in the art based on the teachings herein. As will be understood by those of skill in the art, the X1, X2, X3, and/or X4 amino acids may include additional (1, 2, 3, 4, 5, or more) amino acids at the N-terminus and/or the C-terminus relative to the X1, X2, X3, and X4 domains shown in SEQ ID NOS: 11-94, 190-216, 247, and SEQ ID NOS: 275-300.

TABLE 1

| | Domain | |
|---|---|---|
| | arrange- | |
| Name | ment | Sequence |
| G1_neo2_33 | H1->H4- | STKKWQLQAEHALLDWQMALNKSPEPNENLNRAITAAQSWISTGKIDLD |
| | >H2′- | KAEDIRRNSDQARREAEKRGIDVRDLISNAQVILLEAR (SEQ ID NO: 11) |
| | >H3 | STKKWQLQAEHAILDWQMALNKSPEPNENLNRAITAAQSWISTGKIDLD |
| | | KAEDIRRNSDQARREAEKRGIDVRDLISNAQVILLEAR (SEQ ID NO: 103) |
| G1_neo2_34 | H1->H4- | STKKWQLQAEHALLDWQMALNKSPEPNENLNRAITAAQSCISTGKCDLD |
| | >H2′- | KAEDIRRNSDQARREAEKRGIDVRDLISNAQVILLEAR (SEQ ID NO: 12) |
| | >H3 | STKKWQLQAEHALLDWQMALNKSPEPNENLNRAITAAQSCISTGKCDLD |
| | | KAEDIRRNSDQARREAEKRGIDVRDLISNAQVILLEAR (SEQ ID NO: 104) |
| G1_neo2_35 | H1->H4- | STKKWQLQAEHALLDWQMALNKSPEPNENLNRAITAAQSWISTGKIDCD |
| | >H2′- | KAEDIRRNSDQARREAEKRGIDVRDLISNAQVILLEAC (SEQ ID NO: 13) |
| | >H3 | STKKWQLQAEHALLDWQMALNKSPEPNENLNRAITAAQSWISTGKIDCD |
| | | KAEDIRRNSDQARREAEKRGIDVRDLISNAQVILLEAC (SEQ ID NO: 105) |
| G1_neo2_36 | H1->H4- | STKKLQLQAEHFLLDVQMILNESPEPNEELNRAITDAQSWISTGKIDLD |
| | >H2′- | RAEELARNLEKVRDEALKRGIDVRDLVSNAKVIALELK (SEQ ID NO: 14) |
| | >H3 | STKKLQLQAEHFLLDVQMILNESPEPNEELNRAITDAQSWISTGKIDLD |
| | | RAEELARNLEKVRDEALKRGIDVRDLVSNAKVIALELK (SEQ ID NO: 106) |
| G1_neo2_37 | H1->H4- | STKKLQLQAEHFLLDVQMILNESPEPNEELNRCITDAQSWISTGKIDLD |
| | >H2′- | RAEECARNLEKVRDEALKRGIDVRDLVSNAKVIALELK (SEQ ID NO: 15) |
| | >H3 | STKKLQLQAEHFLLDVQMILNESPEPNEELNRCITDAQSWISTGKIDLD |
| | | RAEECARNLEKVRDEALKRGIDVRDLVSNAKVIALELK (SEQ ID NO: 107) |
| G1_neo2_38 | H1->H4- | STKKLQLQAEHFLLDVQMILNESPEPNEELNRAITDAQSCISTGKCDLD |
| | >H2′- | RAEELAPNLEKVRDEALKRGIDVRDLVSNAKVIALELK (SEQ ID NO: 16) |
| | >H3 | STKKLQLQAEHFLLDVQMILNESPEPNEELNRAITDAQSCISTGKCDLD |
| | | RAEELARNLEKVRDEALKRGIDVRDLVSNAKVIALELK (SEQ ID NO: 108) |
| G1_neo2_39 | H1->H4- | STKKLQLQAEHELLDVQMILNESPEPNEELNRAITDAQSWISTGKIDLD |
| | >H2′- | RAEELCRNLEKVRDEALKRGIDVRDLVSNACVIALELK (SEQ ID NO: 17) |
| | >H3 | STKKLQLQAEHFLLDVQMILNESPEPNEELNRAITDAQSWISTGKIDLD |
| | | RAEELCRNLEKVRDEALKRGIDVRDLVSNACVIALELK (SEQ ID NO: 109) |
| G1_neo2_40 | H1->H4- | STKKLQLQAEHALLDAQMMLNRSPEPNEKLNRIITTMQSWISTGKIDLD |
| | >H2′- | GAKELAKEVEELRQEAEKRGIDVRDLASNLKVILLELA (SEQ ID NO: 18) |
| | >H3 | STKKLQLQAEHALLDAQMMLNRSPEPNEKLNRIITTMQSWISTGKIDLD |
| | | GAKELAKEVEELPQEAEKRGIDVRDLASNLKVILLELA (SEQ ID NO: 110) |
| G1_neo2_41 | H1->H4- | STKKLQLQAEHALLDAQMMLNRSPEPNEKLNRIITTMQSCISTGKCDLD |
| | >H2′- | GAKELAKEVEELRQEAEKRGIDVRDLASNLKVILLELA (SEQ ID NO: 19) |
| | >H3 | STKKLQLQAEHALLDAQMMLNRSPEPNEKLNRIITTMQSCISTGKCDLD |
| | | GAKELAKEVEELRQEAEKRGIDVRDLASNLKVILLELA (SEQ ID NO: 111) |
| G1 neo2_42 | H1->H4- | STKKIQLQLEHALLDVQMALNRSPEPNESLNRMITWLQSWISTGKIDLD |
| | >H2′- | NAQEMAKEAEKIRKEMEKRGIDVPDLISNIIVILLELS (SEQ ID NO: 20) |
| | >H3 | STKKIQLQLEHALLDVQMALNRSPEPNESLNRMITWLQSWISTGKIDLD |
| | | NAQEMANEAEKIRKEMEKRGIDVRDLISNIIVILLELS (SEQ ID NO: 112) |
| G1_neo2_43 | H1->H4- | STKKIQLQLEHALLDVQMALNRSPEPNESLNRMITWLQSCISTGKCDLD |
| | >H2′- | NAQEMAKEAEKIRKEMEKRGIDVRDLISNIIVILLELS (SEQ ID NO: 21) |
| | >H3 | STKKIQLQLEHALLDVQMALNRSPEPNESLNRMITWLQSCISTGKCDLD |
| | | NAQEMAKEAEKIRKEMEKRGIDVRDLISNIIVILLELS (SEQ ID NO: 113) |
| G1_neo2_44 | H1->H4- | STKKIQLQLEHALLDVQMALNRSPEPNESLNRMITWLQSWISTGKIDLD |
| | >H2′- | NAQEMCKEAEKIRKEMEKRGIDVRDLISNICVILLELS (SEQ ID NO: 22) |
| | >H3 | STKKIQLQLEHALLDVQMALNRSPEPNESLNRMITWLQSWISTGKIDLD |
| | | NAQEMCKEAEKIRKEMEKRGIDVRDLISNICVILLELS (SEQ ID NO: 114) |
| G1_neo2_40_1A | H1->H4- | STKKTQLLAEHALLDAFMMLNVVPEPNEKLNRIITTMQSWIYTGKIDAD |
| | >H2′- | GAKELAKEVEELEQEYEKRGIDVEDDASNLKVILLELA (SEQ ID NO: 23) |
| | >H3 | STKKTQLLAERALLDAFMMLNVVPEPNEKLNRIITTMQSWIYTGKIDAD |
| | | GAKELAKEVEELEQEYEKRGIDVEDDASNLKVILLELA (SEQ ID NO: 115) |
| G1_neo2_40_1B | H1->H4- | STKKTQLLAEHALLDAHMMLNMLPEPNEKLNRIITTMQSWIHTGKIDGD |
| | >H2′- | GAQELAKEVEELEQEYEKRGIDVEDEASNLKVILLELA (SEQ ID NO: 24) |
| | >H3 | STKKTQLLAEHALLDAHMMLMMLPEPNEKLNRIITTMQSWIHTGKIDGD |
| | | GAQELAKEVEELEQEYEKRGIDVEDEASNLKVILLELA (SEQ ID NO: 116) |
| G1_neo2_40_1C | H1->H4- | STKKTQLLAERALLDAFMMLNMVPEPNEKLNRIITTMQSWIFTGKIDGD |
| | >H2′- | GAKELAKEVEELEQEFEKRGIDVEDEASNLKVILLELA (SEQ ID NO: 25) |
| | >H3 | STKKTQLLAERALLDAFMMLNMVPEPNEKLNRIITTMQSWIFTGKIDGD |
| | | GAKELAKEVEELEQEFEKRGIDVEDEASNLKVILLELA (SEQ ID NO: 117) |
| G1_neo2_40_1D | H1->H4- | STKKTQLLAEHALLDALMMLNMVPEPNEKLNRIITTMQSWIFTGKIDGD |
| | >H2′- | GAQELAKEVEELEQELEKRGIDVEDYASNLKVILLELA (SEQ ID NO: 26) |
| | >H3 | STKKTQLLAEHALLDALMMLNMVPEPNEKLNRIITTMQSWIFTGKIDGD |
| | | GAQELAKEVEELEQELEKRGIDVEDYASNLKVILLELA (SEQ ID NO: 118) |
| G1_neo2_40_1E | H1->H4- | STKKTQLLAEHALLDALMMLNMVPEPNEKLNRIITTMQSWIFTGKIDGD |
| | >H2′- | GAQELAKEVEELEQELEKRGIDVEDYASNLKVILLELA (SEQ ID NO: 27) |
| | >H3 | STKKTQLLAEHALLDAHMMLNVVPEPNEKLNRIITTMQSWIYTGKIDRD |
| | | GAQELAKEVEELEQELEKRGIDVDDDASNLKVILLELA (SEQ ID NO: 119) |
| G1_neo2_40_1F | H1->H4- | STKKTQLLAEHALLDALMMLNLLPEPNEKLNRIITTMQSWIFTGKIDGD |
| | >H2′- | GAQELAKEVEELEQEHEKRGIDVEDYASNLKVILLELA (SEQ ID NO: 28) |
| | >H3 | STKKTQLLAEHALLDALMMLNLLPEPNEKLNRIITTMQSWIFTGKIDGD |
| | | GAQELAKEVEELEQEHEKRGIDVEDYASNLKVILLELA (SEQ ID NO: 120) |
| G1_neo2_40_1G | H1->H4- | STKKTQLLAEHALLDAYMMLNMVPEPNEKLNRIITTMQSWILTGKIDSD |
| | >H2′- | GAQELAKEVEELEQELEKRGIDVDDDASNLKVILLELA (SEQ ID NO: 29) |
| | >H3 | STKKTQLLAEHALLDAYMMLNNVPEPNEKLNRIITTMQSWILTGKIDSD |
| | | GAQELAKEVEELEQELEKRGIDVDDDASNLKVILLELA (SEQ ID NO: 121) |
| G1_neo2_40_1H | H1->H4- | STKKTHLLAEHALLDAYMMLNVMPEPNEKLNRIITTMQSWIFTGKIDGD |
| | >H2′- | GAKELAKEVEELEQEFEKRGIDVDDDASNLKVILLELA (SEQ ID NO: 30) |
| | >H3 | STKKTHLLAEHALLDAYMMLNVMPEPNEKLNRIITTMQSWIFTGKIDGD |
| | | GAKELAKEVEELEQEFEKRGIDVDDDASNLKVILLELA (SEQ ID NO: 122) |
| Gl_neo2_40_1I | H1->H4- | STKKTQLLAEHALLDAYMMLNLVPEPNEKLNRIITTMQSWIFTGKIDAD |
| | >H2′- | GAQELAIEVEELEQEYEKRGIDVDDYASNLKVILLELA (SEQ ID NO: 31) |
| | >H3 | STKKTQLLAEHALLDAYMMLNLVPEPNEKLNRIITTMQSWIFTGKIDAD |
| | | GAQELAIEVEELEQEYEKRGIDVDDYASNLKVILLELA (SEQ ID NO: 123) |
| G1_neo2_40_1J | H1->H4- | STKKTQLMAEHALLDAFMMLNVLPEPNEKLNRIITTMQSWIFTGKIDGD |
| | >H2′- | DAQELAKEVEELEQELEKRGIDVDDDASNLKVILLELA (SEQ ID NO: 32) |
| | >H3 | STKKTQLMAEHALLDAFMMLNVLPEPNEKLNRIITTMQSWIFTGKIDGD |
| | | DAQELAKEVEELEQELEKRGIDVDDDASNLKVILLELA (SEQ ID NO: 124) |
| G1_neo2_40_1F_H1 | H1->H4- | STKKTQLLIEHALLDALDMSRNLPEPNEKLSRIITTMQSWIFTGKIDGD |
| | >H2′- | GAQQLAKEVEELEQEHEKRGEDVEDEASNLKVILLELA (SEQ ID NO: 33) |
| | >H3 | STKKTQLLIEHALLDALDMSRNLPEPNEKLSRIITTMQSWIFTGKIDGD |
| | | GAQQLAKEVEELEQEHEKRGEDVEDEASNLKVILLELA (SEQ ID NO: 125) |
| G1_neo2_40_1F_H2 | H1->H4- | STKKTQLLLEHALLDALHMRRNLPEPNEKLSRIITTMQSWIFTGKIDGD |
| | >H2′- | GAQELAKEVEELEQEHEKRGRDVEDDASNLKVILLELA (SEQ ID NO: 34) |
| | >H3 | STKKTQLLLEHALLDALHMRRNLPEPNEKLSRIITTMQSWIFTGKIDGD |
| | | GAQELAKEVEELEQEHEKRGRDVEDDASNLKVILLELA (SEQ ID NO: 126) |
| G1_neo2_40_1FH3 | H1->H4- | STKKTQLLIEHALLDALNMRKKLPEPNEKLSRIITDMQSWIFTGKIDGD |
| | >H2′- | GAQQLAKEVEELEQEHEKRGGDVEDYASNLKVILLELA (SEQ ID NO: 35) |
| | >H3 | STKKTQLLIEHALLDALNMRKKLPEPNEKLSRIITDMQSWIFTGKIDGD |
| | | GAQQLAKEVEELEQEHEKRGGDVEDYASNLKVILLELA (SEQ ID NO: 127) |
| G1_neo2_40_1F_H4 | H1->H4- | STKKTQLLLEHALLDALHMSRELPEPNEKLNRIITDMQSWIFTGKIDGD |
| | >H2′- | GAQDLAKEVEELEQEHEKRGGDVEDYASNLKVILLELA (SEQ ID NO: 36) |
| | >H3 | STKKTQLLLEHALLDALHMSRELPEPNEKLNRIITDMQSWIFTGKIDGD |
| | | GAQDLAKEVEELEQEHEKRGGDVEDYASNLKVILLELA (SEQ ID NO: 128) |
| G1_neo2_40_1F_H5 | H1->H4- | STKKTQLLIEHALLDALHMSRKLPEPNEKLSRIITTMQSWIFTGKIDGD |
| | >H2′- | GAQHLAKEVEELEQEHEKRGGEVEDEASNLKVILLELA (SEQ ID NO: 37) |
| | >H3 | STKKTQLLIEHALLDALHMSRKLPEPNEKLSRIITTMQSWIFTGKIDGD |
| | | GAQHLAKEVEELEQEHEKRGGEVEDEASNLKVILLELA (SEQ ID NO: 129) |
| Gl_neo2_40_1F_H6 | H1->H4- | STKKTQLLIEHALLDALHMKRKLPEPNEKLNRIITNMQSWIFTEKIDGD |
| | >H2′- | GAQDLAKEVEELEQEHEKRGQDVEDYASNLKVILLELA (SEQ ID NO: 38) |
| | >H3 | STKKTQLLIEHALLDALHMKRKLPEPNEKLNRIITNMQSWIFTEKIDGD |
| | | GAQDLAKEVEELEQEHEKRGQDVEDYASNLKVILLELA (SEQ ID NO: 130) |
| G1_neo2_40_1F_M1 | H1->H4- | STEKTQLAAEHALRDALMLKHLLNEPNEKLARIITTMQSWQFTGKIDGD |
| | >H2′- | GAQELAKEVEELQQEHEVRGIDVEDYASNLKVILLHLA (SEQ ID NO: 39) |
| | >H3 | STEKTQLAAEHALRDALMLKHLLNEPNEKLARIITTMQSWQFTGKIDGD |
| | | GAQELAKEVEELQQEHEVRGIDVEDYASNIKVILLHLA (SEQ ID NO: 131) |
| G1_neo2_40_1F_M2 | H1->H4- | STKNTQLAAEDALLDALMLRNLLNEPNEKLARIITTMQSWQFTEKIDGD |
| | >H2′- | GAQELAKEVEELQQEHEERGIDVEDYASNLKVILLQLA (SEQ ID NO: 40) |
| | >H3 | STKNTQLAAEDALLDALMLRNLLNEPNEKLARIITTMQSWQFTEKIDGD |
| | | GAQELAKEVEELQQEHEERGIDVEDYASNLKVILLQLA (SEQ ID NO: 132) |
| G1_neo2_40_1FM3 | H1->H4- | STEKTQHAAEDALRDALMLRNLLNEPNEKLARIITTMQSWQFTEKIDGD |
| | >H2′- | GAQELAKEVEELQQEHEVRGIDVEDYASNLKVILLQLA (SEQ ID NO: 41) |
| | >H3 | STEKTQHAAEDALRDALMLRNLLNEPNEKLARIITTMQSWQFTEKIDGD |
| | | GAQELAKEVEELQQEHEVRGIDVEDYASNLKVILLQLA (SEQ ID NO: 133) |
| G1_neo2_40_1F_seq02 | H1->H4- | TQKKQQLLAEHALLDALMILNMLKTSSEAVNRMITIAQSWIFTGTSNPE |
| | >H2′- | EAKEMIKMAEQAEEEARPEGVDTEDYVSNLNVILKEIA (SEQ ID NO: 42) |
| | >H3 | TQKKQQLLAEHALLDALMILNMLKTSSEAVNRMITIAQSWIFTGTSNPE |
| | | EAKEMIKMAEQAEEEARREGVDTEDYVSNLKVILKEIA (SEQ ID NO: 134) |
| G2_neo2_40_1F_seq03 | H1->H4- | TTKKYQLLVEHALLDALMMLNLSSESNEKMNRIITTMQSWIFTGTFDPD |
| | >H2′- | QAEELAKLVEELREEFRKRGIDTEDYASNLKVILKELS (SEQ ID NO: 43) |
| | >H3 | TTKKYQLLVEHALLDALMMLNLSSESNEKMNPIITTMQSWIFTGTFDPD |
| | | QAEELAKLVEELREEFRKRGIDTEDYASNLKVILKELS (SEQ ID NO: 135) |
| G2_neo2_40_1F_seq04 | H1->H4- | TTKKIQLLVEHALLDALMILNLSSESNEKLNRIITTLQSWIFRGEIDPD |
| | >H2′- | RAPELAKILELIKEEMRKRGIDTEDYVSNMIVIIRELA (SEQ ID NO: 44) |
| | >H3 | TTKKIQLLVEHALLDALMILNLSSESNEKLNRIITTLQSWIFRGEIDPD |
| | | RARELAKLLEEIREEMRKRGIDTEDYVSNMIVIIRELA SEQ ID NO: 136) |
| G2_neo2_40_1F_seq05 | H1->H4- | TKKKIQLLAEHVLLDLEMMLNLSSESNEKMNPLITIVQSWIFTGTIDPD |
| | >H2′- | QAEEMAKWVEELREEFRKRGIDTEDYASNVKVILKELS (SEQ ID NO: 45) |
| | >H3 | TKKKIQLLAEHVLLDLLMMLNLSSESNEKMNRLITIVQSWIFTGTIDPD |
| | | QAEEMAKWVEELREEFRKRGIDTEDYASNVKVILKELS (SEQ ID NO: 137) |
| G2_neo2_40_1E_seq06 | H1->H4- | TKKWYQLLIEHLLLDALMVLNMSSESNEKLNRIITILQSWIFTGTWDPD |
| | >H2′- | LAEEMEKLMQEIEEELRPRGIDTEDYMSNMRVIIKELS (SEQ ID NO: 46) |
| | >H3 | TKKKYQLLIEHLLLDALMVLNMSSESNEKLNRIITILQSWIKTGTWDPD |
| | | LAEEMEKLMQEIEEELRRRGIDTEDYMSNMRVIIKELS (SEQ ID NO: 138) |
| G2_neo2_40_1F_seq07 | H1->H4- | TKKKLQLLVEHLLLDMLMILNMSSESNEKLNRLITELQSWIFRGEIDPD |
| | >H2′- | KAEEMWKIMEEIEKELRERGIDTEDYMSNAKVIIKELS (SEQ ID NO: 47) |
| | >H3 | TKKKLQLLVEHLLLDMLMILNMSSESNEKLNRLITELQSWIFRGEIDPD |
| | | KAEEMWKIMEEIEKELRERGIDTEDYMSNAKVIIKELS (SEQ ID NO: 139) |
| G2_neo2_40_1F_seq08 | H1->H4- | TSKKQQLLAEHALLDALMILNISSESSEAVNRAITWLQSWIFKGTVNPD |
| | >H2′- | QAEEMRKLAEQIREEMRKRGIDTEDYVSNLEVIAKELS (SEQ ID NO: 48) |
| | >H3 | TSKKQQLLAEHALLDALMILNISSESSEAVNRAITWLQSWIFKGTVNPD |
| | | QAEEMRKLAEQIREEMRKRGIDTEDYVSNLEVIAKELS (SEQ ID NO: 140) |
| G2_neo2_40_1F_seq09 | H1->H4- | TKKKYQLLIEHLLLDLLMVINMSSESNEKINRLITWLQSWIFTGTYDPD |
| | >H2′- | LAEEMYKILEELREEMRERGIDTEDYMSNMRVIVKELS (SEQ ID NO: 49) |
| | >H3 | TKKKYQLLIEHLLLDLLMVLNMSSESNEKINRLITWLQSWIFTGTYDPD |
| | | LAEEMYKILEELREEMRERGIDTEDYMSNMRVIVKELS (SEQ ID NO: 141) |
| G2_neo2_40_1E_seq10 | H1->H4- | TKKKWQLLIEHLLLDLLMILNLSSESNEKLNRLITWLQSWIFTGTYDPD |
| | >H2′- | LAEEMKKMMDEIEDELRERGIDTEDYMSNAKVIIKELS (SEQ ID NO: 50) |
| | >H3 | TKKKWQLLIEHLLLDLLMILNLSSESNEKLNRLITWLQSWIFTGTYDPD |
| | | LAEEMKKMMDEIEDELRERGIDTEDYMSNAKVIIKELS (SEQ ID NO: 142) |
| G2_neo2_40_1F_seq11 | H1->H4- | TKKKIQLLVEHALLDALMILNLSSESNEKLNRIITTMQSWIFTGTIDPD |
| | >H2′- | QAEELSKLVEEIREEMRKRGIDTEDYVSNLKVILDELS (SEQ ID NO: 51) |
| | >H3 | TKKKIQLLVEHALLDALMILNLSSESNEKLNRIITTMQSWIFTGTIDPD |
| | | QAEELSKLVEEIREEHPKRGIDTEDYVSNLKVILDELS (SEQ ID NO: 143) |
| G2_neo2_40_1E_seq12 | H1->H4- | TEKKLQLLVEHALLDALMILNLWSESNEKLNRIITTMQSWIFTGRIDPD |
| | >H2′- | KAEELAKLVEELREEARERGIDTEDYVSNDKVILKEDS (SEQ ID NO: 52) |
| | >H3 | TEKKLQLLVEHALLDALMILNLWSESNEKLNPIITTMQSWIFTGRIDPD |
| | | KAEELAKLVEELREEARERGIDTEDYVSNLKVILKELS (SEQ ID NO: 144) |
| G2_neo2_40_1F_seq13 | H1->H4- | TKKKYQLLMEHLLLDLLMVLNMSSESNEKLNRLITIIQSWIFTGTWDPD |
| | >H2′- | KAKEEMAMLKEIEDELRERGIDTEDYMSNMIVIMKELS (SEQ ID NO: 53) |
| | >H3 | TKKKYQLLMEHLLLDLLMVLNMSSESNEKLNRLITIIQSWIFTGTWDPD |
| | | KAEEMAKMLNEIEDELRERGIDTEDYMSNMIVIMKELS (SEQ ID NO: 145) |
| G2_neo2_40_1F_seq14 | H1->H4- | TTKKIQLLVEHALLDALMLLNLSSESNEKMNRIITTMQSWIFEGRIDPD |
| | >H2′- | QAQELAKLVEELREEFRKRGIDTEDYVSNLKVILEELS (SEQ ID NO: 54) |
| | >H3 | TTKKIQLLVEHALLDALMLLNESSESNEKMNRIITTMQSWIFEGRIDPD |
| | | QAQELAKLVEELREEFRKRGIDTEDYVSNLKVILEELS (SEQ ID NO: 146) |
| G2_neo20_40-1F_seq15 | H1->H4- | TKKKIQLLVEHALLDALMMLNLSSESNEKLNRIITTMQSWIFTGTIDPD |
| | >H2′- | QAEELAKLVRELREEFRKRGIDTEDYASNLEVILRELS (SEQ ID NO: 55) |
| | >H3 | TKKKIQLLVEHALLDALMMLNLSSESNEKLNRIITTMQSWIFTGTIDPD |
| | | QAEELAKLVRELREEFRKRGIDTEDYASNLEVILRELS (SEQ ID NO: 147) |
| G2_neo2_40_1F_seq16 | H1->H4- | TKKKIQLLVEHALLDALMILNLSSKSNEKLNRIITTMQSWIFNGTIDPD |
| | >H2′- | RARELAKLVEEIRDEMEKNGIDTEDYVSNLKVILEELA (SEQ ID NO: 56) |
| | >H3 | TKKKIQLLVEHALLDALMILNLSSKSNEKLNRIITTMQSWIFNGTIDPD |
| | | RARELAKLVEEIRDEMEKNGIDTEDYVSNLKVILEELA (SEQ ID NO: 148) |
| G2_neo2_40_1F_seq17 | H1->H4- | TKKKYQLLIEHVLLDLLMLLNLSSESNEKMNRLITILQSWIFTGTYDPD |
| | >H2′- | KAEEMAKLLKELREEFRERGIDTEDYISNAIVILKELS (SEQ ID NO: 57) |
| | >H3 | TKKKYQLLIEHVLLDLLMLLNLSSESNEKMNRLITILQSWIFTGTYDPD |
| | | RAEEMAKLLKELPEEFRERGIDTEDYISNAIVILKELS (SEQ ID NO: 149) |
| G2_neo2_40_1F_seq18 | H1->H4- | TKKKIQLLVEHALLDALMMLNLSSESNEKLNRIITTMQSWIFTGTIDPD |
| | >H2′- | RAEELAKLVEELREEFRKRGIDTEDYASNLKVILKELS (SEQ ID NO: 58) |
| | >H3 | TKKKIQLLVEHALLDALMMLNLSSESNEKLNRIITTMQSWIFTGTIDPD |
| | | RAEELAKLVEELREEFRKRGIDTEDYASNLKVILKELS (SEQ ID NO: 150) |
| G2_neo2_40_1F_seq19 | H1->H4- | TKKKIQLLVEHALLDALMMLNLSSESNEKLNRIITTMQSWIFNGTIDPD |
| | >H2′- | QARELAKLVEELREEFRKRGIDTEDYASNLKVILEELA (SEQ ID NO: 59) |
| | >H3 | TKKKIQLLVEHALLDALMMLNLSSESNEKLNRIITTMQSWIFNGTIDPD |
| | | QARELAKLVEELREEFRKRGIDTEDYASNLKVILEELA (SEQ ID NO: 151) |
| G2_neo2_40_1F_seq20 | H1->H4- | TKKNLQLLVEHALLDALMLLNLSSESNEKLNRIITTMQSWIFTGTVDPD |
| | >H2′- | QAEELAKLVEEIREELRKRGIDTEDYVSNLKVILKELS (SEQ ID NO: 60) |
| | >H3 | TKKKLQLLVEHALLDALMLLNLSSESNEKLNRIITTMQSWIFTGTVDPD |
| | | QAEELAKLVEEIREELRKRGIDTEDYVSNLKVILKELS (SEQ ID NO: 152) |
| G2_ne02_40_1F_seq21 | H1->H4- | TTKKYQLLVEHALLDALMILNLSSESNEKLNRIITTMQSWIFTGTFDPD |
| | >H2′- | QAEELAKLVREIREEMRKRGIDTEDYVSNLEVILRELS (SEQ ID NO: 61) |
| | >H3 | TTKKYQLLVEHALLDALMILNLSSESNEKLNRIITTMQSWIFTGTFDPD |
| | | QAEELAKLVREIREEMRKRGIDTEDYVSNLEVILRELS (SEQ ID NO: 153) |
| G2_neo2_40_1F_seq22 | H1->H4- | TKKKIQLLVEHALLDALMILNLSSESNEKLNRIITTMQSWIFTGTIDPD |
| | >H2′- | RAEELAKLVREIREEMRKRGIDTEDYVSNLEVILRELS (SEQ ID NO: 62) |
| | >H3 | TKKKIQLLVEHALLDALMILNLSSESNEKLNRIITTMQSWIFTGTIDPD |
| | | RAEELAKLVREIREEMRKAGIDTEDYVSNLEVILRELS (SEQ ID NO: 154) |
| G2_neo2_40_1F_seq23 | H1->H4- | TKKKYQLLIEHLLLDLLMILNLSSESNEKLNALITWLQSWIFRGEWDPD |
| | >H2′- | KAEEWAKILKEIREELRERGIDTEDYMSNAIVIMKELS (SEQ ID NO: 63) |
| | >H3 | TKKKYQLLIEHLLLDLLMILNLSSESNEKLNRLITWLQSWIFRGEWDPD |
| | | KAEEWAKILKEIREELRERGIDTEDYMSNAIVIMKELS (SEQ ID NO: 155) |
| G2_neo2_40_1F_seq24 | H1->H4- | TDKKLQLLVEHLLLDLLMMLNLSSKSNEKMNRLITIAQSWIFTGKVDPD |
| | >H2′- | LAREMIKLLEETEDENRKNGIDTEDYVSNARVIAKELE (SEQ ID NO: 64) |
| | >H3 | TDKNLQLLVEHLLLDLLMMLNLSSKSNEKMNRLITIAQSWIFTGKVDPD |
| | | LAREMIKLLEETEDENRKNGIDTEDYVSNARVIAKELE (SEQ ID NO: 156) |
| G2_neo2_40_1F_seq25 | H1->H4- | TKKKIQLLVEHALLDALMLLNLSSESNEKMNRIITTMQSWIFTGTIDPD |
| | >H2′- | QAEELAKLVEELKEEFKKRGIDTEDYVSNLKVILKELS (SEQ ID NO: 65) |
| | >H3 | TKKKIQLLVEHALLDALMLLNLSSESNEKMNRIITTMQSWIFTGTIDPD |
| | | QAEELAKLVEELKEEFKKRGIDTEDYVSNLKVILKELS (SEQ ID NO: 157) |
| G2_neo2_40_1F_seq26 | H1->H4- | TKKNYQLLIEHALLDALMILNLWSESNEKLNRIITTMQSWIFTGTYDPD |
| | >H2′- | KAEELEKLAKEIEDEARERGIDTEDYMSNLRVILKELS (SEQ ID NO: 66) |
| | >H3 | TKKKYQLLIEHALLDALMILNLWSESNEKLNPIITTMQSWIFIGTYDPD |
| | | KAEELEKLAKEIEDEARERGIDTEDYMSNLRVILKELS (SEQ ID NO: 158) |
| G2_neo2_40_1F_seq27 | H1->H4- | TKKKAQLLAEHALLDALMLLNLSSESNERLNRIITWLQSIIFTGTYDPD |
| | >H2′- | MVKEAVKLADEIEDEMRKRGIDTEDYVSNLRVILQELA (SEQ ID NO: 67) |
| | >H3 | TKKKAQLLAEHALLDALMLLNLSSESNERLNRIITWLQSIIFTGTYDPD |
| | | MVKEAVKLADEIEDEMRKRGIDTEDYVSNLRVILQELA (SEQ ID NO: 159) |
| G2_neo2_40_1F_seq28 | H1->H4- | TQKKNQLLAEHLLLDALMVLNQSSESSEVANRIITWAQSWIFEGRVDPN |
| | >H2′- | KAEEAKKLAKKLEEEMRKRGIDMEDYISNMKVIAEEMS (SEQ ID NO: 68) |
| | >H3 | TQKKNQLLAEHLLLDALMVLNQSSESSEVANRIITWAQSWIFEGRVDPN |
| | | KAEEAKKLAKKLEEEMRKRGIDMEDYISNMKVIAEEMS (SEQ ID NO: 160) |
| G2_neo2_40_1F_seq29 | H3- | EDYYSNLKVILEELREAMERNGLSDKAEEWRQWKKIVERIRQIRSNNSD |
| | >H2′- | LNEAKELLNRLITYIQSQIFEISERIRETDQEKKEESWKKWQLLLEHAL |
| | >H4->H1 | LDVLMLLND (SEQ ID NO: 69) |
| | | EDYYSNLKVILEELAREMERNGLSDKAEEWRQWKKIVERIRQIRSNNSD |
| | | LNEAKELLNRLITYIQSQIFEISERIRETDQEKKEESWKKWQLLLEHAL |
| | | LDVLMLLND (SEQ ID NO: 161) |
| G2_neo2_40_1F_seq30 | H1->H3- | PEKKRQLLLEHILLDALMLLNLXXXXXXNTESKFEDYISNAEVIAEELA |
| | >H2′- | KLMESXXLSDEAEKKKKIKQWLREVWRIWXXXXWSTLEDKAPELLNRII |
| | >H4 | TTIQSQIFY (SEQ ID NO: 70) |
| | | PEKKRQLLLEHILLDALMLLNLLETNPQNTESKFEDYISNAEVIAEELA |
| | | KLMESLGLSDEAEKKKKIKQWLREVWRIWSSTNWSTLEDKARELLNRII |
| | | TTIQSQIFY (SEQ ID NO: 162) |
| G2_neo2_40_1F_seq31 | H1->H3- | PEKKRQLLLEHILLDLLMILNMXXXXXXNTESEMEDYWSNVRVILRELA |
| | >H2′- | RLMEEXXXKELSELMERMRKIVEKIRQIVTXXXXLDTAREWLNRLITWI |
| | >H4 | QSLIFR (SEQ ID NO: 71) |
| | | PEKKRQLLLEHILLDLLMILNMIETNRENTESEMEDYQSNVRVILRELA |
| | | RLMEELNYKELSELMERMRKIVEKIRQIVTNNSSLDTAREWLNRLITWI |
| | | QSLIFR (SEQ ID NO: 163) |
| G2_neo2_40_1F_seq32 | H1->H3- | PEKKRQLLAEHALLDALMLLNIIETNSKNTESKMEDYVSNLEVILTEFK |
| | >H2′- | KLAEKLNFSEEAERAERMKRWARKAYQMMTLDLSLDKAKEMLNRIITIL |
| | >H4 | QSIIFN (SEQ ID NO: 72) |
| | | PEKKRQLLAEHALLDALMLLNIIETNSKNTESKMEDYVSNLEVILTEFK |
| | | KLAEKLNFSEEAERAERMKRWARNAYQMMTLDLSLDKAKEMLNRIITIL |
| | | QSIIFN (SEQ ID NO: 164) |
| G2_neo2_40_1F_seq33 | H1->H3- | PEKKRQLLAEHLLLDVLMMLNGNASLKDYASNAQVIADEFRELARELGL |
| | >H2′- | TDEAKKAEKIIEALERAREWLLNNKDKEKAKEALNRAITIAQSWIKN |
| | >H4 | (SEQ ID NO: 73) |
| | | PEKKRQLLAEHLLLDVLMMLNGNASLKDYASNAQVIADEFRELARELGL |
| | | TDEAKKAEKIIEALERAREWLLNNKDKEKAKEALNRAITIAQSWIFN |
| | | (SEQ ID NO: 165) |
| G2_neo2_40_1F_seq34 | H1->H3- | PEKKRQLLLEHLLLDLLMILNMLRTNPKNIESDWEDYMSNIEVIIEELR |
| | >H2′- | KIMESLGRSEKAKEWKRMKQWVRRILEIVKNNSDLEEAKEWLNRLITIV |
| | >H4 | QSEIFE (SEQ ID NO: 74) |
| | | PEEKRQLLLEHLLLDLLMILNMLRTNPKNIESDWEDYMSNIEVIIEELR |
| | | KIMESLGRSEKAKEWKRMKQWVRRILEIVKNNSDLEEAKEWLNRLITIV |
| | | QSEIFE (SEQ ID NO: 166) |
| G2_neo2_40_1F_seq35 | H1->H3 | WEKKRQLLLEHLLLDLLMILNMWRTNPQNTESLMEDYMSNAKVIVEELA |
| | >H2′- | PMMRSQGLEDKAREWEEMKKRIEEIRQIIQNNSSKERAKEELNRLITYV |
| | >H4 | QSEIFR (SEQ ID NO: 75) |
| | | WEKKRQLLLEHLLLDLLMILNMWRTNPQNTESLMEDYMSNAKVIVEELA |
| | | RMMRSQGLEDKAREWEEMKKRIEEIRQIIQNNSSKERKEELNRLITYVQ |
| | | SEIFR (SEQ ID NO: 167) |
| G2_neo2_40_1F_seq36 | H1->H3- | PKKKIQLLAEHALLDALMILNIVKTNSQNAEEKLEDYASNVEVILEEIA |
| | >H2′- | RLMESGDQKDEAEKAKRMKEWMKRIKTTASEDEQEEMANRIITLLQSWI |
| | >H4 | FS (SEQ ID NO: 76) |
| | | PKKKIQLLAEHALLDALMILNIVKTNSQNAEEKLEDYASNVEVILEEIA |
| | | RLMESGDQKDEAEKAKRMKEWMKRIKTTASEDEQEEMANRIITLLQSWI |
| | | FS (SEQ ID NO: 168) |
| G2_neo2_40_1F_seq37 | H1->H3- | PEKKRQKLLAEHALLDALMILNXXXXXXQNAEEKLEDYMSNVEVIMEEF |
| | >H2′- | ARMMRXXXXEEAENAERIKKWVRKASSXXXSEEQREMMNRAITLMQSWI |
| | >H4 | FE (SEQ ID NO: 77) |
| | | PEKKRQLLAEHALLDALMILNILQTNPQNAEEKLEDYMSNVEVIMEEFA |
| | | RMMRNGDRSEEAENAERIKKWVRKASSTASSEEQREMMNRAITLMQSWI |
| | | FE (SEQ ID NO: 169) |
| G2_neo2_40_1F_seq38 | H1->H3- | PEKKRQLLAEHLLLDALMVLNMXXXXXXNTEEKLEDYISNMKVIIKEMI |
| | >H2′- | ELMRSLXXXEEAEKWKEALKAVEKIXXXXDSETARELANRIITLAQSAI |
| | >H4 | FY (SEQ ID NO: 78) |
| | | PEKKRQLLAEHLLLDALMVLNMLTTNSKNTEEKLEDYISNMKVIIKEMI |
| | | ELMRSLGRLEEAEKWKEALKAVEKIGSRMDSETARELANRIITLAQSAI |
| | | FY (SEQ ID NO: 170) |
| G2_neo2_40_1F_seq39 | H1->H3- | PEKKRQLLAEHALLDALMFLNLXXXXXXQAEEKIEDYASNLRVIAEELA |
| | >H2′- | RLFENLXXXDEAQKAKDIKELAERARSXXSSEKRKEAMNRAITILQSMI |
| | >H4 | FR (SEQ ID NO: 79) |
| | | PEKKRQLLAEHALLDALMFLNLVETNPDQAEEKIEDYASNLRVIAEELA |
| | | RLFENLGRLDEAQKAKDIKELAEPARSRVSSEKRKEAMNRAITILQSMI |
| | | FR (SEQ ID NO: 171) |
| G2_neo2_40_1F_seq40 | H1->H3- | PEKKRQLLAEHALLDALMILNIIRTNSDNTESKLEDYISNLKVILEEIA |
| | >H2′- | RLMESLGLSDEAEKAKEAMRLADKAGSTASEEEKKEAMNRVITWAQQWI |
| | >H4 | FN (SEQ ID NO: 80) |
| | | PEKKRQLLAEHALLDALMILNIIRTNSDNTESKLEDYISNLKVILEEIA |
| | | RLMESLGLSDEAEKAKEAMRLADKAGSTASEEEKKEAMNRVITWAQSWI |
| | | FN (SEQ ID NO: 172) |
| G2_neo2_40_1F_seq41 | H1->H3- | PEKKRQLLAEHALLDALMMLNILRTNPDNAEEKLEDYWSNLIVILREIA |
| | >H2′- | KLMESLGLTDEAEKAKEAARWAEEARTTASKDQRRELANRIITLLQSWI |
| | >H4 | FS (SEQ ID NO: 81) |
| | | PEKKRQLLAEHALLDALMMLNILRTNPDNAEEKLEDYWSNLIVILREIA |
| | | KLMESLGLTDEAEKAKEAARWAEEARTTASKDQRRELANRIITLLQSWI |
| | | FS (SEQ ID NO: 173) |
| G2_neo2_40_1F_seq42 | H1->H3- | PEKKRQLLAEHLLLDALMILNIIETNEQNAESKLEDYISNAKVILDEFR |
| | >H2′- | EMARDLGLLDEAKKAEKMKRWLEKMRSNASSDERREWANRMITTAQSWI |
| | >H4 | FN (SEQ ID NO: 82) |
| | | PEKKRQLLAEHLLLDALMILNIIETNEQNAESKLEDYISNAKVILDEFR |
| | | EMARDLGLLDEAKKAEKMKRWLEKMRSNASSDERREWANRMITTAQSWI |
| | | FN (SEQ ID NO: 174) |
| G2_neo2_40_1F_seq27_S3 | H1->H4- | TNKKAQLHAEFALHDALMLLNLSSESNERLNRIITWLQSIIFYGTYDPD |
| | >H2′- | MVKEAVKDADEIEDEMRKRGIDTEDYVSNLRLILQELA |
| | >H3 | (SEQ ID NO: 83) |
| | | TNKKAQLHAEFALHDALMLLNLSSESNERLNRIITWLQSIIFYGTYDPD |
| | | MVKEAVKDADEIEDEMRKRGIDTEDYVSNLRLILQELA |
| | | (SEQ ID NO: 245) |
| G2_neo2_40_1F_seq27_S18 | H1->H4- | TNKEAQLHAEFALYDALMLLNLSSESNERLNRIITWLQSIIFYETYDPD |
| | >H2′- | MVKEAVKLADEIEDEMRKRKIDTEDYVVNLRLILQELA |
| | >H3 | (SEQ ID NO: 84) |
| | | TNKEAQLHAEFALYDALMLLNLSSESNERLNRIITWLQSIIFYETYDPD |
| | | MVKEAVKLADEIEDEMRKRKIDTEDYVVNLRLILQELA |
| | | (SEQ ID NO: 175) |
| G2-neo2_40_1F_seq27_S22 | H1->H4- | TKKDAELLAEFALYDALMILNLSSESNERLNEIITWLQSIIFYGTYDPD |
| | >H2′- | MVKEAVKLADEIEDEMRKRGIDTEDYVSNLRLILQELA |
| | >H3 | (SEQ ID NO: 85) |
| | | TKEDAELLAEFALYDALMLLNLSSESNERLNEIITWLQSIIFYGTYDPDM |
| | | VKEAVKLADEIEDEMRKRGIDTEDYVSNLRLILQELA |
| | | (SEQ ID NO: 176) |
| G2_neo2_40_1F_seq27_S24 | H1->H4- | TNKKAQLHAEFALYDALMLLNLSSESNERLNDIITWLQSIIFTGTYDPDM |
| | >H2′- | VKEAVKLADEIEDEMRKRKIDTEDYVVNLRYILQELA |
| | >H3 | (SEQ ID NO: 86) |
| | | TNKKAQLHAEFALYDALMLLNLSSESNERLNDIITWLQSIIFTGTYDPDM |
| | | VKEAVKLADEIEDEMRKPRIDTEDYVVNLRYILQELA |
| | | (SEQ ID NO: 177) |
| G2_neo2_40_1F_seq29_S6 | H3- | EDYYSNLKLILEELAREMERNGLSDKAEEWRQWKKIVERIRQIRSNNSDL |
| | >H2′- | NEAKELLNRLITYIQSQIFEVLHGVGETDQEKKEESWKKWDLLLEHALLD |
| | >H4->H1 | VLMLLND (SEQ ID NO: 87) |
| | | EDYYSNLKLILEELAREMERNGLSDKAEEWRQWKKIVERIRQIRSNNSDL |
| | | NEAKELLNRLITYIQSQIFEVLHGVGETDQEKKEESWKKWDLLLEHALLD |
| | | VLMLLND (SEQ ID NO: 178) |
| G2_neo2_40_1F_seq29_S7 | H3- | EDYYSNLKVILEELAREMERNGLSDKAEEWRQWKKIVERIRQIRSNNSDL |
| | >H2′- | NEAKELLNELITYIQSQIFEVIEPEGETDQEKKEESWKKWELHLEHALLD |
| | >H4->H1 | VLMLLND (SEQ ID NO: 138) |
| | | EDYYSNLKVILEELAREMERNGLSDKAEEWRQWKKIVERIRQIRSNNSDL |
| | | NEAKELLNELITYIQSQIFEVIEREGETDQEKKEESWKKWELHLEHALLD |
| | | VLMLLND (SEQ ID NO: 179) |
| G2_neo2_40_1F_seq29_S8 | H3- | EDYYSNLKLILEELAREMERNGLSDKAEEWRQWKKIVERIRQIRSNNSDL |
| | >H2′- | NEAKELLNRLITYIQSQIFEVLEGVGETDQEKKEESWKKWELHLEHALLD |
| | >H4->H1 | VLMLLND (SEQ ID NO: 89) |
| | | EDYYSNLKLILEELAREMERNGLSDKAEEWRQWKKIVERIRQIRSNNSDL |
| | | NEAKELLNRLITYIQSQIFEVLEGVGETDQEKKEESWKKWELHLEHALLD |
| | | VLMLLND (SEQ ID NO: 130) |
| Neoleukin-2/15 | H1-′H3- | PKKKIQLHAEHALYDALMILNIVKTNSPPAEEKLEDYAFNFELILEEIAR |
| (i.e. G2_neo2_40_1F_seq36_S11) | >H2′- | LFESGDQKDEAEKAKRMKEWMKRIKTTASEDEQEEMANAIITILQSWIFS |
| | >H4 | (SEQ ID NO: 90, version 1) |
| | | PEKKIQLHAEHALYEALMILNIVKTNSPPAEEKLEDYAFNFELILEEIAR |
| | | LFESGDQKDEAEKAKRMKEWMKRIKTTASEDEQEEMANAIITILQSWIFS |
| | | (SEQ ID NO: 90, version 2) |
| | | PKKKIQLHAEHALYDALMILNIVKTNSPPAEEKLEDYAFNFELILEEIAR |
| | | LFESGDQKDEAEKAKRMKEWMKRIKTTASEDEQEEMANAIITILQSWIFS |
| | | (SEQ ID NO: 181) |
| G2_neo2_40_1F_seq36_S12 | H1->H3- | PKKKIQLLAEHALFDLLMILNIVKTNSQNAEEKLEDYAYNAGVILEEIAR |
| | >H2′- | LFESGDQKDEAEKAKRMKEWMKRIKDTASEDEQEEMANEIITILQSWNFS |
| | >H4 | (SEQ ID NO: 91) |
| | | PKKKIQLLAEHALFDLLMILNIVKTNSQNAEEKLEDYAYNAGVILEEIAR |
| | | LFESGDQKDEAEKAKRMKEWMKRIKDTASEDEQEEMANEIITILQSWNFS |
| | | (SEQ ID NO: 182) |
| Neoleukin- | H1->H3- | PKKKIQLYAEHALYDALMILNIVKTNSPPAEETLEDYAFNFELILEEIAR |
| 2/15-H8Y-K33E | >H2′- | LFESGDQKDRAEKAKRMKEWMKRIKTTASEDEQEEMANAIITILQSWIFS |
| | >H4 | (SEQ ID NO: 94) |
| | | PKKKIQLYAEHALYDALMILNIVKTNSPPAEEELEDYAFNFELILEEIAR |
| | | LFESGDQKDEAEKAKRMKEWMKRIKTTASEDEQEEMANAIITILQSWIFS |
| | | (SEQ ID NO: 246) |
| Neoleukin-2/15(K32 | H1->H3- | PKKKIQLHAEHALYDALMILNIVKTNSPPAEEKLEDYAFNFELILEEIAR |
| is considered to be a | >H2′- | LFESGDQKDEAEKAKRMKEWMKRIKTTASEDEQEEMANAIITILQSWIFS |
| residue of the optional | >H4 | (SEQ ID NO: 247) |
| linker in this depicted | | |
| sequence) | | |

| | |
|---|---|
| IL4_G2_neo2_40_1F_seq36_S11 | PKKKIQITAEEALKDALSILNIVKTNSPPAEEQLERFAKRFERNLWGIARLFESGDQKDEAEK |
| | AKRMKEWMKRIKTTASEDEQEEMANAIITILQSWIFS (SEQ ID NO: 92) |
| | PKKKIQITAEEALKDALSILNIVKTNSPPAEEQLERFAKRFERNLWGIARLFESGDQKDEAEK |
| | AKRMKEWMKRIKTTASEDEQEEMANAIITILQSWIFS (SEQ ID NO: 183) |
| Neo1eukin-4(i.e. | PKKKIQIMAEEALKDAISILNIVKTNSPPAEEQLERFAERFERNLWGIARLFESGDQKDEAEK |
| IL4_G2_neo2_40_1F_seq36_S11_MIF) | AKRMIEWMKRIKTTASEDEQEEMANAIITILQSWFFS (SEQ ID NO: 93) |
| | PKKKIQIMAEEALKDALSILNIVKTNSPPAEEQLERFAKRFERNLWGIARLFESGDQKDEAEK |
| | AKRMIEWMKRIKTTASEDEQEEMANAIITILQSWFFS (SEQ ID NO: 184) |
| Neoleukin- | PKKKIQLHAEHALYDALMILNIXXXXXXXXXXXLEDYAFNFELILEEIACLFESGXXKDEAEK |
| 2/15_R50C | AKRMKEWMKRIKTXXXEDEQEEMANAIITILQSWIFS* (SEQ ID NO: 275) |
| Neoleukin- | PKKKIQLHAEHALYDALMILNIXXXXXXXXXXXLEDYAFNFELILEEIARLFCSGXXKDEAEK |
| 2/15_E53C | AKRMKEWMKRIKTXXXEDEQEEMANAIITILQSWIFS* (SEQ ID NO: 276) |
| Neoleukin- | PKKKIQLHAEHALYDALMILNIXXXXXXXXXXXLEDYAFNFELILEEIARLFESGCQKDEAEK |
| 2/15_D56C | AKRMKEWMKRIKTXXXEDEQEEMANAIITILQSWIFS* (SEQ ID NO: 277) |
| Neoleukin- | PKKKIQLHAEHALYDALMILNIXXXXXXXXXXXLEDYAFNFELILEEIARLFESGXXCDEAEK |
| 2/15_K58C | ARRMKEWMKRIKTXXXEDEQEEMANAIITILQSWIFS* (SEQ ID NO: 278) |
| Neoleukin- | PKKKIQLHAEHALYDALMILNIXXXXXXXXXXXLEDYAFNFELTLEEIARLFESGXXKCEAEK |
| 2/15_D59C | AKRMKEWMKRIKTXXXEDEQEEMANAIITILQSWIFS* (SEQ ID NO: 279) |
| Neoleukin- | PKKKIQLHAEHALYDALMILNIXXXXXXXXXXXLEDYAFNFELILEEIARLFESGXXKDEACK |
| 2/15_E62C | AKRMKEWMKRIKTXXXEDEQEEMANAIITILQSWIFS* (SEQ ID NO: 280) |
| Neoleukin- | PKKKIQLHAEHALYDALMILNIXXXXXXXXXXXLEDYAFNFELILEEIARLFESGXXKDEAEK |
| 2/15_R66C | AKCMKEWMKRIKTXXXEDEQEEMANAIITILQSWIFS* (SEQ ID NO: 281) |
| Neoleukin- | PKKKIQLHAEHALYDALMILNIXXXXXXXXXXXLEDYAFNFELILEEIARLFESGXXKDEAEK |
| 2/15_E69C | AKRMKCWMKRIKTXXXEDEQEEMANAIITILQSWIFS* (SEQ ID NO: 282) |
| Neoleukin- | PKKKIQLHAEHALYDALMILNIXXXXXXXXXXXLEDYAFNFEL1LEEIARLFESGXXKDEAEK |
| 2/15_R73C | AKRMKEWMKCIKTXXXEDEQEEMANAIITILQSWIFS* (SEQ ID NO: 283) |
| Neoleukin- | PKKKIQLHAEHALYDALMILNIXXXXXXXXXXXLEDYAFNFELILEEIARLFESGXXKDEAEK |
| 2/15_777C | AKRMKEWMKRIKTCASEDEQEEMANAIITILQSWIFS* (SEQ ID NO: 284) |
| Neoleukin- | PKKKIQLHAEHALYDALMILNIXXXXXXXXXXXLEDYAFNFELILEEIARLFESGXXKDEAEK |
| 2/15_E82C | AKRMKEWMKRIKTXXXEDCQEEMANAIITILQSWIFS* (SEQ ID NO: 285) |
| Neoleukin- | PKKKIQLHAEHALYDALMILNIXXXXXXXXXXXLEDYAFNFELILEEIARLFESGXXKDEAEK |
| 2/15_E85C | AKRMKEWMKRIKTXXXEDEQECMANAIITILQSWIFS* (SEQ ID NO: 286) |
| Neoleukin- | PKKKIQLHAEHALYDALMILNIXXXXXXXXXXXLEDYAFNFELILEEIACLFESGXXKDEAEK |
| 2/15_R50C_R73C | AKRMKEWMKCIKTXXXEDEQEEMANAIITILQSWIFS* (SEQ ID NO: 287) |
| Neoleukin- | PKKKIQLHAEHALYDALMILNIXXXXXXXXXXXLEDYAFNFELILERIARLFCSGXXKDEAEK |
| 2/15_E53C_R73C | AKRMKEWMKCIKTXXXEDEQEEMANAIITILQSWIFS* (SEQ ID NO: 288) |
| Neoleukin- | PKKKIQLHAEHALYDALMILNIXXXXXXXXXXXLEDYAFNFELILEEIARLFESGCQKDEAEK |
| 2/15/56C_R73C | AKRMKEWMKCIKTXXXEDEQEEMANAIITILQSWIFS* (SEQ ID NO: 289) |
| Neoleukin- | PKKKIQLHAEHALYDALMILNIXXXXXXXXXXXLEDYAFNFEL1LEEIARLFESGXXCDEAEK |
| 2/15_K58C_R73C | AKRMKEWMKCIKTXXXEDEQEEMANAIITILQSWIFS* (SEQ ID NO: 290) |
| Neoleukin- | PKKKIQLHAEHALYDALMILNIXXXXXXXXXXXLEDYAFNFELILERIARLFESGXXKCEAEK |
| 2/15_D59C_R73C | AERMKEWMKCIKTXXXEDEQEEMANAIITILQSWIFS* (SEQ ID NO: 291) |
| Neoleukin- | PKKKIQLHAEHALYDALMILNIXXXXXXXXXXXLEDYAFNFELILEEIARLFESGXXKDEAEK |
| 2/15_R66C_R73C | AKCMKEWMKCIKTXXXEDEQEEMANAIITILQSWIFS* (SEQ ID NO: 292) |
| Neoleukin- | PKKKIQLHAEHALYDALMILNIXXXXXXXXXXXLEDYAFNFELILEEIACLFESGXXKDEAEK |
| 2/15_R50C_E82C | AKRMKEWMKRIKTXXXEDCQEEMANAIITILQSWIFS* (SEQ ID NO: 293) |
| Neoleukin- | PKKKIQLHAEHALYDALMILNIXXXXXXXXXXXLEDYAFNFELTLEEIARLFCSGXXKDEAEK |
| 2/15_E53C E82C | AERMKEWMKRIKTXXXEDCQEEMANAIITILQSWIFS* (SEQ ID NO: 294) |
| Neoleukin- | PKKKIQLHAEHALYDALMILNIXXXXXXXXXXXLEDYAFNFELILEEIARLFESGCQKDEAEK |
| 2/15_D56C_E82C | AKRMKEWMKRIKTXXXEDCQEEMANAIITILQSWIFS* (SEQ ID NO: 295) |
| Neoleukin- | PKKKIQLHAEHALYDALMILNIXXXXXXXXXXXLEDYAFNFELILEEIARLFESGXXCDEAEK |
| 2/15_K58C_E82C | AKRMKEWMKRIKTXXXEDCQEEMANAIITILQSWIFS* (SEQ ID NO: 296) |
| Neoleukin- | PKKKIQLHAEHALYDALMILNIXXXXXXXXXXXLEDYAFNFELILEEIARLFESGXXKCEAEK |
| 2/15_D59C_E82C | AFFMKEWMKPIKTXXXEDCQEEMANATITILQSWITS* (SEQ ID NO: 297) |
| Neoleukin- | PKKKIQLHAEHALYDALMILNIXXXXXXXXXXXLEDYAFNFELTLEEIARLFESGXXKDEACK |
| 2/15_E62C_E82C | AKRMFEWMKRIFTXXXEDCQEEMANAIITTLQSWIFS* (SEQ ID NO: 298) |
| Neoleukin- | PKKKIQLHAEHALYDALMILNIXXXXXXXXXXXLEDYAFNFELILEEIARLFESGXXKDEAEK |
| 2/15_R66C_E82C | AKCMKEWMKRIKTXXXEDCQEEMANALLTILQSWIFS* (SEQ ID NO: 299) |
| Neoleukin- | PKKKIQLHAEHALYDALMILNIXXXXXXXXXXXLEDYAFNFELILEEIARLFESGXXKDEAEK |
| 2/15_E69C_E82C | AKRMKCWMKRIKTXXXEDCQEEMANAIITILQSWIFS* (SEQ ID NO: 300) |

In a specific embodiment, X1, X2, X3, and X4, respectively, are 25%, 27%, 30%, 35%, 40%, 45%, 50%, 55%, 60%, 65%, 70%, 75%, 80%, 85%, 90%, 95%, 98%, or 100% identical to, respectively, X1, X2, X3, and X4 domains (as defined in Table 1 (though listed as H1, H2, H3, and H4 domains) present within the amino acid sequence of SEQ ID NO:90 version 1 or 2, which have the same primary amino acid sequence but which differ slightly in optional/variable linker residues. In various embodiments, this embodiment may include variants of X1, X2, X3, and/or X4 domains present in SEQ ID NO:90 version 1 or 2 that incorporate the mutations relative to the SEQ ID NO:90 primary amino acid sequence shown in SEQ ID NOS:275-300.

In one embodiment of any embodiment or combination of embodiments disclosed herein, X1, X2, X3, and X4 are alpha-helical domains. In another embodiment, the amino acid length of each of X1, X2, X3 and X4 is independently at least about 8, 10, 12, 14, 16, 19, or more amino acids in length. In other embodiments, the amino acid length of each of X1, X2, X3 and X4 is independently no more than 1000, 500, 400, 300, 200, 100, or 50 amino acids in length. In various further embodiments, the amino acid length of each of X1, X2, X3 and X4 is independently between about 8-1000, 8-500, 8-400, 8-300, 8-200, 8-100, 8-50, 10-1000, 10-500, 10-400, 10-300, 10-200, 10-100, 10-50, 12-1000, 12-500, 12-400, 12-300, 12-200, 12-100, 12-50, 14-1000, 14-500, 14-400, 14-300, 14-200, 14-100, 14-50, 16-1000, 16-500, 16-400, 16-300, 16-200, 16-100, 16-50, 19-1000, 19-500, 19-400, 19-300, 19-200, 19-100, or about 19-50 amino acids in length.

In one embodiment, the first polypeptide component and/or the second polypeptide component includes at least one disulfide bond.

In another embodiment, the first polypeptide component and the second polypeptide component are non-covalently associated. As noted herein, the first polypeptide component and the second polypeptide component are not active receptor agonists individually, and wherein the first polypeptide component and the second polypeptide interact to form an active agonist of IL-2 receptor β [symbol] _{c} heterodimer (IL-2Rβ [symbol] _{c}), IL-4 receptor α [symbol] _{c}heterodimer (IL-4Rα [symbol] _{c}), IL-13 alpha, or IL-4Ralpha/IL13Ralpha heterodimer. Thus, in this embodiment the first polypeptide and the second polypeptide may interact to form an active agonist. This interaction may be any suitable interaction, such as a non-covalent interaction. The interaction may comprise direct non-covalent binding of the first and second polypeptides, or an indirect interaction In one embodiment, the first polypeptide component and the second polypeptide component are indirectly bound to each other through a receptor, such as an IL-2 receptor [symbol] _{c} heterodimer (IL-2Rβ [symbol] _{c}), an IL-4 receptor α [symbol] _{c}heterodimer (IL-4Rα [symbol] _{c}), IL-13 alpha, or an IL-4Ralpha/IL13Ralpha heterodimer.

Methods of determining binding to receptors are known in the art and described herein, e.g., bio-Layer Interferometry binding assays. In some embodiments when the first polypeptide component and the second polypeptide interact at their intended receptor, they co-localize to bind to that receptor with a binding affinity of 1000 nm or less, 200 nm or less, 100 nm or less, 50 nM or less, or 25 nM or less. For example, a split IL-2 mimetic of the present invention will co-localize to bind to the IL-2 receptor β [symbol] _{c} heterodimer (IL-2Rβ [symbol] _{c}) with a binding affinity of 1000 nm or less, 200 nm or less, 100 nm or less, 50 nM or less, or 25 nM or less. Similarly, as an example, a split IL-4 mimetic of the present invention will co-localize to bind to the IL-4 receptor α [symbol] _{c}heterodimer (IL-4Rα [symbol] _{c}) with a binding affinity of 1000 nm or less, 200 nm or less, 100 nm or less, 50 nM or less, or 25 nM or less. In some aspects, agonism of the receptor to which the split mimetics co-localize and bind is measured by STAT5 phosphorylation.

In another aspect, the disclosure provides polypeptides comprising 1, 2, or 3, but not all 4 domains X1, X2, X3, and X4, wherein:
(a) X1, when present, is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (PKKKIQ)LHAEHALYDAL(MILNI); (SEQ ID NO: 4);
(b) X2, when present, is any helical peptide domain;
(c) X3, when present, is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (LE)DYAFNFELILEE(IARLFESG) (SEQ ID NO:5); and
(d) X4, when present, is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical to the full length of peptide (EDEQEEMANAI)ITILQSWIF(S) (SEQ ID NO:6); and
amino acid residues in parentheses may be present or absent.

The polypeptides of this aspect can be used, for example, to generate the conditionally active receptor agonists of any embodiment or combination of embodiments disclosed herein (i.e.: the polypeptides of this aspect are either the first polypeptide or the second polypeptide of the conditionally active receptor agonists of the disclosure). Thus, as will be clear to those of skill in the art, all embodiments and combinations of embodiments of the first and second polypeptides disclosed above, all embodiments and combinations of embodiments of the X1, X2, X3, and X4 domains described above are equally applicable to the polypeptides of this aspect of the disclosure. In one embodiment,
(a) amino acid (AA) substitutions in X1 relative to the AA sequence of SEQ ID NO:4 occur at no more than 3 AA residues marked in bold font, or occur at no more than 2 AA residues marked in bold font, or occur at no more than 1 AA residue marked in bold font, or do not occur at AA residues marked in bold font;
(b) AA substitutions in X3 relative to the AA sequence of SEQ ID NO:5 occur at no more than 3 AA residues marked in bold font, or occur at no more than 2 AA residues marked in bold font, or occur at no more than 1 AA residue marked in bold font, or do not occur at AA residues marked in bold font; and/or
(c) AA substitutions in X4 relative to the AA sequence of SEQ ID NO:6 occur at no more than 2 AA residues marked in bold font, or occur at no more than 1 AA residue marked in bold font, or do not occur at AA residues marked in bold font.

In another embodiment, AA substitutions in X2 relative to the AA sequence of SEQ ID NO:7 do not occur at AA residues marked in bold font.

In various embodiments, the polypeptide may be selected from the group consisting of:
(i) a polypeptide comprising X1 and excluding X2, X3, and X4;
(ii) a polypeptide comprising X2 and excluding X1, X3, and X4;
(iii) a polypeptide comprising X3 and excluding X1, X2, and X4;
(iv) a polypeptide comprising X4 and excluding X1, X2, and X3;
(v) a polypeptide comprising X1 and X2, and excluding X3 and X4;
(vi) a polypeptide comprising X1 and X3, and excluding X2 and X4;
(vii) a polypeptide comprising X1 and X4, and excluding X2 and X3;
(viii) a polypeptide comprising X2 and X3, and excluding X1 and X4;
(ix) a polypeptide comprising X2 and X4, and excluding X1 and X3;
(x) a polypeptide comprising X3 and X4, and excluding X1 and X2;
(xi) a polypeptide comprising X1, X2, and X3 and excluding X4;
(xii) a polypeptide comprising X1, X2, and X4 and excluding X3;
(xiii) a polypeptide comprising X1, X3, and X4 and excluding X2; and
(xiv) a polypeptide comprising X2, X3, and X4 and excluding X1.

In one embodiment, the polypeptide comprise the amino acid sequence at least 25%, 27%, 30%, 35%, 40%, 45%, 50%, 55%, 60%, 65%, 70%, 75%, 80%, 85%, 90%, 95%, 98%, or 100% identical to, a first polypeptide or a second polypeptide listed below (underlined residues are optional and each optional residue, when present, may comprise any amino acid):

(i)
First polypeptide X1 (Neo2A)
(SEQ ID NO: 256)
PKKKIQLHAEHALYDALMILNIVKTNS
and
Second polypeptide: X3-X2′-X4 (Neo2B)
(SEQ ID NO: 257)
TNSPPAEEKLEDYAFNFELILEEIARLFESGDQKDEAEAKRMKEWMKRIKTTASEDEQEEMANAIIT
ILQSWIFS
(ii)
First polypeptide X1-X3-X2′
(SEQ ID NO: 258)
PKKKIQLHAEHALYDALMILNIVKTNSPPAEEKLEADYAFNFELILEEIARLFESGDQKDEAEKAKRMK
EWMKRIKTTAS
and
Second polypeptide X4
(SEQ ID NO: 259)
TTASEDEQEEMANAIITILQSWIFS;
(iii)
First polypeptide X1-X3
(SEQ ID NO: 260)
PKKKIQLHAEHALYDALMILNIVKTNSPPAEKLEDYAFNFELILEEIARLFESGD
and
Second polypeptide X2-X4
(SEQ ID NO: 261)
DQKDEAEKAKRMKEWMKRIKTTASEDEQEEMANAIITILQSWIFS
(iv)
First polypeptide X1 (Neo4A)
(SEQ ID NO: 262)
PKKKIQIMAEELKDALSILNIVKTNS
Second polypeptide X3-X2′-X4 (Neo4B)
(SEQ ID NO: 263)
TNSPPAEEQLERFAKRFERNLWGIARLEESGDQKDEAEKAKRMIEWMKRIKTTASEDEQEEMANAIIT
ILQSWFFS
(v)
First polypeptide X1 (Neo2A)
(SEQ ID NO: 311)
PKKKIQLHAEHALYDALMILNIXXXXX
and
Second polypeptide: X3-X2′-X4 (Neo2B)
(SEQ ID NO: 264)
XXXXXXXXXLEDYAFNFELILEEIARLFESGXXKDEAEKAKPREEWMKRIKTXXXEDEQEEMANAIIT
ILQSWIFS
(vi)
First polypeptide X1-X3-X2′
(SEQ ID NO: 265)
PKKKIQLHAEHALYDALMILNIXXXXXXXXXXLEDYAFNFELILEEIARLFESGXXKDEAEKAKRMK
EWMKRIKTTTAS
and
Second polypeptide X4
(SEQ ID NO: 266)
XXXXXDEQEEMANIITTILQSWIFS;
(vii)
First polypeptide X1-X3
(SEQ ID NO: 267)
PKEKIQLHAEHALYDADMILNIXXXXXXXXXXXLEDYAFNFELILEEIARLFESXXGD
and
Second polypeptide X2-X4
(SEQ ID NO: 268)
DQKDEAEKAKRMKEWNKRIKTXXXEDEQEEMANAIITILQSWIFS
(viii)
First polypeptide X1 (Neo4A)
(SEQ ID NO: 269)
PKKKIQIMAEEALKDAISILNIXXXXX
Second polypeptide X3-X2′-X4 (Neo4B)
(SEQ ID NO: 270)
XXXXXXXXQLERFAKRFERNLWGIARLFESGXXKDEAEKAKRMIEWMKRIKTXXXEDEQEEMANAIIT
ILQSWFFS
(ix)
First polypeptide >Neo4_H1-H3′
(SEQ ID NO: 312)
PKKKIQIMAEEALKDALSILNIVKTNSPPAEEQLERFAKRFERNLWGIARLFESGD
Second polypeptide >Neo4_H2-H4
(SEQ ID NO: 313)
DQKDEAEKAKRMIEWMKRIKTTASEDEQEEMANAIITILQSWFFS
(x)
First polypeptide >Neo4_H1-H3′
(SEQ ID NO: 314)
PKKKIQIMAEEALKDALSILNIXXXXXXXXXXQLERFAKRFERNLWGIARLFESXX
Second polypeptide >Neo4_H2-H4
(SEQ ID NO: 315)
XXKDEAEKAKRMIEWMKRIKXXXXEDEQEEMANAIITILQSWFFS
(xi)
First polypeptide Neo4_H1-H3′-H2
(SEQ ID NO: 316)
PKKKIQIMAEEALKDALSILNIVKTNSPPAEEQLERFAKRFERNLWGIARLFESGDQKDEAEKAKRMI
EWMKRIKTTA
Second polypeptide Neo4_H4
(SEQ ID NO: 317)
TTASEDEQEEMANAIITILQSWFFS
(xii)
First polypeptide Neo4_H1-H3′-H2
(SEQ ID NO: 318)
PKKKIQIMAEEALKDALSILNIXXXXXXXXXXXLERFAKRFERNLWGIARLFESXXXKDEAEKAKRMI
EWMKRIKXXX
Second polypeptide Neo4_H4
(SEQ ID NO: 319)
XXXXXDEQEEMANAIITILQSWFFS;
(xiii)
First polypeptide (X1)
(SEQ ID NO: 323)
PKKKIQLHAEHALYDALMILNIVGGSS,
or
(SEQ ID NO: 324)
SKEAIQLHAEHALYDALMILNIVKTNS,
or
(SEQ ID NO: 325)
PIQLHAEHALYDALMILNIV
Second polypeptide (X3-X2′-X4)
(SEQ ID NO: 326)
PKLEDYAFNFELILEEIARLFESGDQKDEAEKAKRMKEWMKRIKTTASEDEQEEMANAIITILQ
SWIFS;
or
(SEQ ID NO: 327)
GSGSSGGLEDYAFNFELILEEIARLFESGGSSGGKDEAEKAKRMKEWMKRITGGSSGGDEQEEMANAI
ITILQSWIFS;
or
(SEQ ID NO: 328)
GGSSGGLEDYAFNFELILEEIARLFESGGSSGGGGEAEKAKRMKEWMKRIGGSSGGDEQEEMANAIIT
ILQSWIFS.

In exemplary embodiments, the polypeptide comprises an amino acid sequence at least 80% identical to a first or second polypeptides shown in embodiments (i)-(viii) above 10 (underlined residues or “X” residues” are optional and each residue of the optional domain, when present, may comprise any amino acid).

In exemplary embodiments, the polypeptide comprises an amino acid sequence at least 90% identical to a first or second polypeptides shown in embodiments (i)-(viii) above (underlined residues or “X” residues” are optional and each residue of the optional domain, when present, may comprise any amino acid).

In exemplary embodiments, the polypeptide comprises an amino acid sequence at least 100% identical to a first or second polypeptides shown in embodiments (i)-(viii) above (underlined residues or “X” residues” are optional and each residue of the optional domain, when present, may comprise any amino acid).

In another embodiment, X1, X2, X3, and X4, when present, comprise the amino acid sequence at least 25%, 27%, 30%, 35%, 40%, 45%, 50%, 55%, 60%, 65%, 70%, 75%, 80%, 85%, 90%, 95%, 98%, or 100% identical to, respectively, X1, X2, X3, and X4 domains (as defined in Table 1) present within the amino acid sequence selected from the group consisting of SEQ ID NO:11-94, 190-216, 247, and SEQ ID NOS275-300. In a specific embodiment, X1, X2, X3, and X4, when present, comprise the amino acid sequence at least 25%, 27%, 30%, 35%, 40%, 45%, 50%, 55%, 60%, 65%, 70%, 75%, 80%, 85%, 90%, 95%, 98%, or 100% identical to, respectively, X1, X2, X3, and X4 domains (as defined in Table 1 (though listed as H1, H2, H3, and H4 domains) present within the amino acid sequence of SEQ ID NO:90 versions 1 or 2, which have the same primary amino acid sequence but which differ slightly in optional/variable linker residues. In various embodiments, this embodiment may include variants of X1, X2, X3, and/or X4 domains present SEQ ID NO:90 version 1 or 2 that incorporate the mutations relative to the SEQ ID NO:90 primary amino acid sequence shown SEQ ID NOS:275-300.

The first polypeptides, second polypeptides, and polypeptides described herein may be chemically synthesized or recombinantly expressed (when the polypeptide is genetically encodable). The polypeptides may be linked to other compounds, such as stabilization compounds to promote an increased half-life in vivo, including but not limited to albumin, PEGylation (attachment of one or more polyethylene glycol chains), HESylation, PASylation, glycosylation, or may be produced as an Fc-fusion or in deimmunized variants. Such linkage can be covalent or non-covalent. For example, addition of polyethylene glycol (“PEG”) containing moieties may comprise attachment of a PEG group linked to maleimide group (“PEG-MAL”) to a cysteine residue of the polypeptide. Suitable examples of PEG-MAL are methoxy PEG-MAL 5 kD; methoxy PEG-MAL 20 kD; methoxy (PEG)2-MAL 40 kD; methoxy PEG(MAL)2 5 kD; methoxy PEG(MAL)2 20 kD; methoxy PEG(MAL)2 40 kD; or any combination thereof. See also U.S. Pat. No. 8,148,109. In other embodiments, the PEG may comprise branched chain PEGs and/or multiple PEG chains.

In one embodiment, the stabilization compound, including but not limited to a PEG-containing moiety, is linked at a cysteine residue in the polypeptide. In another embodiment, the cysteine residue is present in the X2 domain. In some embodiments, the cysteine residue is present, for example, in any one of a number of positions in the X2 domain. In some such embodiments, the X2 domain is at least 19 amino acids in length and the cysteine residue is at positions 1, 2, 5, 9 or 16 relative to those 19 amino acids. In a further embodiment, the stabilization compound, including but not limited to a PEG-containing moiety, is linked to the cysteine residue via a maleimide group.

In a further embodiment, the first polypeptides, second polypeptides, and polypeptides may further comprise a targeting domain. In this embodiment, the conditionally receptor agonist can be directed to a target of interest. The targeting domain may be covalently or non-covalently bound to the first polypeptide, second polypeptide, and/or polypeptide. In embodiments where the targeting domain is non-covalently bound, any suitable means for such non-covalent binding may be used, including but not limited to streptavidin-biotin linkers.

In another embodiment, the targeting domain, when present, is a translational fusion with the polypeptide. In this embodiment, the polypeptide and the targeting domain may directly abut each other in the translational fusion or may be linked by a polypeptide linker suitable for an intended purpose. Exemplary such linkers include, but are not limited, to those disclosed in WO2016178905, WO2018153865 (in particular, at page 13), and WO 2018170179 (in particular, at paragraphs [0316]-[0317]). In other embodiments, suitable linkers include, but are not limited to peptide linkers, such as GGGGG (SEQ ID NO: 95), GSGGG (SEQ ID NO: 96), GGGGGG (SEQ ID NO: 97), GGSGGG (SEQ ID NO: 98), GGSGGSGGGSGGSGSG (SEQ ID NO: 99), GSGGSGGGSGGSGSG (SEQ ID NO: 100), GGSGGSGGGSGGSGGGGSGGSGGGSGGGGS (SEQ ID NO: 101), and [GGGGX]ₙ (SEQ ID NO: 102), where X is Q, E or S and n is 2-5.

The targeting domains are polypeptide domains or small molecules that bind to a target of interest. In one non-limiting embodiment, the targeting domain binds to a cell surface protein; in this embodiment, the cell may be any cell type of interest that includes a surface protein that can be bound by a suitable targeting domain. In one embodiment, the cell surface proteins are present on the surface of cells selected from the group consisting of tumor cells, tumor vascular component cells, tumor microenvironment cells (e.g. fibroblasts, infiltrating immune cells, or stromal elements), other cancer cells and immune cells (including but not limited to CD8+ T cells, T-regulatory cells, dendritic cells, NK cells, or macrophages). When the cell surface protein is on the surface of a tumor cell, vascular component cell, or tumor microenvironment cell (e.g. fibroblasts, infiltrating immune cells, or stromal elements), any suitable tumor cell, vascular component cell, or tumor microenvironment cell surface marker may be targeted, including but not limited to EGFR, EGFRvIII, Her2, HER3, EpCAM, MSLN, MUC16, PSMA, TROP2, ROR1, RON, PD-L1, CD47, CTLA-4, CD5, CD19, CD20, CD25, CD37, CD30, CD33, CD40, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185LHER2, IL-2 receptor, EGFRvIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, bcr-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase LX, PAX5, OY-TESL Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorf186, RON, LY6E, FRA, DLL3, PTK7, STRA6, TMPRSS3, TMPRSS4, TMEM238, UPK1B, VTCN1, LIV1, ROR1, Fos-related antigen 1, BMPR1B (bone morphogenetic protein receptor-type IB, Genbank accession no. NM. 001203); E16 (LAT1, SLC7A5, Genbank accession no. NM-003486); STEAP1 (six transmembrane epithelial antigen of prostate, Genbank accession no. NM 012449); 0772P (CA125, MUC16, Genbank accession no. AF361486); MPF (MPF, MSLN, SMR, megakaryocyte potentiating factor, mesothelin, Genbank accession no. NM 005823); Napi3b (NAPI-3B, NPTIIb, SLC34A2, solute carrier family 34 (sodium phosphate), member 2, type II sodium-dependent phosphate transporter 3b, Genbank accession no. NM 006424); Sema 5b (FLJ10372, KIAA1445, Mm. 42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B, Genbank accession no. AB040878); PSCA hlg (2700050C12Rik, C530008016Rik, RIKEN cDNA 2700050C12, RIKEN cDNA 2700050C12 gene, Genbank accession no. AY358628); ETBR (Endothelin type B receptor, Genbank accession no. AY275463); MSG783 (RNF124, hypothetical protein FLJ20315, Genbank accession no. NM 017763); STEAP2 (HGNC.sub.-8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138); TrpM4 (BR22450, FLJ20041, TRPM4, TRPM4B, transient receptor potential cation channel, subfamily M, member 4, Genbank accession no. NM 017636); CRIPTO (CR, CR1, CRGF, CRIPTO, TDGF1, teratocarcinoma-derived growth factor, Genbank accession no. NP003203 or NM003212); CD21 (CR2 (Complement receptor 2) or C3DR(C3d/Epstein Barr virus receptor) or Hs. 73792, Genbank accession no. M26004); CD79b (IGb (immunoglobulin-associated beta), B29, Genbank accession no. NM 000626); FcRH2 (IFGP4, IRTA4, SPAP1A (SH2 domain containing phosphatase anchor protein 1a), SPAP1B, SPAP1C, Genbank accession no. NM030764); HER2 (Genbank accession no. M11730); NCA (Genbank accession no. M18728); MDP (Genbank accession no. BC017023); IL20R.alpha. (Genbank accession no. AF184971); Brevican (Genbank accession no. AF229053); Ephb2R (Genbank accession no. NM004442); ASLG659 (Genbank accession no. AX092328); PSCA (Genbank accession no. AJ297436); GEDA (Genbank accession no. AY260763); BAFF-R (Genbank accession no. NP443177.1); CD22 (Genbank accession no. NP-001762.1); CD79a (CD79A, CD79.alpha., immunoglobulin-associated alpha, a B cell-specific protein that covalently interacts with Ig beta (CD79B) and forms a complex on the surface with Ig M molecules, transduces a signal involved in B-cell differentiation, Genbank accession No. NP001774.1); CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia, Genbank accession No. NP001707.1); HLA-DOB (Beta subunit of MHC class II molecule (Ia antigen) that binds peptides and presents them to CD4+T lymphocytes, Genbank accession No. NP002111.1); P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability, Genbank accession No. NP002552.2); CD72 (B-cell differentiation antigen CD72, Lyb-2, Genbank accession No. NP001773.1); LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis, Genbank accession No. NP005573.1); FCRH1 (Fc receptor-like protein 1, a putative receptor for the immunoglobulin Fc domain that contains C2 type Ig-like and ITAM domains, may have a role in B-lymphocyte differentiation, Genbank accession No. NP443170.1); or IRTA2 (Immunoglobulin superfamily receptor translocation associated 2, a putative immunoreceptor with possible roles in B cell development and lymphomagenesis; deregulation of the gene by translocation occurs in some B cell malignancies, Genbank accession No. NP112571.1).

In another embodiment, the targeting domain binds to immune cell surface markers. In this embodiment, the target may be cell surface proteins on any suitable immune cell, including but not limited to CD8+ T cells, T-regulatory cells, dendritic cells, NK cells or macrophages. The targeting domain may target any suitable immune cell surface marker (whether an endogenous or an engineered immune cell, including but not limited to engineered CAR-T cells), including but not limited to CD3, CD4, CD8, CD19, CD20, CD21, CD25, CD37, CD30, CD33, CD40, CD68, CD123, CD254, PD-1, B7-H3, and CTLA-4. In another embodiment, the targeting domain binds to PD-1, PDL-1, CTLA-4, TROP2, B7-H3, CD33, CD22, carbonic anhydrase IX, CD123, Nectin-4, tissue factor antigen, CD154, B7-H3, B7-H4, FAP (fibroblast activation protein) or MUC16, and/or wherein the targeting domain binds to PD-1, PDL-1, CTLA-4, TROP2, B7-H3, CD33, CD22, carbonic anhydrase IX, CD123, Nectin-4, tissue factor antigen, CD154, B7-H3, B7-H4, FAP (fibroblast activation protein) or MUC16.

In all these embodiments, the targeting domains can be any suitable polypeptides that bind to targets of interest and can be incorporated into the polypeptide of the disclosure. In non-limiting embodiments, the targeting domain may include but is not limited to an scFv, a F(ab), a F(ab′)₂, a B cell receptor (BCR), a DARPin, an affibody, a monobody, a nanobody, diabody, an antibody (including a monospecific or bispecific antibody); a cell-targeting oligopeptide including but not limited to RGD integrin-binding peptides, de novo designed binders, aptamers, a bicycle peptide, conotoxins, small molecules such as folic acid, and a virus that binds to the cell surface.

The first polypeptides, second polypeptides, and polypeptides of the disclosure may include additional residues at the N-terminus, C-terminus, or both that are not present in the first polypeptides, second polypeptides, and polypeptides of the disclosure; these additional residues are not included in determining the percent identity of the polypeptides or peptide domains of the disclosure relative to the reference polypeptide. Such residues may be any residues suitable for an intended use, including but not limited to detection tags (i.e.: fluorescent proteins, antibody epitope tags, etc.), adaptors, ligands suitable for purposes of purification (His tags, etc.), other peptide domains that add functionality to the polypeptides, etc. Residues suitable for attachment of such groups may include cysteine, lysine or p-acetylphenylalanine residues or can be tags, such as amino acid tags suitable for reaction with transglutaminases as disclosed in U.S. Pat. Nos. 9,676,871 and 9,777,070.

In a further aspect, the present invention provides nucleic acids, including isolated nucleic acids, encoding the first polypeptides, second polypeptides, and polypeptides of the present disclosure that can be genetically encoded. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the invention operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit. In non-limiting embodiments, the expression vectors can be used to transfect or transduce cell therapeutic targets (including but not limited to CAR-T cells or tumor cells) to effect the therapeutic methods disclosed herein.

In a further aspect, the present disclosure provides host cells that comprise the expression vectors and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney. 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

In another aspect, the present disclosure provides pharmaceutical compositions, comprising one or more conditionally active receptor agonist, polypeptide, nucleic acids, expression vectors, and/or host cells of any aspect or embodiment of the disclosure and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The conditionally active receptor agonists, polypeptides, nucleic acids, expression vectors, and/or host cells may be the sole active agent in the pharmaceutical composition, or the composition may further comprise one or more other active agents suitable for an intended use.

In a further aspect, the present disclosure provides methods for treating cancer, comprising administering to a subject in need thereof a therapeutically effective amount of the conditionally active receptor agonist, polypeptide, nucleic acids, expression vectors, and/or host cells of any embodiment or combination of embodiments disclosed herein under conditions wherein the first polypeptide component and the second polypeptide component interact at cells of the tumor to treat the cancer. In embodiments for administering the conditionally active receptor agonists, the first and second polypeptide may be administered together, or may be administered in separate pharmaceutical formulations.

As used herein, “treat” or “treating” means accomplishing one or more of the following: (a) reducing the size or volume of tumors and/or metastases in the subject; (b) limiting any increase in the size or volume of tumors and/or metastases in the subject; (c) increasing survival; (d) reducing the severity of symptoms associated with cancer; (e) limiting or preventing development of symptoms associated with cancer; and (f) inhibiting worsening of symptoms associated with cancer.

The methods can be used to treat any suitable cancer, including but not limited to colon cancer, melanoma, renal cell cancer, head and neck squamous cell cancer, gastric cancer, urothelial carcinoma, Hodgkin lymphoma, non-small cell lung cancer, small cell lung cancer, hepatocellular carcinoma, pancreatic cancer, Merkel cell carcinoma colorectal cancer, acute myeloid leukemia, acute lymphoblastic leukemia, chronic lymphocytic leukemia, non-Hodgkin lymphoma, multiple myeloma, ovarian cancer, cervical cancer, and any tumor types selected by a diagnostic test, such as microsatellite instability, tumor mutational burden, PD-L1 expression level, or the immunoscore assay (as developed by the Society for Immunotherapy of Cancer).

The subject may be any subject that has cancer. In one embodiment, the subject is a mammal, including but not limited to humans, dogs, cats, horses, cattle, etc.

In one embodiment, the first targeting domain binds to a cell marker and the second targeting domain binds to a second and distinct cell marker, and wherein co-expression of these two markers on the same or nearby cells occurs more commonly in the tumor than in other tissues, and wherein the first polypeptide and the second polypeptide interact only after binding of the first targeting domain to the first cell marker and binding of the second targeting domain to the second cell marker. This embodiment, for example, employs two targeting domains that bind to separate markers that aren't themselves enriched on tumor cells, but for which the co-expression is enriched in the tumor.

In another embodiment, the first polypeptide component comprises a first targeting domain and the second polypeptide component comprises a second targeting domain, wherein the first targeting domain binds to a first tumor cell marker and the second targeting domain binds to a second tumor cell marker which may be the same or different than the first tumor cell marker, and wherein the first polypeptide and the second polypeptide interact only after binding of the first targeting domain to the first tumor cell marker and binding of the second targeting domain to the second tumor cell marker.

In another embodiment, the first polypeptide component comprises a first targeting domain and the second polypeptide component comprises a second targeting domain, wherein the first targeting domain binds to a tumor cell marker and the second targeting domain binds to an immune cell marker (including but not limited to CD8+ T cells, T-regulatory cells, dendritic cells, or macrophages), and wherein the first polypeptide and the second polypeptide interact only after binding of the first targeting domain to the tumor cell marker and binding of the second targeting domain to the immune cell marker.

In one embodiment, the first targeting domain binds to a cell marker and the second targeting domain binds to a second and distinct cell marker, and wherein co-expression of these two markers occur more commonly on tumor cells than on some other types of cells, and wherein the first polypeptide and the second polypeptide interact only after binding of the first targeting domain to the first cell marker and binding of the second targeting domain to the second cell marker on the same cell. This embodiment, for example, employs two targeting domains that bind to separate markers that aren't themselves enriched on tumor cells, but for which the co-expression is enriched in the tumor

In a further embodiment, the first polypeptide component comprises a first targeting domain and the second polypeptide component comprises a second targeting domain, wherein the first targeting domain binds to a first immune cell marker (including but not limited to CD8+ T cells, T-regulatory cells, dendritic cells, or macrophages) and the second targeting domain binds to a second immune cell marker (including but not limited to CD8+ T cells, T-regulatory cells, dendritic cells, or macrophages) which may be the same or different than the first immune cell marker, and wherein the first polypeptide and the second polypeptide interact only after binding of the first targeting domain to the first immune cell marker and binding of the second targeting domain to the second immune cell marker.

In a further aspect, the present disclosure provides methods for modulating an immune response in a subject by administering to a subject a conditionally active receptor agonist, polypeptide, nucleic acid, expression vector, host cell, or the pharmaceutical composition of the present disclosure. In one embodiment, the method comprises administering to a subject a conditionally active receptor agonist under conditions wherein the first polypeptide component and the second polypeptide component interact at immune cells to modulate an immune response.

As used herein, an “immune response” being modulated refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments of the compositions and methods described herein, an immune response being modulated is T-cell mediated.

In some aspects, the immune response is an anti-cancer immune response. In some such aspects, a conditionally active IL-2 mimetic described herein is administered to a subject having cancer to modulate an anti-cancer immune response in the subject.

In some aspects, the immune response is a tissue reparative immune response. In some such aspects, a conditionally active IL-4 mimetic described here is administered to a subject in need thereof to modulate a tissue reparative immune response in the subject.

In some aspects, the immune response is a wound healing immune response. In some such aspects, a conditionally active IL-4 mimetic described here is administered to a subject in need thereof to modulate a wound healing immune response in the subject.

In some aspects, methods are provided for modulating an immune response to a second therapeutic agent in a subject. In some such aspects, the method comprises administering a polypeptide of the present disclosure in combination with an effective amount of the second therapeutic agent to the subject. The second therapeutic agent can be, for example, a chemotherapeutic agent or an antigen-specific immunotherapeutic agent. In some aspects, the antigen-specific immunotherapeutic agent comprises chimeric antigen receptor T cells (CAR-T cells). In some aspects, the polypeptide of the present disclosure enhances the immune response of the subject to the therapeutic agent. The immune response can be enhanced, for example, by improving the T cell response (including CAR-T cell response), augmenting the innate T cell immune response, decreasing inflammation, inhibiting T regulatory cell activity, or combinations thereof.

In some aspects, a conditionally active cytokine mimetic of the present invention, e.g., a conditionally active IL-4 mimetic as described herein, will be impregnated to or otherwise associated with a biomaterial and the biomaterial will be introduced to a subject. In some aspects, the biomaterial will be a component of an implantable medical device and the device will be, for example, coated with the biomaterial. Such medical devices include, for example, vascular and arterial grafts. Conditionally active IL-4 and/or IL-4 associated biomaterials can be used, for example, to promote wound healing and/or tissue repair and regeneration.

In another aspect, the disclosure provides methods for agonizing the IL-2 receptor or the IL-4 receptor, comprising administering to a subject the conditionally active receptor agonist of any embodiment or combination of embodiments disclosed herein, under conditions wherein the first polypeptide component and the second polypeptide component interact at the receptor.

As used herein, a “therapeutically effective amount” refers to an amount of the conditionally active receptor agonist, polypeptide, nucleic acids, expression vectors, and/or host cells that is effective for treating and/or limiting the disease to be treated (e.g., cancer). The conditionally active receptor agonist, polypeptides, nucleic acids, expression vectors, and/or host cells are typically formulated as a pharmaceutical composition, such as those disclosed above, and can be administered via any suitable route, including but not limited to orally, by inhalation spray, ocularly, intravenously, subcutaneously, intraperitoneally, and intravesicularly in dosage unit formulations containing conventional pharmaceutically acceptable carriers, adjuvants, and vehicles. In one particular embodiment, the polypeptides, nucleic acids, expression vectors, and/or host cells are administered mucosally, including but not limited to intraocular, inhaled, or intranasal administration. In another particular embodiment, the polypeptides, nucleic acids, expression vectors, and/or host cells are administered orally. Such particular embodiments can be administered via droplets, nebulizers, sprays, or other suitable formulations.

Any suitable dosage range may be used as determined by attending medical personnel. Dosage regimens can be adjusted to provide the optimum desired response (e.g., a therapeutic or prophylactic response). A suitable dosage range for the conditionally active receptor agonists or polypeptides may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. In some embodiments, the recommended dose could be lower than 0.1 mcg/kg, especially if administered locally. In other embodiments, the recommended dose could be based on weight/m² (i.e. body surface area), and/or it could be administered at a fixed dose (e.g., 0.05-100 mg). The conditionally active receptor agonists, polypeptides, nucleic acids, expression vectors, and/or host cells can be delivered in a single bolus, or may be administered more than once (e.g., 2, 3, 4, 5, or more times) as determined by an attending physician.

The conditionally active receptor agonists, polypeptides, nucleic acids, expression vectors, and/or host cells made be administered as the sole prophylactic or therapeutic agent, or may be administered together with (i.e.: combined or separately) one or more other prophylactic or therapeutic agents, including but not limited to tumor resection, chemotherapy, radiation therapy, immunotherapy, etc.

Numerous modifications and variations of the present disclosure are possible in light of the above teachings.

### EXAMPLES

### Example 1

A computational approach for designing de novo cytokine mimetics is described that recapitulate the functional sites of the natural cytokines, but otherwise are unrelated in topology or amino acid sequence. This strategy was used to design de novo non-split mimetics of IL-2 and interleukin-15 (IL-15) that bind to the IL-2 receptor β [symbol] _{c}V, heterodimer (IL-2Rβ [symbol] _{c}), but have no binding site for IL-2Rα or IL-15Rα. The designs are hyper-stable, bind to human and mouse IL-2Rβ [symbol] _{c} with higher affinity than the natural cytokines, and elicit downstream cell signaling independent of IL-2Rα and IL-15Rα. Crystal structures of an experimentally optimized mimetic, neoleukin-2/15, are very close to the design model and provide the first structural information on the murine IL-2Rβ [symbol] _{c} complex. Neoleukin-2/15 has highly efficacious therapeutic activity compared to IL-2 in murine models of melanoma and colon cancer, with reduced toxicity and no signs of immunogenicity.

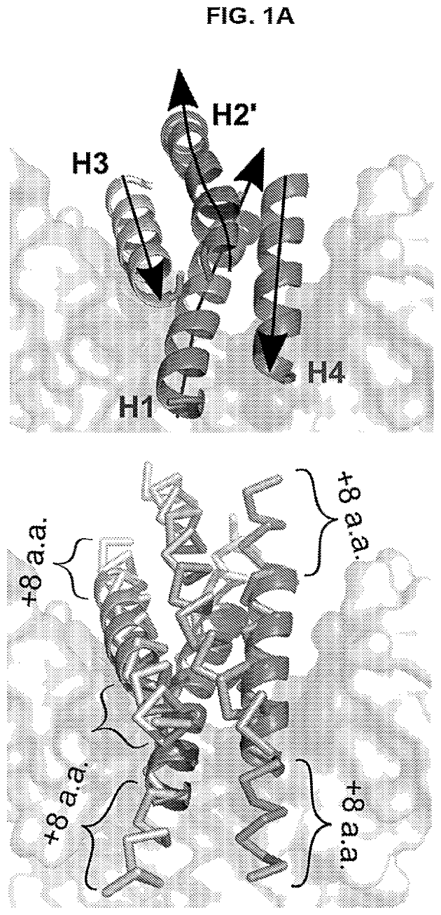

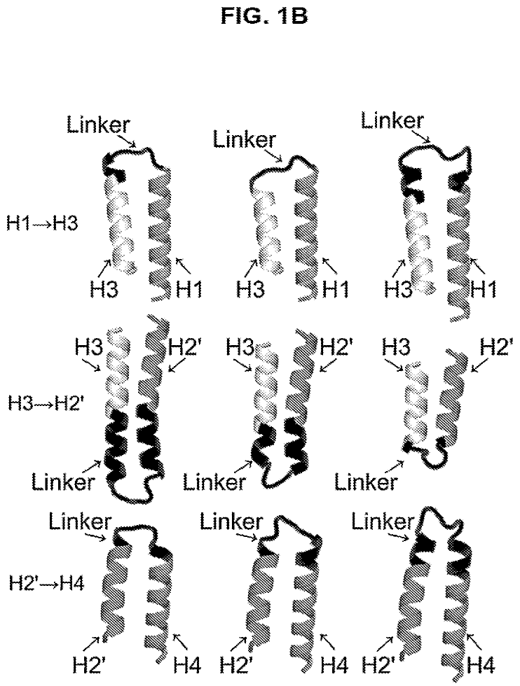

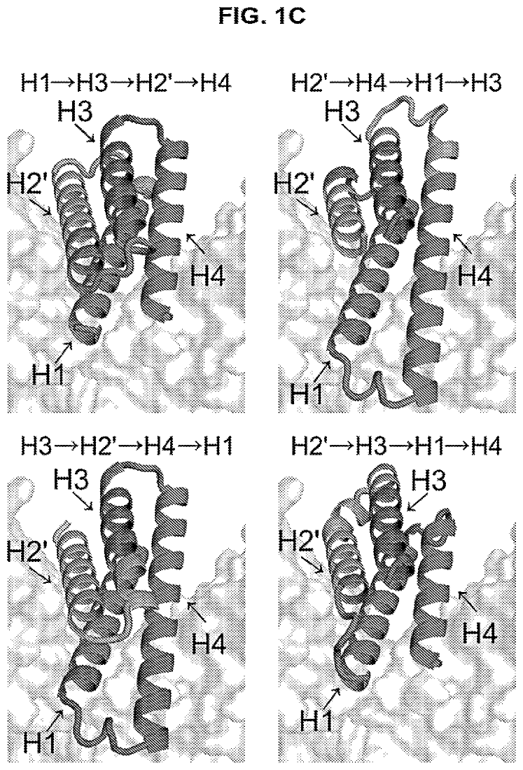

Many cytokines interact with multiple different receptor subunits, and like most naturally occurring proteins, contain non-ideal structural features that compromise stability but are important for function. A computational protocol was developed in which the structural elements interacting with the desired receptor subunit(s) are fixed in space, and an idealized globular protein structure is built to support these elements. Combinatorial fragment assembly was used to support short linear epitopes with parametric construction of disembodied helices coupled with knowledge-based loop closure (FIG. 1a-b). The approach was tested by attempting to de novo design stable idealized proteins with interaction surfaces mimicking those of human IL-2 (hIL-2) and human IL-15 (hIL-15) for the human IL-2Rβ [symbol] _{c} (hIL-2R Y), but entirely lacking the IL-2 receptor alpha (IL-2Rα) interaction surface.

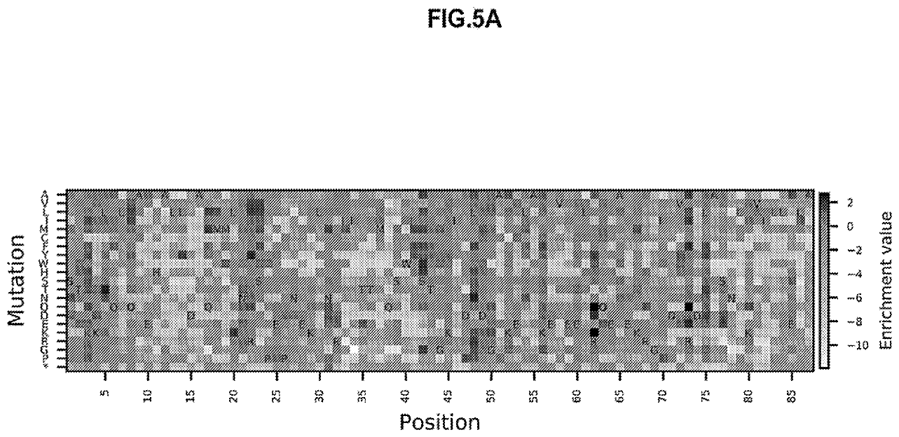

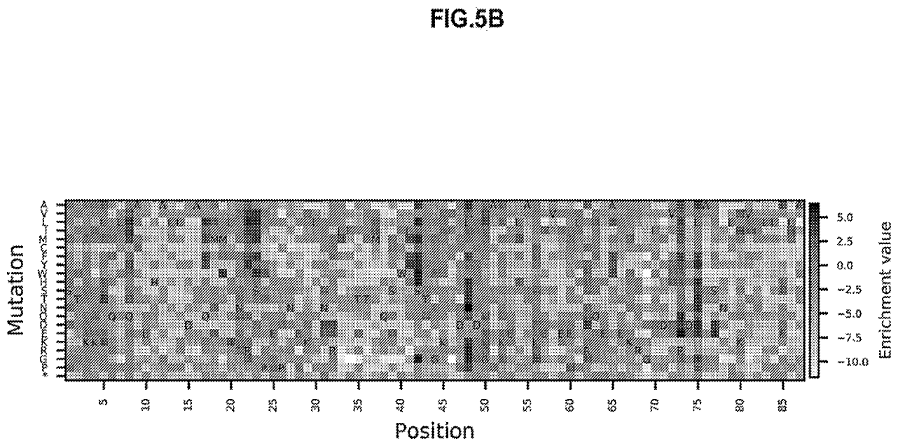

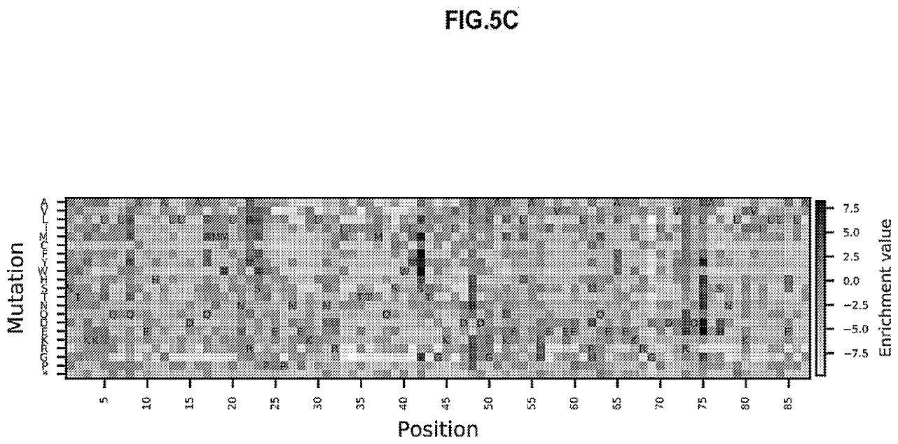

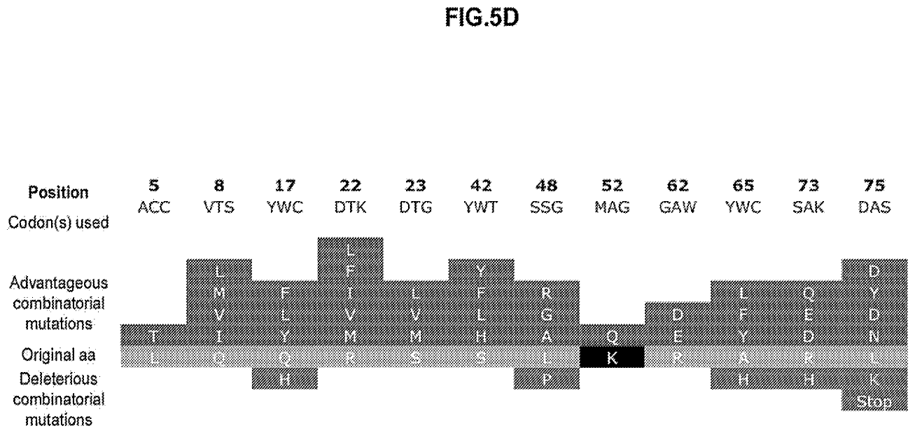

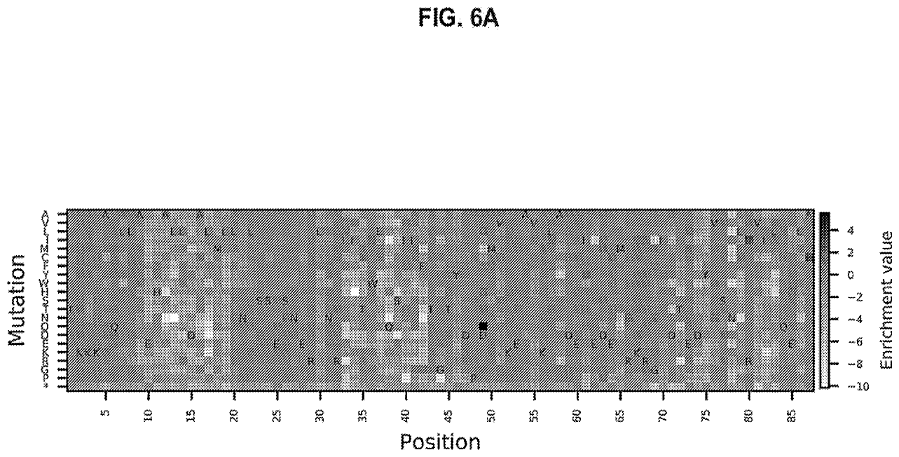

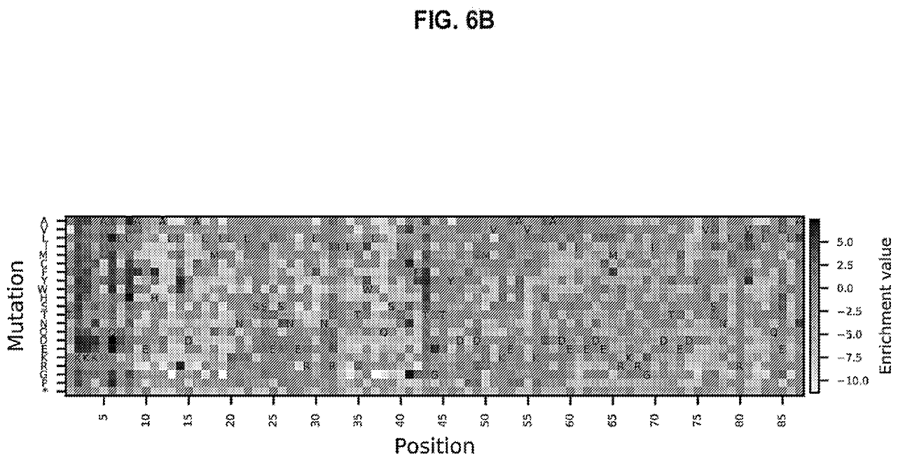

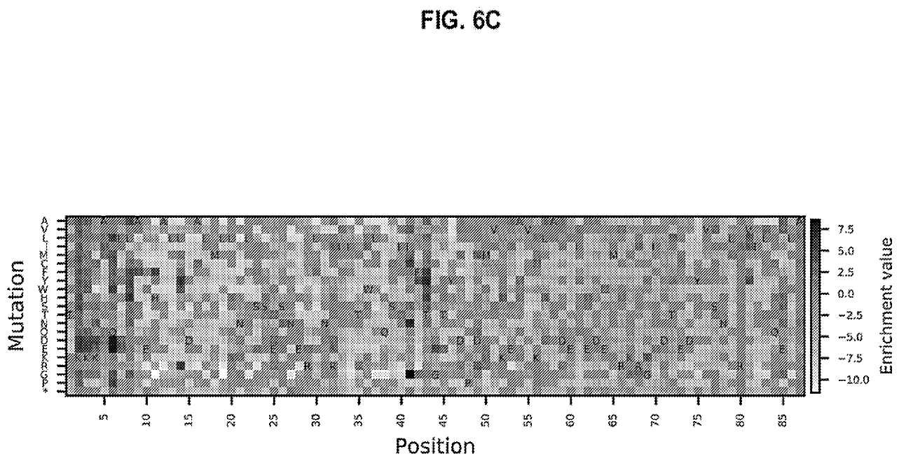

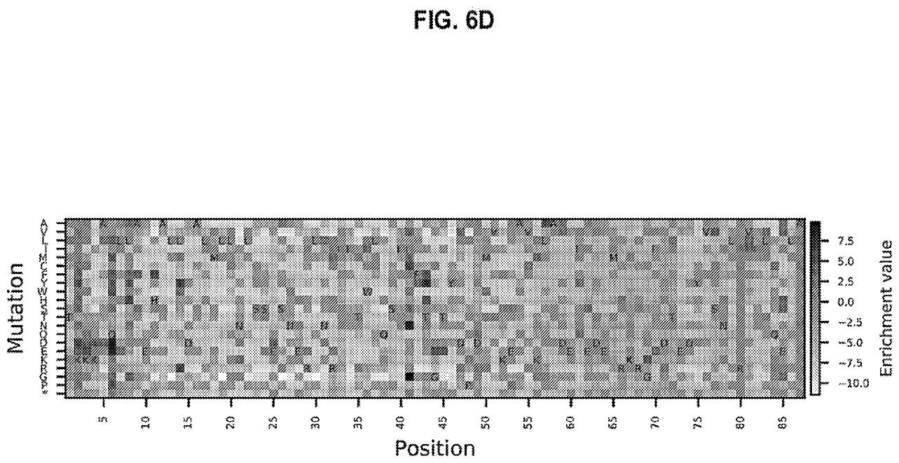

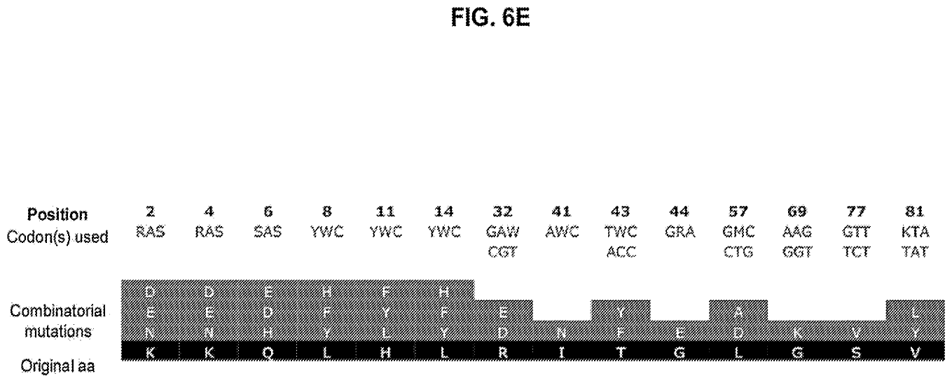

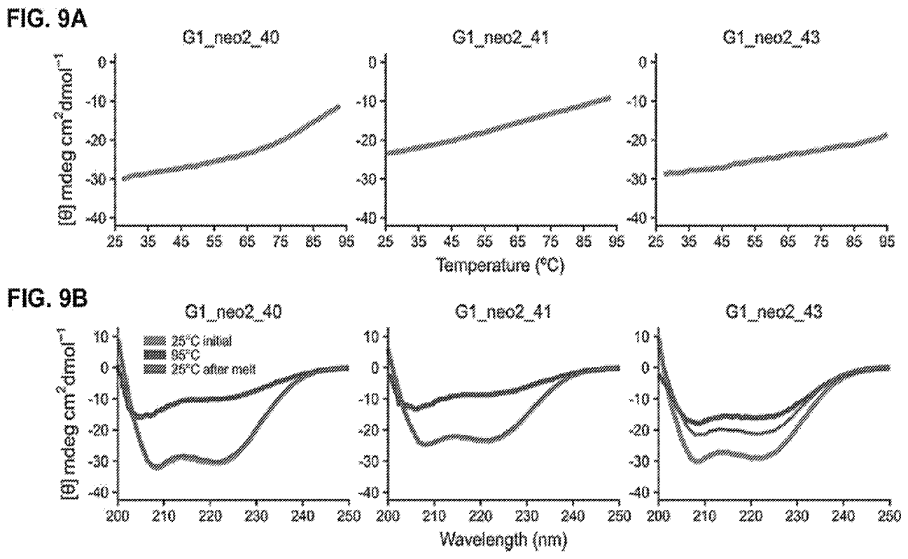

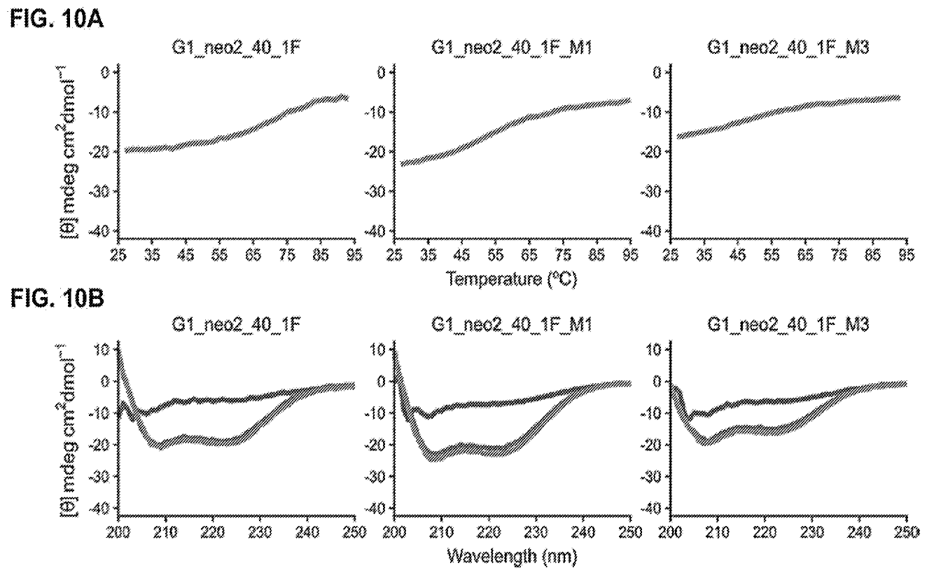

Computational design of non-split IL-2/IL-15 mimetics that bind and activate IL-2Rβ [symbol] _{c}: Native hIL-2 comprises four helices connected by long irregular loops. The N-terminal helix (H1) interacts with both the beta and gamma subunits of the IL-2 receptor, the third helix (H3) interacts with the beta subunit, and the C-terminal helix (H4) with the gamma subunit; the alpha subunit interacting surface is formed by the irregular second helix (H2) and two long loops, one connecting H1 to H2 and the other connecting H3 and H4. An idealized protein was designed that recapitulates the interface formed by H1, H3 and H4 with beta and gamma and to replace H2 with a regular helix that offers better packing. The helices H1, H3 and H4 (see FIG. 1a) were used as a template for the binding site, while helix H2 was reconstructed (H2′) using a database off highly-represented clustered-fragments (see Methods). Pairs of helices were connected with loops extracted from the same database (see FIG. 1b), the resulting helical hairpins combined into fully connected backbones (see FIG. 1c), and Rosetta™ combinatorial flexible backbone sequence design calculations were carried out in the presence of hIL-2Rβ [symbol] _{c} (see Methods). The top four computational designs and eight single-disulfide stapled variations (see Table 2) were selected for experimental characterization by yeast display (see Methods). Eight designs were found to bind fluorescently-tagged beta-gamma chimeric IL-2 receptor at low-nanomolar concentrations. The best non-disulfide design (G1_neo2_40) was subjected to site saturation mutagenesis followed by selection and combination of affinity-increasing substitutions for the murine IL-2Rβ [symbol] _{c} (mIL-2Rβ [symbol] _{c}, see FIG. 5). Optimized designs (were expressed recombinantly in E. coli and found to elicit pSTAT5 signaling in vitro on IL-2-responsive murine cells at low-nanomolar or even picomolar concentrations (Table 3, see Silva et al., Nature 565, pg. 186, Jan. 10, 2019), but had relatively low thermal stability (Tm˜<45° C., see FIGS. 9 and 10). To improve stability, the computational design protocol was repeated starting from the backbone of the highest affinity first round design (G1_neo2_40_1F, topology: H1->H4->H2′->H3), coupling the loop building process with parametric variation in helix length (+/−8 amino acids, see FIG. 1a bottom panel). This second approach improved the quality of the models by enabling the exploration of substantially more combinations of loops connecting each pair of helices. The fourteen best designs of the second generation, along with twenty-seven Rosetta™ sequence redesigns of G1_neo2_40_1F (see Table 4), were experimentally characterized and all but one were found to bind IL-2 receptor at low-nanomolar concentrations. The three highest affinity and stability designs (one sequence redesign and two new mimetics) were subjected to site saturation mutagenesis for mIL-2Rβ [symbol] _{c} binding (FIGS. 6-8), followed by selection and combination of affinity-increasing substitutions for both human and mouse IL-2Rβ [symbol] _{c}. The matured designs (see Table 5) showed enhanced binding while retaining hyper-stability ((see Silva et al., Nature 565, pg. 186, Jan. 10, 2019)). The top design, neoleukin-2/15 (also referred to herein as Neo-2/15), is a 100 residue protein with a new topology and sequence quite different from human or murine IL-2 (29% sequence identity to hIL-2 over 89 residues, and 16% sequence identity to mIL-2 over 76 aligned residues, in structural topology-agnostic based alignment).

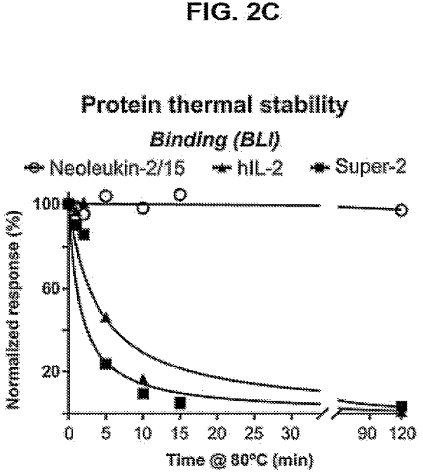

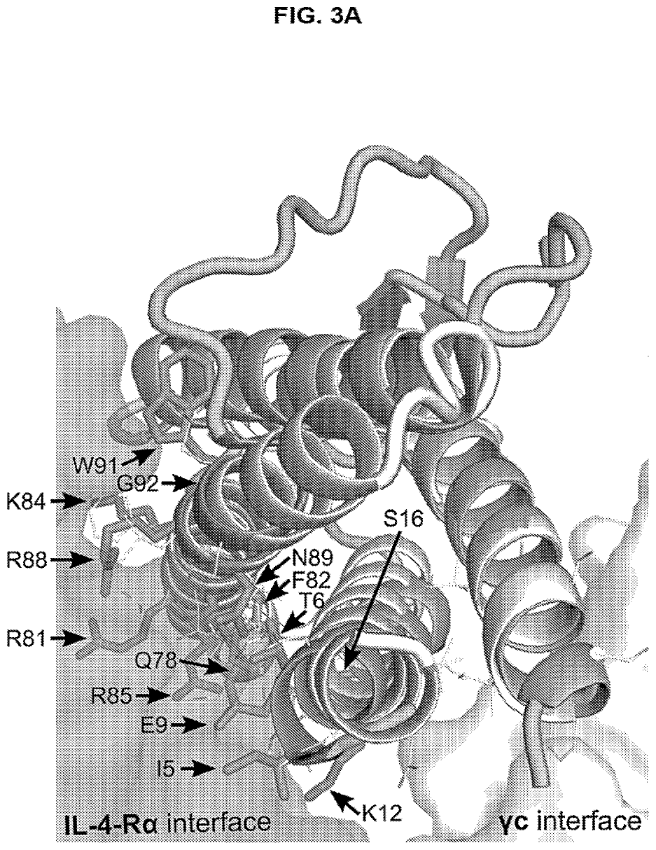

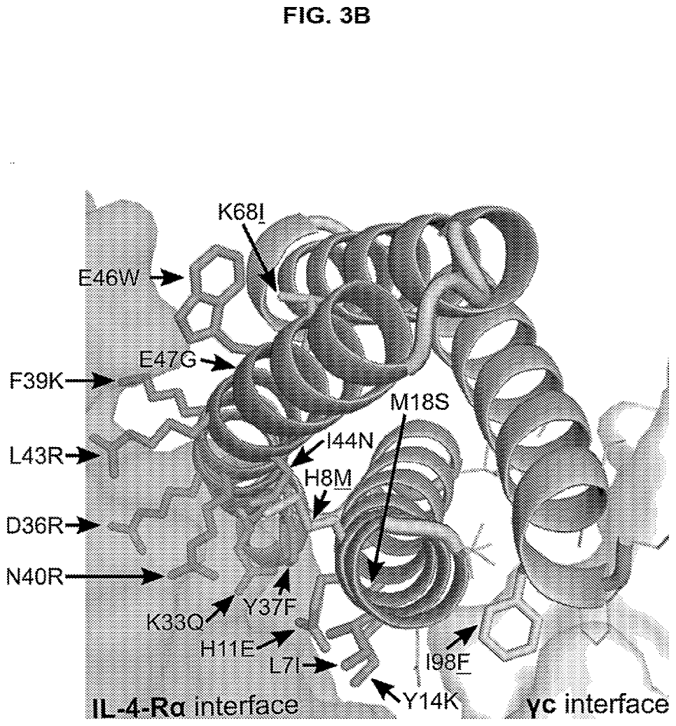

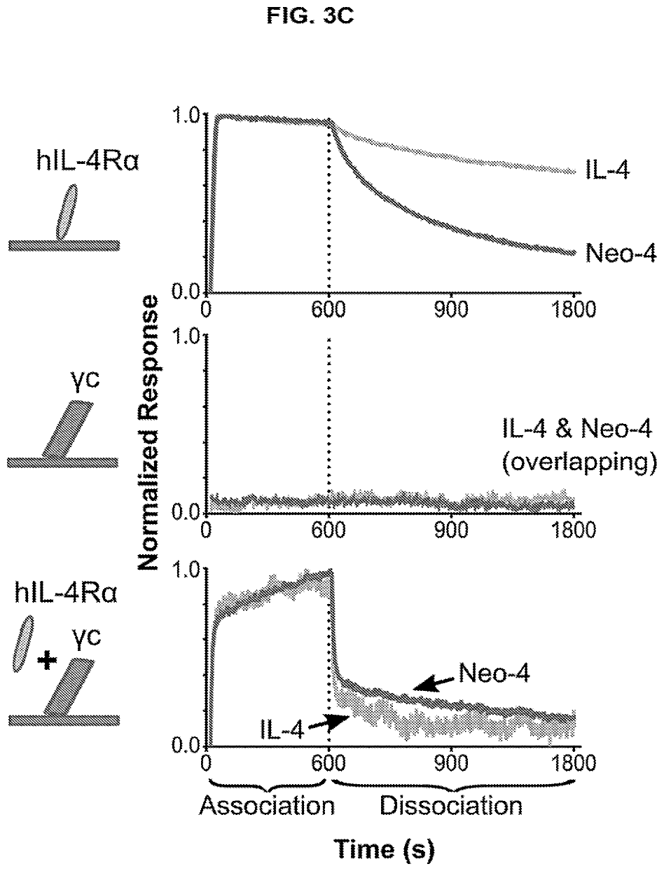

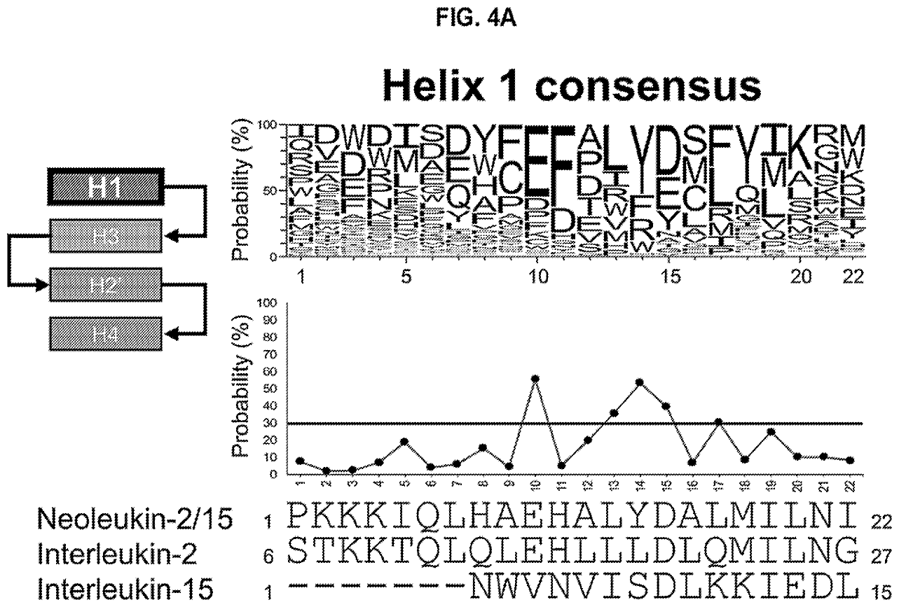

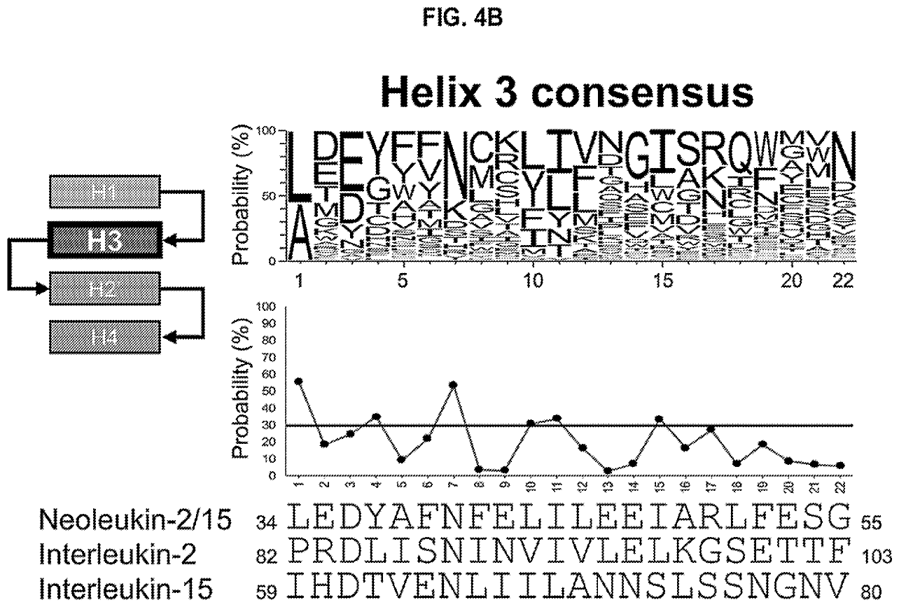

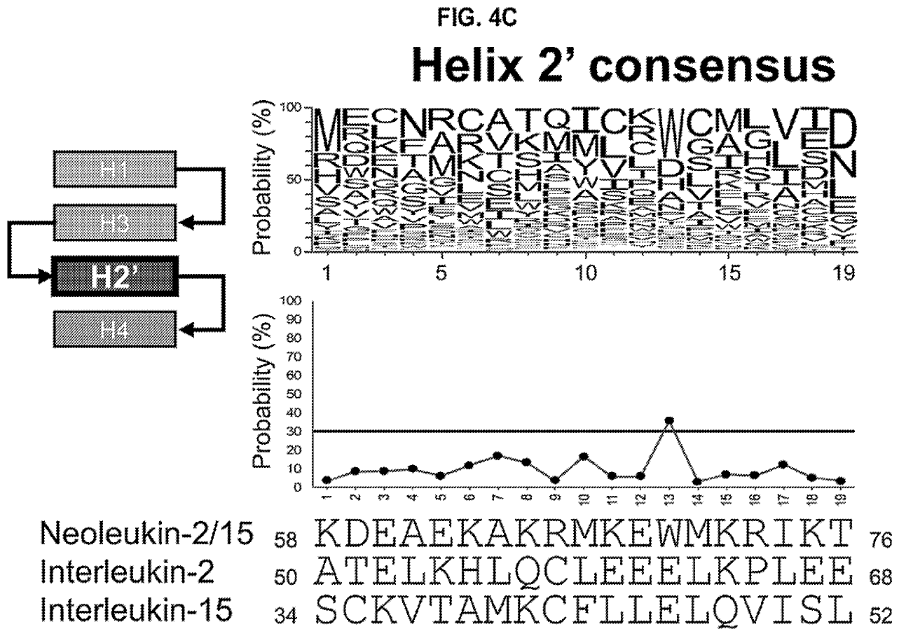

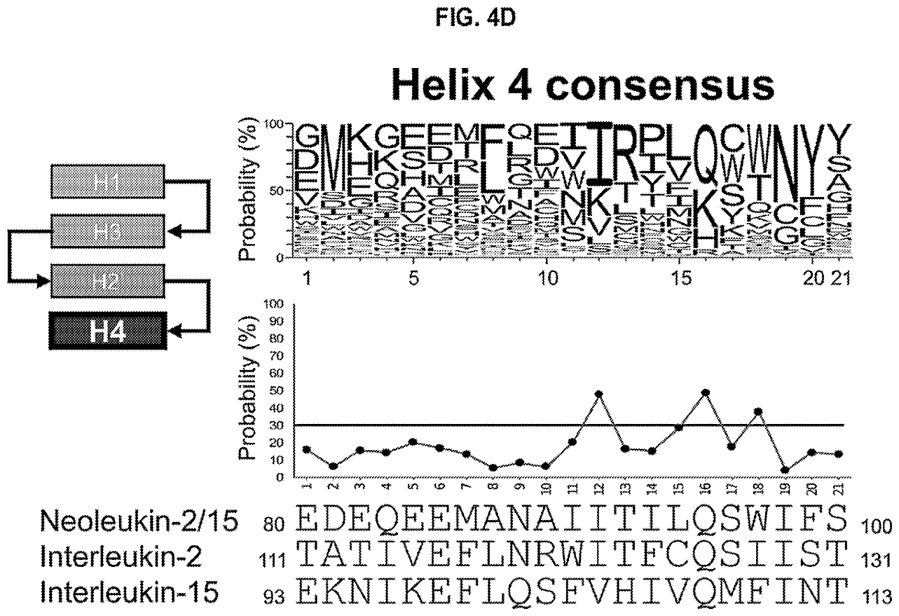

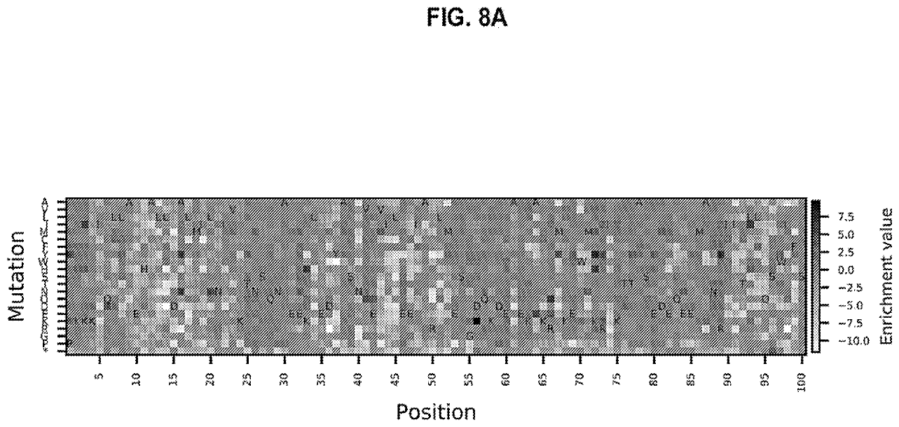

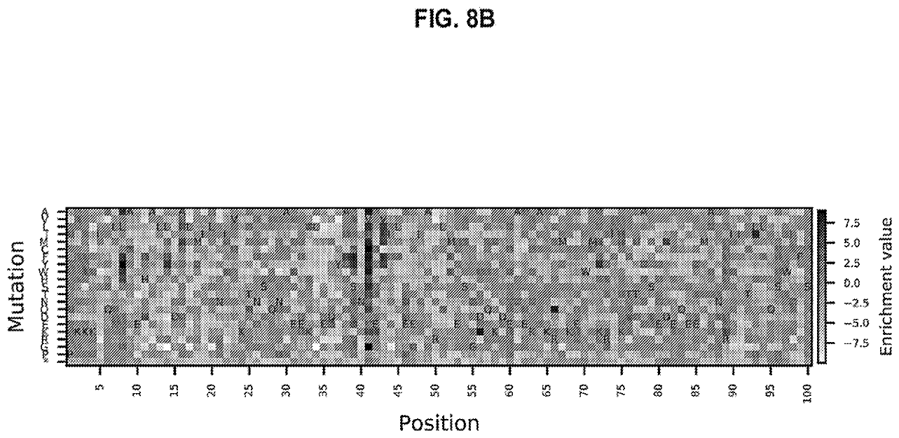

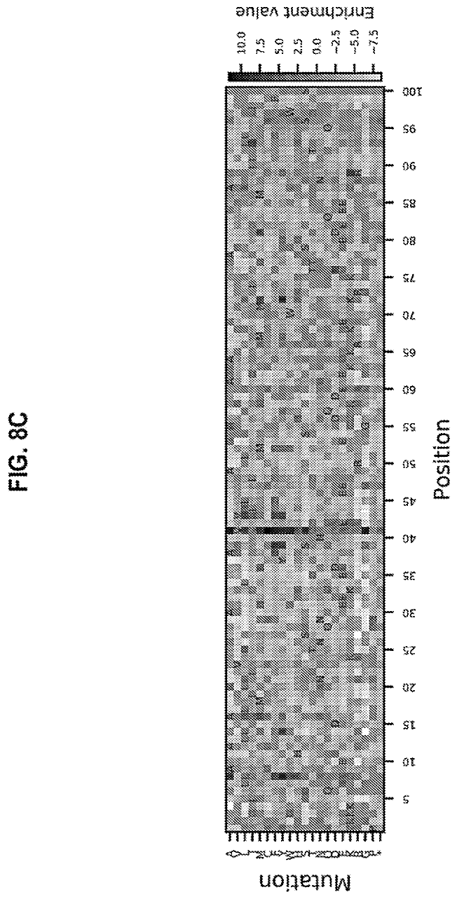

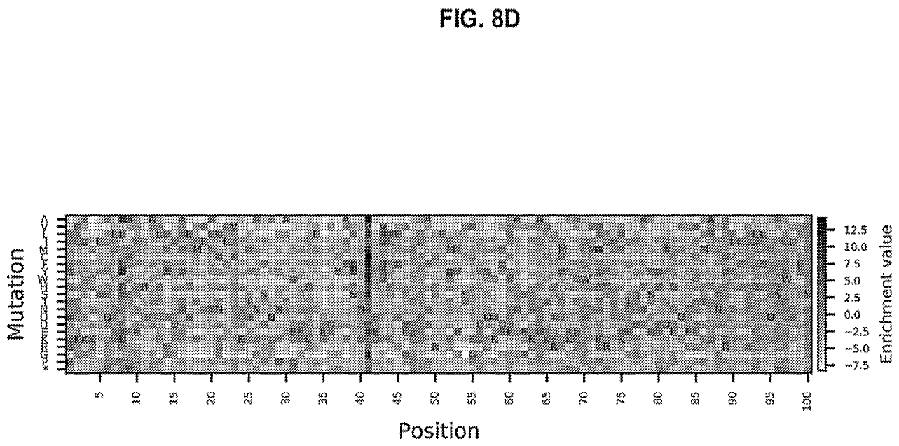

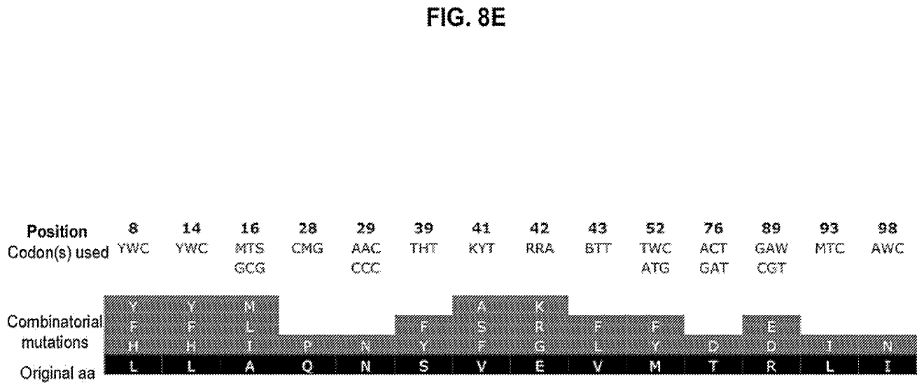

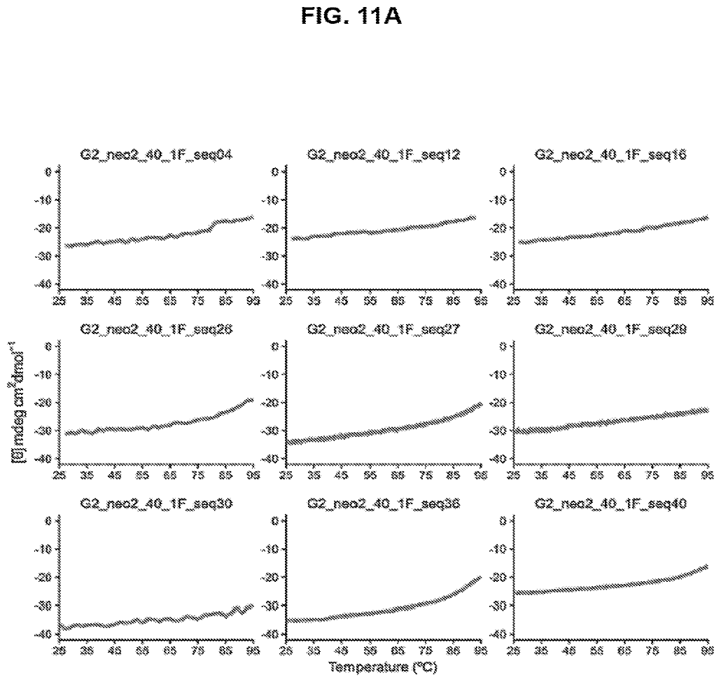

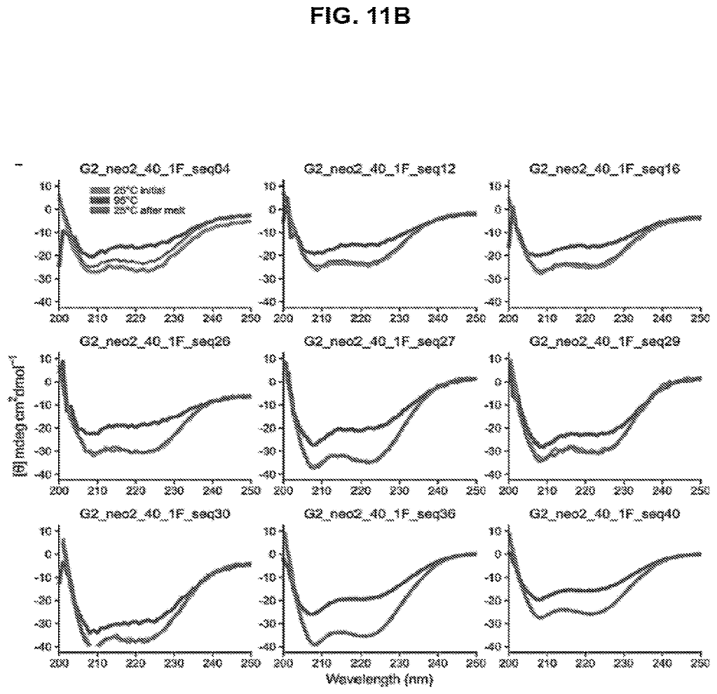

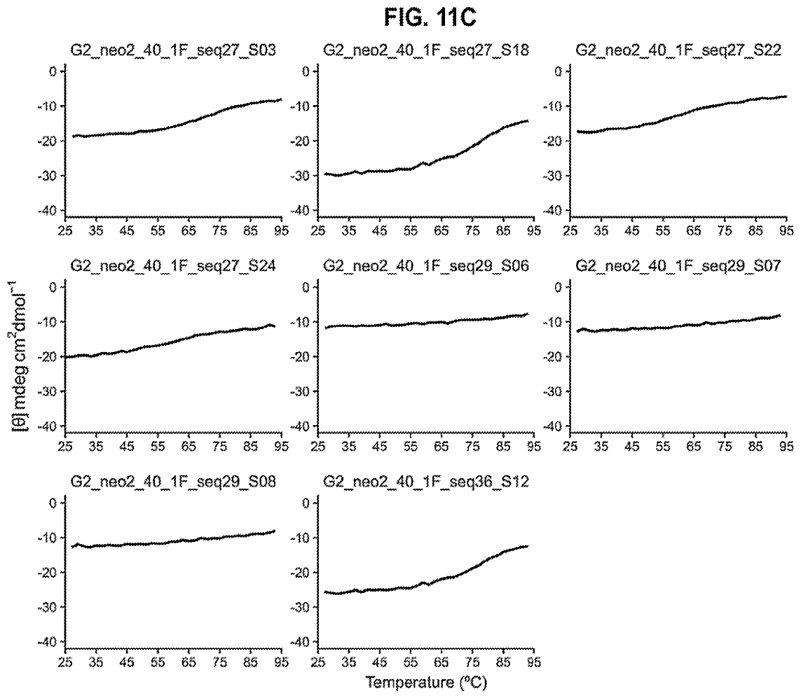

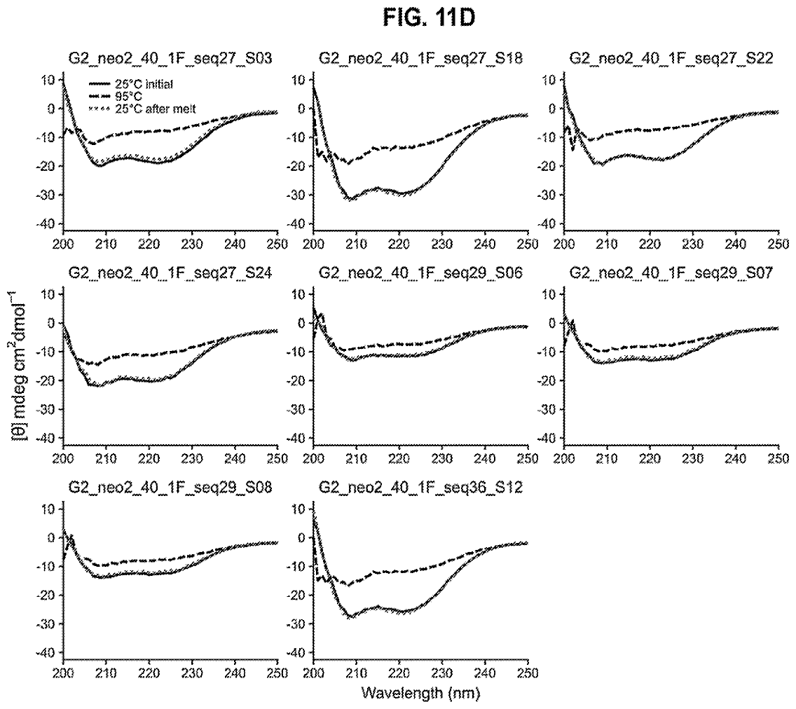

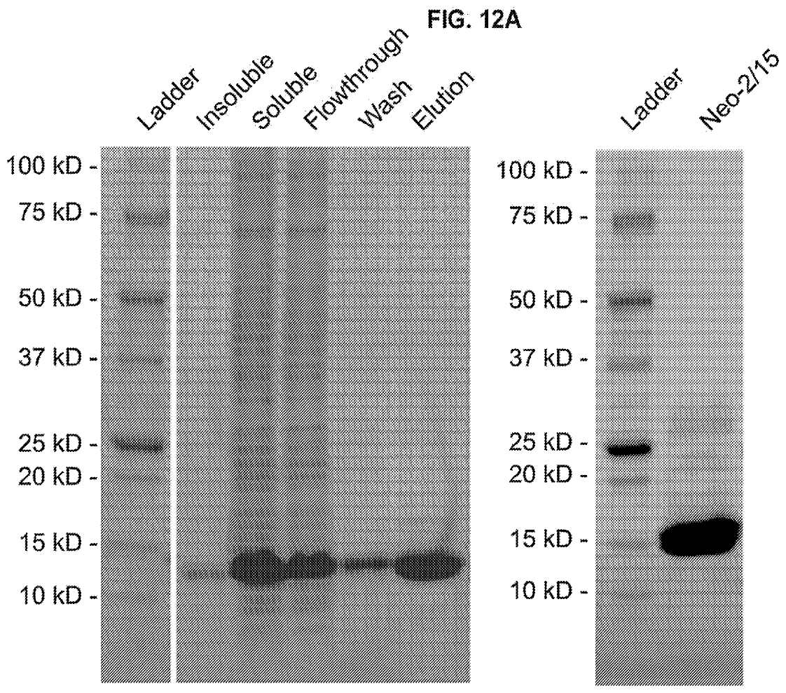

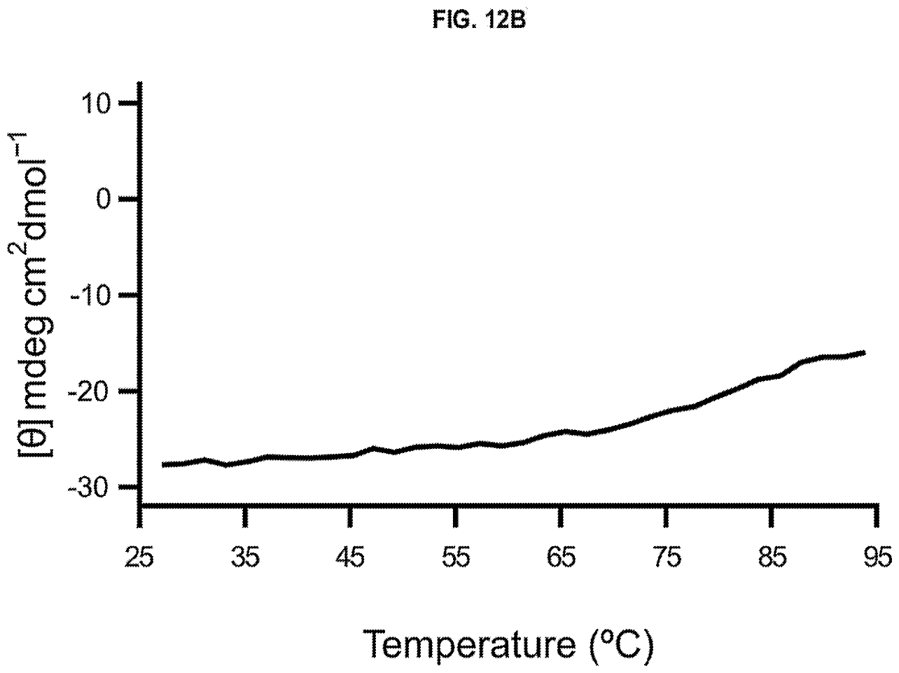

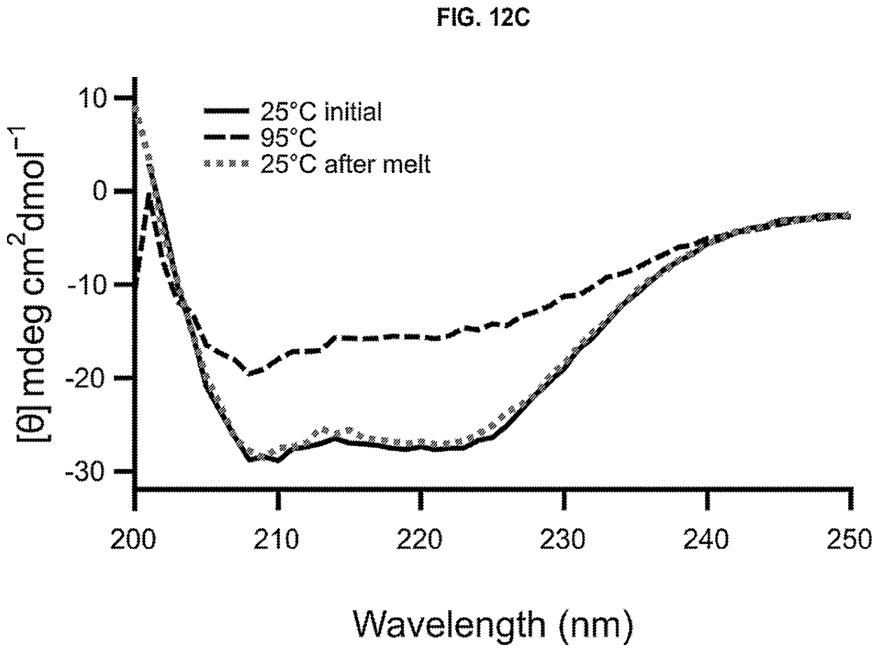

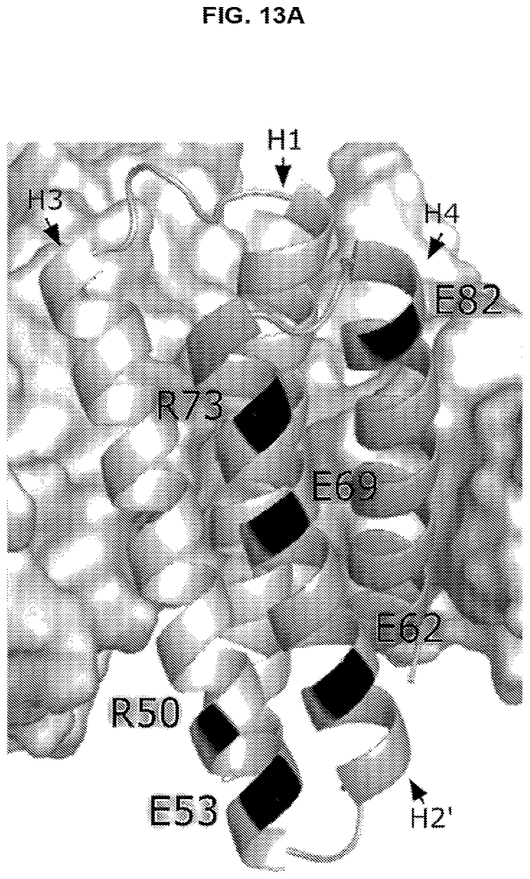

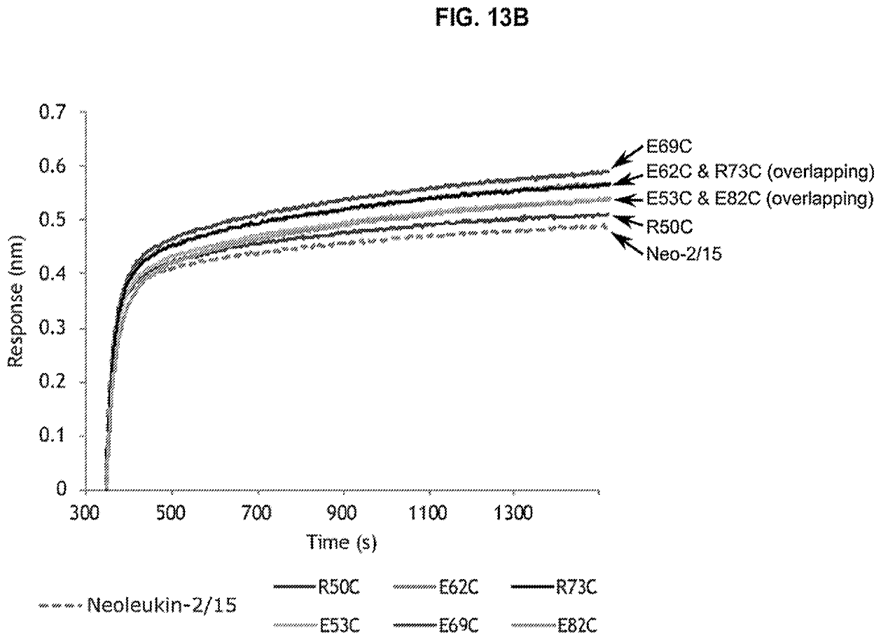

Functional characterization of neoleukin-2/15: Neoleukin-2/15 binds with high affinity to human and mouse IL-2Rβ [symbol] _{c}, but does not interact with IL-2Rα (see Silva et al., Nature 565, pg. 186, Jan. 10, 2019). The affinities of Neoleukin-2/15 for the human and mouse IL-2 receptors (IL-2Rβ and IL-2Rβ [symbol] _{c}) are significantly higher than those of the corresponding native IL-2 cytokines. In contrast with native IL-2, Neoleukin-2/15 elicits IL-2Rα-independent signaling in both human and murine IL-2-responsive cells, and in murine primary T cells (see Silva et al., Nature 565, pg. 186, Jan. 10, 2019). Neoleukin-2/15 activates IL-2Rα− cells more potently than native human or murine IL-2 in accordance with its higher binding affinity. In primary cells, neoleukin-2/15 is more active on IL-2Rα− cells and less active on IL-2Rα+ compared to Super-2, presumably due to its complete lack of IL-2Rα binding. Neoleukin-2/15 is hyper-stable (see FIG. 12) and does not lose binding affinity for hIL-2Rβ [symbol] _{c} following incubation at 80° C. for 2 hours, while hIL-2 and Super-2 are completely inactivated after 10 minutes (half-inactivation time=˜4.2 min and ˜2.6 min, respectively, FIG. 2). Similarly, in ex vivo primary cell cultures, neoleukin-2/15 drove T cell survival effectively after being boiled for 60 minutes at 95° C., while these conditions inactivated both IL-2 and Super-2 (see Silva et al., Nature 565, pg. 186, Jan. 10, 2019). Thermal denaturation studies were carried out on many other of the designed mimetics, demonstrating their thermal stability as well (see FIG. 11). This unprecedented stability for a cytokine-like molecule, beyond eliminating the requirement for cold chain storage, suggests a robustness to mutations (see FIGS. 8 and 13), genetic fusions and chemical modification greatly exceeding that of native IL-2 (see FIG. 3).

Therapeutic applications of neoleukin-2/15: The clinical use of IL-2 has been mainly limited by toxicity. Although the interactions responsible for IL-2 toxicity in humans are incompletely understood, in murine models toxicity is T cell independent and ameliorated in animals deficient in the IL-2Rα chain (CD25+). Thus, many efforts have been directed to reengineer IL-2 to weaken interactions with IL-2Rα, but mutations in the CD25 binding site can be highly destabilizing. The inherent low stability of IL-2 and its tightly evolved dependence on CD25 have been barriers to the translation of reengineered IL-2 compounds. Other efforts have focused on IL-15, since it elicits similar signaling to IL-2 by dimerizing the IL-2Rβ [symbol] _{c} but has no affinity for CD25. However, IL-15 is dependent on trans presentation by the IL-15α (CD215) receptor that is displayed primarily on antigen-presenting cells and natural killer cells. The low stability of native IL-15 and its dependence on trans presentation have also been substantial barriers to reengineering efforts.

Dose escalation studies on naive mice show that mIL-2 preferentially expands regulatory T cells, consistent with preferential binding to CD25+ cells, while neoleukin-2/15 primarily drives expansion of CD8+ T cells and does not induce or minimally induces expansion of regulatory T cells only at the highest dose tested. Similarly, in a murine model of airway inflammation, which normally induces a small percentage of tissue resident CD8+ T cells, neoleukin-2/15 produces an increase in Thy1.2⁻CD44⁺CD8⁺ T cells without increasing CD4⁺ Foxp3⁺ antigen-specific Tregs in the lymphoid organs (data not shown; see Silva et al., Nature 565, pg. 186, Jan. 10, 2019).

The therapeutic efficacy of neoleukin-2/15 was tested in the poorly immunogenic B16F10 melanoma and the more immunogenic CT26 colon cancer mouse models. Single agent treatment with neoleukin-2/15 led to dose-dependent delays in tumour growth in both cancer models. In CT26 colon cancer, single agent treatment showed improved efficacy to that observed for recombinant mIL-2 (see Silva et al., Nature 565, pg. 186, Jan. 10, 2019). In B16F10 melanoma, co-treatment with the anti-melanoma antibody TA99 (anti-TRP1) led to significant tumour growth delays, while TA99 treatment alone had little effect (see Silva et al., Nature 565, pg. 186, Jan. 10, 2019). In long term survival experiments (8 weeks), neoleukin-2/15 in combination with TA99 showed substantially reduced toxicity and an overall superior therapeutic effect compared to mIL-2 (see Silva et al., Nature 565, pg. 186, Jan. 10, 2019). Mice treated with the combination mIL-2 and TA99 steadily lost weight and their overall health declined to the point of requiring euthanasia, whereas little decline was observed with the combination of neoleukin-2/15 and TA99 (see Silva et al., Nature 565, pg. 186, Jan. 10, 2019). Consistent with a therapeutic benefit, neoleukin-2/15 treatment led to a significant increase in intratumoral CD8:T_{reg} ratios (see Silva et al., Nature 565, pg. 186, Jan. 10, 2019), which has been previously correlated with effective antitumor immune responses 58. The increases of CD8:T_{reg} ratios by neoleukin-2/15 are dose and antigen dependent (see Silva et al., Nature 565, pg. 186, Jan. 10, 2019); optimum therapeutic effects were obtained at higher doses and in combination with other immunotherapies (see Silva et al., Nature 565, pg. 186, Jan. 10, 2019). Altogether, these data show that neoleukin-2/15 exhibits the predicted homeostatic benefit derived from its IL-2 like immunopotentiator activity, but without the adverse effects associated with CD25⁺ preferential binding. The therapeutic efficacy of neoleukin-2/15 was tested in a CAR-T model. NSG mice inoculated with 0.5×10⁶ RAJI tumor cells were left untreated, were treated with 0.8×10⁶ anti-CD19 CAR-T cells (infused 7 days after inoculation of tumor cells), or were similarly treated with anti-CD19 CAR-T cells plus 20 pg/day of either human IL-2 or neoleukin-2/15 on days 8-14 after tumor inoculation. As expected, Neoleukin-2/15 was shown to significantly enhance the anti-tumor effect of CAR-T cell therapy, slowing growth of the tumor and extending the survival of the mouse ((see Silva et al., Nature 565, pg. 186, Jan. 10, 2019)).

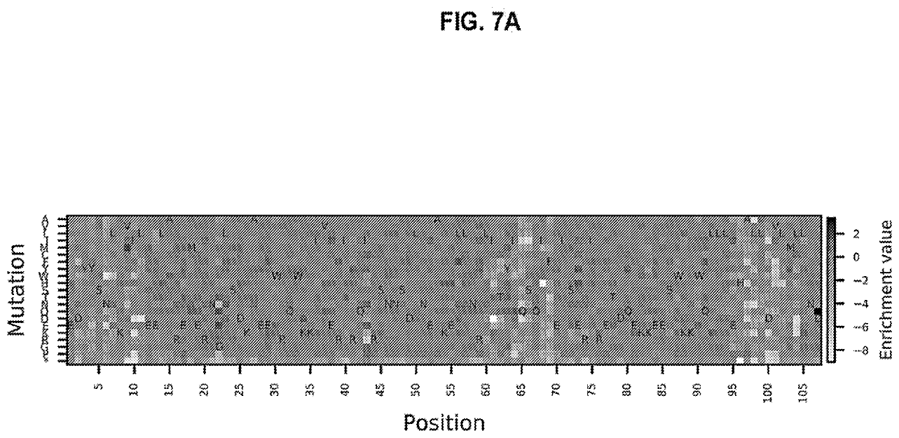

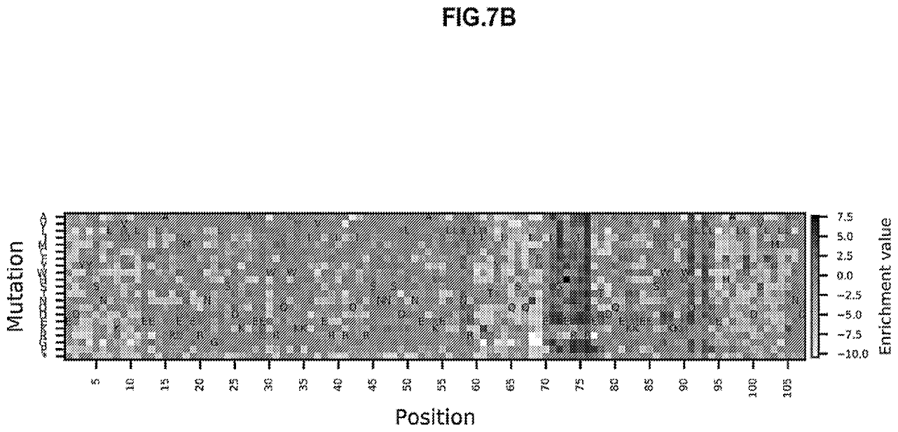

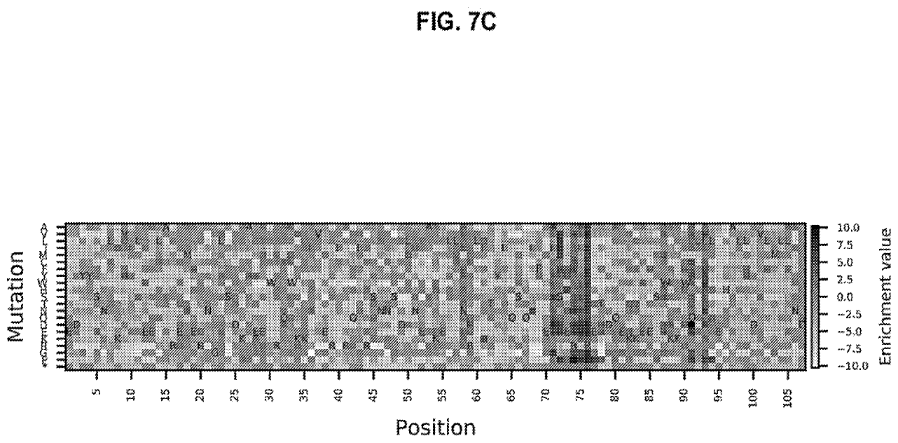

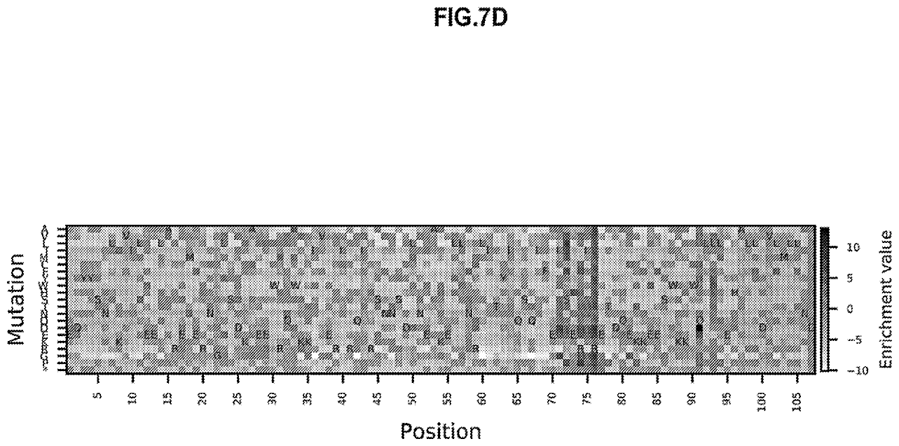

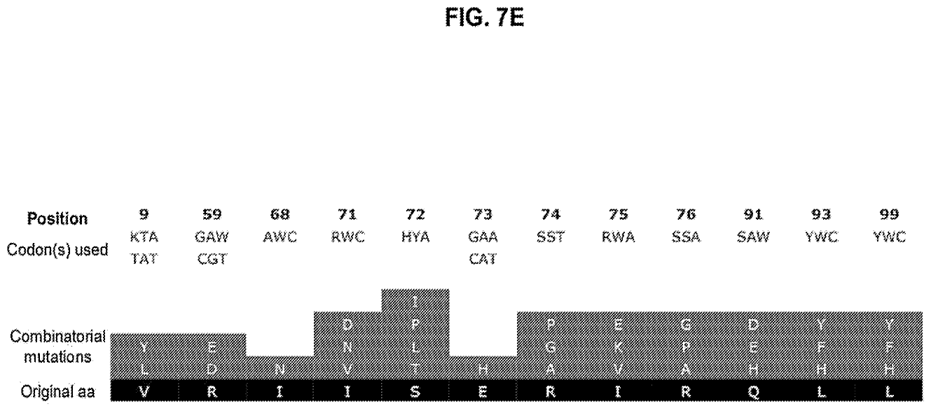

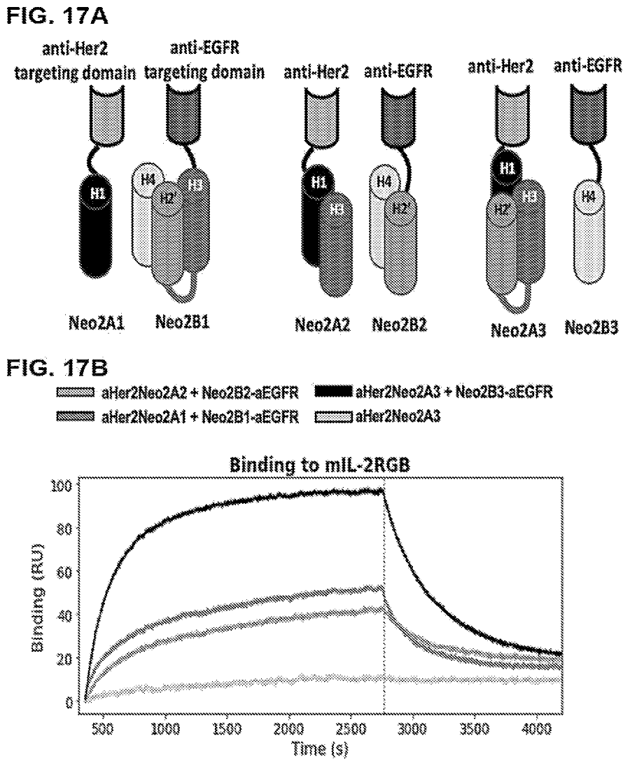

De novo design of protein mimetics has the potential to transform the field of protein-based therapeutics, enabling the development of biosuperior molecules with enhanced therapeutic properties and reduced side-effects, Unlike recombinant IL-2 and engineered variants of hIL-2, neoleukin-2/15 can be solubly expressed in E. coli (see FIG. 17), retains activity at high temperature, does not interact with IL-2Rα and is robust to substantial sequence changes that allow the engineering of new functions (FIG. 7).

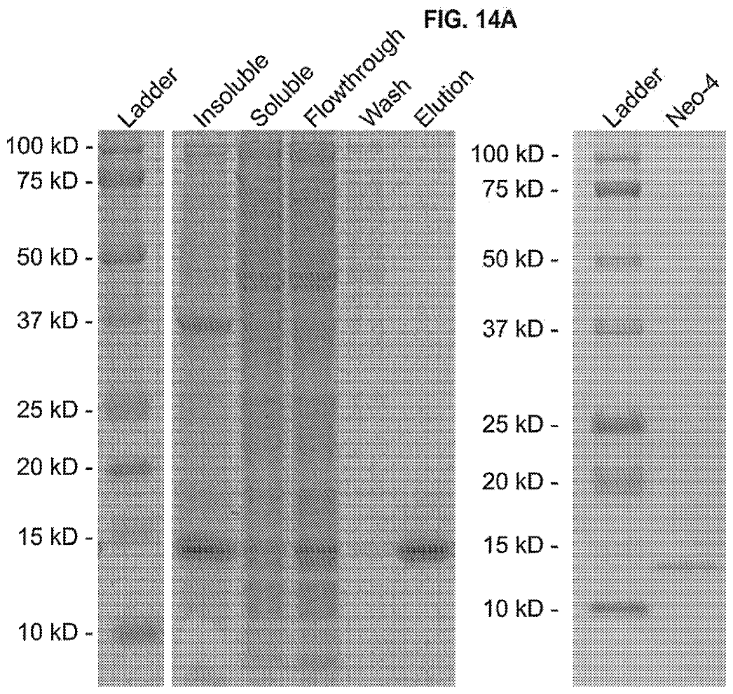

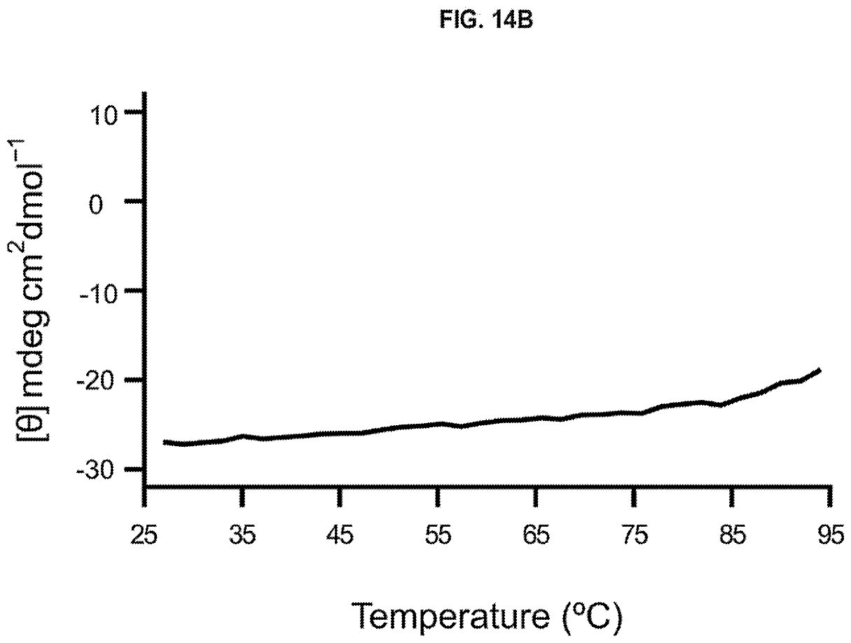

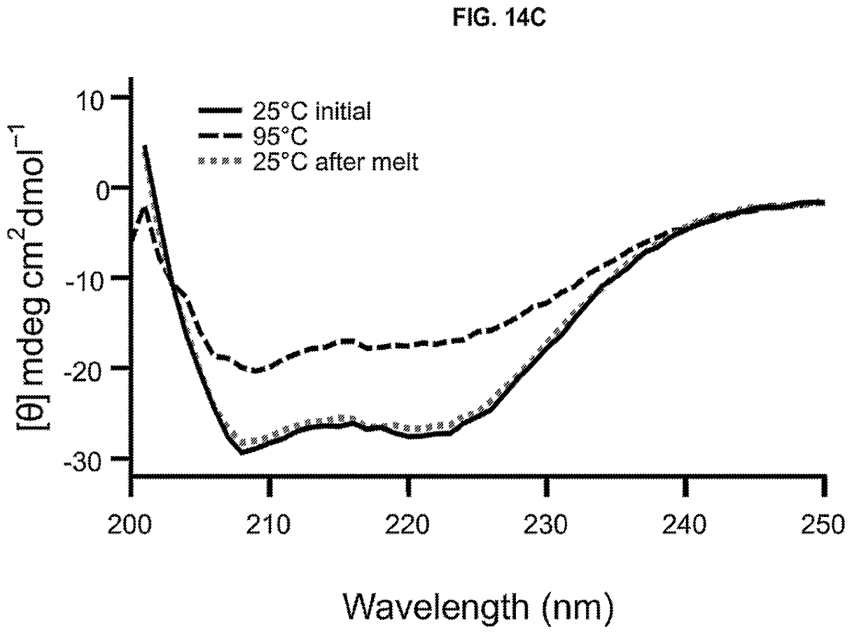

Robust modularity of neoleukin-2/15. Disulfide-stapling and reengineering into an IL-4 mimetic: Neoleukin-2/15 is highly modular, allowing to easily tune its properties, such as increasing its stability or modify its binding preference. This modularity and robustness was taken advantage of by introducing, by computational design, stability enhancing single-disulfide staples that preserve the function of neoleukin-2/15. In one example, a disulfide bridge was introduced by searching pairs of positions with favorable geometrical arrangements followed by flexible backbone minimization. The final design introduced a single disulfide between residues 38 and 75, which stabilizes helices H3 and H2. This strategy increased the stability of neoleukin-2/15 (Tm>95° C.), while retaining its sequence and function mostly unaffected (see Silva et al., Nature 565, pg. 186, Jan. 10, 2019). The modularity properties of neoleukin-2/15 were used to modify its binding preference. All cytokines in the interleukin-2 family interact with the [symbol] _{c} and share a common architecture. Therefore, it was hypothesized that neoleukin-2/15 could be transformed into another cytokine mimetic of the IL-2 family by changing only amino acids in the half of the binding-site that interacts with IL-2Rβ (helices H1 and H3). As proof of a concept, human interleukin-4 (hIL-4) was chosen as target, since it shares extensive structural homology with IL-2 and has potential applications in regenerative medicine. Neo-2/15 was modified to bind to the human IL-4 receptor (comprising IL-4Rα and [symbol] _{c}) and not to the human IL-2 receptor (comprising IL-2Rβ and [symbol] _{c}) by aligning the Neo-2/15 model into the structure of human IL-4 bound to its IL-4 receptor, and mutating 14 residues in Neo-2/15 to match the amino-acids of IL-4 at those structural positions that mediate interactions between IL-4 and IL4r (FIG. 3). Binding was further optimized by directed evolution using random mutagenesis and screening for high binding affinity variants, which introduced two additional amino acid substitutions and modified one of the fourteen original residues grafted from the IL-4 protein, thereby creating a new protein Neoleukin-4 with a total of sixteen mutations from Neoleukin-2/15. The resulting optimized design, neoleukin-4 (see Table 6), was recombinantly expressed and purified from E. coli and tested for binding. Neoleukin-4 binds with high affinity to IL-4Rα receptor, binds cooperatively to IL-4Rα [symbol] _{c} (see FIG. 3), and does not bind with any affinity to the IL-2 receptor (data not shown) Neoleukin-4 retains the superior thermostable properties of neoleukin-2/15 (see FIG. 14b,c), and binds to the IL-13 receptor as expected given the natural cross-reactivity of IL-4 to IL-13 receptor (data not shown).

### Methods

Computational design of de novo cytokine mimetics: The design of de novo cytokine mimetics began by defining a the structure of hIL-2 in the quaternary complex with the IL-2Rβ [symbol] _{c} receptor as template for the design. After inspection, the residues composing the binding-site were defined as hotspots using Rosetta™ metadata (PDBInfoLabels). The structure was feed into the new mimetic design protocol that is programmed in PyRosetta™, and which can automatically detect the core-secondary structure elements that compose the target-template and produce the resulting de novo mimetic backbones with full RosettaScripts™ compatible information for design. Briefly, the mimetic building algorithm works as follows. For the first generation of designs, each of the core-elements was idealized by reconstruction using loops from a clustered database of highly-ideal fragments (fragment-size 4 amino acids). After idealization, the mimetic building protocol aims to reconnect the idealized elements by pairs in all possible combinations. To do this it uses combinatorial fragment assembly of sequence-agnostic fragments from the database, followed by cartesian-constrained backbone minimization for potential solutions (i.e. where the N- and C-ends of the built fragment are close enough to link the two secondary structures). After minimization, the solutions are verified to contain highly ideal fragments (i.e. that every overlapping fragment that composes the two connected elements is also contained within the database) and no backbone clashes with the target (context) receptor. Passing backbone solutions were then profiled using the same database of fragments in order to determine the most probable amino acids at each position (this information was encoded in metadata on the design). Next, solutions for pairs of connected secondary structures were combinatorially recombined to produce fully connected backbones by using graph theory connected components. Since the number of solutions grows exponentially with each pair of elements, at each fragment combination step we ranked the designs to favor those with shorter interconnections between pairs of core elements, and kept only the top solutions to proceed to the next step. Fully connected solutions were then profiled by layer (interface, core, non-core-surface, surface), in order to restrict the identities of the possible amino acids to be layer-compatible. Finally, all the information on hotspots, compatible built-fragment amino acids and layers were combined (hotspot has precedence to amino acid probability, and amino acid probability took precedence to layer). These fully profiled backbones were then passed to RosettaScripts™ for flexible backbone design and filtering. For the second generation of designs, two approaches were followed. In the first approach, sequence redesigns of the best first generation optimized design were executed (G1_neo2_40_1F,). In the second approach new mimetics were engineered using G1_neo2_40_1F as the target template. The mimetic design protocol in this second generation was similar to the one described for the first generation, but with two key differences. Firstly, the core-fragments were no longer built from fragments, but instead by discovering parametric equations of repetitive phi and psi angles (omega fixed to 180°) that result in repetitive secondary structures that recapitulated each of the target helices as close as possible, a “pitch” on the phi and psi angles was allowed every X-amino acids in order to allow the helices the possibility to have curvature (final parameters: H1:, H2:, H3, H4), the sue of these parametric equations allowed to change the size of each of the core-elements in the target structure at will (either increase or decrease the size), which was coupled (max/min 8.a.a.) with the loop building process, and reductions in the size of the core elements were not allowed to remove hotspots from the binding site. The second difference in the second generation designs, is that instead of reconnecting the secondary structure core-elements we used a fragment-size of 7 amino acids, and no combinatorial assembly of more than one fragment was allowed (i.e. a single fragment has to be able to close a pair of secondary structures). The rest of the design algorithm was in essence similar to the one followed in the generation one. The Rosetta™ energy functions used were “talaris2013” and “talaris2014”, for the first and second generation of designs, respectively.

The databases of highly ideal fragments used for the design of the backbones for the de novo mimetics were constructed with the new Rosetta™ application “keenters_clustering_of_fragments” using an extensive database of non-redundant publicly available protein structures from the RCSB protein data bank, which was comprised of 16767 PDBs for the 4-mer database used for the first generation designs, and 7062 PDBs for the 7-mer database used for the second generation designs.

Yeast display: Yeast were transformed with genes encoding the proteins to be displayed together with linearized pETcon3 vector. The vector was linearized by 100 fold overdigestion by NdeI and XhoI (New England Biolabs) and then purified by gel extraction (Qiagen). The genes included 50 bases of overlap with the vector on both the 5′ and 3′ ends such that homologous recombination would place the genes in frame between the AGA2 gene and the myc tag on the vector. Yeast were grown in C-Trp-Ura media prior to induction in SGCAA media as previously described. 12-18 hours after induction, cells were washed in chilled display buffer (50 mM NaPO₄ pH 8, 20 mM NaCl, 0.5% BSA) and incubated with varying concentrations of biotinylated receptor (either human or murine IL-2Rα, IL-2Rβ, IL-2Rγ, or human IL-4Rα) while being agitated at 4° C. After approximately 30 minutes, cells were washed again in chilled buffer, and then incubated on ice for 5 minutes with FITC-conjugated anti-c-Myc antibody (1 uL per 3×10⁶ cells) and streptavidin-phycoerythrin (1 uL per 100 uL volume of yeast). Yeast were then washed and counted by flow cytometry (Accuri C6) or sorted by FACS (Sony SH800). For experiments in which the initial receptor incubation was conducted with a combination of biotinylated IL-2Rγ and non-biotinylated IL-4Rα, the non-biotinylated receptor was provided in molar excess.

Mutagenesis and affinity maturation: For error-prone PCR based mutagenesis, the design to be mutated was cloned into pETcon3 vector and amplified using the MutaGene™ II mutagenesis kit (Invitrogen) per manufacturer's instructions to yield a mutation frequency of approximately 1% per nucleotide. 1 μg of this mutated gene was electroporated into EBY100 yeast together with 1 μg of linearized pETcon3 vector, with a transformation efficiency on the order of 108. The yeast were induced and sorted multiple times in succession with progressively decreasing concentrations of receptor until convergence of the population. The yeast were regrown in C-Trp-Ura media between each sort.

Site-saturation mutagenesis (SSM) libraries were constructed from synthetic DNA from Genscript. For each amino acid on each design template, forward primers and reverse primers were designed such that PCR amplification would result in a 5′ PCR product with a degenerate NNK codon and a 3′ PCR product, respectively. Amplification of “left” and “right” products by COF and COR primers yielded a series of template products each consisting of a degenerate NNK codon at a different residue position. For each design, these products were pooled to yield the SSM library. SSM libraries were transformed by electroporation into conditioned Saccharomyces cerevisiae strain EBY100 cells, along with linearized pETCON3 vector, using the protocol previously described by Benatuil et al.

Combinatorial libraries were constructed from synthetic DNA from Genscript containing ambiguous nucleotides and similarly transformed into linearized pETCON3 vector.

Protein expression: Genes encoding the designed protein sequences were synthesized and cloned into pET-28b(+) E. coli plasmid expression vectors (GenScript, N-terminal 6×His tag and thrombin cleavage site). Plasmids were then transformed into chemically competent E. coli Lemo21 cells (NEB). Protein expression was performed using Terrific Broth™ and M salts, cultures were grown at 37° C. until OD⁶¹⁰ reached approximately 0.8, then expression was induced with 1 mM of isopropyl β-D-thiogalactopyranoside (IPTG), and temperature was lowered to 18° C. After expression for approximately 18 hours, cells were harvested and lysed with a Microfluidics M110P microfluidizer at 18,000 psi, then the soluble fraction was clarified by centrifugation at 24,000 g for 20 minutes. The soluble fraction was purified by Immobilized Metal Affinity Chromatograpy (Qiagen) followed by FPLC size-exclusion chromatography (Superdex™ 75 10/300 GL, GE Healthcare). The purified neoleukin-2/15 was characterized by Mass Spectrum (MS) verification of the molecular weight of the species in solution (Thermo Scientific), Size Exclusion—MultiAngle Laser Light Scattering (SEC-MALLS) in order to verify monomeric state and molecular weight (Agilent, Wyatt), SDS-PAGE, and endotoxin levels (Charles River).

Human and mouse IL-2 complex components including hIL-2 (a.a. 1-133), hIL-2Rα (a.a. 1-217), hIL-2Rβ (a.a. 1-214) hIL-2Rγ (a.a. 1-232), mIL-2 (a.a. 1-149), mIL-2Rα ectodomain (a.a. 1-213), mIL-2Rβ ectodomain (a.a. 1-215), and myc ectodomain (a.a. 1-233) were secreted and purified using a baculovirus expression system, as previously described 17,49. All proteins were purified to >98% homogeneity with a Superdex™ 200 sizing column (GE Healthcare) equilibrated in HBS. Purity was verified by SDS-PAGE analysis. For expression of biotinylated human IL-2 and mouse IL-2 receptor subunits, proteins containing a C-terminal biotin acceptor peptide (BAP)-LNDIFEAQKIEWHE (SEQ ID NO:303) were expressed and purified as described via Ni-NTA affinity chromatography and then biotinylated with the soluble BirA ligase enzyme in 0.5 mM Bicine pH 8.3, 100 mM ATP, 100 mM magnesium acetate, and 500 mM biotin (Sigma). Excess biotin was removed by size exclusion chromatography on a Superdex 200 column equilibrated in HBS.

Circular dichroism (CD): Far-ultraviolet CD measurements were carried out with an AVIV spectrometer model 420 in PBS buffer (pH 7.4) in a 1 mm path-length cuvette with protein concentration of ˜0.20 mg/ml (unless otherwise mentioned in the text). Temperature melts where from 25 to 95° C. and monitored absorption signal at 222 nm (steps of 2° C./min, 30 s of equilibration by step). Wavelength scans (195-260 nm) were collected at 25° C. and 95° C., and again at 25° C. after fast refolding (˜5 min).

STAT5 phosphorylation studies: In vitro studies: Approximately 2×10⁵ YT-1, IL-2Rα⁺ YT-1, or CTLL-2 cells are plated in each well of a 96-well plate and re-suspended in RPMI complete medium containing serial dilutions of hIL-2, mIL-2, Super-2, or engineered IL-2 mimetics. Cells are stimulated for 15 min at 37° C. and immediately fixed by addition of formaldehyde to 1.5% and 10 min incubation at room temperature. Permeabilization of cells is achieved by resuspension in ice-cold 100% methanol for 30 min at 4° C. Fixed and permeabilized cells are washed twice with FACS buffer (phosphate-buffered saline [PBS] pH 7.2 containing 0.1% bovine serum albumin) and incubated with Alexa Fluor®647-conjugated anti-STAT5 pY694 (BD Biosciences) diluted in FACS buffer for 2 hours at room temperature. Cells are then washed twice in FACS buffer and MFI was determined on a CytoFLEX™ flow cytometer (Beckman-Coulter). Dose-response curves are fitted to a logistic model and half-maximal effective concentration (EC₅₀ values) are calculated using GraphPad Prism data analysis software after subtraction of the mean fluorescence intensity (MFI) of unstimulated cells and normalization to the maximum signal intensity. Experiments are conducted in triplicate and performed three times with similar results. Ex vivo studies: Spleens and lymph nodes are harvested from wild-type C57BL/6J or B6; 129S4-Il2ra^{tm1Dw} (CD25KO) mice and made into a single cell suspension in sort buffer (2% Fetal Calf Serum in pH 7.2 phosphate-buffered saline). CD4+ T cells are enriched through negative selection by staining the cell suspension with biotin-conjugated anti-B220, CD8, NK1.1, CD11b, CD11c, Ter119, and CD19 antibodies at 1:100 for 30 min on ice. Following a wash with sort buffer, anti-biotin MicroBeads™ (Miltenyi Biotec) is added to the cell suspension at 20 μL per 10⁷ total cells and incubated on ice for 20 minutes. Cells are washed, resuspended and negative selection is then performed using EasySep™ Magnets (STEMCELL Technologies). Approximately 1×10⁵ enriched cells are added to each well of a 96-well plate in RPMI complete medium with 5% FCS with 10-fold serial dilutions of mIL-2, Super-2, or Neoleukin-2/15. Cells are stimulated for 20 minutes at 37° C. in 5% CO₂ fixed with 4% PFA and incubated for 30 minutes at 4° C. Following fixation, cells are harvested and washed twice with sort buffer and again fixed in 500 μL 90% ice-cold methanol in dH₂O for 30 minutes on ice for permeabilization. Cells are washed twice with Perm/Wash Buffer (BD Biosciences) and stained with anti-CD4-PerCP in Perm/Wash buffer (1:300), anti-CD44-Alexa Fluor 700 (1:200), anti-CD25-PE-Cy7 (1:200), and 5 μL per sample of anti-pSTAT5-PE pY694 for 45 min at room temperature in the dark. Cells are washed with Perm/Wash and re-suspended in sort buffer for analysis on a BD LSR II flow cytometer (BD Biosciences).

### Data Tables

TABLE 2
Amino acid sequences for the best twelve first-round designs.
Ten of the designs were (G1_neo2_35-44) were experimentally
characterized by yeast display and all but two
(G1_neo2_35 and G1_neo2_44) were found to bind fluorescently
labeled chimeric ILRβγ_{c} at low nanomolar concentrations via flow
cytometry screening of designed first-roundprotein binders.
Designs indicated were expressed on yeast and incubated
with 2 nM hIL-2Rβ [symbol] or 0 nM IL-2Rβ [symbol] (data not shown).

| Design | Sequence |
|---|---|
| G1_neo2_33 | STKKWQLQAEHALLDWQMALNKSPEPNENLNRAITAAQSWISTGKIDLDKAEDIRR |
| | NSDQARREAEKRGIDVRDLISNAQVILLEAR |
| | (SEQ ID NO: 103) |
| G1_neo2_34 | STKKWQLQAEHALLDWQMALNKSPEPNENLNRAITAAQSCISTGKCDLDKAEDIRR |
| | NSDQARREAEKRGIDVRDLISNAQVILLEAR |
| | (SEQ ID NO: 104) |
| G1_neo2_35 | STKKWQLQAEHALLDWQMALNKSPEPNENLNRAITAAQSWISTGKIDCDKAEDIRR |
| | NSDQARREAEKRGIDVRDLISNAQVILLEAC |
| | (SEQ ID NO: 105) |
| G1_neo2_36 | STKKLQLQAEHFLLDVQMILNESPEPNEELNRAITDAQSWISTGKIDLDRAEELAR |
| | NLEKVRDEALKRGIDVRDLVSNAKVIALELK |
| | (SEQ ID NO: 106) |
| G1_neo2_37 | STKKLQLQAEHFLLDVQMILNESPEPNEELNRCITDAQSWISTGKIDLDRAEECAR |
| | NLEKVRDEALKRGIDVRDLVSNAKVIALELK |
| | (SEQ ID NO: 107) |
| G1_neo2_38 | STKKLQLQAEHFLLDVQMILNESPEPNEELNRAITDAQSCISTGKCDLDRAEELAR |
| | NLEKVRDEALKRGIDVRDLVSNAKVIALELK |
| | (SEQ ID NO: 108) |
| G1_neo2_39 | STKKLQLQAEHFLLDVQMILNESPEPNEELNRAITDAQSWISTGKIDLDRAEELCR |
| | NLEKVRDEALKRGIDVRDLVSNACVIALELK |
| | (SEQ ID NO: 109) |
| G1_neo2_40 | STKKLQLQAEHALLDAQMMLNRSPEPNEKLNRIITTMQSWISTGKIDLDGAKELAK |
| | EVEELRQEAEKRGIDVRDLASNLKVILLELA |
| | (SEQ ID NO: 110) |
| G1_neo2_41 | STKKLQLQAEHALLDAQMMLNRSPEPNEKLNRIITTMQSCISTGKCDLDGAKELAK |
| | EVEELRQEAEKRGIDVRDLASNLKVILLELA |
| | (SEQ ID NO: 111) |
| G1_neo2_42 | STKKIQLQLEHALLDVQMALNRSPEPNESLNRMITWLQSWISTGKIDLDNAQEMAK |
| | EAEKIRKEMEKRGIDVRDLISNIIVILLELS |
| | (SEQ ID NO: 112) |
| G1_neo2_43 | STKKIQLQLEHALLDVQMALNRSPEPNESLNRMITWLQSCISTGKCDLDNAQEMAK |
| | EAEKIRKEMEKRGIDVRDLISNIIVILLELS |
| | (SEQ ID NO: 113) |
| G1_neo2_44 | STKKIQLQLEHALLDVQMALNRSPEPNESLNRMITWLQSWISTGKIDLDNAQEMCK |
| | EAEKIRKEMEKRGIDVRDLISNICVILLELS |
| | (SEQ ID NO: 114) |

TABLE 3
Amino acid sequences for the experimentally optimized first-round designs.

| Design | Sequence |
|---|---|
| G1_neo2_40_1A | STKKTQLLAEHALLDAFMMLNVVPEPNEKLNRIITTMQSWIYTGKIDADGAKELAKEVEELEQEYEKRGIDVEDD |
| | ASNLKVILLELA |
| | (SEQ ID NO: 115) |
| G1_neo2_40_1B | STKKTQLLAEHALLDAHMMLNMLPEPNEKLNRIITTMQSWIHTGKIDGDGAQELAKEVEELEQEYEKRGIDVEDE |
| | ASNLKVILLELA |
| | (SEQ ID NO: 116) |
| G1_neo2_40_1C | STKKTQLLAEHALLDAFMMLNMVPEPNEKLNRIITTMQSWIFTGKIDGDGAKELAKEVEELEQEFEKRGIDVEDE |
| | ASNLKVILLELA |
| | (SEQ ID NO: 117) |
| G1_neo2_40_1D | STKKTQLLAEHALLDALMMLNMVPEPNEKLNRIITTMQSWIFTGKIDGDGAQELAKEVEELEQELEKRGIDVEDY |
| | ASNLKVILLELA |
| | (SEQ ID NO: 118) |
| G1_neo2_40_1E | STKKTQLLAEHALLDAHMMLNVVPEPNEKLNRIITTMQSWIYTGKIDRDGAQELAKEVEELEQELEKRGIDVDDD |
| | ASNLKVILLELA |
| | (SEQ ID NO: 119) |
| G1_neo2_40_1F | STKKTQLLAEHALLDALMMLNLLPEPNEKLNRIITTMQSWIFTGKIDGDGAQELAKEVEELEQEHEKRGIDVEDY |
| | ASNLKVILLELA |
| | (SEQ ID NO: 120) |
| G1_neo2_40_1G | STKKTQLLAEHALLDAYMMLNMVPEPNEKLNRIITTMQSWILTGKIDSDGAQELAKEVEELEQELEKRGIDVDDD |
| | ASNLKVILLELA |
| | (SEQ ID NO: 121) |
| G1_neo2_40_1H | STKKTHLLAEHALLDAYMMLNVMPEPNEKLNRIITTMQSWIFTGKIDGDGAKELAKEVEELEQEFEKRGIDVDDD |
| | ASNLKVILLELA |
| | (SEQ ID NO: 122) |
| G1_neo2_40_1I | STKKTQLLAEHALLDAYMMLNLVPEPNEKLNRIITTMQSWIFTGKIDADGAQELAIEVEELEQEYEKRGIDVDDY |
| | ASNLKVILLELA |
| | (SEQ ID NO: 123) |
| G1_neo2_40_1J | STKKTQLMAEHALLDAFMMLNVLPEPNEKLNRIITTMQSWIFTGKIDGDDAQELAKEVEELEQELEKRGIDVDDD |
| | ASNLKVILLELA |
| | (SEQ ID NO: 124) |
| G1_neo2_40_1F_H1 | STKKTQLLIEHALLDALDMSRNLPEPNEKLSRIITTMQSWIFTGKIDGDGAQQLAKEVEELEQEHEKRGEDVEDE |
| | ASNLKVILLELA |
| | (SEQ ID NO: 125) |
| G1_neo2_40_1F_H2 | STKKTQLLLEHALLDALHMRRNLPEPNEKLSRIITTMQSWIFTGKIDGDGAQELAKEVEELEQEHEKRGRDVEDD |
| | ASNLKVILLELA |
| | (SEQ ID NO: 126) |
| G1_neo2_40_1F_H3 | STKKTQLLIEHALLDALNMRKKLPEPNEKLSRIITDMQSWIFTGKIDGDGAQQLAKEVEELEQEHEKRGGDVEDY |
| | ASNLKVILLELA |
| | (SEQ ID NO: 127) |
| G1_neo2_40_1F_H4 | STKKTQLLLEHALLDALHMSRELPEPNEKLNRIITDMQSWIFTGKIDGDGAQDLAKEVEELEQEHEKRGGDVEDY |
| | ASNLKVILLELA |
| | (SEQ ID NO: 128) |
| G1_neo2_40_1F_H5 | STKKTQLLIEHALLDALHMSRKLPEPNEKLSRIITTMQSWIFTGKIDGDGAQHLAKEVEELEQEHEKRGGEVEDE |
| | ASNLKVILLELA |
| | (SEQ ID NO: 129) |
| G1_neo2_40_1F_H6 | STKKTQLLIEHALLDALHMKRKLPEPNEKLNRIITNMQSWIFTEKIDGDGAQDLAKEVEELEQEHEKRGQDVEDY |
| | ASNLKVILLELA |
| | (SEQ ID NO: 130) |
| G1_neo2_40_1F_M1 | STEKTQLAAEHALRDALMLKHLLNEPNEKLARIITTMQSWQFTGKIDGDGAQELAKEVEELQQEHEVRGIDVEDY |
| | ASNLKVILLHLA |
| | (SEQ ID NO: 131) |
| G1_neo2_40_1F_M2 | STKNTQLAAEDALLDALMLRNLLNEPNEKLARIITTMQSWQFTEKIDGDGAQELAKEVEELQQEHEERGIDVEDY |
| | ASNLKVILLQLA |
| | (SEQ ID NO: 132) |
| G1_neo2_40_1F_M3 | STEKTQHAAEDALRDALMLRNLLNEPNEKLARIITTMQSWQFTEKIDGDGAQELAKEVEELQQEHEVRGIDVEDY |
| | ASNLKVILLQLA |
| | (SEQ ID NO: 133) |

TABLE 4
Amino acid sequences for second-round designs. G2_neo2_40_1F_seq02
to G2_neo2_40_1F_seq28 correspond to the 27 Rosetta™ sequence redesigns of
G1_neo2_40_1F; G2_neo2_40_1F_seq29 to G2_neo2_40_1F_seq42 represent the 14 new
de novo mimetic designs.

| Design | Sequence |
|---|---|
| G2_neo2_40_1F_seq02 | TQKKQQLLAEHALLDALMILNMLKTSSEAVNRMITIAQSWIFTGTSNPEEAKEMIKMAEQAEEEARREGV |
| | DTEDYVSNLKVILKEIA |
| | (SEQ ID NO: 134) |
| G2_neo2_40_1F_seq03 | TTKKYQLLVEHALLDALMMLNLSSESNEKMNRIITTMQSWIFTGTFDPDQAEELAKLVEELREEFRKRGI |
| | DTEDYASNLKVILKELS |
| | (SEQ ID NO: 135) |
| G2_neo2_40_1F_seq04 | TTKKIQLLVEHALLDALMILNLSSESNEKLNRIITTLQSWIFRGEIDPDRARELAKLLEEIREEMRKRGI |
| | DTEDYVSNMIVIIRELA |
| | (SEQ ID NO: 136) |
| G2_neo2_40_1F_seq05 | TKKKIQLLAEHVLLDLLMMLNLSSESNEKMNRLITIVQSWIFTGTIDPDQAEEMAKWVEELREEFRKRGI |
| | DTEDYASNVKVILKELS |
| | (SEQ ID NO: 137) |
| G2_neo2_40_1F_seq06 | TKKKYQLLIEHLLLDALMVLNMSSESNEKLNRIITILQSWIFTGTWDPDLAEEMEKLMQEIEEELRRRGI |
| | DTEDYMSNMRVIIKELS |
| | (SEQ ID NO: 138) |
| G2_neo2_40_1F_seq07 | TKKKLQLLVEHLLLDMLMILNMSSESNEKLNRLITELQSWIFRGEIDPDKAEEMWKIMEEIEKELRERGI |
| | DTEDYMSNAKVIIKELS |
| | (SEQ ID NO: 139) |
| G2_neo2_40_1F_seq08 | TSKKQQLLAEHALLDALMILNISSESSEAVNRAITWLQSWIFKGTVNPDQAEEMRKLAEQIREEMRKRGI |
| | DTEDYVSNLEVIAKELS |
| | (SEQ ID NO: 140) |
| G2_neo2_40_1F_seq09 | TKKKYQLLIEHLLLDLLMVLNMSSESNEKINRLITWLQSWIFTGTYDPDLAEEMYKILEELREEMRERGI |
| | DTEDYMSNMRVIVKELS |
| | (SEQ ID NO: 141) |
| G2_neo2_40_1F_seq10 | TKKKWQLLIEHLLLDLLMILNLSSESNEKLNRLITWLQSWIFTGTYDPDLAEEMKKMMDEIEDELRERGI |
| | DTEDYMSNAKVIIKELS |
| | (SEQ ID NO: 142) |
| G2_neo2_40_1F_seq11 | TKKKIQLLVEHALLDALMILNLSSESNEKLNRIITTMQSWIFTGTIDPDQAEELSKLVEEIREEMRKRGI |
| | DTEDYVSNLKVILDELS |
| | (SEQ ID NO: 143) |
| G2_neo2_40_1F_seq12 | TEKKLQLLVEHALLDALMILNLWSESNEKLNRIITTMQSWIFTGRIDPDKAEELAKLVEELREEARERGI |
| | DTEDYVSNLKVILKELS |
| | (SEQ ID NO: 144) |
| G2_neo2_40_1F_seq13 | TKKKYQLLMEHLLLDLLMVLNMSSESNEKLNRLITIIQSWIFTGTWDPDKAEEMAKMLKEIEDELRERGI |
| | DTEDYMSNMIVINKELS |
| | (SEQ ID NO: 145) |
| G2_neo2_40_1F_seq14 | TTKKIQLLVEHALLDALMLLNLSSESNEKMNRIITTMQSWIFEGRIDPDQAQELAKLVEELREEFRKRGI |
| | DTEDYVSNLKVILEELS |
| | (SEQ ID NO: 146) |
| G2_neo2_40_1F_seq15 | TKKKIQLLVEHALLDALMMLNLSSESNEKLNRIITTMQSWIFTGTIDPDQAEELAKLVRELREEFRNRGI |
| | DTEDYASNLEVILRELS |
| | (SEQ ID NO: 147) |
| G2_neo2_40_1F_seq16 | TKKKIQLLVEHALLDALMILNLSSKSNEKLNRIITTMQSWIFNGTIDPDRARELAKLVEEIRDEMEKNGI |
| | DTEDYVSNLKVILEELA |
| | (SEQ ID NO: 148) |
| G2_neo2_40_1F_seq17 | TKKKYQLLIEHVLLDLLMLLNLSSESNEKMNRLITILQSWIFTGTYDPDKAEEMAKLLKELREEFRERGI |
| | DTEDYISNAIVILKELS |
| | (SEQ ID NO: 149) |
| G2_neo2_40_1F_seq18 | TKKKIQLLVEHALLDALMMLNLSSESNEKLNRIITTMQSWIFTGTIDPDRAEELAKLVEELREEFRKRGI |
| | DTEDYASNLKVILKELS |
| | (SEQ ID NO: 150) |
| G2_neo2_40_1F_seq19 | TKKKIQLLVEHALLDALMMLNLSSESNEKLNRIITTMQSWIFNGTIDPDQARELAKLVEELREEFRKRGI |
| | DTEDYASNLKVILEELA |
| | (SEQ ID NO: 151) |
| G2_neo2_40_1F_seq20 | TKKKLQLLVEHALLDALMLLNLSSESNEKLNRIITTMQSWIFTGTVDPDQAEELAKLVEEIREELRKRGI |
| | DTEDYVSNLKVILKELS |
| | (SEQ ID NO: 152) |
| G2_neo2_40_1F_seq21 | TTKKYQLLVEHALLDALMILNLSSESNEKLNRIITTMQSWIFTGTFDPDQAEELAKLVREIREEMRKRGI |
| | DTEDYVSNLEVILRELS |
| | (SEQ ID NO: 153) |
| G2_neo2_40_1F_seq22 | TKKKIQLLVEHALLDALMILNLSSESNEKLNRIITTMQSWIFTGTIDPDRAEELAKLVREIREEMRKRGI |
| | DTEDYVSNLEVILRELS |
| | (SEQ ID NO: 154) |
| G2_neo2_40_1F_seq23 | TKKKYQLLIEHLLLDLLMILNLSSESNEKLNRLITWLQSWIFRGEWDPDKAEEWAKILKEIREELRERGI |
| | DTEDYMSNAIVIMKELS |
| | (SEQ ID NO: 155) |
| G2_neo2_40_1F_seq24 | TDKKLQLLVEHLLLDLLMMLNLSSKSNEKMNRLITIAQSWIFTGKVDPDLAREMIKLLEETEDENRKNGI |
| | DTEDYVSNARVIAKELE |
| | (SEQ ID NO: 156) |
| G2_neo2_40_1F_seq25 | TKKKIQLLVEHALLDALMLLNLSSESNEKMNRIITTMQSWIFTGTIDPDQAEELAKLVEELKEEFKKRGI |
| | DTEDYVSNLKVILKELS |
| | (SEQ ID NO: 157) |
| G2_neo2_40_1F_seq26 | TKKKYQLLIEHALLDALMILNLWSESNEKLNRIITTMQSWIFTGTYDPDKAEELEKLAKEIEDEARERGI |
| | DTEDYMSNLRVILKELS |
| | (SEQ ID NO: 158) |
| G2_neo2_40_1F_seq27 | TKKKAQLLAEHALLDALMLLNLSSESNERLNRIITWLQSIIFTGTYDPDMVKEAVKLADEIEDEMRKRGI |
| | DTEDYVSNLRVILQELA |
| | (SEQ ID NO: 159) |
| G2_neo2_40_1F_seq28 | TQKKNQLLAEHLLLDALMVLNQSSESSEVANRIITWAQSWIFEGRVDPNKAEEAKKLAKKLEEEMRKRGI |
| | DMEDYISNMKVIAEEMS |
| | (SEQ ID NO: 160) |
| G2_neo2_40_1F_seq29 | EDYYSNLKVILEELAREMERNGLSDKAEEWRQWKKIVERIRQIRSNNSDLNEAKELLNRLITYIQSQIFE |
| | ISERIRETDQEKKEESWKKWQLLLEHALLDVLMLLND |
| | (SEQ ID NO: 161) |
| G2_neo2_40_1F_seq30 | PEKKRQLLLEHILLDALMLLNLLETNPQNTESKFEDYISNAEVIAEELAKLMESLGLSDEAEKFKKIKQW |
| | LREVWRIWSSTNWSTLEDKARELLNRIITTIQSQIFY |
| | (SEQ ID NO: 162) |
| G2_neo2_40_1F_seq31 | PEKKRQLLLEHILLDLLMILNMIETNRENTESEMEDYWSNVRVILRELARLMEELNYKELSELMERMRKI |
| | VEKIRQIVTNNSSLDTAREWLNRLITWIQSLIFR |
| | (SEQ ID NO: 163) |
| G2_neo2_40_1F_seq32 | PEKKRQLLAEHALLDALMLLNIIETNSKNTESKMEDYVSNLEVILTEFKKLAEKLNFSEEAERAERMKRW |
| | ARKAYQMMTLDLSLDKAKEMLNRIITILQSIIFN |
| | (SEQ ID NO: 164) |
| G2_neo2_40_1F_seq33 | PEKKRQLLAEHLLLDVLMMLNGNASLKDYASNAQVIADEFRELARELGLTDEAKKAEKIIEALERAREWL |
| | LNNKDKEKAKEALNRAITIAQSWIFN |
| | (SEQ ID NO: 165) |
| G2_neo2_40_1F_seq34 | PEKKRQLLLEHLLLDLLMILNMLRTNPKNIESDWEDYMSNIEVIIEELRKIMESLGRSEKAKEWKRMKQW |
| | VRRILEIVKNNSDLEEAKEWLNRLITIVQSEIFE |
| | (SEQ ID NO: 166) |
| G2_neo2_40_1F_seq35 | WEKKRQLLLEHLLLDLLMILNMWRTNPQNTESLMEDYMSNAKVIVEELARMMRSQGLEDKAREWEEMKKR |
| | IEEIRQIIQNNSSKERAKEELNRLITYVQSEIFR |
| | (SEQ ID NO: 167) |
| G2_neo2_40_1F_seq36 | PKKKIQLLAEHALLDALMILNIVKTNSQNAEEKLEDYASNVEVILEEIARLMESGDQKDEAEKAKRMKEW |
| | MKRIKTTASEDEQEEMANRIITLLQSWIFS |
| | (SEQ ID NO: 168) |
| G2_neo2_40_1F_seq37 | PEKKRQLLAEHALLDALMILNILQTNPQNAEEKLEDYMSNVEVIMEEFARMMRNGDRSEEAENAERIKKW |
| | VRKASSTASSEEQREMMNRAITLMQSWIFE |
| | (SEQ ID NO: 169) |
| G2_neo2_40_1F_seq38 | PEKKRQLLAEHLLLDALMVLNMLTTNSKNTEEKLEDYISNMKVIIKEMIELMRSLGRLEEAEKWKEALKA |
| | VEKIGSRMDSETARELANRIITLAQSAIFY |
| | (SEQ ID NO: 170) |
| G2_neo2_40_1F_seq39 | PEKKRQLLAEHALLDALMFLNLVETNPDQAEEKIEDYASNLRVIAEELARLFENLGRLDEAQKAKDIKEL |
| | AERARSRVSSEKRKEAMNRAITILQSMIFR |
| | (SEQ ID NO: 171) |
| G2_neo2_40_1F_seq40 | PEKKRQLLAEHALLDALMILNIIRTNSDNTESKLEDYISNLKVILEEIARLMESLGLSDEAEKAKEAMRL |
| | ADKAGSTASEEEKKEAMNRVITWAQSWIFN |
| | (SEQ ID NO: 172) |
| G2_neo2_40_1F_seq41 | PEKKRQLLAEHALLDALMMLNILRTNPDNAEEKLEDYWSNLIVILREIAKLMESLGLTDEAEKAKEAARW |
| | AEEARTTASKDQRRELANRIITLLQSWIFS |
| | (SEQ ID NO: 173) |
| G2_neo2_40_1F_seq42 | PEKKRQLLAEKLLLDALMILNIIETNEQNAESKLEDYISNAKVILDEFREMARDLGLLDEAKKAEKMKRW |
| | LEKMRSNASSDERREWANRMITTAQSWIFN |
| | (SEQ ID NO: 174) |

TABLE 5
Amino acid sequences for the experimentally optimized second-round designs.

| Design | Sequence |
|---|---|
| G2_neo2_40_1F_seq27_S18 | TNKEAQLHAEFALYDALMLLNLSSESNERLNRIITWLQSIIFYETYDPDMVKEAVKLADEIEDEMR |
| | KRKIDTEDYVVNLRLILQELA |
| | (SEQ ID NO: 175) |
| G2_neo2_40_1F_seq27_S22 | TKKDAELLAEFALYDALMLLNLSSESNERLNEIITWLQSIIFYGTYDPDMVKEAVKLADEIEDEMR |
| | KRGIDTEDYVSNLRLILQELA |
| | (SEQ ID NO: 176) |
| G2_neo2_40_1F_seq27_S24 | TNKKAQLHAEFALYDALMLLNLSSESNERLNDIITWLQSIIFTGTYDPDMVKEAVKLADEIEDEMR |
| | KRKIDTEDYVVNLRYILQELA |
| | (SEQ ID NO: 177) |
| G2_neo2_40_1F_seq29_S6 | EDYYSNLKLILEELAREMERNGLSDKAEEWRQWKKIVERIRQIRSNNSDLNEAKELLNRLITYIQS |
| | QIFEVLHGVGETDQEKKEESWKKWDLLLEHALLDVLMLLND |
| | (SEQ ID NO: 178) |
| G2_neo2_40_1F_seq29_S7 | EDYYSNLKVILEELAREMERNGLSDKAEEWRQWKKIVERIRQIRSNNSDLNEAKELLNELITYIQS |
| | QIFEVIEREGETDQEKKEESWKKWELHLEHALLDVLMLLND |
| | (SEQ ID NO: 179) |
| G2_neo2_40_1F_seq29_S8 | EDYYSNLKLILEELAREMERNGLSDKAEEWRQWKKIVERIRQIRSNNSDLNEARELLNRLITYIQS |
| | QIFEVLEGVGETDQEKKEESWKKWELHLEHALLDVLMLLND |
| | (SEQ ID NO: 180) |
| Neolukin-2/15 (i.e. | PKKKIQLHAEHALYDALMILNIVKTNSPPAEEKLEDYAFNFELILEEIARLFESGDQKDEAEKAKR |
| G2_neo2_40_1F_seq36_S11) | MKEWMKRIKTTASEDEQEEMANAIITILQSWIFS |
| | (SEQ ID NO: 181) |
| G2_neo2_40_1F_seq36_S12 | PKKKIQLLAEHALFDLLMILNIVKTNSQNAEEKLEDYAYNAGVILEEIARLFESGDQKDEAEKAKR |
| | MKEWMKRIKDTASEDEQEEMANEIITILQSWNFS |
| | (SEQ ID NO: 182) |

Neoleukin-2/15-H8Y-K33E: H1->H3->H2′->H4
PKKKIQLYAEHALYDALMILNIVKTNSPPAEEELEDYAFNFELILEEIARLFESGDQKDEAEKAKRMKEWMKRIKTTASEDEQEEMANAIITILQSWIFS (SEQ ID NO: 94)

Neoleukin-2/15-H8Y-K33E: H1->H3->H2′ ->H4
(SEQ ID NO: 94)
PKKKIQLYAEHALYDALMILNIVKTNSPPAEEELEDYAFNFELILEEIAR
LFESGDQKDEAEKAKRMKEWMKRIKTTASEDEQEEMANAIITILQSWIFS

Binding of Neoleukin-2/15-HSY-K33E to the IL2 receptor was measured by biolayer interferometry, and it was found to have higher binding affinity than Neoleukin-2 for IL2-Rbeta, both when tested against IL2Rbeta alone and when tested against the IL2Rbeta-gamma complex. This increased affinity was attributable mostly to an improved off-rate from IL2-Rbeta.

TABLE 6
Amino acid sequences for the inter1eukin-4 mimetic designs based on
reengineering of neolukin-2/15.

| Design | Sequence |
|---|---|
| IL4_G2_neo2_40_1F_seq36_S11 | PKKKIQITAEEALKDALSILNIVKTNSPPAEEQLERFAKRFERNLWGIARLFESGDQKDEAE |
| | KAKRMKEWMKRIKTTASEDEQEEMANAIITILQSWIFS |
| | (SEQ ID NO: 183) |
| Neoleukin-4 (i.e. | PKKKIQIMAEEALKDALSILNIVKTNSPPAEEQLERFAKRFERNLWGIARLFESGDQKDEAE |
| IL4_G2_neo2_40_1F_seq36_S11_MIF) | KAKRMIEWMKRIKTTASEDEQEEMANAIITILQSWFFS |
| | (SEQ ID NO: 184) |

### Example 2: Split Cytokine Mimics for Targeted Immunotherapy

De novo proteins are designed following the rules of an ideal protein structure providing them with unusual biochemical properties, such as extreme thermostability and mutational robustness. Therefore, de novo designed proteins are ideal candidates to use for the development of conditionally active protein therapeutics. Here, we report the development of split cytokine mimetics for highly-targeted immunotherapy based on the recently developed de novo designed IL-2 mimetic protein, Neoleukin-2/15. This system enables the delivery of conditionally active therapeutic proteins that reconstitute their activity by colocalization on the surface of target cells. We identified potential split sites and demonstrated successful reconstitution of Neoleukin-2/15 activity by binding to the IL-2 Receptors, cell signaling and colocalization-dependent activation on the surface of target tumor cells. We also demonstrate this application to another de novo designed cytokine mimic, Neoleukin-4.

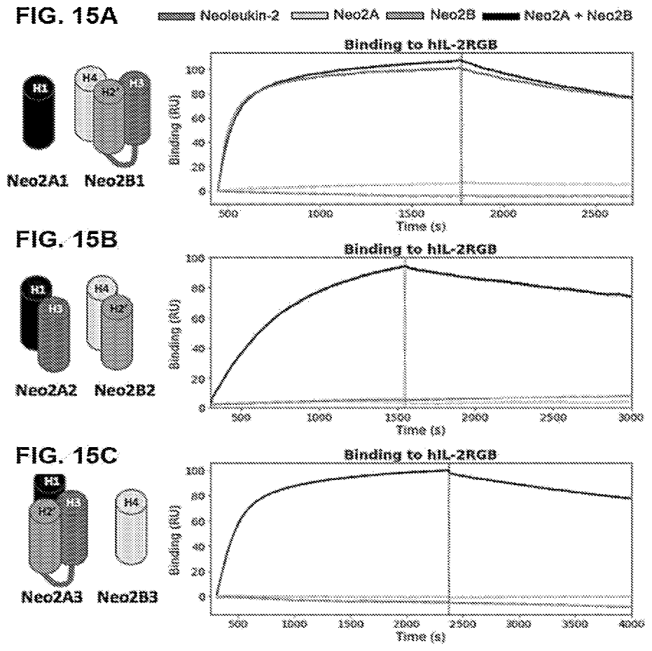

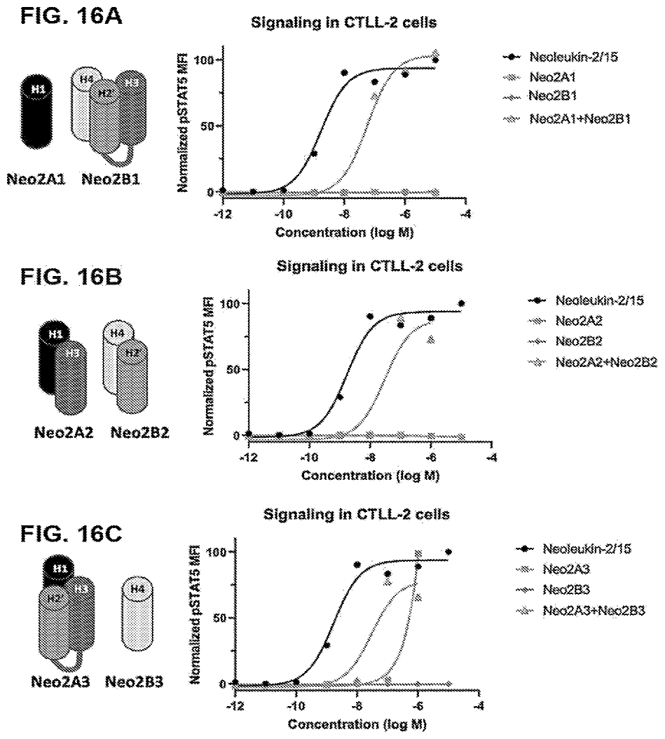

In order to identify potential split sites of Neoleukin-2/15 (Neo2), we evaluated the protein structure to find split positions that would minimize detrimental effects on the function of the protein. As a result, we defined three exemplary split positions: (i) between the helical elements H1 (Neo2A1) and H3′-H2-H4 (Neo2B1), (ii) between the helical elements H1-H3′(Neo2A2) and H2-H4 (Neo2B2), (iii) between the helical elements H1-H3′-H2 (Neo2A3) and H4 (Neo2B3)(FIG. 15). The split-Neo2 fragments were characterized by analyzing their binding capacity to human IL-2 Receptor (FIG. 15) and signaling on IL-2 responsive CTTL-2 cells (FIG. 16). The individual split fragments were shown to have negligible binding to IL-2Rβγ and signaling capacity in most cases, but potent activity upon equimolar combination of the complementary split fragments.

The development of split-Neo2, enables co-localization-dependent reconstitution of the protein, and thus, conditional-activation of split-Neo2. In order to enable the colocalization of Neo2A and Neo2B fragments, we firstly performed genetic fusions to targeting domains (FIG. 17). Neo2A1, Neo2A2 and Neo2A3 were fused to the C-terminus of a Her2-targeted designed ankyrin repeat protein (DARPin) by a 15-residue flexible linker (GSGGSGGGSGGSGSG; SEQ ID NO:100). Neo2B1, Neo2B2 and Neo2B3 were fused to the N-terminus of an EGFR-targeted designed ankyrin repeat protein (DARPin) by a 15-residue flexible linker (GSGGSGGGSGGSGSG; SEQ ID NO:100). Then, we evaluated their ability to reconstitute binding to IL-2Rβ7 after combination of the split fragments, demonstrating that the fusions did not interfere with their reconstitution capacity of splitNeo-2 (FIG. 17).

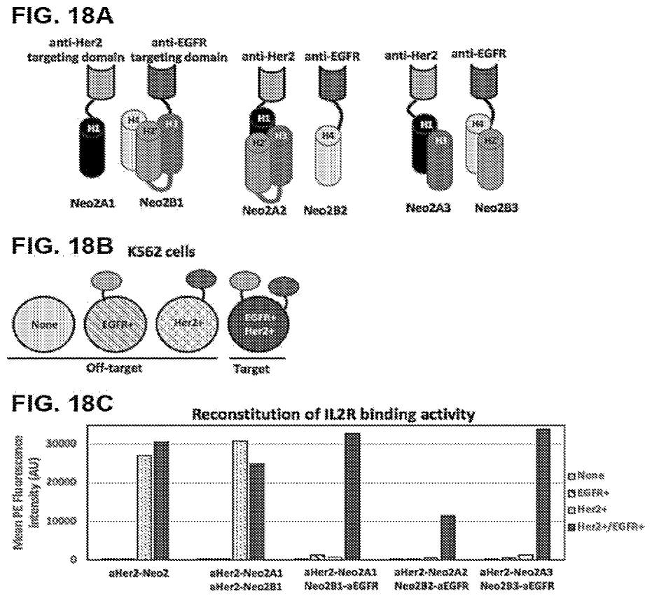

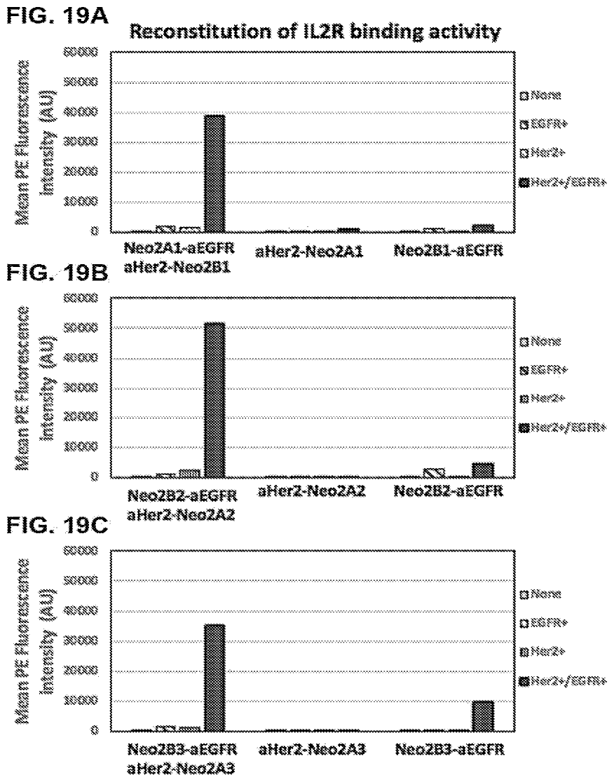

To evaluate the colocalization-dependent activation and targeting selectivity of the split-Neo2 system, we performed an in vitro assay with split Neo-2/15 targeted to EGFR and Her2 (FIG. 18a) on the surface of engineered K562 cell lines. We used four engineered cell lines: (i) no surface receptors, (ii) Her2+/eGFP+, (iii) EGFR+/iRFP+, (iv) Her2+/eGFP+EGFR+/iRFP+(FIG. 18b). The four cell lines were mixed in equivalent ratios and incubated with the targeted split-Neo2 fragments for 15 minutes. Subsequently, the cells were washed and incubated with a mixture of biotinylated IL-2Receptor and a fluorescent streptavidin-phycoerythrin conjugate (SAPE). Finally, the cells were analyzed by Fluorescence-activate cell sorting (FACS). Successful reconstitution of Neo-2 activity resulted in fluorescent labelling with PE on the surface of target cells (FIG. 18c). We observed strong IL-2 Receptor binding on the surface of Her2+ and Her2+/EGFR+ when full Neo2 was targeted with a single anti-Her2 targeting domain (aHer2-Neo2). Similar binding was observed when Neo2A1 and Neo2B1 split fragments were both targeted to Her2 (aHer2-Neo2A1+aHer2-Neo2B1), demonstrating that both fragments could be delivered to the same cell surface marker if desired. Additionally, we observed highly selective reconstitution on the surface of double positive Her2+/EGFR+ when each split fragment was targeted to either Her2 or EGFR (aHer2-Neo2A1+Neo2B1-aEGFR, aHer2-Neo2A2+Neo2B2-aEGFR, aHer2-Neo2A3+Neo2B3-aEGFR). Further studies demonstrated that for each combination of split-Neo2 fragments, the simultaneous presence of both fragments was necessary to enable IL-2Receptor recruitment on the surface of K562 cells (FIG. 19). These results demonstrate the high selectivity of the split cytokine mimetics to reconstitute the desired activity specifically on the surface of target cells.

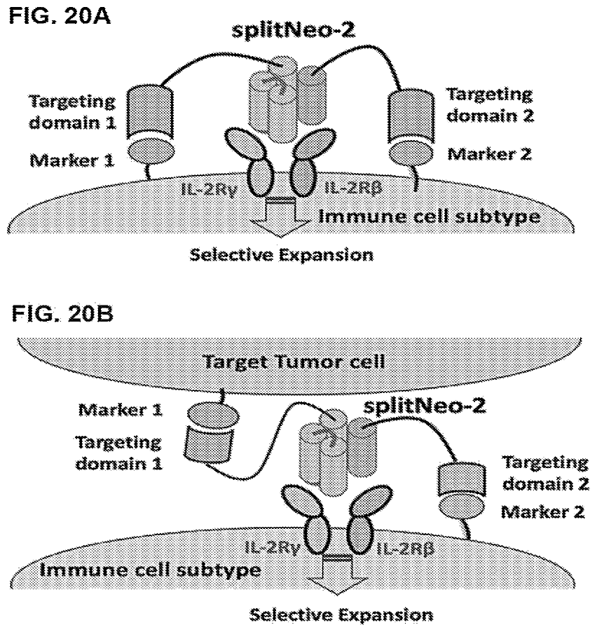

The applications of the split cytokine mimic technology are not only limited to targeting tumor associated antigens. For given applications, targeting specific subsets of immune cells to be selectively stimulated would be beneficial to direct the immune response to treat disease. For instance, this application would be useful to specifically augment the expansion of CD8+ cytotoxic T cells, Natural Killer cells or engineered CAR T-cells to potentiate their anti-tumor response, but also, targeting to regulatory T-cells to suppress a strong immune response (FIG. 20a). Furthermore, in some scenarios, mixed targeting approaches can be deployed, i.e. target one split fragment to the tumor cells or tumor microenvironment, while the other fragment is delivered to immune cells (FIG. 20b)

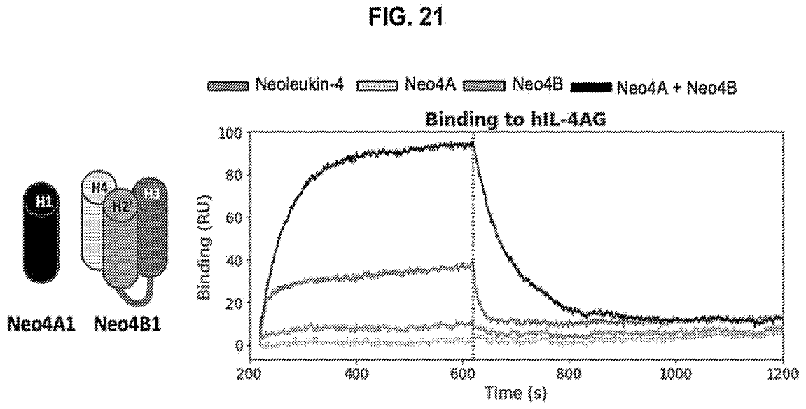

Finally, to demonstrate the transferability of the methodology described here to other de novo designed interleukins, we created a new conditionally active IL-4 mimetic (FIG. 21). To achieve this, we successfully introduced specific mutations on splitNeo-2 that modify the binding interface to the hIL-2Beta receptor, to bind hIL-4alpha receptor.

Exemplary Split Neoleukin-2/15 variants sequences
used in Example 2
H1 (Neo2A1)
(SEQ ID NO: 256)
PKKKIQLHAEHALYDALMILNIVKINS
H3-H2'-H4 Neo2B1)
(SEQ ID NO: 257)
TNSPPAEEKLEDYAFNFELILEEIARLFESGDQKDEAEKAKRMKEWMKRI
KTTASEDEQEEMANAIITILQSWIFS
H1-H3-H2' Neo2A3
(SEQ ID NO: 258)
PKKKIQLHAEHALYDALMILNIVKINSPPAEEKLEDYAFNFELILEEIAR
LFESGDQKDEAEKAKRMKEWMKRIKTTAS
H4 (Neo2B3)
(SEQ ID NO: 259)
TTASEDEQEEMANAIITILQSWIFS
H1-H3 (Neo2A2)
(SEQ ID NO: 260)
PKKKIQLHAEHALYDALMILNIVKINSPPAEEKLEDYAFNFELILEEIAR
LFESGD
H2'-H4 (Neo2B2)
(SEQ ID NO: 261)
DQKDEAEKAKRMKEWMKRIKTTASEDEQEEMANAIITILQSWIFS
Exemplary Split Neoleukin-4 sequences
used in Example 2
H1 Neo4A1)
(SEQ ID NO: 262)
PKKKIQIMAEEALKDALSILNIVKINS
H3-H2'-H4 (a.k.a. Neo4B1)
(SEQ ID NO: 263)
TNSPPAEEQLERFAKRFERNLWGIARLFESGDQKDEAEKAKRMIEWMK
RIKTTASEDEQEEMANAIITILQSWFFS
Exemplary constructs used in Example 2
X2-Z1-X3-Z2-X4-E01_EGFR_DARPin
(SEQ ID NO: 304)
(MGSHHHHHHGSGSENLYFQGSGGG)TNSPPAEEKLEDYAFNFELILE
EIARLFESGDQKDEAEKAKRMKEWMKRIKTTASEDEQEEMANAIITIL
QSWIFS(GSGGSGGGSGGSGSGGSGGG)DLGKKLLEAARAGQDDEVRI
LMANGADVNADDTWGWTPLHLAAYQGHLEIVEVLLKNGADVNAYDYIG
WTPLHLAADGHLEIVEVLLKNGADVNASDYIGDTPLHLAAHNGHLEIV
EVLLKHGADVNAQDKFGKTAFDISIDNGNEDLAEILQKLN
G3_Her2_DARPin_X1
(SEQ ID NO: 305)
(MGSHHHHHHGSGSENLYFQGSGSG)DLGKKLLEAARAGQDDEVRILM
ANGADVNAKDEYGLTPLYLATAHGHLEIVEVLLKNGADVNAVDAIGFT
PLHLAAFIGHLEIAEVLLKHGADVNAQDKFGKTAFDISIGNGNEDLAE
ILQKLN(GSGGSGGGSGGSGSG)PKKKIQLHAEHALYDALMILNIVKT
NS
G3_Her2_DARPin_X2-Z1-X3-Z3-X4
(SEQ ID NO: 306)
(MGSHHHHHHGSGSENLYFQGSGSG)DLGKKLLEAARAGQDDEVRILM
ANGADVNAKDEYGLTPLYLATAHGHLEIVEVLLKNGADVNAVDAIGFT
PLHLAAFIGHLEIAEVLLKHGADVNAQDKFGKTAFDISIGNGNEDLAE
ILQKLN(GSGGSGGGSGGSGSG)TNSPPAEEKLEDYAFNFELILEEIA
RLFESGDQKDEAEKAKRMKEWMKRIKTTASEDEQEEMANAIITILQSW
IFS
X2-Z1-X3-Z2-X4-E01_EGFR_DARPin
(SEQ ID NO: 307)
(MGSHHHHHHGSGSENLYFQGSGGG)TNSPPAEEKLEDYAFNFELILE
EIARLFESGDQKDEAEKAKRMKEWMKRIKTTASEDEQEEMANAIITIL
QSWIFS(GSGGSGGGSGGSGSGGSGGG)DLGKKLLEAARAGQDDEVRI
LMANGADVNADDTWGWTPLHLAAYQGHLEIVEVLLKNGADVNAYDYIG
WTPLHLAADGHLEIVEVLLKNGADVNASDYIGDTPLHLAAHNGHLEIV
EVLLKHGADVNAQDKFGKTAFDISIDNGNEDLAEILQKLN
X2-Z2-X4-E01_EGFR_DARPin
(SEQ ID NO : 308)
(MGSHHHHHHGSGSENLYFQGSGGG)DQKDEAEKAKRMKEWMKRIKTT
ASEDEQEEMANAIITILQSWIFS(GSGGSGGGSGGSGSGGSGGG)DLG
KKLLEAARAGQDDEVRILMANGADVNADDTWGWTPLHLAAYQGHLEIV
EVLLKNGADVNAYDYIGWTPLHLAADGHLEIVEVLLKNGADVNASDYI
GDTPLHLAAHNGHLEIVEVLLKHGADVNAQDKFGKTAFDISIDNGNED
LAEILQKLN
X4-E01_EGFR_DARPin
(SEQ ID NO: 309)
(MGSHHHHHHGSGSENLYFQGSGGG)TTASEDEQEEMANAIITILQSW
IFS(GSGGSGGGSGGSGSGGSGGG)DLGKKLLEAARAGQDDEVRILMA
NGADVNADDTWGWTPLHLAAYQGHLEIVEVLLKNGADVNAYDYIGWTP
LHLAADGHLEIVEVLLKNGADVNASDYIGDTPLHLAAHNGHLEIVEVL
LKHGADVNAQDKFGKTAFDISIDNGNEDLAEILQKLN
G3_Her2_DARPin_X1-Z1-X3
SEQ ID NO : 310)
(MGSHHHHHHGSGSENLYFQGSGSG)DLGKKLLEAARAGQDDEVRIL
MANGADVNAKDEYGLTPLYLATAHGHLEIVEVLLKNGADVNAVDAIG
FTPLHLAAFIGHLEIAEVLLKHGADVNAQDKFGKTAFDISIGNGNED
LAEILQKLN(GSGGSGGGSGGSGSG)PKKKIQLHAEHALYDALMILN
IVKTNSPPAEEKLEDYAFNFELILEEIARLFESG
G3_Her2_DARPin_X1-Z1-X3-Z2-X2
(SEQ ID NO: 311)
(MGSHHHHHHGSGSENLYFQGSGSG)DLGKKLLEAARAGQDDEVRIL
MANGADVNAKDEYGLTPLYLATAHGHLEIVEVLLKNGADVNAVDAIG
FTPLHLAAFIGHLEIAEVLLKHGADVNAQDKFGKTAFDISIGNGNED
LAEILQKLN(GSGGSGGGSGGSGSG)PKKKIQLHAEHALYDALMILN
IVKTNSPPAEEKLEDYAFNFELILEEIARLFESGDQKDEAEKAKRMK
EWMKRIKTTA

We claim:

1. A non-naturally occurring conditionally active receptor agonist, comprising a first polypeptide component and a second polypeptide component, wherein the first polypeptide component and the second polypeptide component are not present in a fusion protein, wherein in total the first polypeptide component and the second polypeptide component comprise domains X1, X2, X3, and X4, wherein:
(a) X1 is a peptide comprising an amino acid sequence at least 75% identical to the amino acid sequence (PKKKIQ)LHAEHALYDAL(MILNI) (SEQ ID NO: 4);
(b) X2 is any helical peptide domain;
(c) X3 is a peptide comprising an amino acid sequence at least 75% identical to the amino acid sequence (LE)DYAFNFELILEE(IARLFESG) (SEQ ID NO:5); and
(d) X4 is a peptide comprising an amino acid sequence at least 75% identical to the amino acid sequence (EDEQEEMANAI)ITILQSWIF(S) (SEQ ID NO:6).
wherein:
(i) amino acid residues in parentheses may be present or absent;
(ii) the first polypeptide component comprises at least one of X1, X2, X3, and X4 but does not comprise each of X1, X2, X3, and X4; and
(iii) the second polypeptide component comprises each of X1, X2, X3, and X4 that is not present in the first polypeptide component;
wherein the first polypeptide component further comprises a first targeting domain and the second polypeptide component further comprises a second targeting domain;
wherein the first polypeptide component and the second polypeptide component are not active receptor agonists individually, and wherein the first polypeptide component and the second polypeptide interact to form an active agonist of IL-2 receptor β [symbol] _{c} heterodimer (IL-2Rβ [symbol] _{c}).

2. The conditionally active receptor agonist of claim 1, wherein:
(a) X1 is a peptide comprising an amino acid sequence at least 85% identical to the amino acid sequence PKKKIQLHAEHALYDAL(MILNI (SEQ ID NO: 4) or QLHAEHALYDAL(MILNI (SEQ ID NO:320);
(b) X3 is a peptide comprising an amino acid sequence at least 85% identical to the amino acid sequence LEDYAFNFELILEEIARLFESG (SEQ ID NO:5) or LEDYAFNFELILEEIARLFES (SEQ ID NO:321); and
(c) X4 is a peptide comprising an amino acid sequence at least 85% identical to the amino acid sequence EDEQEEMANAIITILQSWIF(S) (SEQ ID NO:6) or DEQEEMANAIITILQSWIF(S) (SEQ ID NO:322).

3. The conditionally active receptor agonist of claim 1, wherein:
X2 is a peptide comprising an amino acid sequence at least 75% identical to the amino acid sequence KDEAEKAKRMKEWMKRIK(T) (SEQ ID NO:7), wherein amino acid residues in parentheses may be present or absent.

4. The conditionally active receptor agonist of claim 1, wherein:
(i) the first polypeptide component includes one of X1, X2, X3, and X4, and the second polypeptide component includes the three of X1, X2, X3, and X4 that are not present in the first polypeptide component; or
(ii) the first polypeptide component includes two of X1, X2, X3, and X4, and the second polypeptide component includes the two of X1, X2, X3, and X4 that are not present in the first polypeptide component.

5. The conditionally active receptor agonist of claim 1, wherein
(i) the first polypeptide comprises X1 and the second polypeptide comprises X2, X3, and X4;
(ii) the first polypeptide comprises X2 and the second polypeptide comprises X1, X3, and X4;
(iii) the first polypeptide comprises X3 and the second polypeptide comprises X1, X2, and X4;
(iv) the first polypeptide comprises X4 and the second polypeptide comprises X1, X2, and X3;
(v) the first polypeptide comprises X1 and X2, and the second polypeptide comprises X3 and X4;
(vi) the first polypeptide comprises X1 and X3, and the second polypeptide comprises X2 and X4;
(vii) the first polypeptide comprises X1 and X4, and the second polypeptide comprises X2 and X3;
(viii) the first polypeptide comprises X2 and X3, and the second polypeptide comprises X1 and X4;
(ix) the first polypeptide comprises X2 and X4, and the second polypeptide comprises X1 and X3;
(x) the first polypeptide comprises X3 and X4, and the second polypeptide comprises X1 and X2;
(xi) the first polypeptide comprises X1, X2, and X3 and the second polypeptide comprises X4;
(xii) the first polypeptide comprises X1, X2, and X4 and the second polypeptide comprises X3;
(xiii) the first polypeptide comprises X1, X3, and X4 and the second polypeptide comprises X2; or
(xiv) the first polypeptide comprises X2, X3, and X4 and the second polypeptide comprises X1.

6. The conditionally active receptor agonist of claim 1, wherein:
(a) the first polypeptide comprises X1 and excludes X2, X3, and X4; and the second polypeptide is a fusion protein comprising X3-Z1-X2-Z2-X4 and excluding X1;
(b) the first polypeptide comprises X4 and excludes X1, X2, and X3; and the second polypeptide is a fusion protein comprising X1-Z1-X3-Z2-X2 and excluding X4; or
(c) the first polypeptide is a fusion protein comprising X1-Z1-X3 and excluding X2 and X4; and the second polypeptide is a fusion protein comprising X2-Z1-X4 and excluding X1 and X3;
wherein each of Z1 and Z2 independently are an optional amino acid linker.

7. The conditionally active receptor agonist of claim 1, wherein the first polypeptide and the second polypeptide comprise an amino acid sequence having at least 75% identity to the amino acid sequence of a pair of first and second polypeptides selected from options (i)-(iii), (v)-(vii) or (xiii), wherein underlined residues or “X” residues” are optional and each optional residue, when present, may comprise any amino acid:
```
(i)
First polypeptide X1 (Neo2A)
(SEQ ID NO: 256)
PKKKIQLHAEHALYDALMILNIVKTNS
and
Second polypeptide: X3-X2'-X4 (Neo2B)
(SEQ ID NO: 257)
TNSPPAEEKLEDYAFNFELILEEIARLFESGDQKDEAEKAKRMK
EWMKRIKTTASEDEQEEMANAIITILQSWIFS
(ii)
First polypeptide X1-X3-X2'
(SEQ ID NO: 258)
PKKKIQLHAEHALYDALMILNIVKTNSPPAEEKLEDYAFNFELI
LEEIARLFESGDQKDEAEKAKRMKEWMKRIKTTAS
and
(SEQ ID NO: 259)
Second polypeptide X4
TTASEDEQEEMANAIITILQSWIFS;
(iii)
First polypeptide X1-X3
(SEQ ID NO: 260)
PKKKIQLHAEHALYDALMILNIVKTNSPPAEEKLEDYAFNFELI
LEEIARLFESGD
and
Second polypeptide X2-X4
(SEQ ID NO: 261)
DQKDEAEKAKRMKEWMKRIKTTASEDEQEEMANAIITILQSWIFS
(v)
First polypeptide X1
(SEQ ID NO: 311)
PKKKIQLHAEHALYDALMILNIXXXXX
and
Second polypeptide: X3-X2'-X4
(SEQ ID NO: 264)
XXXXXXXXXLEDYAFNFELILEEIARLFESGXXKDEAEKAKRMKE
WMKRIKTXXXEDEQEEMANAIITILQSWIFS
(vi)
First polypeptide X1-X3-X2'
(SEQ ID NO: 265)
PKKKIQLHAEHALYDALMILNIXXXXXXXXXXXLEDYAFNFELIL
EEIARLFESGXXKDEAEKAKRMKEWMKRIKTTAS
and
Second polypeptide X4
  (SEQ ID NO: 266)
XXXXXDEQEEMANAIITILQSWIFS;
(vii)
First polypeptide X1-X3
(SEQ ID NO: 267)
PKKKIQLHAEHALYDALMILNIXXXXXXXXXXXLEDYAFNFELI
LEEIARLFESXXGD
and
Second polypeptide X2-X4
(SEQ ID NO: 268)
DQKDEAEKAKRMKEWMKRIKTXXXEDEQEEMANAIITILQSWIFS;
or
(xiii)
First polypeptide (X1)
(SEQ ID NO: 323)
PKKKIQLHAEHALYDALMILNIVGGSS,
or
  (SEQ ID NO: 324)
SKEAIQLHAEHALYDALMILNIVKINS,
or
(SEQ ID NO: 325)
PIQLHAEHALYDALMILNIV
Second polypeptide (X3-X2'-X4)
  (SEQ ID NO: 326)
PKLEDYAFNFELILEEIARLFESGDQKDEAEKAKRMKEWMKRIKTT
ASEDEQEEMANAIITILQSWIFS;
or
(SEQ ID NO: 327)
GGSSGGLEDYAFNFELILEEIARLFESGGSSGGKDEAEKAKRMKEW
MKRITGGSSGGDEQEEMANAIITILQSWIFS;
or
  (SEQ ID NO: 328)
GGSSGGLEDYAFNFELILEEIARLFESGGSSGGGGEAEKAKRMKEW
MKRIGGSSGGDEQEEMANAIITILQSWIFS.
```

8. The conditionally active receptor agonist of claim 1, wherein the first polypeptide component and the second polypeptide component are non-covalently associated.

9. The conditionally active receptor agonist of claim 1, wherein the first polypeptide component and the second polypeptide component are indirectly bound to each other through a receptor.

10. The conditionally active receptor agonist of claim 1, wherein the first targeting domain is a translational fusion with the first polypeptide, and wherein the second targeting domain is a translational fusion with the second polypeptide.

11. The conditionally active receptor agonist of claim 10, further comprising a first polypeptide linker connecting the first polypeptide component to the first targeting domain and/or a second polypeptide linker connecting the second polypeptide component to the second targeting domain.

12. The conditionally active receptor agonist of claim 1, wherein the first targeting domain and the second targeting domain are the same.

13. The conditionally active receptor agonist of claim 1, wherein the first targeting domain and/or the second targeting domain each bind to a cell surface protein.

14. The conditionally active receptor agonist of claim 1, wherein the first targeting domain and/or the second targeting domain are selected from the group consisting of an scFv, a F(ab), a F(ab′)2, a B cell receptor (BCR), a DARPin, an affibody, a monobody, a nanobody, diabody, and an antibody.

15. The conditionally active receptor agonist of claim 1, wherein the first targeting domain and the second targeting domain each bind to a tumor cell, tumor vascular component cell, or tumor microenvironment cell surface marker.

16. The conditionally active receptor agonist of claim 1, wherein the first and/or the second targeting domain binds to an immune cell surface marker.

17. The conditionally active receptor agonist of claim 16, wherein the first and or the second targeting domain binds to PD-1, PD-L1, CTLA-4, TROP2, B7-H3, CD33, CD22, carbonic anhydrase IX, CD123, Nectin-4, tissue factor antigen, CD154, B7-H4, FAP (fibroblast activation protein) or MUC16.

18. The conditionally active receptor agonist of claim 16, wherein the immune cell marker is selected from CD3, CD4, CD5, CD19, CD20, CD21, CD25, CD37, CD30, CD33, CD40, CD68, CD123, CD254, PD-1, B7-H3 and CTLA-4.

19. A pharmaceutical composition comprising the conditionally active receptor agonist of claim 1 and a pharmaceutically acceptable carrier.


SEQUENCE LISTING (328 sequences)

SEQ ID NO: 1 - protein, 8 aa, synthetic construct
  REGION 1..8: note = Synthetic
EHALYDAL

SEQ ID NO: 2 - protein, 8 aa, synthetic construct
  REGION 1..8: note = Synthetic
YAFNFELI

SEQ ID NO: 3 - protein, 9 aa, synthetic construct
  REGION 1..9: note = Synthetic
ITILQSWIF

SEQ ID NO: 4 - protein, 22 aa, synthetic construct
  REGION 1..22: note = Synthetic
PKKKIQLHAEHALYDALMILNI

SEQ ID NO: 5 - protein, 22 aa, synthetic construct
  REGION 1..22: note = Synthetic
LEDYAFNFELILEEIARLFESG

SEQ ID NO: 6 - protein, 21 aa, synthetic construct
  REGION 1..21: note = Synthetic
EDEQEEMANAIITILQSWIFS

SEQ ID NO: 7 - protein, 19 aa, synthetic construct
  REGION 1..19: note = Synthetic
KDEAEKAKRMKEWMKRIKT

SEQ ID NO: 8 - protein, 22 aa, synthetic construct
  REGION 1..22: note = Synthetic
PKKKIQIMAEEALKDALSILNI

SEQ ID NO: 9 - protein, 22 aa, synthetic construct
  REGION 1..22: note = Synthetic
LERFAKRFERNLWGIARLFESG

SEQ ID NO: 10 - protein, 21 aa, synthetic construct
  REGION 1..21: note = Synthetic
EDEQEEMANAIITILQSWFFS

SEQ ID NO: 11 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKWQLQAEHALLDWQMALNKXXXXXENLNRAITAAQSWISXXXXXLDKAEDIRRNSDQARREAEKXXXXXRDLISNAQVILLEAR

SEQ ID NO: 12 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKWQLQAEHALLDWQMALNKXXXXXENLNRAITAAQSCISXXXXXLDKAEDIRRNSDQARREAEKXXXXXRDLISNAQVILLEAR

SEQ ID NO: 13 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKWQLQAEHALLDWQMALNKXXXXXENLNRAITAAQSWISXXXXXCDKAEDIRRNSDQARREAEKXXXXXRDLISNAQVILLEAC

SEQ ID NO: 14 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKLQLQAEHFLLDVQMILNEXXXXXEELNRAITDAQSWISXXXXXLDRAEELARNLEKVRDEALKXXXXXRDLVSNAKVIALELK

SEQ ID NO: 15 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKLQLQAEHFLLDVQMILNEXXXXXEELNRCITDAQSWISXXXXXLDRAEECARNLEKVRDEALKXXXXXRDLVSNAKVIALELK

SEQ ID NO: 16 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKLQLQAEHFLLDVQMILNEXXXXXEELNRAITDAQSCISXXXXXLDRAEELARNLEKVRDEALKXXXXXRDLVSNAKVIALELK

SEQ ID NO: 17 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKLQLQAEHFLLDVQMILNEXXXXXEELNRAITDAQSWISXXXXXLDRAEELCRNLEKVRDEALKXXXXXRDLVSNACVIALELK

SEQ ID NO: 18 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKLQLQAEHALLDAQMMLNRXXXXXEKLNRIITTMQSWISXXXXXLDGAKELAKEVEELRQEAEKXXXXXRDLASNLKVILLELA

SEQ ID NO: 19 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKLQLQAEHALLDAQMMLNRXXXXXEKLNRIITTMQSCISXXXXXLDGAKELAKEVEELRQEAEKXXXXXRDLASNLKVILLELA

SEQ ID NO: 20 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKIQLQLEHALLDVQMALNRXXXXXESLNRMITWLQSWISXXXXXLDNAQEMAKEAEKIRKEMEKXXXXXRDLISNIIVILLELS

SEQ ID NO: 21 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKIQLQLEHALLDVQMALNRXXXXXESLNRMITWLQSCISXXXXXLDNAQEMAKEAEKIRKEMEKXXXXXRDLISNIIVILLELS

SEQ ID NO: 22 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKIQLQLEHALLDVQMALNRXXXXXESLNRMITWLQSWISXXXXXLDNAQEMCKEAEKIRKEMEKXXXXXRDLISNICVILLELS

SEQ ID NO: 23 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLAEHALLDAFMMLNVXXXXXEKLNRIITTMQSWIYXXXXXADGAKELAKEVEELEQEYEKXXXXXEDDASNLKVILLELA

SEQ ID NO: 24 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLAEHALLDAHMMLNMXXXXXEKLNRIITTMQSWIHXXXXXGDGAQELAKEVEELEQEYEKXXXXXEDEASNLKVILLELA

SEQ ID NO: 25 - protein, 87 aa, synthetic construct
  REGION 1..87: note = Synthetic
  VARIANT 23..37: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absentt
STKKTQLLAEHALLDAFMMLNMXXXXXEKLNRIITTMQSWIFXXXXXGDGAKELAKEVEELEQEFEKXXXXXEDEASNLKVILLELA

SEQ ID NO: 26 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLAEHALLDALMMLNMXXXXXEKLNRIITTMQSWIFXXXXXGDGAQELAKEVEELEQELEKXXXXXEDYASNLKVILLELA

SEQ ID NO: 27 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLAEHALLDAHMMLNVXXXXXEKLNRIITTMQSWIYXXXXXRDGAQELAKEVEELEQELEKXXXXXDDDASNLKVILLELA

SEQ ID NO: 28 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLAEHALLDALMMLNLXXXXXEKLNRIITTMQSWIFXXXXXGDGAQELAKEVEELEQEHEKXXXXXEDYASNLKVILLELA

SEQ ID NO: 29 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLAEHALLDAYMMLNMXXXXXEKLNRIITTMQSWILXXXXXSDGAQELAKEVEELEQELEKXXXXXDDDASNLKVILLELA

SEQ ID NO: 30 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTHLLAEHALLDAYMMLNVXXXXXEKLNRIITTMQSWIFXXXXXGDGAKELAKEVEELEQEFEKXXXXXDDDASNLKVILLELA

SEQ ID NO: 31 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLAEHALLDAYMMLNLXXXXXEKLNRIITTMQSWIFXXXXXADGAQELAIEVEELEQEYEKXXXXXDDYASNLKVILLELA

SEQ ID NO: 32 - protein, 87 aa, synthetic construct
  REGION 1..87: note = Synthetic
  VARIANT 22..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLMAEHALLDAFMMLNXXXXXXEKLNRIITTMQSWIFXXXXXGDDAQELAKEVEELEQELEKXXXXXDDDASNLKVILLELA

SEQ ID NO: 33 - protein, 87 aa, synthetic construct
  REGION 1..87: note = Synthetic
  VARIANT 22..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLIEHALLDALDMSRXXXXXXEKLSRIITTMQSWIFXXXXXGDGAQQLAKEVEELEQEHEKXXXXXEDEASNLKVILLELA

SEQ ID NO: 34 - protein, 87 aa, synthetic construct
  REGION 1..87: note = Synthetic
  VARIANT 22..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLLEHALLDALHMRRXXXXXXEKLSRIITTMQSWIFXXXXXGDGAQELAKEVEELEQEHEKRGRDVEDDASNLKVILLELA

SEQ ID NO: 35 - protein, 87 aa, synthetic construct
  REGION 1..87: note = Synthetic
  VARIANT 22..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLIEHALLDALNMRKXXXXXXEKLSRIITDMQSWIFXXXXXGDGAQQLAKEVEELEQEHEKXXXXXEDYASNLKVILLELA

SEQ ID NO: 36 - protein, 87 aa, synthetic construct
  REGION 1..87: note = Synthetic
  VARIANT 22..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLLEHALLDALHMSRXXXXXXEKLNRIITDMQSWIFXXXXXGDGAQDLAKEVEELEQEHEKXXXXXEDYASNLKVILLELA

SEQ ID NO: 37 - protein, 87 aa, synthetic construct
  REGION 1..87: note = Synthetic
  VARIANT 22..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLIEHALLDALHMSRXXXXXXEKLSRIITTMQSWIFXXXXXGDGAQHLAKEVEELEQEHEKXXXXXEDEASNLKVILLELA

SEQ ID NO: 38 - protein, 87 aa, synthetic construct
  REGION 1..87: note = Synthetic
  VARIANT 22..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKKTQLLIEHALLDALHMKRXXXXXXEKLNRIITNMQSWIFXXXXXGDGAQDLAKEVEELEQEHEKXXXXXEDYASNLKVILLELA

SEQ ID NO: 39 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STEKTQLAAEHALRDALMLKHLXXXXXEKLARIITTMQSWQFXXXXXGDGAQELAKEVEELQQEHEVXXXXXEDYASNLKVILLHLA

SEQ ID NO: 40 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STKNTQLAAEDALLDALMLRNLXXXXXEKLARIITTMQSWQFXXXXXGDGAQELAKEVEELQQEHEEXXXXXEDYASNLKVILLQLA

SEQ ID NO: 41 - protein, 87 aa, synthetic construct
  REGION 1..87: note = Synthetic
  VARIANT 23..27: note = Xaa, when present, can be any naturally occurring amino acid or is optionally absent
  VARIANT 43..47: note = Xaa, when present, can be any naturally occurring amino acid or is optionally absent
  VARIANT 68..72: note = Xaa, when present, can be any naturally occurring amino acid or is optionally absent
STEKTQHAAEDALRDALMLRNLXXXXXEKLARIITTMQSWQFXXXXXGDGAQELAKEVEELQQEHEVXXXXXEDYASNLKVILLQLA

SEQ ID NO: 42 - protein, 87 aa, synthetic construct
  REGION 1..87: note = Synthetic
  VARIANT 23..30: note = Xaa, when present, can be any naturally occurring amino acid or is optionally absent
  VARIANT 45..49: note = Xaa, when present, can be any naturally occurring amino acid or is optionally absent
  VARIANT 69..72: note = Xaa, when present, can be any naturally occurring amino acid or is optionally absent
TQKKQQLLAEHALLDALMILNMXXXXXXXXNRMITIAQSWIFTGXXXXXEAKEMIKMAEQAEEEARREXXXXEDYVSNLKVILKEIA

SEQ ID NO: 43 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TTKKYQLLVEHALLDALMMLNLXXXXXXKMNRIITTMQSWIFXXXXXXDQAEELAKLVEELREEFRKXXXXXXDYASNLKVILKELS

SEQ ID NO: 44 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TTKKIQLLVEHALLDALMILNLXXXXXXKLNRIITTLQSWIFXXXXXXDRARELAKLLEEIREEMRKXXXXXXDYVSNMIVIIRELA

SEQ ID NO: 45 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLAEHVLLDLLMMLNLXXXXXXKMNRLITIVQSWIFXXXXXXDQAEEMAKWVEELREEFRKXXXXXXDYASNVKVILKELS

SEQ ID NO: 46 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKYQLLIEHLLLDALMVLNMXXXXXXKLNRIITILQSWIFXXXXXXDLAEEMEKLMQEIEEELRRXXXXXXDYMSNMRVIIKELS

SEQ ID NO: 47 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKLQLLVEHLLLDMLMILNMXXXXXXKLNRLITELQSWIFXXXXXXDKAEEMWKIMEEIEKELREXXXXXXDYMSNAKVIIKELS

SEQ ID NO: 48 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TSKKQQLLAEHALLDALMILNIXXXXXXAVNRAITWLQSWIFXXXXXXDQAEEMRKLAEQIREEMRKXXXXXXDYVSNLEVIAKELS

SEQ ID NO: 49 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKYQLLIEHLLLDLLMVLNMXXXXXXKINRLITWLQSWIFXXXXXXDLAEEMYKILEELREEMREXXXXXXDYMSNMRVIVKELS

SEQ ID NO: 50 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKWQLLIEHLLLDLLMILNLXXXXXXKLNRLITWLQSWIFXXXXXXDLAEEMKKMMDEIEDELREXXXXXXDYMSNAKVIIKELS

SEQ ID NO: 51 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLVEHALLDALMILNLXXXXXXKLNRIITTMQSWIFXXXXXXDQAEELSKLVEEIREEMRKXXXXXXDYVSNLKVILDELS

SEQ ID NO: 52 - protein, 87 aa, synthetic construct
  REGION 1..87: note = Synthetic
  VARIANT 24..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TEKKLQLLVEHALLDALMILNLWXXXXXKLNRIITTMQSWIFXXXXXXDKAEELAKLVEELREEAREXXXXXXDYVSNLKVILKELS

SEQ ID NO: 53 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKYQLLMEHLLLDLLMVLNMXXXXXXKLNRLITIIQSWIFXXXXXXDKAEEMAKMLKEIEDELREXXXXXXDYMSNMIVIMKELS

SEQ ID NO: 54 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TTKKIQLLVEHALLDALMLLNLXXXXXXKMNRIITTMQSWIFXXXXXXDQAQELAKLVEELREEFRKXXXXXXDYVSNLKVILEELS

SEQ ID NO: 55 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLVEHALLDALMMLNLXXXXXXKLNRIITTMQSWIFXXXXXXDQAEELAKLVRELREEFRKXXXXXXDYASNLEVILRELS

SEQ ID NO: 56 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLVEHALLDALMILNLXXXXXXKLNRIITTMQSWIFXXXXXXDRARELAKLVEEIRDEMEKXXXXXXDYVSNLKVILEELA

SEQ ID NO: 57 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKYQLLIEHVLLDLLMLLNLXXXXXXKMNRLITILQSWIFXXXXXXDKAEEMAKLLKELREEFREXXXXXXDYISNAIVILKELS

SEQ ID NO: 58 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLVEHALLDALMMLNLXXXXXXKLNRIITTMQSWIFXXXXXXDRAEELAKLVEELREEFRKXXXXXXDYASNLKVILKELS

SEQ ID NO: 59 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLVEHALLDALMMLNLXXXXXXKLNRIITTMQSWIFXXXXXXDQARELAKLVEELREEFRKXXXXXXDYASNLKVILEELA

SEQ ID NO: 60 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = vnt
  VARIANT 68..73: note = Xaa, when present, can be any naturally occurring amino acid or is optionally absent
TKKKLQLLVEHALLDALMLLNLXXXXXXKLNRIITTMQSWIFXXXXXXDQAEELAKLVEEIREELRKXXXXXXDYVSNLKVILKELS

SEQ ID NO: 61 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TTKKYQLLVEHALLDALMILNLXXXXXXKLNRIITTMQSWIFXXXXXXDQAEELAKLVREIREEMRKXXXXXXDYVSNLEVILRELS

SEQ ID NO: 62 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLVEHALLDALMILNLXXXXXXKLNRIITTMQSWIFXXXXXXDRAEELAKLVREIREEMRKXXXXXXDYVSNLEVILRELS

SEQ ID NO: 63 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKYQLLIEHLLLDLLMILNLXXXXXXKLNRLITWLQSWIFRXXXXXDKAEEWAKILKEIREELREXXXXXXDYMSNAIVIMKELS

SEQ ID NO: 64 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TDKKLQLLVEHLLLDLLMMLNLXXXXXXKMNRLITIAQSWIFTXXXXXDLAREMIKLLEETEDENRKXXXXXXDYVSNARVIAKELE

SEQ ID NO: 65 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKIQLLVEHALLDALMLLNLXXXXXXKMNRIITTMQSWIFXXXXXXDQAEELAKLVEELKEEFKKXXXXXXDYVSNLKVILKELS

SEQ ID NO: 66 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKYQLLIEHALLDALMILNLXXXXXXKLNRIITTMQSWIFTXXXXXDKAEELEKLAKEIEDEAREXXXXXXDYMSNLRVILKELS

SEQ ID NO: 67 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TKKKAQLLAEHALLDALMLLNLXXXXXXRLNRIITWLQSIIFTXXXXXDMVKEAVKLADEIEDEMRKXXXXXXDYVSNLRVILQELA

SEQ ID NO: 68 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TQKKNQLLAEHLLLDALMVLNQXXXXXXVANRIITWAQSWIFEXXXXXNKAEEAKKLAKKLEEEMRKXXXXXXDYISNMKVIAEEMS

SEQ ID NO: 69 - protein, 107 aa, synthetic construct
  REGION 1..107: note = Synthetic
  VARIANT 21..29: note = Xaa, when present, can be any naturally occurring amino acid or is optionally absent
  VARIANT 46..50: note = Xaa, when present, can be any naturally occurring amino acid or is optionally absent
  VARIANT 77..80: note = Xaa, when present, can be any naturally occurring amino acid or is optionally absent
EDYYSNLKVILEELAREMERXXXXXXXXXWRQWKKIVERIRQIRSXXXXXNEAKELLNRLITYIQSQIFEISERIRXXXXEKKEESWKKWQLLLEHALLDVLMLLND

SEQ ID NO: 70 - protein, 107 aa, synthetic construct
  REGION 1..107: note = Synthetic
  VARIANT 23..28: note = Xaa, when present, can be any naturally occurring amino acid or is optionally absent
  VARIANT 55..56: note = Xaa, when present, can be any naturally occurring amino acid or is optionally absent
  VARIANT 79..82: note = Xaa, when present, can be any naturally occurring amino acid or is optionally absent
PEKKRQLLLEHILLDALMLLNLXXXXXXNTESKFEDYISNAEVIAEELAKLMESXXLSDEAEKFKKIKQWLREVWRIWXXXXWSTLEDKARELLNRIITTIQSQIFY

SEQ ID NO: 71 - protein, 104 aa, synthetic construct
  REGION 1..104: note = Synthetic
  VARIANT 25..31: note = Xaa, when present, can be any naturally occurring amino acid or is optionally absent
  VARIANT 55..57: note = Xaa, when present, can be any naturally occurring amino acid or is optionally absent
  VARIANT 80..83: note = Xaa, when present, can be any naturally occurring amino acid or is optionally absent
PEKKRQLLLEHILLDLLMILNMIEXXXXXXXSEMEDYWSNVRVILRELARLMEEXXXKELSELMERMRKIVEKIRQIVTXXXXLDTAREWLNRLITWIQSLIFR

SEQ ID NO: 72 - protein, 104 aa, synthetic construct
  REGION 1..104: note = Synthetic
  VARIANT 23..28: note = Xaa, when present, can be any naturally occurring amino acid or is optionally absent
  VARIANT 55..57: note = Xaa, when present, can be any naturally occurring amino acid or is optionally absent
  VARIANT 80..83: note = Xaa, when present, can be any naturally occurring amino acid or is optionally absent
PEKKRQLLLEHILLDLLMILNMXXXXXXNTESEMEDYWSNVRVILRELARLMEEXXXKELSELMERMRKIVEKIRQIVTXXXXLDTAREWLNRLITWIQSLIFR

SEQ ID NO: 73 - protein, 96 aa, synthetic construct
  REGION 1..96: note = Synthetic
  VARIANT 22..27: note = Xaa, when present, can be any naturally occurring amino acid or is optionally absent
  VARIANT 47..50: note = Xaa, when present, can be any naturally occurring amino acid or is optionally absent
  VARIANT 72..75: note = Xaa, when present, can be any naturally occurring amino acid or is optionally absent
PEKKRQLLAEHLLLDVLMMLNXXXXXXDYASNAQVIADEFRELAREXXXXDEAKKAEKIIEALERAREWLLXXXXKEKAKEALNRAITIAQSWIFN

SEQ ID NO: 74 - protein, 104 aa, synthetic construct
  REGION 1..104: note = Synthetic
  VARIANT 22..27: note = Xaa, when present, can be any naturally occurring amino acid or is optionally absent
  VARIANT 55..57: note = Xaa, when present, can be any naturally occurring amino acid or is optionally absent
  VARIANT 80..83: note = Xaa, when present, can be any naturally occurring amino acid or is optionally absent
PEKKRQLLLEHLLLDLLMILNXXXXXXKNIESDWEDYMSNIEVIIEELRKIMESXXXSEKAKEWKRMKQWVRRILEIVKXXXXLEEAKEWLNRLITIVQSEIFE

SEQ ID NO: 75 - protein, 104 aa, synthetic construct
  REGION 1..104: note = Synthetic
  VARIANT 22..27: note = Xaa, when present, can be any naturally occurring amino acid or is optionally absent
  VARIANT 55..57: note = Xaa, when present, can be any naturally occurring amino acid or is optionally absent
  VARIANT 80..83: note = Xaa, when present, can be any naturally occurring amino acid or is optionally absent
WEKKRQLLLEHLLLDLLMILNXXXXXXQNTESLMEDYMSNAKVIVEELARMMRSXXXEDKAREWEEMKKRIEEIRQIIQXXXXKERAKEELNRLITYVQSEIFR

SEQ ID NO: 76 - protein, 100 aa, synthetic construct
  REGION 1..100: note = Synthetic
  VARIANT 22..27: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLLAEHALLDALMILNXXXXXXQNAEEKLEDYASNVEVILEEIARLMESGXXKDEAEKAKRMKEWMKRIKTXXXEDEQEEMANRIITLLQSWIFS

SEQ ID NO: 77 - protein, 100 aa, synthetic construct
  REGION 1..100: note = Synthetic
  VARIANT 22..27: note = Xaa, when present, can be any naturally occurring amino acid or is optionally absent
  VARIANT 54..57: note = Xaa, when present, can be any naturally occurring amino acid or is optionally absent
  VARIANT 77..79: note = Xaa, when present, can be any naturally occurring amino acid or is optionally absent
PEKKRQLLAEHALLDALMILNXXXXXXQNAEEKLEDYMSNVEVIMEEFARMMRXXXXSEEAENAERIKKWVRKASSXXXSEEQREMMNRAITLMQSWIFE

SEQ ID NO: 78 - protein, 100 aa, synthetic construct
  REGION 1..100: note = Synthetic
  VARIANT 23..28: note = Xaa, when present, can be any naturally occurring amino acid or is optionally absent
  VARIANT 56..58: note = Xaa, when present, can be any naturally occurring amino acid or is optionally absent
  VARIANT 75..78: note = Xaa, when present, can be any naturally occurring amino acid or is optionally absent
PEKKRQLLAEHLLLDALMVLNMXXXXXXNTEEKLEDYISNMKVIIKEMIELMRSLXXXEEAEKWKEALKAVEKIXXXXDSETARELANRIITLAQSAIFY

SEQ ID NO: 79 - protein, 100 aa, synthetic construct
  REGION 1..100: note = Synthetic
  VARIANT 23..28: note = Xaa, when present, can be any naturally occurring amino acid or is optionally absent
  VARIANT 56..58: note = Xaa, when present, can be any naturally occurring amino acid or is optionally absent
  VARIANT 77..78: note = Xaa, when present, can be any naturally occurring amino acid or is optionally absent
PEKKRQLLAEHALLDALMFLNLXXXXXXQAEEKIEDYASNLRVIAEELARLFENLXXXDEAQKAKDIKELAERARSXXSSEKRKEAMNRAITILQSMIFR

SEQ ID NO: 80 - protein, 100 aa, synthetic construct
  REGION 1..100: note = Synthetic
  VARIANT 23..27: note = Xaa, when present, can be any naturally occurring amino acid or is optionally absent
  VARIANT 56..58: note = Xaa, when present, can be any naturally occurring amino acid or is optionally absentt
  VARIANT 75..78: note = Xaa, when present, can be any naturally occurring amino acid or is optionally absent
PEKKRQLLAEHALLDALMILNIXXXXXDNTESKLEDYISNLKVILEEIARLMESLXXXDEAEKAKEAMRLADKAXXXXSEEEKKEAMNRVITWAQSWIFN

SEQ ID NO: 81 - protein, 100 aa, synthetic construct
  REGION 1..100: note = Synthetic
  VARIANT 23..29: note = Xaa, when present, can be any naturally occurring amino acid or is optionally absent
  VARIANT 56..58: note = Xaa, when present, can be any naturally occurring amino acid or is optionally absent
  VARIANT 77..79: note = Xaa, when present, can be any naturally occurring amino acid or is optionally absent
PEKKRQLLAEHALLDALMMLNIXXXXXXXAEEKLEDYWSNLIVILREIAKLMESLXXXDEAEKAKEAARWAEEARTXXXKDQRRELANRIITLLQSWIFS

SEQ ID NO: 82 - protein, 99 aa, synthetic construct
  REGION 1..99: note = Synthetic
  VARIANT 55..57: note = Xaa, when present, can be any naturally occurring amino acid or is optionally absent
  VARIANT 76..78: note = Xaa, when present, can be any naturally occurring amino acid or is optionally absent
EKKRQLLAEHLLLDALMILNIIETNEQNAESKLEDYISNAKVILDEFREMARDLXXXDEAKKAEKMKRWLEKMRSXXXSDERREWANRMITTAQSWIFN

SEQ ID NO: 83 - protein, 87 aa, synthetic construct
  REGION 1..87: note = Synthetic
  VARIANT 23..28: note = Xaa, when present, can be any naturally occurring amino acid or is optionally absent
  VARIANT 44..48: note = Xaa, when present, can be any naturally occurring amino acid or is optionally absent
  VARIANT 68..73: note = Xaa, when present, can be any naturally occurring amino acid or is optionally absent
TNKKAQLHAEFALHDALMLLNLXXXXXXRLNRIITWLQSIIFYXXXXXDMVKEAVKDADEIEDEMRKXXXXXXDYVSNLRLILQELA

SEQ ID NO: 84 - protein, 87 aa, synthetic construct
  REGION 1..87: note = Synthetic
  VARIANT 23..28: note = Xaa, when present, can be any naturally occurring amino acid or is optionally absent
  VARIANT 44..48: note = Xaa, when present, can be any naturally occurring amino acid or is optionally absent
  VARIANT 68..73: note = Xaa, when present, can be any naturally occurring amino acid or is optionally absent
TNKEAQLHAEFALYDALMLLNLXXXXXXRLNRIITWLQSIIFYXXXXXDMVKEAVKLADEIEDEMRKXXXXXXDYVVNLRLILQELA

SEQ ID NO: 85 - protein, 87 aa, synthetic construct
  REGION 1..87: note = Synthetic
  VARIANT 23..28: note = Xaa, when present, can be any naturally occurring amino acid or is optionally absent
  VARIANT 44..48: note = Xaa, when present, can be any naturally occurring amino acid or is optionally absent
  VARIANT 68..73: note = Xaa, when present, can be any naturally occurring amino acid or is optionally absent
TKKDAELLAEFALYDALMLLNLXXXXXXRLNEIITWLQSIIFYXXXXXDMVKEAVKLADEIEDEMRKXXXXXXDYVSNLRLILQELA

SEQ ID NO: 86 - protein, 87 aa, synthetic construct
  REGION 1..87: note = Synthetic
  VARIANT 23..28: note = Xaa, when present, can be any naturally occurring amino acid or is optionally absent
  VARIANT 43..48: note = Xaa, when present, can be any naturally occurring amino acid or is optionally absent
  VARIANT 68..73: note = Xaa, when present, can be any naturally occurring amino acid or is optionally absent
TNKKAQLHAEFALYDALMLLNLXXXXXXRLNDIITWLQSIIFXXXXXXDMVKEAVKLADEIEDEMRKXXXXXXDYVVNLRYILQELA

SEQ ID NO: 87 - protein, 107 aa, synthetic construct
  REGION 1..107: note = Synthetic
  VARIANT 21..24: note = Xaa, when present, can be any naturally occurring amino acid or is optionally absent
  VARIANT 46..50: note = Xaa, when present, can be any naturally occurring amino acid or is optionally absent
  VARIANT 77..80: note = Xaa, when present, can be any naturally occurring amino acid or is optionally absent
EDYYSNLKLILEELAREMERXXXXDKAEEWRQWKKIVERIRQIRSXXXXXNEAKELLNRLITYIQSQIFEVLHGVGXXXXEKKEESWKKWDLLLEHALLDVLMLLND

SEQ ID NO: 88 - protein, 107 aa, synthetic construct
  REGION 1..107: note = Synthetic
  VARIANT 21..24: note = Xaa, when present, can be any naturally occurring amino acid or is optionally absent
  VARIANT 46..50: note = Xaa, when present, can be any naturally occurring amino acid or is optionally absent
  VARIANT 77..80: note = Xaa, when present, can be any naturally occurring amino acid or is optionally absent
EDYYSNLKVILEELAREMERXXXXDKAEEWRQWKKIVERIRQIRSXXXXXNEAKELLNELITYIQSQIFEVIEREGXXXXEKKEESWKKWELHLEHALLDVLMLLND

SEQ ID NO: 89 - protein, 107 aa, synthetic construct
  REGION 1..107: note = Synthetic
  VARIANT 21..24: note = Xaa, when present, can be any naturally occurring amino acid or is optionally absent
  VARIANT 46..50: note = Xaa, when present, can be any naturally occurring amino acid or is optionally absent
  VARIANT 77..80: note = Xaa, when present, can be any naturally occurring amino acid or is optionally absent
EDYYSNLKLILEELAREMERXXXXDKAEEWRQWKKIVERIRQIRSXXXXXNEAKELLNRLITYIQSQIFEVLEGVGXXXXEKKEESWKKWELHLEHALLDVLMLLND

SEQ ID NO: 90 - protein, 100 aa, synthetic construct
  REGION 1..100: note = Synthetic
  VARIANT 23..32: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXKLEDYAFNFELILEEIARLFESGXXKDEAEKAKRMKEWMKRIKTXXXEDEQEEMANAIITILQSWIFS

SEQ ID NO: 91 - protein, 100 aa, synthetic construct
  REGION 1..100: note = Synthetic
  VARIANT 23..32: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLLAEHALFDLLMILNIXXXXXXXXXXKLEDYAYNAGVILEEIARLFESGXXKDEAEKAKRMKEWMKRIKDXXXEDEQEEMANEIITILQSWNFS

SEQ ID NO: 92 - protein, 100 aa, synthetic construct
  REGION 1..100: note = Synthetic
  VARIANT 23..32: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQITAEEALKDALSILNIXXXXXXXXXXQLERFAKRFERNLWGIARLFESGXXKDEAEKAKRMKEWMKRIKTXXXEDEQEEMANAIITILQSWIFS

SEQ ID NO: 93 - protein, 100 aa, synthetic construct
  REGION 1..100: note = Synthetic
  VARIANT 23..32: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQIMAEEALKDALSILNIXXXXXXXXXXQLERFAKRFERNLWGIARLFESGXXKDEAEKAKRMIEWMKRIKTXXXEDEQEEMANAIITILQSWFFS

SEQ ID NO: 94 - protein, 100 aa, synthetic construct
  REGION 1..100: note = Synthetic
  VARIANT 23..32: note = Xaa, when present, can be any naturally occurring amino acid or is optionally absent
  VARIANT 55..56: note = Xaa, when present, can be any naturally occurring amino acid or is optionally absent
  VARIANT 77..79: note = Xaa, when present, can be any naturally occurring amino acid or is optionally absent
PKKKIQLYAEHALYDALMILNIXXXXXXXXXXELEDYAFNFELILEEIARLFESXXQKDEAEKAKRMKEWMKRIKTXXXEDEQEEMANAIITILQSWIFS

SEQ ID NO: 95 - protein, 5 aa, synthetic construct
  REGION 1..5: note = Synthetic
GGGGG

SEQ ID NO: 96 - protein, 5 aa, synthetic construct
  REGION 1..5: note = Synthetic
GSGGG

SEQ ID NO: 97 - protein, 6 aa, synthetic construct
  REGION 1..6: note = Synthetic
GGGGGG

SEQ ID NO: 98 - protein, 6 aa, synthetic construct
  REGION 1..6: note = Synthetic
GGSGGG

SEQ ID NO: 99 - protein, 16 aa, synthetic construct
  REGION 1..16: note = Synthetic
GGSGGSGGGSGGSGSG

SEQ ID NO: 100 - protein, 15 aa, synthetic construct
  REGION 1..15: note = Synthetic
GSGGSGGGSGGSGSG

SEQ ID NO: 101 - protein, 30 aa, synthetic construct
  REGION 1..30: note = Synthetic
GGSGGSGGGSGGSGGGGSGGSGGGSGGGGS

SEQ ID NO: 102 - protein, 5 aa, synthetic construct
  REGION 1..5: note = Synthetic
  VARIANT 5: note = X is Q, E, or S
GGGGX

SEQ ID NO: 103 - protein, 87 aa, synthetic construct
  REGION 1..87: note = Synthetic
STKKWQLQAEHALLDWQMALNKSPEPNENLNRAITAAQSWISTGKIDLDKAEDIRRNSDQARREAEKRGIDVRDLISNAQVILLEAR

SEQ ID NO: 104 - protein, 87 aa, synthetic construct
  REGION 1..87: note = Synthetic
STKKWQLQAEHALLDWQMALNKSPEPNENLNRAITAAQSCISTGKCDLDKAEDIRRNSDQARREAEKRGIDVRDLISNAQVILLEAR

SEQ ID NO: 105 - protein, 87 aa, synthetic construct
  REGION 1..87: note = Synthetic
STKKWQLQAEHALLDWQMALNKSPEPNENLNRAITAAQSWISTGKIDCDKAEDIRRNSDQARREAEKRGIDVRDLISNAQVILLEAC

SEQ ID NO: 106 - protein, 87 aa, synthetic construct
  REGION 1..87: note = Synthetic
STKKLQLQAEHFLLDVQMILNESPEPNEELNRAITDAQSWISTGKIDLDRAEELARNLEKVRDEALKRGIDVRDLVSNAKVIALELK

SEQ ID NO: 107 - protein, 87 aa, synthetic construct
  REGION 1..87: note = Synthetic
STKKLQLQAEHFLLDVQMILNESPEPNEELNRCITDAQSWISTGKIDLDRAEECARNLEKVRDEALKRGIDVRDLVSNAKVIALELK

SEQ ID NO: 108 - protein, 87 aa, synthetic construct
  REGION 1..87: note = Synthetic
STKKLQLQAEHFLLDVQMILNESPEPNEELNRAITDAQSCISTGKCDLDRAEELARNLEKVRDEALKRGIDVRDLVSNAKVIALELK

SEQ ID NO: 109 - protein, 87 aa, synthetic construct
  REGION 1..87: note = Synthetic
STKKLQLQAEHFLLDVQMILNESPEPNEELNRAITDAQSWISTGKIDLDRAEELCRNLEKVRDEALKRGIDVRDLVSNACVIALELK

SEQ ID NO: 110 - protein, 87 aa, synthetic construct
  REGION 1..87: note = Synthetic
STKKLQLQAEHALLDAQMMLNRSPEPNEKLNRIITTMQSWISTGKIDLDGAKELAKEVEELRQEAEKRGIDVRDLASNLKVILLELA

SEQ ID NO: 111 - protein, 87 aa, synthetic construct
  REGION 1..87: note = Synthetic
STKKLQLQAEHALLDAQMMLNRSPEPNEKLNRIITTMQSCISTGKCDLDGAKELAKEVEELRQEAEKRGIDVRDLASNLKVILLELA

SEQ ID NO: 112 - protein, 87 aa, synthetic construct
  REGION 1..87: note = Synthetic
STKKIQLQLEHALLDVQMALNRSPEPNESLNRMITWLQSWISTGKIDLDNAQEMAKEAEKIRKEMEKRGIDVRDLISNIIVILLELS

SEQ ID NO: 113 - protein, 87 aa, synthetic construct
  REGION 1..87: note = Synthetic
STKKIQLQLEHALLDVQMALNRSPEPNESLNRMITWLQSCISTGKCDLDNAQEMAKEAEKIRKEMEKRGIDVRDLISNIIVILLELS

SEQ ID NO: 114 - protein, 87 aa, synthetic construct
  REGION 1..87: note = Synthetic
STKKIQLQLEHALLDVQMALNRSPEPNESLNRMITWLQSWISTGKIDLDNAQEMCKEAEKIRKEMEKRGIDVRDLISNICVILLELS

SEQ ID NO: 115 - protein, 87 aa, synthetic construct
  REGION 1..87: note = Synthetic
STKKTQLLAEHALLDAFMMLNVVPEPNEKLNRIITTMQSWIYTGKIDADGAKELAKEVEELEQEYEKRGIDVEDDASNLKVILLELA

SEQ ID NO: 116 - protein, 87 aa, synthetic construct
  REGION 1..87: note = Synthetic
STKKTQLLAEHALLDAHMMLNMLPEPNEKLNRIITTMQSWIHTGKIDGDGAQELAKEVEELEQEYEKRGIDVEDEASNLKVILLELA

SEQ ID NO: 117 - protein, 87 aa, synthetic construct
  REGION 1..87: note = Synthetic
STKKTQLLAEHALLDAFMMLNMVPEPNEKLNRIITTMQSWIFTGKIDGDGAKELAKEVEELEQEFEKRGIDVEDEASNLKVILLELA

SEQ ID NO: 118 - protein, 87 aa, synthetic construct
  REGION 1..87: note = Synthetic
STKKTQLLAEHALLDALMMLNMVPEPNEKLNRIITTMQSWIFTGKIDGDGAQELAKEVEELEQELEKRGIDVEDYASNLKVILLELA

SEQ ID NO: 119 - protein, 87 aa, synthetic construct
  REGION 1..87: note = Synthetic
STKKTQLLAEHALLDAHMMLNVVPEPNEKLNRIITTMQSWIYTGKIDRDGAQELAKEVEELEQELEKRGIDVDDDASNLKVILLELA

SEQ ID NO: 120 - protein, 87 aa, synthetic construct
  REGION 1..87: note = Synthetic
STKKTQLLAEHALLDALMMLNLLPEPNEKLNRIITTMQSWIFTGKIDGDGAQELAKEVEELEQEHEKRGIDVEDYASNLKVILLELA

SEQ ID NO: 121 - protein, 87 aa, synthetic construct
  REGION 1..87: note = Synthetic
STKKTQLLAEHALLDAYMMLNMVPEPNEKLNRIITTMQSWILTGKIDSDGAQELAKEVEELEQELEKRGIDVDDDASNLKVILLELA

SEQ ID NO: 122 - protein, 87 aa, synthetic construct
  REGION 1..87: note = Synthetic
STKKTHLLAEHALLDAYMMLNVMPEPNEKLNRIITTMQSWIFTGKIDGDGAKELAKEVEELEQEFEKRGIDVDDDASNLKVILLELA

SEQ ID NO: 123 - protein, 87 aa, synthetic construct
  REGION 1..87: note = Synthetic
STKKTQLLAEHALLDAYMMLNLVPEPNEKLNRIITTMQSWIFTGKIDADGAQELAIEVEELEQEYEKRGIDVDDYASNLKVILLELA

SEQ ID NO: 124 - protein, 87 aa, synthetic construct
  REGION 1..87: note = Synthetic
STKKTQLMAEHALLDAFMMLNVLPEPNEKLNRIITTMQSWIFTGKIDGDDAQELAKEVEELEQELEKRGIDVDDDASNLKVILLELA

SEQ ID NO: 125 - protein, 87 aa, synthetic construct
  REGION 1..87: note = Synthetic
STKKTQLLIEHALLDALDMSRNLPEPNEKLSRIITTMQSWIFTGKIDGDGAQQLAKEVEELEQEHEKRGEDVEDEASNLKVILLELA

SEQ ID NO: 126 - protein, 87 aa, synthetic construct
  REGION 1..87: note = Synthetic
STKKTQLLLEHALLDALHMRRNLPEPNEKLSRIITTMQSWIFTGKIDGDGAQELAKEVEELEQEHEKRGRDVEDDASNLKVILLELA

SEQ ID NO: 127 - protein, 87 aa, synthetic construct
  REGION 1..87: note = Synthetic
STKKTQLLIEHALLDALNMRKKLPEPNEKLSRIITDMQSWIFTGKIDGDGAQQLAKEVEELEQEHEKRGGDVEDYASNLKVILLELA

SEQ ID NO: 128 - protein, 87 aa, synthetic construct
  REGION 1..87: note = Synthetic
STKKTQLLLEHALLDALHMSRELPEPNEKLNRIITDMQSWIFTGKIDGDGAQDLAKEVEELEQEHEKRGGDVEDYASNLKVILLELA

SEQ ID NO: 129 - protein, 87 aa, synthetic construct
  REGION 1..87: note = Synthetic
STKKTQLLIEHALLDALHMSRKLPEPNEKLSRIITTMQSWIFTGKIDGDGAQHLAKEVEELEQEHEKRGGEVEDEASNLKVILLELA

SEQ ID NO: 130 - protein, 87 aa, synthetic construct
  REGION 1..87: note = Synthetic
STKKTQLLIEHALLDALHMKRKLPEPNEKLNRIITNMQSWIFTEKIDGDGAQDLAKEVEELEQEHEKRGQDVEDYASNLKVILLELA

SEQ ID NO: 131 - protein, 87 aa, synthetic construct
  REGION 1..87: note = Synthetic
STEKTQLAAEHALRDALMLKHLLNEPNEKLARIITTMQSWQFTGKIDGDGAQELAKEVEELQQEHEVRGIDVEDYASNLKVILLHLA

SEQ ID NO: 132 - protein, 87 aa, synthetic construct
  REGION 1..87: note = Synthetic
STKNTQLAAEDALLDALMLRNLLNEPNEKLARIITTMQSWQFTEKIDGDGAQELAKEVEELQQEHEERGIDVEDYASNLKVILLQLA

SEQ ID NO: 133 - protein, 87 aa, synthetic construct
  REGION 1..87: note = Synthetic
STEKTQHAAEDALRDALMLRNLLNEPNEKLARIITTMQSWQFTEKIDGDGAQELAKEVEELQQEHEVRGIDVEDYASNLKVILLQLA

SEQ ID NO: 134 - protein, 87 aa, synthetic construct
  REGION 1..87: note = Synthetic
TQKKQQLLAEHALLDALMILNMLKTSSEAVNRMITIAQSWIFTGTSNPEEAKEMIKMAEQAEEEARREGVDTEDYVSNLKVILKEIA

SEQ ID NO: 135 - protein, 87 aa, synthetic construct
  REGION 1..87: note = Synthetic
TTKKYQLLVEHALLDALMMLNLSSESNEKMNRIITTMQSWIFTGTFDPDQAEELAKLVEELREEFRKRGIDTEDYASNLKVILKELS

SEQ ID NO: 136 - protein, 87 aa, synthetic construct
  REGION 1..87: note = Synthetic
TTKKIQLLVEHALLDALMILNLSSESNEKLNRIITTLQSWIFRGEIDPDRARELAKLLEEIREEMRKRGIDTEDYVSNMIVIIRELA

SEQ ID NO: 137 - protein, 87 aa, synthetic construct
  REGION 1..87: note = Synthetic
TKKKIQLLAEHVLLDLLMMLNLSSESNEKMNRLITIVQSWIFTGTIDPDQAEEMAKWVEELREEFRKRGIDTEDYASNVKVILKELS

SEQ ID NO: 138 - protein, 87 aa, synthetic construct
  REGION 1..87: note = Synthetic
TKKKYQLLIEHLLLDALMVLNMSSESNEKLNRIITILQSWIFTGTWDPDLAEEMEKLMQEIEEELRRRGIDTEDYMSNMRVIIKELS

SEQ ID NO: 139 - protein, 87 aa, synthetic construct
  REGION 1..87: note = Synthetic
TKKKLQLLVEHLLLDMLMILNMSSESNEKLNRLITELQSWIFRGEIDPDKAEEMWKIMEEIEKELRERGIDTEDYMSNAKVIIKELS

SEQ ID NO: 140 - protein, 87 aa, synthetic construct
  REGION 1..87: note = Synthetic
TSKKQQLLAEHALLDALMILNISSESSEAVNRAITWLQSWIFKGTVNPDQAEEMRKLAEQIREEMRKRGIDTEDYVSNLEVIAKELS

SEQ ID NO: 141 - protein, 87 aa, synthetic construct
  REGION 1..87: note = Synthetic
TKKKYQLLIEHLLLDLLMVLNMSSESNEKINRLITWLQSWIFTGTYDPDLAEEMYKILEELREEMRERGIDTEDYMSNMRVIVKELS

SEQ ID NO: 142 - protein, 87 aa, synthetic construct
  REGION 1..87: note = Synthetic
TKKKWQLLIEHLLLDLLMILNLSSESNEKLNRLITWLQSWIFTGTYDPDLAEEMKKMMDEIEDELRERGIDTEDYMSNAKVIIKELS

SEQ ID NO: 143 - protein, 87 aa, synthetic construct
  REGION 1..87: note = Synthetic
TKKKIQLLVEHALLDALMILNLSSESNEKLNRIITTMQSWIFTGTIDPDQAEELSKLVEEIREEMRKRGIDTEDYVSNLKVILDELS

SEQ ID NO: 144 - protein, 87 aa, synthetic construct
  REGION 1..87: note = Synthetic
TEKKLQLLVEHALLDALMILNLWSESNEKLNRIITTMQSWIFTGRIDPDKAEELAKLVEELREEARERGIDTEDYVSNLKVILKELS

SEQ ID NO: 145 - protein, 87 aa, synthetic construct
  REGION 1..87: note = Synthetic
TKKKYQLLMEHLLLDLLMVLNMSSESNEKLNRLITIIQSWIFTGTWDPDKAEEMAKMLKEIEDELRERGIDTEDYMSNMIVIMKELS

SEQ ID NO: 146 - protein, 87 aa, synthetic construct
  REGION 1..87: note = Synthetic
TTKKIQLLVEHALLDALMLLNLSSESNEKMNRIITTMQSWIFEGRIDPDQAQELAKLVEELREEFRKRGIDTEDYVSNLKVILEELS

SEQ ID NO: 147 - protein, 87 aa, synthetic construct
  REGION 1..87: note = Synthetic
TKKKIQLLVEHALLDALMMLNLSSESNEKLNRIITTMQSWIFTGTIDPDQAEELAKLVRELREEFRKRGIDTEDYASNLEVILRELS

SEQ ID NO: 148 - protein, 87 aa, synthetic construct
  REGION 1..87: note = Synthetic
TKKKIQLLVEHALLDALMILNLSSKSNEKLNRIITTMQSWIFNGTIDPDRARELAKLVEEIRDEMEKNGIDTEDYVSNLKVILEELA

SEQ ID NO: 149 - protein, 87 aa, synthetic construct
  REGION 1..87: note = Synthetic
TKKKYQLLIEHVLLDLLMLLNLSSESNEKMNRLITILQSWIFTGTYDPDKAEEMAKLLKELREEFRERGIDTEDYISNAIVILKELS

SEQ ID NO: 150 - protein, 87 aa, synthetic construct
  REGION 1..87: note = Synthetic
TKKKIQLLVEHALLDALMMLNLSSESNEKLNRIITTMQSWIFTGTIDPDRAEELAKLVEELREEFRKRGIDTEDYASNLKVILKELS

SEQ ID NO: 151 - protein, 87 aa, synthetic construct
  REGION 1..87: note = Synthetic
TKKKIQLLVEHALLDALMMLNLSSESNEKLNRIITTMQSWIFNGTIDPDQARELAKLVEELREEFRKRGIDTEDYASNLKVILEELA

SEQ ID NO: 152 - protein, 87 aa, synthetic construct
  REGION 1..87: note = Synthetic
TKKKLQLLVEHALLDALMLLNLSSESNEKLNRIITTMQSWIFTGTVDPDQAEELAKLVEEIREELRKRGIDTEDYVSNLKVILKELS

SEQ ID NO: 153 - protein, 87 aa, synthetic construct
  REGION 1..87: note = Synthetic
TTKKYQLLVEHALLDALMILNLSSESNEKLNRIITTMQSWIFTGTFDPDQAEELAKLVREIREEMRKRGIDTEDYVSNLEVILRELS

SEQ ID NO: 154 - protein, 87 aa, synthetic construct
  REGION 1..87: note = Synthetic
TKKKIQLLVEHALLDALMILNLSSESNEKLNRIITTMQSWIFTGTIDPDRAEELAKLVREIREEMRKRGIDTEDYVSNLEVILRELS

SEQ ID NO: 155 - protein, 87 aa, synthetic construct
  REGION 1..87: note = Synthetic
TKKKYQLLIEHLLLDLLMILNLSSESNEKLNRLITWLQSWIFRGEWDPDKAEEWAKILKEIREELRERGIDTEDYMSNAIVIMKELS

SEQ ID NO: 156 - protein, 87 aa, synthetic construct
  REGION 1..87: note = Synthetic
TDKKLQLLVEHLLLDLLMMLNLSSKSNEKMNRLITIAQSWIFTGKVDPDLAREMIKLLEETEDENRKNGIDTEDYVSNARVIAKELE

SEQ ID NO: 157 - protein, 87 aa, synthetic construct
  REGION 1..87: note = Synthetic
TKKKIQLLVEHALLDALMLLNLSSESNEKMNRIITTMQSWIFTGTIDPDQAEELAKLVEELKEEFKKRGIDTEDYVSNLKVILKELS

SEQ ID NO: 158 - protein, 87 aa, synthetic construct
  REGION 1..87: note = Synthetic
TKKKYQLLIEHALLDALMILNLWSESNEKLNRIITTMQSWIFTGTYDPDKAEELEKLAKEIEDEARERGIDTEDYMSNLRVILKELS

SEQ ID NO: 159 - protein, 87 aa, synthetic construct
  REGION 1..87: note = Synthetic
TKKKAQLLAEHALLDALMLLNLSSESNERLNRIITWLQSIIFTGTYDPDMVKEAVKLADEIEDEMRKRGIDTEDYVSNLRVILQELA

SEQ ID NO: 160 - protein, 87 aa, synthetic construct
  REGION 1..87: note = Synthetic
TQKKNQLLAEHLLLDALMVLNQSSESSEVANRIITWAQSWIFEGRVDPNKAEEAKKLAKKLEEEMRKRGIDMEDYISNMKVIAEEMS

SEQ ID NO: 161 - protein, 107 aa, synthetic construct
  REGION 1..107: note = Synthetic
EDYYSNLKVILEELAREMERNGLSDKAEEWRQWKKIVERIRQIRSNNSDLNEAKELLNRLITYIQSQIFEISERIRETDQEKKEESWKKWQLLLEHALLDVLMLLND

SEQ ID NO: 162 - protein, 107 aa, synthetic construct
  REGION 1..107: note = Synthetic
PEKKRQLLLEHILLDALMLLNLLETNPQNTESKFEDYISNAEVIAEELAKLMESLGLSDEAEKFKKIKQWLREVWRIWSSTNWSTLEDKARELLNRIITTIQSQIFY

SEQ ID NO: 163 - protein, 104 aa, synthetic construct
  REGION 1..104: note = Synthetic
PEKKRQLLLEHILLDLLMILNMIETNRENTESEMEDYWSNVRVILRELARLMEELNYKELSELMERMRKIVEKIRQIVTNNSSLDTAREWLNRLITWIQSLIFR

SEQ ID NO: 164 - protein, 104 aa, synthetic construct
  REGION 1..104: note = Synthetic
PEKKRQLLAEHALLDALMLLNIIETNSKNTESKMEDYVSNLEVILTEFKKLAEKLNFSEEAERAERMKRWARKAYQMMTLDLSLDKAKEMLNRIITILQSIIFN

SEQ ID NO: 165 - protein, 96 aa, synthetic construct
  REGION 1..96: note = Synthetic
PEKKRQLLAEHLLLDVLMMLNGNASLKDYASNAQVIADEFRELARELGLTDEAKKAEKIIEALERAREWLLNNKDKEKAKEALNRAITIAQSWIFN

SEQ ID NO: 166 - protein, 104 aa, synthetic construct
  REGION 1..104: note = Synthetic
PEKKRQLLLEHLLLDLLMILNMLRTNPKNIESDWEDYMSNIEVIIEELRKIMESLGRSEKAKEWKRMKQWVRRILEIVKNNSDLEEAKEWLNRLITIVQSEIFE

SEQ ID NO: 167 - protein, 104 aa, synthetic construct
  REGION 1..104: note = Synthetic
WEKKRQLLLEHLLLDLLMILNMWRTNPQNTESLMEDYMSNAKVIVEELARMMRSQGLEDKAREWEEMKKRIEEIRQIIQNNSSKERAKEELNRLITYVQSEIFR

SEQ ID NO: 168 - protein, 100 aa, synthetic construct
  REGION 1..100: note = Synthetic
PKKKIQLLAEHALLDALMILNIVKTNSQNAEEKLEDYASNVEVILEEIARLMESGDQKDEAEKAKRMKEWMKRIKTTASEDEQEEMANRIITLLQSWIFS

SEQ ID NO: 169 - protein, 100 aa, synthetic construct
  REGION 1..100: note = Synthetic
PEKKRQLLAEHALLDALMILNILQTNPQNAEEKLEDYMSNVEVIMEEFARMMRNGDRSEEAENAERIKKWVRKASSTASSEEQREMMNRAITLMQSWIFE

SEQ ID NO: 170 - protein, 100 aa, synthetic construct
  REGION 1..100: note = Synthetic
PEKKRQLLAEHLLLDALMVLNMLTTNSKNTEEKLEDYISNMKVIIKEMIELMRSLGRLEEAEKWKEALKAVEKIGSRMDSETARELANRIITLAQSAIFY

SEQ ID NO: 171 - protein, 100 aa, synthetic construct
  REGION 1..100: note = Synthetic
PEKKRQLLAEHALLDALMFLNLVETNPDQAEEKIEDYASNLRVIAEELARLFENLGRLDEAQKAKDIKELAERARSRVSSEKRKEAMNRAITILQSMIFR

SEQ ID NO: 172 - protein, 100 aa, synthetic construct
  REGION 1..100: note = Synthetic
PEKKRQLLAEHALLDALMILNIIRTNSDNTESKLEDYISNLKVILEEIARLMESLGLSDEAEKAKEAMRLADKAGSTASEEEKKEAMNRVITWAQSWIFN

SEQ ID NO: 173 - protein, 100 aa, synthetic construct
  REGION 1..100: note = Synthetic
PEKKRQLLAEHALLDALMMLNILRTNPDNAEEKLEDYWSNLIVILREIAKLMESLGLTDEAEKAKEAARWAEEARTTASKDQRRELANRIITLLQSWIFS

SEQ ID NO: 174 - protein, 100 aa, synthetic construct
  REGION 1..100: note = Synthetic
PEKKRQLLAEHLLLDALMILNIIETNEQNAESKLEDYISNAKVILDEFREMARDLGLLDEAKKAEKMKRWLEKMRSNASSDERREWANRMITTAQSWIFN

SEQ ID NO: 175 - protein, 87 aa, synthetic construct
  REGION 1..87: note = Synthetic
TNKEAQLHAEFALYDALMLLNLSSESNERLNRIITWLQSIIFYETYDPDMVKEAVKLADEIEDEMRKRKIDTEDYVVNLRLILQELA

SEQ ID NO: 176 - protein, 87 aa, synthetic construct
  REGION 1..87: note = Synthetic
TKKDAELLAEFALYDALMLLNLSSESNERLNEIITWLQSIIFYGTYDPDMVKEAVKLADEIEDEMRKRGIDTEDYVSNLRLILQELA

SEQ ID NO: 177 - protein, 87 aa, synthetic construct
  REGION 1..87: note = Synthetic
TNKKAQLHAEFALYDALMLLNLSSESNERLNDIITWLQSIIFTGTYDPDMVKEAVKLADEIEDEMRKRKIDTEDYVVNLRYILQELA

SEQ ID NO: 178 - protein, 107 aa, synthetic construct
  REGION 1..107: note = Synthetic
EDYYSNLKLILEELAREMERNGLSDKAEEWRQWKKIVERIRQIRSNNSDLNEAKELLNRLITYIQSQIFEVLHGVGETDQEKKEESWKKWDLLLEHALLDVLMLLND

SEQ ID NO: 179 - protein, 107 aa, synthetic construct
  REGION 1..107: note = Synthetic
EDYYSNLKVILEELAREMERNGLSDKAEEWRQWKKIVERIRQIRSNNSDLNEAKELLNELITYIQSQIFEVIEREGETDQEKKEESWKKWELHLEHALLDVLMLLND

SEQ ID NO: 180 - protein, 107 aa, synthetic construct
  REGION 1..107: note = Synthetic
EDYYSNLKLILEELAREMERNGLSDKAEEWRQWKKIVERIRQIRSNNSDLNEAKELLNRLITYIQSQIFEVLEGVGETDQEKKEESWKKWELHLEHALLDVLMLLND

SEQ ID NO: 181 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 182 - protein, 100 aa, synthetic construct
  REGION 1..100: note = Synthetic
PKKKIQLLAEHALFDLLMILNIVKTNSQNAEEKLEDYAYNAGVILEEIARLFESGDQKDEAEKAKRMKEWMKRIKDTASEDEQEEMANEIITILQSWNFS

SEQ ID NO: 183 - protein, 100 aa, synthetic construct
  REGION 1..100: note = Synthetic
PKKKIQITAEEALKDALSILNIVKTNSPPAEEQLERFAKRFERNLWGIARLFESGDQKDEAEKAKRMKEWMKRIKTTASEDEQEEMANAIITILQSWIFS

SEQ ID NO: 184 - protein, 100 aa, synthetic construct
  REGION 1..100: note = Synthetic
PKKKIQIMAEEALKDALSILNIVKTNSPPAEEQLERFAKRFERNLWGIARLFESGDQKDEAEKAKRMIEWMKRIKTTASEDEQEEMANAIITILQSWFFS

SEQ ID NO: 185 - skipped

SEQ ID NO: 186 - protein, 130 aa, synthetic construct
  REGION 1..130: note = Synthetic
GPGSHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQAAA

SEQ ID NO: 187 - protein, 133 aa, synthetic construct
  REGION 1..133: note = Synthetic
APTSSSTKKTQLQLEHLLLDLQMILNGINNYKNPKLTRMLTFKFYMPKKATELKHLQCLEEELKPLEEVLNLAHSKNFHFDPRDVVSNINVFVLELKGSETTFMCEYADETATIVEFLNRWLTFCQSIISTLT

SEQ ID NO: 188 - protein, 100 aa, synthetic construct; identical to SEQ ID NO: 168
  REGION 1..100: note = Synthetic

SEQ ID NO: 189 - protein, 101 aa, synthetic construct
  REGION 1..101: note = Synthetic
PKKKIQLHAEHALYDALKMILNIVKTNSPPAEEKLEDYAFNFELILEEIARLFESGDQKDEAEKAKRMKEWMKRIKTTASEDEQEEMANAIITILQSWIFS

SEQ ID NO: 190 - protein, 100 aa, synthetic construct
  REGION 1..100: note = Synthetic
PKKKIQLHAEHALYDALMILNIVKTNSPPAEEKLEDYAFNFELILEEIACLFESGDQKDEAEKAKRMKEWMKRIKTTASEDEQEEMANAIITILQSWIFS

SEQ ID NO: 191 - protein, 100 aa, synthetic construct
  REGION 1..100: note = Synthetic
PKKKIQLHAEHALYDALMILNIVKTNSPPAEEKLEDYAFNFELILEEIARLFCSGDQKDEAEKAKRMKEWMKRIKTTASEDEQEEMANAIITILQSWIFS

SEQ ID NO: 192 - protein, 100 aa, synthetic construct
  REGION 1..100: note = Synthetic
PKKKIQLHAEHALYDALMILNIVKTNSPPAEEKLEDYAFNFELILEEIARLFESGCQKDEAEKAKRMKEWMKRIKTTASEDEQEEMANAIITILQSWIFS

SEQ ID NO: 193 - protein, 100 aa, synthetic construct
  REGION 1..100: note = Synthetic
PKKKIQLHAEHALYDALMILNIVKTNSPPAEEKLEDYAFNFELILEEIARLFESGDQCDEAEKAKRMKEWMKRIKTTASEDEQEEMANAIITILQSWIFS

SEQ ID NO: 194 - protein, 100 aa, synthetic construct
  REGION 1..100: note = Synthetic
PKKKIQLHAEHALYDALMILNIVKTNSPPAEEKLEDYAFNFELILEEIARLFESGDQKCEAEKAKRMKEWMKRIKTTASEDEQEEMANAIITILQSWIFS

SEQ ID NO: 195 - protein, 100 aa, synthetic construct
  REGION 1..100: note = Synthetic
PKKKIQLHAEHALYDALMILNIVKTNSPPAEEKLEDYAFNFELILEEIARLFESGDQKDEACKAKRMKEWMKRIKTTASEDEQEEMANAIITILQSWIFS

SEQ ID NO: 196 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKCMKEWMKRIKTTASEDEQEEMANAIITILQSWIFS

SEQ ID NO: 197 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKRMKCWMKRIKTTASEDEQEEMANAIITILQSWIFS

SEQ ID NO: 198 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKRMKEWMKCIKTTASEDEQEEMANAIITILQSWIFS

SEQ ID NO: 199 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKRMKEWMKRIKTCASEDEQEEMANAIITILQSWIFS

SEQ ID NO: 200 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKRMKEWMKRIKTTASEDCQEEMANAIITILQSWIFS

SEQ ID NO: 201 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKRMKEWMKRIKTTASEDEQECMANAIITILQSWIFS

SEQ ID NO: 202 - protein, 100 aa, synthetic construct
  REGION 1..100: note = Synthetic
PKKKIQLHAEHALYDALMILNIVKTNSPPAEEKLEDYAFNFELILEEIACLFESGDQKDEAEKAKRMKEWMKCIKTTASEDEQEEMANAIITILQSWIFS

SEQ ID NO: 203 - protein, 100 aa, synthetic construct
  REGION 1..100: note = Synthetic
PKKKIQLHAEHALYDALMILNIVKTNSPPAEEKLEDYAFNFELILEEIARLFCSGDQKDEAEKAKRMKEWMKCIKTTASEDEQEEMANAIITILQSWIFS

SEQ ID NO: 204 - protein, 100 aa, synthetic construct
  REGION 1..100: note = Synthetic
PKKKIQLHAEHALYDALMILNIVKTNSPPAEEKLEDYAFNFELILEEIARLFESGCQKDEAEKAKRMKEWMKCIKTTASEDEQEEMANAIITILQSWIFS

SEQ ID NO: 205 - protein, 100 aa, synthetic construct
  REGION 1..100: note = Synthetic
PKKKIQLHAEHALYDALMILNIVKTNSPPAEEKLEDYAFNFELILEEIARLFESGDQCDEAEKAKRMKEWMKCIKTTASEDEQEEMANAIITILQSWIFS

SEQ ID NO: 206 - protein, 100 aa, synthetic construct
  REGION 1..100: note = Synthetic
PKKKIQLHAEHALYDALMILNIVKTNSPPAEEKLEDYAFNFELILEEIARLFESGDQKCEAEKAKRMKEWMKCIKTTASEDEQEEMANAIITILQSWIFS

SEQ ID NO: 207 - protein, 100 aa, synthetic construct
  REGION 1..100: note = Synthetic
PKKKIQLHAEHALYDALMILNIVKTNSPPAEEKLEDYAFNFELILEEIARLFESGDQKDEACKAKRMKEWMKCIKTTASEDEQEEMANAIITILQSWIFS

SEQ ID NO: 208 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKCMKEWMKCIKTTASEDEQEEMANAIITILQSWIFS

SEQ ID NO: 209 - protein, 100 aa, synthetic construct
  REGION 1..100: note = Synthetic
PKKKIQLHAEHALYDALMILNIVKTNSPPAEEKLEDYAFNFELILEEIACLFESGDQKDEAEKAKRMKEWMKRIKTTASEDCQEEMANAIITILQSWIFS

SEQ ID NO: 210 - protein, 100 aa, synthetic construct
  REGION 1..100: note = Synthetic
PKKKIQLHAEHALYDALMILNIVKTNSPPAEEKLEDYAFNFELILEEIARLFCSGDQKDEAEKAKRMKEWMKRIKTTASEDCQEEMANAIITILQSWIFS

SEQ ID NO: 211 - protein, 100 aa, synthetic construct
  REGION 1..100: note = Synthetic
PKKKIQLHAEHALYDALMILNIVKTNSPPAEEKLEDYAFNFELILEEIARLFESGCQKDEAEKAKRMKEWMKRIKTTASEDCQEEMANAIITILQSWIFS

SEQ ID NO: 212 - protein, 100 aa, synthetic construct
  REGION 1..100: note = Synthetic
PKKKIQLHAEHALYDALMILNIVKTNSPPAEEKLEDYAFNFELILEEIARLFESGDQCDEAEKAKRMKEWMKRIKTTASEDCQEEMANAIITILQSWIFS

SEQ ID NO: 213 - protein, 100 aa, synthetic construct
  REGION 1..100: note = Synthetic
PKKKIQLHAEHALYDALMILNIVKTNSPPAEEKLEDYAFNFELILEEIARLFESGDQKCEAEKAKRMKEWMKRIKTTASEDCQEEMANAIITILQSWIFS

SEQ ID NO: 214 - protein, 100 aa, synthetic construct
  REGION 1..100: note = Synthetic
PKKKIQLHAEHALYDALMILNIVKTNSPPAEEKLEDYAFNFELILEEIARLFESGDQKDEACKAKRMKEWMKRIKTTASEDCQEEMANAIITILQSWIFS

SEQ ID NO: 215 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKCMKEWMKRIKTTASEDCQEEMANAIITILQSWIFS

SEQ ID NO: 216 - protein, 100 aa, synthetic construct
  REGION 1..100: note = Synthetic
PKKKIQLHAEHALYDALMILNIVKTNSPPAEEKLEDYAFNFELILEEIARLFESGDQKDEAEKAKRMKCWMKRIKTTASEDCQEEMANAIITILQSWIFS

SEQ ID NO: 217 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIACLFESGXXKDEAEKAKRMKEWMKRIKTXXXEDEQEEMANAIITILQSWIFS

SEQ ID NO: 218 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFCSGXXKDEAEKAKRMKEWMKRIKTXXXEDEQEEMANAIITILQSWIFS

SEQ ID NO: 219 - protein, 100 aa, synthetic construct
  REGION 1..100: note = Synthetic
  VARIANT 23..33: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGCQKDEAEKAKRMKEWMKRIKTXXXEDEQEEMANAIITILQSWIFS

SEQ ID NO: 220 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXCDEAEKAKRMKEWMKRIKTXXXEDEQEEMANAIITILQSWIFS

SEQ ID NO: 221 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKCEAEKAKRMKEWMKRIKTXXXEDEQEEMANAIITILQSWIFS

SEQ ID NO: 222 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEACKAKRMKEWMKRIKTXXXEDEQEEMANAIITILQSWIFS

SEQ ID NO: 223 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKCMKEWMKRIKTXXXEDEQEEMANAIITILQSWIFS

SEQ ID NO: 224 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKRMKCWMKRIKTXXXEDEQEEMANAIITILQSWIFS

SEQ ID NO: 225 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKRMKEWMKCIKTXXXEDEQEEMANAIITILQSWIFS

SEQ ID NO: 226 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKRMKEWMKRIKTCASEDEQEEMANAIITILQSWIFS

SEQ ID NO: 227 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKRMKEWMKRIKTXXXEDCQEEMANAIITILQSWIFS

SEQ ID NO: 228 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKRMKEWMKRIKTXXXEDEQECMANAIITILQSWIFS

SEQ ID NO: 229 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIACLFESGXXKDEAEKAKRMKEWMKCIKTXXXEDEQEEMANAIITILQSWIFS

SEQ ID NO: 230 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFCSGXXKDEAEKAKRMKEWMKCIKTXXXEDEQEEMANAIITILQSWIFS

SEQ ID NO: 231 - protein, 100 aa, synthetic construct
  REGION 1..100: note = Synthetic
  VARIANT 23..33: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGCQKDEAEKAKRMKEWMKCIKTXXXEDEQEEMANAIITILQSWIFS

SEQ ID NO: 232 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXCDEAEKAKRMKEWMKCIKTXXXEDEQEEMANAIITILQSWIFS

SEQ ID NO: 233 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKCEAEKAKRMKEWMKCIKTXXXEDEQEEMANAIITILQSWIFS

SEQ ID NO: 234 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEACKAKRMKEWMKCIKTXXXEDEQEEMANAIITILQSWIFS

SEQ ID NO: 235 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKCMKEWMKCIKTXXXEDEQEEMANAIITILQSWIFS

SEQ ID NO: 236 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIACLFESGXXKDEAEKAKRMKEWMKRIKTXXXEDCQEEMANAIITILQSWIFS

SEQ ID NO: 237 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFCSGXXKDEAEKAKRMKEWMKRIKTXXXEDCQEEMANAIITILQSWIFS

SEQ ID NO: 238 - protein, 100 aa, synthetic construct
  REGION 1..100: note = Synthetic
  VARIANT 23..33: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGCQKDEAEKAKRMKEWMKRIKTXXXEDCQEEMANAIITILQSWIFS

SEQ ID NO: 239 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXCDEAEKAKRMKEWMKRIKTXXXEDCQEEMANAIITILQSWIFS

SEQ ID NO: 240 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKCEAEKAKRMKEWMKRIKTXXXEDCQEEMANAIITILQSWIFS

SEQ ID NO: 241 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEACKAKRMKEWMKRIKTXXXEDCQEEMANAIITILQSWIFS

SEQ ID NO: 242 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKCMKEWMKRIKTXXXEDCQEEMANAIITILQSWIFS

SEQ ID NO: 243 - protein, 100 aa, synthetic construct
  REGION 1..100: note = Synthetic
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKRMKCWMKRIKTXXXEDCQEEMANAIITILQSWIFS

SEQ ID NO: 244 - protein, 726 aa, synthetic construct
  REGION 1..726: note = Synthetic
  VARIANT 1..25: note = Optional His tag
  VARIANT 26..609: note = Mouse serum albumin
  VARIANT 610..626: note = Linker
  VARIANT 627..726: note = Neo2/15
GSDGGSHHHHHHGSGSENLYFQGSGEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGSGGSGGGSGGSGSGPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 245 - protein, 87 aa, synthetic construct
  REGION 1..87: note = Synthetic
TNKKAQLHAEFALHDALMLLNLSSESNERLNRIITWLQSIIFYGTYDPDMVKEAVKDADEIEDEMRKRGIDTEDYVSNLRLILQELA

SEQ ID NO: 246 - protein, 100 aa, synthetic construct
  REGION 1..100: note = Synthetic
PKKKIQLYAEHALYDALMILNIVKTNSPPAEEELEDYAFNFELILEEIARLFESGDQKDEAEKAKRMKEWMKRIKTTASEDEQEEMANAIITILQSWIFS

SEQ ID NO: 247 - protein, 100 aa, synthetic construct
  REGION 1..100: note = Synthetic peptide
  VARIANT 23..33: note = Xaa, when present, can be any naturally occurring amino acid or is optionally absent
  VARIANT 56..57: note = Xaa, when present, can be any naturally occurring amino acid or is optionally absent
  VARIANT 77..79: note = Xaa, when present, can be any naturally occurring amino acid or is optionally absent
PKKKIQLHAEHALYDALMILNIXXXXXXXXXXXLEDYAFNFELILEEIARLFESGXXKDEAEKAKRMKEWMKRIKTXXXEDEQEEMANAIITILQSWIFS

SEQ ID NO: 248 - protein, 22 aa, synthetic construct
  REGION 1..22: note = Synthetic peptide
STKKTQLQLEHLLLDLQMILNG

SEQ ID NO: 249 - protein, 15 aa, synthetic construct
  REGION 1..15: note = Synthetic peptide
NWVNVISDLKKIEDL

SEQ ID NO: 250 - protein, 22 aa, synthetic construct
  REGION 1..22: note = Synthetic peptide
PRDLISNINVIVLELKGSETTF

SEQ ID NO: 251 - protein, 22 aa, synthetic construct
  REGION 1..22: note = Synthetic peptide
IHDTVENLIILANNSLSSNGNV

SEQ ID NO: 252 - protein, 19 aa, synthetic construct
  REGION 1..19: note = Synthetic peptide
ATELKHLQCLEEELKPLEE

SEQ ID NO: 253 - protein, 19 aa, synthetic construct
  REGION 1..19: note = Synthetic peptide
SCKVTAMKCFLLELQVISL

SEQ ID NO: 254 - protein, 21 aa, synthetic construct
  REGION 1..21: note = Synthetic peptide
TATIVEFLNRWITFCQSIIST

SEQ ID NO: 255 - protein, 21 aa, synthetic construct
  REGION 1..21: note = Synthetic peptide
EKNIKEFLQSFVHIVQMFINT

SEQ ID NO: 256 - protein, 27 aa, synthetic construct
  REGION 1..27: note = Synthetic peptide
  VARIANT 23..27: note = Optional
PKKKIQLHAEHALYDALMILNIVKTNS

SEQ ID NO: 257 - protein, 76 aa, synthetic construct
  REGION 1..76: note = Synthetic peptide
  VARIANT 1..9: note = Optional
  VARIANT 32..33: note = Optional
  VARIANT 53..55: note = Optional
TNSPPAEEKLEDYAFNFELILEEIARLFESGDQKDEAEKAKRMKEWMKRIKTTASEDEQEEMANAIITILQSWIFS

SEQ ID NO: 258 - protein, 79 aa, synthetic construct
  REGION 1..79: note = Synthetic peptide
  VARIANT 23..33: note = Optional
  VARIANT 56..57: note = Optional
PKKKIQLHAEHALYDALMILNIVKTNSPPAEEKLEDYAFNFELILEEIARLFESGDQKDEAEKAKRMKEWMKRIKTTAS

SEQ ID NO: 259 - protein, 25 aa, synthetic construct
  REGION 1..25: note = Synthetic peptide
  VARIANT 1..5: note = Optional
TTASEDEQEEMANAIITILQSWIFS

SEQ ID NO: 260 - protein, 56 aa, synthetic construct
  REGION 1..56: note = Synthetic peptide
  VARIANT 23..33: note = Optional
  VARIANT 55..56: note = Optional
PKKKIQLHAEHALYDALMILNIVKTNSPPAEEKLEDYAFNFELILEEIARLFESGD

SEQ ID NO: 261 - protein, 45 aa, synthetic construct
  REGION 1..45: note = Synthetic peptide
  VARIANT 22..24: note = Optional
DQKDEAEKAKRMKEWMKRIKTTASEDEQEEMANAIITILQSWIFS

SEQ ID NO: 262 - protein, 27 aa, synthetic construct
  REGION 1..27: note = Synthetic peptide
  VARIANT 23..27: note = Optional
PKKKIQIMAEEALKDALSILNIVKTNS

SEQ ID NO: 263 - protein, 76 aa, synthetic construct
  REGION 1..76: note = Synthetic peptide
  VARIANT 1..8: note = Optional
  VARIANT 32..33: note = Optional
  VARIANT 53..55: note = Optional
TNSPPAEEQLERFAKRFERNLWGIARLFESGDQKDEAEKAKRMIEWMKRIKTTASEDEQEEMANAIITILQSWFFS

SEQ ID NO: 264 - protein, 76 aa, synthetic construct
  REGION 1..76: note = Synthetic peptide
  VARIANT 1..9: note = Xaa can be any naturally occurring amino acid
  VARIANT 32..33: note = Xaa can be any naturally occurring amino acid
  VARIANT 53..55: note = Xaa can be any naturally occurring amino acid
XXXXXXXXXLEDYAFNFELILEEIARLFESGXXKDEAEKAKRMKEWMKRIKTXXXEDEQEEMANAIITILQSWIFS

SEQ ID NO: 265 - protein, 79 aa, synthetic construct
  REGION 1..79: note = Synthetic peptide
  VARIANT 23..33: note = Xaa can be any naturally occurring amino acid
  VARIANT 56..57: note = Xaa can be any naturally occurring amino acid
PKKKIQLHAEHALYDALMILNIXXXXXXXXXXXLEDYAFNFELILEEIARLFESGXXKDEAEKAKRMKEWMKRIKTTAS

SEQ ID NO: 266 - protein, 25 aa, synthetic construct
  REGION 1..25: note = Synthetic peptide
  VARIANT 1..5: note = Xaa can be any naturally occurring amino acid
XXXXXDEQEEMANAIITILQSWIFS

SEQ ID NO: 267 - protein, 58 aa, synthetic construct
  REGION 1..58: note = Synthetic peptide
  VARIANT 23..33: note = Xaa can be any naturally occurring amino acid
  VARIANT 55..56: note = Xaa can be any naturally occurring amino acid
PKKKIQLHAEHALYDALMILNIXXXXXXXXXXXLEDYAFNFELILEEIARLFESXXGD

SEQ ID NO: 268 - protein, 45 aa, synthetic construct
  REGION 1..45: note = Synthetic peptide
  VARIANT 22..24: note = Xaa can be any naturally occurring amino acid
DQKDEAEKAKRMKEWMKRIKTXXXEDEQEEMANAIITILQSWIFS

SEQ ID NO: 269 - protein, 27 aa, synthetic construct
  REGION 1..27: note = Synthetic peptide
  VARIANT 23..27: note = Xaa can be any naturally occurring amino acid
PKKKIQIMAEEALKDALSILNIXXXXX

SEQ ID NO: 270 - protein, 76 aa, synthetic construct
  REGION 1..76: note = Synthetic peptide
  VARIANT 1..8: note = Xaa can be any naturally occurring amino acid
  VARIANT 32..33: note = Xaa can be any naturally occurring amino acid
  VARIANT 53..55: note = Xaa can be any naturally occurring amino acid
XXXXXXXXQLERFAKRFERNLWGIARLFESGXXKDEAEKAKRMIEWMKRIKTXXXEDEQEEMANAIITILQSWFFS

SEQ ID NO: 271 - protein, 22 aa, synthetic construct
  REGION 1..22: note = Synthetic peptide
STKKWQLQAEHALLDWQMALNK

SEQ ID NO: 272 - protein, 15 aa, synthetic construct
  REGION 1..15: note = Synthetic peptide
ENLNRAITAAQSWIS

SEQ ID NO: 273 - protein, 20 aa, synthetic construct
  REGION 1..20: note = Synthetic peptide
LDKAEDIRRNSDQARREAEK

SEQ ID NO: 274 - protein, 15 aa, synthetic construct
  REGION 1..15: note = Synthetic peptide
RDLISNAQVILLEAR

SEQ ID NO: 275 - protein, 100 aa, synthetic construct; identical to SEQ ID NO: 217
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 276 - protein, 100 aa, synthetic construct; identical to SEQ ID NO: 218
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 277 - protein, 100 aa, synthetic construct; identical to SEQ ID NO: 219
  REGION 1..100: note = Synthetic peptide
  VARIANT 23..33: note = Xaa can be any naturally occurring amino acid
  VARIANT 77..79: note = Xaa can be any naturally occurring amino acid

SEQ ID NO: 278 - protein, 100 aa, synthetic construct; identical to SEQ ID NO: 220
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 279 - protein, 100 aa, synthetic construct; identical to SEQ ID NO: 221
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 280 - protein, 100 aa, synthetic construct; identical to SEQ ID NO: 222
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 281 - protein, 100 aa, synthetic construct; identical to SEQ ID NO: 223
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 282 - protein, 100 aa, synthetic construct; identical to SEQ ID NO: 224
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 283 - protein, 100 aa, synthetic construct; identical to SEQ ID NO: 225
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 284 - protein, 100 aa, synthetic construct; identical to SEQ ID NO: 226
  REGION 1..100: note = Synthetic peptide
  VARIANT 23..33: note = Xaa can be any naturally occurring amino acid
  VARIANT 56..57: note = Xaa can be any naturally occurring amino acid

SEQ ID NO: 285 - protein, 100 aa, synthetic construct; identical to SEQ ID NO: 227
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 286 - protein, 100 aa, synthetic construct; identical to SEQ ID NO: 228
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 287 - protein, 100 aa, synthetic construct; identical to SEQ ID NO: 229
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 288 - protein, 100 aa, synthetic construct; identical to SEQ ID NO: 230
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 289 - protein, 100 aa, synthetic construct; identical to SEQ ID NO: 231
  REGION 1..100: note = Synthetic peptide
  VARIANT 23..33: note = Xaa can be any naturally occurring amino acid
  VARIANT 77..79: note = Xaa can be any naturally occurring amino acid

SEQ ID NO: 290 - protein, 100 aa, synthetic construct; identical to SEQ ID NO: 232
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 291 - protein, 100 aa, synthetic construct; identical to SEQ ID NO: 233
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 292 - protein, 100 aa, synthetic construct; identical to SEQ ID NO: 235
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 293 - protein, 100 aa, synthetic construct; identical to SEQ ID NO: 236
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 294 - protein, 100 aa, synthetic construct; identical to SEQ ID NO: 237
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 295 - protein, 100 aa, synthetic construct; identical to SEQ ID NO: 238
  REGION 1..100: note = Synthetic peptide
  VARIANT 23..33: note = Xaa can be any naturally occurring amino acid
  VARIANT 77..79: note = Xaa can be any naturally occurring amino acid

SEQ ID NO: 296 - protein, 100 aa, synthetic construct; identical to SEQ ID NO: 239
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 297 - protein, 100 aa, synthetic construct; identical to SEQ ID NO: 240
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 298 - protein, 100 aa, synthetic construct; identical to SEQ ID NO: 241
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 299 - protein, 100 aa, synthetic construct; identical to SEQ ID NO: 242
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 300 - protein, 100 aa, synthetic construct; identical to SEQ ID NO: 243
  REGION 1..100: note = Synthetic peptide
  VARIANT 23..33: note = Xaa can be any naturally occurring amino acid
  VARIANT 56..57: note = Xaa can be any naturally occurring amino acid
  VARIANT 77..79: note = Xaa can be any naturally occurring amino acid

SEQ ID NO: 301 - protein, 27 aa, synthetic construct
  REGION 1..27: note = Synthetic peptide
  VARIANT 23..27: note = Xaa can be any naturally occurring amino acid
PKKKIQLHAEHALYDALMILNIXXXXX

SEQ ID NO: 302 - skipped

SEQ ID NO: 303 - protein, 14 aa, synthetic construct
  REGION 1..14: note = Synthetic peptide
LNDIFEAQKIEWHE

SEQ ID NO: 304 - protein, 276 aa, synthetic construct
  REGION 1..276: note = Synthetic peptide
  VARIANT 1..24: note = Optional
  VARIANT 101..120: note = Optional
MGSHHHHHHGSGSENLYFQGSGGGTNSPPAEEKLEDYAFNFELILEEIARLFESGDQKDEAEKAKRMKEWMKRIKTTASEDEQEEMANAIITILQSWIFSGSGGSGGGSGGSGSGGSGGGDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLN

SEQ ID NO: 305 - protein, 190 aa, synthetic construct
  REGION 1..190: note = Synthetic peptide
  VARIANT 1..24: note = Optional
  VARIANT 149..163: note = Optional
MGSHHHHHHGSGSENLYFQGSGSGDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSGGSGGGSGGSGSGPKKKIQLHAEHALYDALMILNIVKTNS

SEQ ID NO: 306 - protein, 239 aa, synthetic construct
  REGION 1..239: note = Synthetic peptide
  VARIANT 1..24: note = Optional
  VARIANT 149..163: note = Optional
MGSHHHHHHGSGSENLYFQGSGSGDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSGGSGGGSGGSGSGTNSPPAEEKLEDYAFNFELILEEIARLFESGDQKDEAEKAKRMKEWMKRIKTTASEDEQEEMANAIITILQSWIFS

SEQ ID NO: 307 - protein, 276 aa, synthetic construct; identical to SEQ ID NO: 304
  REGION 1..276: note = Synthetic peptide
  VARIANT 1..24: note = Optional
  VARIANT 101..120: note = Optional

SEQ ID NO: 308 - protein, 245 aa, synthetic construct
  REGION 1..245: note = Synthetic peptide
  VARIANT 1..24: note = Optional
  VARIANT 70..89: note = Optional
MGSHHHHHHGSGSENLYFQGSGGGDQKDEAEKAKRMKEWMKRIKTTASEDEQEEMANAIITILQSWIFSGSGGSGGGSGGSGSGGSGGGDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLN

SEQ ID NO: 309 - protein, 225 aa, synthetic construct
  REGION 1..225: note = Synthetic peptide
  VARIANT 1..24: note = Optional
  VARIANT 49..69: note = Optional
MGSHHHHHHGSGSENLYFQGSGGGTTASEDEQEEMANAIITILQSWIFSGSGGSGGGSGGSGSGGSGGGDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLN

SEQ ID NO: 310 - protein, 218 aa, synthetic construct
  REGION 1..218: note = Synthetic peptide
  VARIANT 1..24: note = Optional
  VARIANT 149..163: note = Optional
MGSHHHHHHGSGSENLYFQGSGSGDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSGGSGGGSGGSGSGPKKKIQLHAEHALYDALMILNIVKTNSPPAEEKLEDYAFNFELILEEIARLFESG

SEQ ID NO: 311 - protein, 241 aa, synthetic construct
  REGION 1..241: note = Synthetic peptide
  VARIANT 1..24: note = Optional
  VARIANT 148..163: note = Optional
MGSHHHHHHGSGSENLYFQGSGSGDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSGGSGGGSGGSGSGPKKKIQLHAEHALYDALMILNIVKTNSPPAEEKLEDYAFNFELILEEIARLFESGDQKDEAEKAKRMKEWMKRIKTTA

SEQ ID NO: 312 - protein, 56 aa, synthetic construct
  REGION 1..56: note = Synthetic peptide
  VARIANT 23..32: note = Optional
  VARIANT 55..56: note = Optional
PKKKIQIMAEEALKDALSILNIVKTNSPPAEEQLERFAKRFERNLWGIARLFESGD

SEQ ID NO: 313 - protein, 45 aa, synthetic construct
  REGION 1..45: note = Synthetic peptide
  VARIANT 1..2: note = Optional
  VARIANT 21..25: note = Optional
DQKDEAEKAKRMIEWMKRIKTTASEDEQEEMANAIITILQSWFFS

SEQ ID NO: 314 - protein, 56 aa, synthetic construct
  REGION 1..56: note = Synthetic peptide
  VARIANT 23..32: note = Xaa can be any naturally occurring amino acid
  VARIANT 55..56: note = Xaa can be any naturally occurring amino acid
PKKKIQIMAEEALKDALSILNIXXXXXXXXXXQLERFAKRFERNLWGIARLFESXX

SEQ ID NO: 315 - protein, 45 aa, synthetic construct
  REGION 1..45: note = Synthetic peptide
  VARIANT 1..2: note = Optional
  VARIANT 21..24: note = Xaa can be any naturally occurring amino acid
XXKDEAEKAKRMIEWMKRIKXXXXEDEQEEMANAIITILQSWFFS

SEQ ID NO: 316 - protein, 78 aa, synthetic construct
  REGION 1..78: note = Synthetic peptide
  VARIANT 23..33: note = Optional
  VARIANT 55..57: note = Optional
  VARIANT 76..78: note = Optional
PKKKIQIMAEEALKDALSILNIVKTNSPPAEEQLERFAKRFERNLWGIARLFESGDQKDEAEKAKRMIEWMKRIKTTA

SEQ ID NO: 317 - protein, 25 aa, synthetic construct
  REGION 1..25: note = Synthetic peptide
  VARIANT 1..5: note = Optional
TTASEDEQEEMANAIITILQSWFFS

SEQ ID NO: 318 - protein, 78 aa, synthetic construct
  REGION 1..78: note = Synthetic peptide
  VARIANT 23..33: note = Xaa can be any naturally occurring amino acid
  VARIANT 55..57: note = Xaa can be any naturally occurring amino acid
  VARIANT 76..78: note = Xaa can be any naturally occurring amino acid
PKKKIQIMAEEALKDALSILNIXXXXXXXXXXXLERFAKRFERNLWGIARLFESXXXKDEAEKAKRMIEWMKRIKXXX

SEQ ID NO: 319 - protein, 25 aa, synthetic construct
  REGION 1..25: note = Synthetic peptide
  VARIANT 1..5: note = Optional
XXXXXDEQEEMANAIITILQSWFFS

SEQ ID NO: 320 - protein, 17 aa, synthetic construct
  REGION 1..17: note = Synthetic peptide
QLHAEHALYDALMILNI

SEQ ID NO: 321 - protein, 21 aa, synthetic construct
  REGION 1..21: note = Synthetic peptide
LEDYAFNFELILEEIARLFES

SEQ ID NO: 322 - protein, 20 aa, synthetic construct
  REGION 1..20: note = Synthetic peptide
DEQEEMANAIITILQSWIFS

SEQ ID NO: 323 - protein, 27 aa, synthetic construct
  REGION 1..27: note = Synthetic peptide
  VARIANT 23..27: note = Optional
PKKKIQLHAEHALYDALMILNIVGGSS

SEQ ID NO: 324 - protein, 27 aa, synthetic construct
  REGION 1..27: note = Synthetic peptide
  VARIANT 1..4: note = Optional
SKEAIQLHAEHALYDALMILNIVKTNS

SEQ ID NO: 325 - protein, 20 aa, synthetic construct
  REGION 1..20: note = Synthetic peptide
  VARIANT 1: note = Optional
PIQLHAEHALYDALMILNIV

SEQ ID NO: 326 - protein, 69 aa, synthetic construct
  REGION 1..69: note = Synthetic peptide
  VARIANT 1..2: note = Optional
  VARIANT 25..26: note = Optional
  VARIANT 46..48: note = Optional
PKLEDYAFNFELILEEIARLFESGDQKDEAEKAKRMKEWMKRIKTTASEDEQEEMANAIITILQSWIFS

SEQ ID NO: 327 - protein, 77 aa, synthetic construct
  REGION 1..77: note = Synthetic peptide
  VARIANT 1..6: note = Optional
  VARIANT 28..33: note = Optional
  VARIANT 52..57: note = Optional
GGSSGGLEDYAFNFELILEEIARLFESGGSSGGKDEAEKAKRMKEWMKRITGGSSGGDEQEEMANAIITILQSWIFS

SEQ ID NO: 328 - protein, 76 aa, synthetic construct
  REGION 1..76: note = Synthetic peptide
  VARIANT 1..6: note = Optional
  VARIANT 28..35: note = Optional
  VARIANT 51..56: note = Optional
GGSSGGLEDYAFNFELILEEIARLFESGGSSGGGGEAEKAKRMKEWMKRIGGSSGGDEQEEMANAIITILQSWIFS